(19) United States
(12) Patent Application Publication
(10) Pub. No.: US 2022/0056480 A1
(43) Pub. Date: Feb. 24, 2022

(54) MODIFIED ORTHOPOXVIRUS VECTORS

(71) Applicants: OTTAWA HOSPITAL RESEARCH INSTITUTE, Ottawa (CA); TURNSTONE BIOLOGICS CORP., New York, NY (US)

(72) Inventors: John C. BELL, Ottawa (CA); Michael S. HUH, Ottawa (CA); Matthew Y. TANG, Orleans (CA); Adrian PELIN, San Francisco, CA (US); Caroline J. BREITBACH, Somerville, MA (US); Michael F. BURGESS, Chester, NJ (US); Steven H. BERNSTEIN, Lawrenceville, NJ (US)

(73) Assignees: OTTAWA HOSPITAL RESEARCH INSTITUTE, Ottawa, ON (CA); TURNSTONE BIOLOGICS CORP., New York, NY (US)

(21) Appl. No.: 17/415,575

(22) PCT Filed: Dec. 20, 2019
(86) PCT No.: PCT/CA2019/051898

§ 371 Date: Jun. 17, 2021

Related U.S. Application Data
(60) Provisional application No. 62/784,372, filed on Dec. 21, 2018.
(60) Provisional application No. 62/872,699, filed on Jul. 10, 2019.
(60) Provisional application No. 62/930,524, filed on Nov. 4, 2019.

(51) Int. Cl. C12N 15/86; C07K 16/28; C07K 14/54

(52) U.S. Cl. CPC C12N 15/86; C07K 16/2818; C07K 14/5434; C12N 2710/24143; C12N 2710/24122; C12N 2710/24132; C12N 2710/24121

(57) ABSTRACT

The disclosure relates to modified orthopoxvirus vectors, as well as methods of using the same for the treatment of various cancers. The disclosure provides modified orthopoxvirus vectors that exhibit various beneficial therapeutic activities, including enhanced oncolytic activity, spread of infection, immune evasion, tumor persistence, capacity for incorporation of exogenous DNA sequences, amenability for large scale manufacturing, and safety.

### CROSS-REFERENCE TO RELATED APPLICATIONS

This application claims the benefit of priority to U.S. Provisional Patent Application No. 62/930,524, filed Nov. 4, 2019, U.S. Provisional Patent Application No. 62/872,699, filed Jul. 10, 2019, and U.S. Provisional Patent Application No. 62/784,372, filed Dec. 21, 2018, the disclosure of each of which is incorporated by reference herein in its entirety.

### SEQUENCE LISTING

This application incorporates by reference a Sequence Listing submitted with this application as an ASCII text file, entitled 14596-050-228_SL.txt, created on Dec. 18, 2019, and having a size of 1,252,729 bytes.

### 1. Field

The invention relates to the field of immunotherapy, e.g., for the treatment of cell proliferation disorders, such as cancers. Particularly, the invention relates to genetically modified orthopoxviruses, as well as methods of making and using the same.

### 2. Background

The immune system may be stimulated to identify tumor cells and target them for destruction. Immunotherapy employing oncolytic orthopoxviruses is a rapidly evolving area in cancer research. New approaches are needed to engineer and/or enhance tumor-selectivity for oncolytic viruses in order to maximize efficiency and safety. This selectivity is especially important when potentially toxic therapeutic agents or genes are added to the viruses.

Although the use of orthopoxviruses as clinical oncolytic vectors is a promising paradigm for cancer treatment, due to toxicity, such as pox lesions in patients, and immunosuppressive side effects, most current clinical candidates have shown only modest clinical success. There exists a need for methods to engineer orthopoxviruses that exhibit more robust virus replication, cancer cell killing, and spreading from the point of infection. The present invention addresses this need and provides a solution to selectivity and safety limitations by employing a modified orthopoxvirus.

### 3. SUMMARY

The present disclosure describes the use of orthopoxviruses for the treatment of cancer. In particular, the disclosure is based in part on the enhanced oncolytic activity, spread of infection, and safety results engendered when an orthopoxvirus is genetically modified to contain deletions in one or more, or all, of the following genes: the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes. Genetically modified orthopoxviruses, such as vaccinia viruses (e.g., Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16mO, Tashkent, Tian Tan, and WAU86/88-1 viruses) that exhibit mutations in one or more, or all, of these genes may exhibit an array of beneficial features, such as improved oncolytic ability, replication in tumors, infectivity, immune evasion, tumor persistence, capacity for incorporation of exogenous DNA sequences, and/or amenability for large scale manufacturing. The present disclosure describes orthopox viruses further genetically modified to contain deletions in the B8R gene. In various embodiments, the modified orthopoxvirus expresses at least one of three transgenes: Interleukin 12 containing a transmembrane domain (IL-12-TM), FMS-like tyrosine kinase 3 ligand (FLT3-L) and anti-Cytotoxic T-lymphocyte Associated Protein 4 (CTLA-4) antibody.

In one aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ inverted terminal repeat (ITR): B21R, B22R, B23R, B24R, B25R, B26R, B27R, B28R, and B29R; and (c) a first transgene comprising a first nucleotide sequence encoding an antibody or antigen-binding fragment thereof that specifically binds to Cytotoxic T-lymphocyte Associated Protein 4 (CTLA-4); wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter.

In some embodiments, the first nucleotide sequence is in the same orientation as the endogenous vaccinia virus genes that flank the first nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; and (d) a nucleotide sequence comprising at least one promoter operably linked to the first nucleotide sequence, wherein the at least one promoter operably linked to the first nucleotide sequence is an H5R promoter, a pS promoter, or a LEO promoter; wherein the deletions in the C2L, F3L, B14R, and B29R vaccinia genes are partial deletions. In a particular embodiment, the nucleic acid comprises a recombinant vaccinia virus genome that comprises a deletion in the B8R gene.

In certain embodiments, the at least one promoter operably linked to the first nucleotide sequence is an H5R promoter.

In some embodiments, the first nucleotide sequence is in the same orientation as the endogenous vaccinia virus genes that flank the first nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; and (c) a second transgene comprising a second nucleotide sequence encoding an Interleukin 12 (IL-12) polypeptide; wherein the deletions in the C2L, F3L, B14R, and B29R vaccinia genes are partial deletions.

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, the second nucleotide sequence is in the same orientation as the endogenous vaccinia virus genes that flank the second nucleotide sequence.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a nucleotide sequence comprising at least one promoter operably linked to the second nucleotide sequence, wherein the at least one promoter operably linked to the second nucleotide sequence is a late promoter; wherein the deletions in the C2L, F3L, B14R, and B29R vaccinia genes are partial deletions. In a particular embodiment, the nucleic acid comprises a recombinant vaccinia virus genome that comprises a deletion in the B8R gene.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, the second nucleotide sequence is in the same orientation as the endogenous vaccinia virus genes that flank the second nucleotide sequence.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; and (c) a third transgene comprising a third nucleotide sequence encoding FMS-like tyrosine kinase 3 ligand (FLT3L); wherein the deletions in the C2L, F3L, B14R, and B29R vaccinia genes are partial deletions.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, the third nucleotide sequence is in the same orientation as the endogenous vaccinia virus genes that flank the third nucleotide sequence. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a third transgene comprising a third nucleotide sequence encoding FLT3L; and (d) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter; wherein the deletions in the C2L, F3L, B14R, and B29R vaccinia genes are partial deletions. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, the third nucleotide sequence is in the same orientation as the endogenous vaccinia virus genes that flank the third nucleotide sequence. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; and (d) a second transgene comprising a second nucleotide sequence encoding an IL-12 polypeptide; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter.

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, the first nucleotide sequence is in the same orientation as endogenous vaccinia virus genes that flank the first nucleotide sequence, and the second nucleotide sequence is in the same orientation as endogenous vaccinia virus genes that flank the second nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; and (d) a second transgene comprising a second nucleotide sequence encoding an IL-12 polypeptide; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or (ii) a nucleotide sequence comprising at least one promoter operably linked to the second nucleotide sequence, wherein the at least one promoter operably linked to the second nucleotide sequence is a late promoter. In a particular embodiment, the nucleic acid comprises a recombinant vaccinia virus genome that comprises a deletion in the B8R gene.

In certain embodiments, the at least one promoter operably linked to the first nucleotide sequence is an H5R promoter.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, the first nucleotide sequence is in the same orientation as endogenous vaccinia virus genes that flank the first nucleotide sequence, and the second nucleotide sequence is in the same orientation as endogenous vaccinia virus genes that flank the second nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; and (d) a third transgene comprising a third nucleotide sequence encoding FLT3L; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, the first nucleotide sequence is in the same orientation as endogenous vaccinia virus genes that flank the first nucleotide sequence, and the third nucleotide sequence is in the same orientation as endogenous vaccinia virus genes that flank the third nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; and (d) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the first nucleotide sequence is an H5R promoter.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, the first nucleotide sequence is in the same orientation as endogenous vaccinia virus genes that flank the first nucleotide sequence, and the third nucleotide sequence is in the same orientation as endogenous vaccinia virus genes that flank the third nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a third transgene comprising a third nucleotide sequence encoding FLT3L; wherein the deletions in the C2L, F3L, B14R, and B29R vaccinia genes are partial deletions.

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided here is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the second nucleotide sequence, wherein the at least one promoter operably linked to the second nucleotide sequence is a late promoter; and/or (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter.

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, the first nucleotide sequence, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; (ii) a nucleotide sequence comprising at least one promoter operably linked to the second nucleotide sequence, wherein the at least one promoter operably linked to the second nucleotide sequence is a late promoter; and/or (iii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the first nucleotide sequence is an H5R promoter.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 or IL-12 p70. In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In a specific embodiment, the first transgene is inserted between the partial C2L and F3L vaccinia genes (that is, is present between the partial C2L and F3L genes), and the second transgene and the third transgene are inserted into the locus of the deletion in the B8R gene (that is, are present in the locus of the deletion in the B8R gene). In a specific embodiment, the first transgene is inserted between the portion of the C2L vaccinia gene that remains and the portion of the F3L vaccinia gene that remains (that is, is present between the portion of the C2L vaccinia gene that remains and the portion of the F3L vaccinia gene that remains), and the second transgene and the third transgene are inserted into the locus of the deletion in the B8R gene (that is, are present in the locus of the deletion in the B8R gene). In a further specific embodiment, the third transgene is upstream of the second transgene.

In some embodiments of the various embodiments and aspects described herein, the deletion in the B8R gene is a deletion of at least 50% of the B8R gene sequence. In other embodiments, the deletion in the B8R gene is a deletion of at least 60% of the B8R gene sequence. In other embodiments, the deletion in the B8R gene is a deletion of at least 70% of the B8R gene sequence. In other embodiments, the deletion in the B8R gene is a deletion of at least 80% of the B8R gene sequence. In a specific embodiment, the deletion in the B8R gene is a deletion of about 75% of the B8R gene sequence. In another specific embodiment, the deletion in the B8R gene is a deletion of about 80% of the B8R gene sequence.

In certain embodiments of the various embodiments and aspects described herein, the recombinant vaccinia virus genome is derived from the genome of a Copenhagen strain vaccinia virus.

In certain embodiments of the various embodiments and aspects described herein, the recombinant vaccinia virus genome comprises the nucleotide sequence of SEQ ID NO: 210.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4; (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In specific embodiments, the first transgene is inserted between the partial C2L and F3L vaccinia genes, and the second transgene and the third transgene are inserted into the locus of the deletion in the B8R gene. In specific embodiments, the first transgene is inserted between the partial B14R and B29R vaccinia genes, and the second transgene and the third transgene are inserted into the locus of the deletion in the B8R gene. In specific embodiments, the first transgene is inserted between the portion of the B14R vaccinia gene that remains and the portion of the B29R vaccinia gene that remains, and the second transgene and the third transgene are inserted into the locus of the deletion in the B8R gene. In specific embodiments, the third transgene is upstream of the second transgene. In specific embodiments, the third transgene is downstream of the second transgene. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is inserted between the partial C2L and F3L vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L, wherein the third nucleotide sequence is in the same orientation as endogenous vaccinia virus genes that flank the third nucleotide sequence, wherein the third transgene is inserted into the locus of the deletion in the B8R gene, and wherein the third transgene is upstream of the second transgene; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is inserted between the partial C2L and F3L vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is downstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In a specific embodiment, any one, two or three of the first transgene, the second transgene and the third transgene is/are inserted between the partial C2L and F3L vaccinia genes (that is, is/are present between the partial C2L and F3L genes). In a specific embodiment, any one, two or three of the the first transgene, the second transgene and the third transgene is/are inserted between the portion of the C2L vaccinia gene that remains and the portion of the F3L vaccinia gene that remains.

In a specific embodiment, any one, two or three of the first transgene, the second transgene and the third transgene is/are inserted in the locus of the deletion in the B8R gene (that is, is/are present in the locus of the B8R gene).

In a specific embodiment, any one, two or three of the first transgene, the second transgene and the third transgene is/are inserted between the partial B13R and B29R vaccinia genes (that is, is/are present between the partial B13R and B29R genes). In a specific embodiment, any one, two or three of the the first transgene, the second transgene and the third transgene is/are inserted between the portion of the B13R vaccinia gene that remains and the portion of the B29R vaccinia gene that remains.

In a specific embodiment, the first transgene is inserted between the partial C2L and F3L vaccinia genes (that is, is present between the partial C2L and F3L genes), the second transgene is inserted into the locus of the deletion in the B8R gene (that is, is present in the locus of the deletion in the B8R gene) and the third transgene is inserted between the partial B14R and B29R vaccinia genes (that is, is present between the partial B14R and B29R vaccinia genes). In a specific embodiment, the first transgene is inserted between the portion of the C2L vaccinia gene that remains and the portion of the F3L vaccinia gene that remains, the second transgene is inserted into the locus of the deletion in the B8R gene and the third transgene is inserted between the portion of the B14R vaccinia gene that remains and portion of the B29R vaccinia gene that remains.

In a specific embodiment, the second transgene is inserted between the partial C2L and F3L vaccinia genes (that is, is present between the partial C2L and F3L genes), the third transgene is inserted into the locus of the deletion in the B8R gene (that is, is present in the locus of the deletion in the B8R gene) and the first transgene is inserted between the partial B14R and B29R vaccinia genes (that is, is present between the partial B14R and B29R vaccinia genes). In a specific embodiment, the second transgene is inserted between the portion of the C2L vaccinia gene that remains and the portion of the F3L vaccinia gene that remains, the second transgene is inserted into the locus of the deletion in the B8R gene and the first transgene is inserted between the portion of the B14R vaccinia gene that remains and portion of the B29R vaccinia gene that remains.

In a specific embodiment, the third transgene is inserted between the partial C2L and F3L vaccinia genes (that is, is present between the partial C2L and F3L genes), the second transgene is inserted into the locus of the deletion in the B8R gene (that is, is present in the locus of the deletion in the B8R gene) and the first transgene is inserted between the partial B14R and B29R vaccinia genes (that is, is present between the partial B14R and B29R vaccinia genes). In a specific embodiment, the third transgene is inserted between the portion of the C2L vaccinia gene that remains and the portion of the F3L vaccinia gene that remains, the second transgene is inserted into the locus of the deletion in the B8R gene and the first transgene is inserted between the portion of the B14R vaccinia gene that remains and portion of the B29R vaccinia gene that remains.

In a specific embodiment, the first transgene and the second transgene are inserted between the partial C2L and F3L vaccinia genes (that is, are present between the partial C2L and F3L genes), and the third transgene is inserted into the locus of the deletion in the B8R gene (that is, is present in the locus of the deletion in the B8R gene). In a specific embodiment, the first transgene and the second transgene are inserted between the portion of the C2L vaccinia gene that remains and the portion of the F3L vaccinia gene that remains, and the third transgene is inserted into the locus of the deletion in the B8R gene

In a specific embodiment, the first transgene and the second transgene are inserted between the partial C2L and F3L vaccinia genes (that is, are present between the partial C2L and F3L genes), and the third transgene is inserted between the partial B14R and B29R vaccinia genes (that is, is present between the partial B14R and B29R vaccinia genes). In a specific embodiment, the first transgene and the second transgene are inserted between the portion of the C2L vaccinia gene that remains and the portion of the F3L vaccinia gene that remains, and the third transgene is inserted between the portion of the B14R vaccinia gene that remains and portion of the B29R vaccinia gene that remains.

In a specific embodiment, the third transgene is inserted between the partial C2L and F3L vaccinia genes (that is, is present between the partial C2L and F3L genes), and the first transgene and the second transgene are inserted into the locus of the deletion in the B8R gene (that is, is are present in the locus of the deletion in the B8R gene). In a specific embodiment, the third transgene is inserted between the portion of the C2L vaccinia gene that remains and the portion of the F3L vaccinia gene that remains, and the first transgene and the second transgene are inserted into the locus of the deletion in the B8R gene

In a specific embodiment, the third transgene is inserted between the partial C2L and F3L vaccinia genes (that is, is present between the partial C2L and F3L genes), and the first transgene and the second transgene are inserted between the partial B14R and B29R vaccinia genes (that is, are present between the partial B14R and B29R vaccinia genes). In a specific embodiment, the third transgene is inserted between the portion of the C2L vaccinia gene that remains and the portion of the F3L vaccinia gene that remains, and the first transgene and the second transgene are inserted between the portion of the B14R vaccinia gene that remains and portion of the B29R vaccinia gene that remains.

In a specific embodiment, the third transgene is inserted into the locus of the deletion in the B8R gene (that is, is present in the locus of the deletion in the B8R gene) and the first transgene and the second transgene are inserted between the partial B13R and B29R vaccinia genes (that is, are present between the partial B13R and B29R genes). In a specific embodiment, the third transgene is inserted into the locus of the deletion in the B8R gene and the first transgene and the second transgene are inserted between the portion of the B13R vaccinia gene that remains and the portion of the B29R vaccinia gene that remains. In a specific embodiment, the third transgene is inserted between the partial B13R and B29R vaccinia genes (that is, is present between the partial B13R and B29R genes) and the first transgene and the second transgene are inserted into the locus of the deletion in the B8R gene (that is, are present in the locus of the deletion in the B8R gene). In a specific embodiment, the third transgene is inserted between the portion of the B13R vaccinia gene that remains and portion of the B29R vaccinia gene that remains and the first transgene and the second transgene are inserted into the locus of the deletion in the B8R gene.

In a specific embodiment, the first transgene, the second transgene and the third transgene are inserted between the partial C2L and F3L vaccinia genes (that is, are present between the partial C2L and F3L genes). In a specific embodiment, the first transgene, the second transgene and the third transgene are inserted between the portion of the C2L vaccinia gene that remains and the portion of the F3L vaccinia gene that remains.

In a specific embodiment, the first transgene, the second transgene and the third transgene are inserted into the locus of the deletion in the B8R gene (that is, are present in the locus of the B8R gene).

In a specific embodiment, the first transgene, the second transgene and the third transgene are inserted between the partial B14R and B29R vaccinia genes (that is, are present between the partial B14R and B29R genes). In a specific embodiment, the first transgene, the second transgene and the third transgene are inserted between the portion of the B14R vaccinia gene that remains and the portion of the B29R vaccinia gene that remains.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is inserted between the partial B14R and B29R vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is upstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is inserted between the partial B14R and B29R vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is downstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to Cytotoxic T-lymphocyte Associated Protein 4 (CTLA-4), wherein the first nucleotide sequence is set forth in SEQ ID NO: 214; (c) a second transgene comprising a second nucleotide sequence encoding an Interleukin 12 (IL-12) polypeptide, wherein the second nucleotide sequence is set forth in SEQ ID NO: 215; and (d) a third transgene comprising a third nucleotide sequence encoding FMS-like tyrosine kinase 3 ligand (FLT3L), wherein the third nucleotide sequence is set forth in SEQ ID NO: 216.

In some embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

In certain embodiments, the nucleic acid further comprises a nucleotide sequence comprising at least one promoter operably linked to the first nucleotide sequence encoding the anti-CTLA-4 antibody. In a specific embodiment, the at least one promoter operably linked to the first nucleotide sequence encoding the anti-CTLA-4 antibody is an H5R promoter, a pS promoter, or a LEO promoter. In another specific embodiment, the at least one promoter operably linked to the first nucleotide sequence encoding the anti-CTLA-4 antibody is an H5R promoter.

In certain embodiments, the nucleic acid further comprises a nucleotide sequence comprising at least one promoter operably linked to the second nucleotide sequence encoding the IL-12 polypeptide. In a specific embodiment, the at least one promoter operably linked to the second nucleotide sequence encoding the IL-12 polypeptide is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence encoding FLT3L. In a specific embodiment, the at least one promoter operably linked to the third nucleotide sequence encoding FLT3L is a B8R promoter, a B19R promoter, a E3L promoter, an F11L promoter, or a B2R promoter. In another specific embodiment, the at least one promoter operably linked to the third nucleotide sequence encoding FLT3L is a B8R promoter. In another specific embodiment, the at least one promoter operably linked to the third nucleotide sequence encoding FLT3L is a B19R promoter. In another specific embodiment, the at least one promoter operably linked to the third nucleotide sequence encoding FLT3L is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214; (c) a second transgene comprising a second nucleotide sequence encoding an IL-12 polypeptide, wherein the second nucleotide sequence is set forth in SEQ ID NO: 215; and (d) a third transgene comprising a third nucleotide sequence encoding FLT3L, wherein the third nucleotide sequence is set forth in SEQ ID NO: 216; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence. In specific embodiments, the first transgene is inserted between the partial C2L and F3L vaccinia genes in SEQ ID NO: 210, and the second transgene and the third transgene are inserted into the locus of the deletion in the B8R gene. In specific embodiments, the first transgene is inserted between the partial B14R and B29R vaccinia genes in SEQ ID NO: 210, and the second transgene and the third transgene are inserted into the locus of the deletion in the B8R gene. In specific embodiments, the third transgene is upstream of the second transgene. In specific embodiments, the third transgene is downstream of the second transgene.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In some embodiments of the various embodiments and aspects described herein wherein the nucleic acid comprises the first transgene, the first transgene is inserted between the partial C2L and F3L vaccinia genes. In other embodiments of the various embodiments and aspects described herein wherein the nucleic acid comprises the first transgene, the first transgene is inserted into the locus of the deletion in the B8R gene. In other embodiments of the various embodiments and aspects described herein wherein the nucleic acid comprises the first transgene, the first transgene is inserted between the partial B14R and B29R vaccinia genes.

In some embodiments of the various embodiments and aspects described herein wherein the nucleic acid comprises the second transgene, the second transgene is inserted between the partial C2L and F3L vaccinia genes. In other embodiments of the various embodiments and aspects described herein wherein the nucleic acid comprises the second transgene, the second transgene is inserted into the locus of the deletion in the B8R gene. In other embodiments of the various embodiments and aspects described herein wherein the nucleic acid comprises the second transgene, the second transgene is inserted between the partial B14R and B29R vaccinia genes.

In some embodiments of the various embodiments and aspects described herein wherein the nucleic acid comprises the third transgene, the third transgene is inserted between the partial C2L and F3L vaccinia genes. In other embodiments of the various embodiments and aspects described herein wherein the nucleic acid comprises the third transgene, the third transgene is inserted into the locus of the deletion in the B8R gene. In other embodiments of the various embodiments and aspects described herein wherein the nucleic acid comprises the third transgene, the third transgene is inserted between the partial B14R and B29R vaccinia genes.

In some embodiments of the various embodiments and aspects described herein wherein the nucleic acid comprises the first transgene and the second transgene, the first transgene and the second transgene are inserted between the partial C2L and F3L vaccinia genes. In other embodiments of the various embodiments and aspects described herein wherein the nucleic acid comprises the first transgene and the second transgene, the first transgene and the second transgene are inserted into the locus of the deletion in the B8R gene. In other embodiments of the various embodiments and aspects described herein wherein the nucleic acid comprises the first transgene and the second transgene, the first transgene and the second transgene are inserted between the partial B14R and B29R vaccinia genes.

In some embodiments of the various embodiments and aspects described herein wherein the nucleic acid comprises the first transgene and the third transgene, the first transgene and the third transgene are inserted between the partial C2L and F3L vaccinia genes. In other embodiments of the various embodiments and aspects described herein wherein the nucleic acid comprises the first transgene and the third transgene, the first transgene and the third transgene are inserted into the locus of the deletion in the B8R gene. In other embodiments of the various embodiments and aspects described herein wherein the nucleic acid comprises the first transgene and the third transgene, the first transgene and the third transgene are inserted between the partial B14R and B29R vaccinia genes.

In some embodiments of the various embodiments and aspects described herein wherein the nucleic acid comprises the second transgene and the third transgene, the second transgene and the third transgene are inserted between the partial C2L and F3L vaccinia genes. In other embodiments of the various embodiments and aspects described herein wherein the nucleic acid comprises the second transgene and the third transgene, the second transgene and the third transgene are inserted into the locus of the deletion in the B8R gene. In other embodiments of the various embodiments and aspects described herein wherein the nucleic acid comprises the second transgene and the third transgene, the second transgene and the third transgene are inserted between the partial B14R and B29R vaccinia genes.

In some embodiments of the various embodiments and aspects described herein wherein the nucleic acid comprises the first transgene and the second transgene, the first transgene is inserted between the partial C2L and F3L vaccinia genes, and the second transgene is inserted into the locus of the deletion in the B8R gene. In other embodiments of the various embodiments and aspects described herein wherein the nucleic acid comprises the first transgene and the second transgene, the second transgene is inserted between the partial C2L and F3L vaccinia genes, and the first transgene is inserted into the locus of the deletion in the B8R gene. In other embodiments of the various embodiments and aspects described herein wherein the nucleic acid comprises the first transgene and the second transgene, the first transgene is inserted between the partial C2L and F3L vaccinia genes, and the second transgene is inserted between the partial B14R and B29R vaccinia genes. In other embodiments of the various embodiments and aspects described herein wherein the nucleic acid comprises the first transgene and the second transgene, the second transgene is inserted between the partial C2L and F3L vaccinia genes, and the first transgene is inserted between the partial B14R and B29R vaccinia genes. In other embodiments of the various embodiments and aspects described herein wherein the nucleic acid comprises the first transgene and the second transgene, the first transgene is inserted into the locus of the deletion in the B8R gene, and the second transgene is inserted between the partial B14R and B29R vaccinia genes. In other embodiments of the various embodiments and aspects described herein wherein the nucleic acid comprises the first transgene and the second transgene, the second transgene is inserted into the locus of the deletion in the B8R gene, and the first transgene is inserted between the partial B14R and B29R vaccinia genes.

In some embodiments of the various embodiments and aspects described herein wherein the nucleic acid comprises the first transgene and the third transgene, the first transgene is inserted between the partial C2L and F3L vaccinia genes, and the third transgene is inserted into the locus of the deletion in the B8R gene. In other embodiments of the various embodiments and aspects described herein wherein the nucleic acid comprises the first transgene and the third transgene, the third transgene is inserted between the partial C2L and F3L vaccinia genes, and the first transgene is inserted into the locus of the deletion in the B8R gene. In other embodiments of the various embodiments and aspects described herein wherein the nucleic acid comprises the first transgene and the third transgene, the first transgene is inserted between the partial C2L and F3L vaccinia genes, and the third transgene is inserted between the partial B14R and B29R vaccinia genes. In other embodiments of the various embodiments and aspects described herein wherein the nucleic acid comprises the first transgene and the third transgene, the third transgene is inserted between the partial C2L and F3L vaccinia genes, and the first transgene is inserted between the partial B14R and B29R vaccinia genes. In other embodiments of the various embodiments and aspects described herein wherein the nucleic acid comprises the first transgene and the third transgene, the first transgene is inserted into the locus of the deletion in the B8R gene, and the third transgene is inserted between the partial B14R and B29R vaccinia genes. In other embodiments of the various embodiments and aspects described herein wherein the nucleic acid comprises the first transgene and the third transgene, the third transgene is inserted into the locus of the deletion in the B8R gene, and the first transgene is inserted between the partial B14R and B29R vaccinia genes.

In some embodiments of the various embodiments and aspects described herein wherein the nucleic acid comprises the second transgene and the third transgene, the second transgene is inserted between the partial C2L and F3L vaccinia genes, and the third transgene is inserted into the locus of the deletion in the B8R gene. In other embodiments of the various embodiments and aspects described herein wherein the nucleic acid comprises the second transgene and the third transgene, the third transgene is inserted between the partial C2L and F3L vaccinia genes, and the second transgene is inserted into the locus of the deletion in the B8R gene. In other embodiments of the various embodiments and aspects described herein wherein the nucleic acid comprises the second transgene and the third transgene, the second transgene is inserted between the partial C2L and F3L vaccinia genes, and the third transgene is inserted between the partial B14R and B29R vaccinia genes. In other embodiments of the various embodiments and aspects described herein wherein the nucleic acid comprises the second transgene and the third transgene, the third transgene is inserted between the partial C2L and F3L vaccinia genes, and the second transgene is inserted between the partial B14R and B29R vaccinia genes. In other embodiments of the various embodiments and aspects described herein wherein the nucleic acid comprises the second transgene and the third transgene, the second transgene is inserted into the locus of the deletion in the B8R gene, and the third transgene is inserted between the partial B14R and B29R vaccinia genes. In other embodiments of the various embodiments and aspects described herein wherein the nucleic acid comprises the second transgene and the third transgene, the third transgene is inserted into the locus of the deletion in the B8R gene, and the second transgene is inserted between the partial B14R and B29R vaccinia genes.

In some embodiments of the various embodiments and aspects described herein wherein the nucleic acid comprises the first transgene, the second transgene and the third transgene, the first transgene, the second transgene, and the third transgene are inserted between the partial C2L and F3L vaccinia genes. In other embodiments of the various embodiments and aspects described herein wherein the nucleic acid comprises the first transgene, the second transgene and the third transgene, the first transgene, the second transgene, and the third transgene are inserted into the locus of the deletion in the B8R gene. In other embodiments of the various embodiments and aspects described herein wherein the nucleic acid comprises the first transgene, the second transgene and the third transgene, the first transgene, the second transgene, and the third transgene are inserted between the partial B14R and B29R vaccinia genes.

In other embodiments of the various embodiments and aspects described herein wherein the nucleic acid comprises the first transgene, the second transgene and the third transgene, the first transgene is inserted between the partial C2L and F3L vaccinia genes, and the second transgene and the third transgene are inserted into the locus of the deletion in the B8R gene. In other embodiments of the various embodiments and aspects described herein wherein the nucleic acid comprises the first transgene, the second transgene and the third transgene, the second transgene is inserted between the partial C2L and F3L vaccinia genes, and the first transgene and the third transgene are inserted into the locus of the deletion in the B8R gene. In other embodiments of the various embodiments and aspects described herein wherein the nucleic acid comprises the first transgene, the second transgene and the third transgene, the third transgene is inserted between the partial C2L and F3L vaccinia genes, and the first transgene and the second transgene are inserted into the locus of the deletion in the B8R gene. In other embodiments of the various embodiments and aspects described herein wherein the nucleic acid comprises the first transgene, the second transgene and the third transgene, the first transgene and the second transgene are inserted between the partial C2L and F3L vaccinia genes, and the third transgene is inserted into the locus of the deletion in the B8R gene. In other embodiments of the various embodiments and aspects described herein wherein the nucleic acid comprises the first transgene, the second transgene and the third transgene, the first transgene and the third transgene are inserted between the partial C2L and F3L vaccinia genes, and the second transgene is inserted into the locus of the deletion in the B8R gene. In other embodiments of the various embodiments and aspects described herein wherein the nucleic acid comprises the first transgene, the second transgene and the third transgene, the second transgene and the third transgene are inserted between the partial C2L and F3L vaccinia genes, and the first transgene is inserted into the locus of the deletion in the B8R gene. In other embodiments of the various embodiments and aspects described herein wherein the nucleic acid comprises the first transgene, the second transgene and the third transgene, the first transgene is inserted within between the partial C2L and F3L vaccinia genes, and the second transgene and the third transgene are inserted between the partial B14R and B29R vaccinia genes. In other embodiments of the various embodiments and aspects described herein wherein the nucleic acid comprises the first transgene, the second transgene and the third transgene, the second transgene is inserted between the partial C2L and F3L vaccinia genes, and the first transgene and the third transgene are inserted between the partial B14R and B29R vaccinia genes. In other embodiments of the various embodiments and aspects described herein wherein the nucleic acid comprises the first transgene, the second transgene and the third transgene, the third transgene is inserted between the partial C2L and F3L vaccinia genes, and the first transgene and the second transgene are inserted between the partial B14R and B29R vaccinia genes. In other embodiments of the various embodiments and aspects described herein wherein the nucleic acid comprises the first transgene, the second transgene and the third transgene, the first transgene and the second transgene are inserted between the partial C2L and F3L vaccinia genes, and the third transgene is inserted between the partial B14R and B29R vaccinia genes. In other embodiments of the various embodiments and aspects described herein wherein the nucleic acid comprises the first transgene, the second transgene and the third transgene, the first transgene and the third transgene are inserted between the partial C2L and F3L vaccinia genes, and the second transgene is inserted between the partial B14R and B29R vaccinia genes. In other embodiments of the various embodiments and aspects described herein wherein the nucleic acid comprises the first transgene, the second transgene and the third transgene, the second transgene and the third transgene are inserted between the partial C2L and F3L vaccinia genes, and the first transgene is inserted between the partial B14R and B29R vaccinia genes. In other embodiments of the various embodiments and aspects described herein wherein the nucleic acid comprises the first transgene, the second transgene and the third transgene, the first transgene is inserted into the locus of the deletion in the B8R gene, and the second transgene and the third transgene are inserted between the partial B14R and B29R vaccinia genes. In other embodiments of the various embodiments and aspects described herein wherein the nucleic acid comprises the first transgene, the second transgene and the third transgene, the second transgene is inserted into the locus of the deletion in the B8R gene, and the first transgene and the third transgene are inserted between the partial B14R and B29R vaccinia genes. In other embodiments of the various embodiments and aspects described herein wherein the nucleic acid comprises the first transgene, the second transgene and the third transgene, the third transgene is inserted into the locus of the deletion in the B8R gene, and the first transgene and the second transgene are inserted between the partial B14R and B29R vaccinia genes. In other embodiments of the various embodiments and aspects described herein wherein the nucleic acid comprises the first transgene, the second transgene and the third transgene, the first transgene and the second transgene are inserted into the locus of the deletion in the B8R gene, and the third transgene is inserted between the partial B14R and B29R vaccinia genes. In other embodiments of the various embodiments and aspects described herein wherein the nucleic acid comprises the first transgene, the second transgene and the third transgene, the first transgene and the third transgene are inserted into the locus of the deletion in the B8R gene, and the second transgene is inserted between the partial B14R and B29R vaccinia genes. In other embodiments of the various embodiments and aspects described herein wherein the nucleic acid comprises the first transgene, the second transgene and the third transgene, the second transgene and the third transgene are inserted into the locus of the deletion in the B8R gene, and the first transgene is inserted between the partial B14R and B29R vaccinia genes.

In other embodiments of the various embodiments and aspects described herein wherein the nucleic acid comprises the first transgene, the second transgene and the third transgene, the first transgene is inserted between the partial C2L and F3L vaccinia genes, the second transgene is inserted into the locus of the deletion in the B8R gene, and the third transgene is inserted between the partial B14R and B29R vaccinia genes. In other embodiments of the various embodiments and aspects described herein wherein the nucleic acid comprises the first transgene, the second transgene and the third transgene, the first transgene is inserted between the partial C2L and F3L vaccinia genes, the third transgene is inserted into the locus of the deletion in the B8R gene, and the second transgene is inserted between the partial B14R and B29R vaccinia genes. In other embodiments of the various embodiments and aspects described herein wherein the nucleic acid comprises the first transgene, the second transgene and the third transgene, the second transgene is inserted between the partial C2L and F3L vaccinia genes, the first transgene is inserted into the locus of the deletion in the B8R gene, and the third transgene is inserted between the partial B14R and B29R vaccinia genes. In other embodiments of the various embodiments and aspects described herein wherein the nucleic acid comprises the first transgene, the second transgene and the third transgene, the second transgene is inserted between the partial C2L and F3L vaccinia genes, the third transgene is inserted into the locus of the deletion in the B8R gene, and the first transgene is inserted between the partial B14R and B29R vaccinia genes. In other embodiments of the various embodiments and aspects described herein wherein the nucleic acid comprises the first transgene, the second transgene and the third transgene, the third transgene is inserted between the partial C2L and F3L vaccinia genes, the first transgene is inserted into the locus of the deletion in the B8R gene, and the second transgene is inserted between the partial B14R and B29R vaccinia genes. In other embodiments of the various embodiments and aspects described herein wherein the nucleic acid comprises the first transgene, the second transgene and the third transgene, the third transgene is inserted between the partial C2L and F3L vaccinia genes, the second transgene is inserted into the locus of the deletion in the B8R gene, and the first transgene is inserted between the partial B14R and B29R vaccinia genes.

In various embodiments and aspects described herein wherein the nucleic acid comprises the nucleotide sequence of SEQ ID NO: 210, the partial C2L and F3L vaccinia genes are partial C2L and F3L vaccinia genes in SEQ ID NO: 210. In various embodiments and aspects described herein wherein the nucleic acid comprises the nucleotide sequence of SEQ ID NO: 210, the partial B14R and B29R vaccinia genes are partial B14R and B29R vaccinia genes in SEQ ID NO: 210.

In one aspect, provided herein is a virus comprising the nucleic acid described herein.

In one aspect, provided are packaging cell lines comprising nucleic acids or viruses disclosed herein.

In one aspect, provided herein are pharmaceutical compositions comprising a virus disclosed herein and a physiologically acceptable carrier.

In one aspect, provided are methods of treating cancer in a mammalian patient, said method comprising administering a therapeutically effective amount of a virus as disclosed herein to said patient. In another aspect, provided are methods of treating cancer in a mammalian patient, said method comprising administering a therapeutically effective amount of a pharmaceutical composition as disclosed herein to said patient. In some embodiments, the mammalian patient is a human patient.

In some embodiments, the virus is used as a prime in a prime:boost treatment. In some embodiments, the virus is used as a boost in a prime:boost treatment.

In some embodiments, the mammalian patient has cancer. For example, in some embodiments, the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In some embodiments, the cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin's lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi's sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

In some embodiments, provided methods further comprise administering to said patient an immune checkpoint inhibitor. In some embodiments, the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In some embodiments, the immune checkpoint inhibitor is an anti-PD1 antibody or antigen-binding fragment thereof or an anti-CTLA-4 antibody or antigen-binding fragment thereof. In some embodiments, the immune checkpoint inhibitor is an anti-PD1 antibody or antigen-binding fragment thereof. In some embodiments, the immune checkpoint inhibitor is an anti-PD-L1 antibody or antigen-binding fragment thereof. In some embodiments, the immune checkpoint inhibitor is an anti-PD1 or anti-PD-L1 antibody or antigen-binding fragment thereof. In some embodiments, the immune checkpoint inhibitor is an anti-CTLA-4 antibody or antigen-binding fragment thereof.

In some embodiments, provided methods further comprise administering to said patient an interleukin.

In some embodiments, said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In some embodiments, the interleukin is selected from the group consisting of IL-12 p35, IL-12 p40, and IL-12 p70. In some embodiments, the interleukin is membrane-bound.

In some embodiments, the method further comprises administering to said patient an interferon. In some embodiments, the interferon is selected from the group consisting of IFN-alpha, IFN-beta, IFN-delta, IFN-epsilon, IFN-tau, IFN-omega, IFN-zeta, and IFN-gamma.

In some embodiments, provided methods further comprises administering to said patient a cytokine. In some embodiments, the cytokine is a TNF superfamily member protein. In some embodiments, the TNF superfamily member protein is selected from the group consisting of TRAIL, Fas ligand, LIGHT (TNFSF-14), TNF-alpha, and 4-1BB ligand. In some embodiments, the cytokine is selected from the group consisting of GM-CSF, Flt3 ligand, CD40 ligand, TGF-beta, VEGF-R2, and cKit. In some embodiments, the cytokine is Flt3 ligand.

In one aspect, provided are kits comprising a nucleic acid or virus as disclosed herein and a package insert instructing a user of said kit to express said nucleic acid or said virus in a host cell.

In one aspect, provided are kits comprising a virus as disclosed herein and a package insert instructing a user to administer a therapeutically effective amount of said virus to a mammalian patient having cancer, thereby treating said cancer. In some embodiments, the mammalian patient is a human patient.

### 3.1. Definitions

As used herein, the term “about” refers to a value that is no more than 10% above or below the value being described. For example, the term “about 5 nM” indicates a range of from 4.5 nM to 5.5 nM.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rIgG, and scFv fragments. Moreover, unless otherwise indicated, the term “monoclonal antibody” (mAb) is meant to include both intact molecules, as well as, antibody fragments (such as, for example, Fab and F(ab′)2 fragments) that are capable of specifically binding to a target protein. Fab and F(ab′)2 fragments lack the Fc fragment of an intact antibody, clear more rapidly from the circulation of the animal, and may have less non-specific tissue binding than an intact antibody (see Wahl et al., J. Nucl. Med. 24:31 6, 1 983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)2, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, VH, C_{L}, and C_{H}1 domains; (ii) a F(ab′)2 fragment, a bivalent fragment comprising two Fab fragments linked by a disulfide bridge at the hinge region; (iii) a Fd fragment consisting of the VH and C_{H}1 domains; (iv) a Fv fragment consisting of the VL and VH domains of a single arm of an antibody, (v) a dAb including VH and VL domains; (vi) a dAb fragment (Ward et al., Nature 341:544-546, 1 989), which consists of a VH domain; (vii) a dAb which consists of a VH or a VL domain; (viii) an isolated complementarity determining region (CDR); and (ix) a combination of two or more isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “bispecific antibodies” refers to monoclonal, often human or humanized antibodies that have binding specificities for at least two different antigens.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations.

As used herein, the term “chimeric” antibody refers to an antibody having variable sequences derived from an immunoglobulin of one source organism, such as rat or mouse, and constant regions derived from an immunoglobulin of a different organism (e.g., a human). Methods for producing chimeric antibodies are known in the art. See, e.g., Morrison, 1985, Science 229(4719): 1202-7; Oi et al., 1986, BioTechniques 4:214-221; Gillies et al., 1985, J. Immunol. Methods 125:191 -202; U.S. Pat. Nos. 5,807,715; 4,816,567; and 4,816,397; incorporated herein by reference.

As used herein, the term “complementarity determining region” (CDR) refers to a hypervariable region found both in the light chain and the heavy chain variable domains. The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four framework regions that primarily adopt a (3-sheet configuration, connected by three CDRs, which form loops that connect, and in some cases form part of, the (3-sheet structure. The CDRs in each chain are held together in close proximity by the FR regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest (National Institute of Health, Bethesda, Md. 1987; incorporated herein by reference).

As used herein, numbering of immunoglobulin amino acid residues is done according to the immunoglobulin amino acid residue numbering system of Kabat et al., unless otherwise indicated.

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below. From this table it is appreciated that the conservative amino acid families include (i) G, A, V, L and I; (ii) D and E; (iii) C, Sand T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

TABLE 1
Representative physicochemical properties of
naturally occurring amino acids

| | | | | Electrostatic | |
|---|---|---|---|---|---|
| | 3 | 1 | Side- | character at | |
| Amino | Letter | Letter | chain | physiological | Steric |
| Acid | Code | Code | Polarity | pH (7.4) | Volume^{†} |
| Alanine | Ala | A | non- | neutral | small |
| | | | polar | | |
| Arginine | Arg | R | polar | cationic | large |
| Asparagine | Asn | N | polar | neutral | intermediate |
| Aspartic | Asp | D | polar | anionic | intermediate |
| acid | | | | | |
| Cysteine | Cys | C | non- | neutral | intermediate |
| | | | polar | | |
| Glutamic | Glu | E | polar | anionic | intermediate |
| acid | | | | | |
| Glutamine | Gln | Q | polar | neutral | intermediate |
| Glycine | Gly | G | non- | neutral | small |
| | | | polar | | |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | non- | neutral | large |
| | | | polar | | |
| Leucine | Leu | L | non- | neutral | large |
| | | | polar | | |
| Lysine | Lys | K | polar | cationic | large |
| Methionine | Met | M | non- | neutral | large |
| | | | polar | | |
| Phenyl- | Phe | F | non- | neutral | large |
| alanine | | | polar | | |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | non- | neutral | bulky |
| | | | polar | | |
| Tyrosine | Tyr | Y | polar | neutral | large |
| Valine | Val | V | non- | neutral | intermediate |
| | | | polar | | |

^{†}based on volume in A³: 50-100 is small, 100-150 is intermediate, 150-200 is large, and >200 is bulky

As used herein, the terms “delete,” “deletion,” and the like refer to modifications to a gene or a regulatory element associated therewith or operatively linked thereto (e.g., a transcription factor-binding site, such as a promoter or enhancer element) that remove the gene or otherwise render the gene nonfunctional. Exemplary deletions, as described herein, include the removal of the entirety of a nucleic acid encoding a gene of interest, from the start codon to the stop codon of the target gene. Other examples of deletions as described herein include the removal of a portion of the nucleic acid encoding the target gene (e.g., one or more codons, or a portion thereof, such as a single nucleotide deletion) such that, upon expression of the partially-deleted target gene, the product (e.g., RNA transcript, protein product, or regulatory RNA) is nonfunctional or less functional then a wild-type form of the target gene. Exemplary deletions as described herein include the removal of all or a portion of the regulatory element(s) associated with a gene of interest, such as all or a portion of the promoter and/or enhancer nucleic acids that regulate expression of the target gene.

In specific embodiments, the recombinant vaccinia virus genome described in this disclosure comprises deletions in one or more of the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, B20R, B21R (in 3′ ITR), B22R (in 3′ ITR), B23R (in 3′ ITR), B24R (in 3′ ITR), B25R (in 3′ ITR), B26R (in 3′ ITR), B27R (in 3′ ITR), B28R (in 3′ ITR), and B29R (in 3′ ITR). In a specific embodiment, the recombinant vaccinia virus genome described in this disclosure comprises deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; and deletions in the following genes in the 3′ ITR: B21R, B22R, B23R, B24R, B25R, B26R, B27R, B28R, and B29R. In specific embodiments, the recombinant vaccinia virus genome described in this disclosure comprises deletions in one or more of the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, B20R, B21R (in 3′ ITR), B22R (in 3′ ITR), B23R (in 3′ ITR), B24R (in 3′ ITR), B25R (in 3′ ITR), B26R (in 3′ ITR), B27R (in 3′ ITR), B28R (in 3′ ITR), and B29R (in 3′ ITR), and also comprises a deletion in the B8R gene.

In some embodiments, a gene deletion removes the entire sequence of the gene. In other embodiments, a gene deletion is a partial deletion, that is, one that removes part of the sequence of the gene. In one embodiment, a gene deletion is a partial deletion that removes at least 30%, at least 40%, at least 50%, at least 60%, at least 70%, at least 80%, at least 90% or at least 95% of the sequence of the gene. In one embodiment, a gene deletion is a partial deletion that removes at least 30%, at least 40%, at least 50%, at least 60%, at least 70%, at least 80%, or at least 90% of the protein coding sequence of the gene. In other embodiments, a gene deletion removes 100% of the sequence of the gene. In yet other embodiments, a gene deletion removes 100% of the protein coding sequence of the gene. In one embodiment, a gene deletion removes at least 50, at least 100, at least 200, at least 300, at least 400, at least 500, at least 600, at least 700, at least 800, at least 900, or at least 1000 nucleotides of the sequence of the gene. In another embodiment, a gene deletion is a partial deletion that removes at least 50, at least 100, at least 200, at least 300, at least 400, at least 500, at least 600, at least 700, at least 800, at least 900, or at least 1000 nucleotides of the sequence of the gene. In a specific embodiment, a partial deletion in a gene results in a partial gene.

As used herein, the term “derivatized antibodies” refers to antibodies that are modified by a chemical reaction so as to cleave residues or add chemical moieties not native to an isolated antibody. Derivatized antibodies can be obtained by glycosylation, acetylation, pegylation, phosphorylation, amidation, derivatization by addition of known chemical protecting/blocking groups, proteolytic cleavage, linkage to a cellular ligand or other protein. Any of a variety of chemical modifications can be carried out by known techniques, including, without limitation, specific chemical cleavage, acetylation, formylation, metabolic synthesis of tunicamycin, etc. using established procedures. Additionally, the derivative can contain one or more non-natural amino acids, e.g., using amber suppression technology (see, e.g., U.S. Pat. No. 6,964,859; incorporated herein by reference).

As used herein, the term “diabodies” refers to bivalent antibodies comprising two polypeptide chains, in which each polypeptide chain includes VH and VL domains joined by a linker that is too short (e.g., a linker composed of five amino acids) to allow for intramolecular association of VH and VL domains on the same peptide chain. This configuration forces each domain to pair with a complementary domain on another polypeptide chain so as to form a homodimeric structure. Accordingly, the term “triabodies” refers to trivalent antibodies comprising three peptide chains, each of which contains one VH domain and one VL domain joined by a linker that is exceedingly short (e.g., a linker composed of 1-2 amino acids) to permit intramolecular association of VH and VL domains within the same peptide chain. In order to fold into their native structure, peptides configured in this way typically trimerize so as to position the VH and VL domains of neighboring peptide chains spatially proximal to one another to permit proper folding (see Holliger et al., Proc. Natl. Acad. Sci. USA 90:6444-48, 1993; incorporated herein by reference). As used herein, a “dual variable domain immunoglobulin” (“DVD-Ig”) refers to an

antibody that combines the target-binding variable domains of two monoclonal antibodies via linkers to create a tetravalent, dual-targeting single agent. (Gu et al., Meth. Enzymol., 502:25-41, 2012; incorporated by reference herein).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “framework region” or “FW region” includes amino acid residues that are adjacent to the CDRs. FW region residues may be present in, for example, human antibodies, rodent-derived antibodies (e.g., murine antibodies), humanized antibodies, primatized antibodies, chimeric antibodies, antibody fragments (e.g., Fab fragments), single-chain antibody fragments (e.g., scFv fragments), antibody domains, and bispecific antibodies, among others.

As used herein, the term “heterospecific antibodies” refers to monoclonal, preferably human or humanized, antibodies that have binding specificities for at least two different antigens. Traditionally, the recombinant production of heterospecific antibodies is based on the co-expression of two immunoglobulin heavy chain-light chain pairs, where the two heavy chains have different specificities (Milstein et al., Nature 305:537, 1 983). Similar procedures are disclosed, e.g., in WO 93/08829, U.S. Pat. Nos. 6,210,668; 6,193,967; 6,132,992; 6,106,833; 6,060,285; 6,037,453; 6,010,902; 5,989,530; 5,959,084; 5,959,083; 5,932,448; 5,833,985; 5,821,333; 5,807,706; 5,643,759, 5,601,819; 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986); incorporated herein by reference. Heterospecific antibodies can include Fc mutations that enforce correct chain association in multi-specific antibodies, as described by Klein et al., mAbs 4(6):653-663, 2012; incorporated herein by reference.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A human antibody can be produced in a human cell (e.g., by recombinant expression), or by a non-human animal or a prokaryotic or eukaryotic cell that is capable of expressing functionally rearranged human immunoglobulin (e.g., heavy chain and/or light chain) genes. Further, when a human antibody is a single-chain antibody, it can include a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. See U.S. Pat. Nos. 4,444,887 and 4,716,111; and PCT publications WO 1998/46645; WO 1998/50433; WO 1998/24893; WO 1998/16654; WO 1996/34096; WO 1996/33735; and WO 1991/10741; incorporated herein by reference. Human antibodies can also be produced using transgenic mice that are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625, 126; 5,633,425; 5,569,825; 5,661,016; 5,545,806; 5,814,318; 5,885,793; 5,916,771; and 5,939,598; incorporated by reference herein.

As used herein, the term “humanized” antibodies refers to forms of non-human (e.g., murine) antibodies that are chimeric immunoglobulins, immunoglobulin chains or fragments thereof (such as Fv, Fab, Fab′, F(ab′)2 or other target-binding subdomains of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin. All or substantially all of the FR regions may also be those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., Nature 332:323-7, 1988; U.S. Pat. Nos.: 5,530,101; 5,585,089; 5,693,761; 5,693,762; and 6,180,370 to Queen et al; EP239400; PCT publication WO 91/09967; U.S. Pat. No. 5,225,539; EP592106; and EP519596; incorporated herein by reference.

As used herein, the term “monoclonal antibody” refers to an antibody that is derived from a single clone, including any eukaryotic, prokaryotic, or phage clone, and not the method by which it is produced.

As used herein, the term “multi-specific antibodies” refers to antibodies that exhibit affinity for more than one target antigen. Multi-specific antibodies can have structures similar to full immunoglobulin molecules and include Fc regions, for example IgG Fc regions. Such structures can include, but not limited to, IgG-Fv, IgG-(scFv)2, DVD-Ig, (scFv)2-(scFv)2-Fc and (scFv)2-Fc-(scFv)2. In case of IgG-(scFv)2, the scFv can be attached to either the N-terminal or the C-terminal end of either the heavy chain or the light chain. Exemplary multi-specific molecules have been reviewed by Kontermann, 2012, mAbs 4(2):182-197, Yazaki et al., 2013, Protein Engineering, Design & Selection 26(3):1 87-1 93, and Grote et al., 2012, in Proetzel & Ebersbach (eds.), Antibody Methods and Protocols, Methods in Molecular Biology vol. 901, chapter 16:247-263; incorporated herein by reference. Exemplary multi-specific molecules that lack Fc regions and into which antibodies or antibody fragments can be incorporated include scFv dimers (diabodies), trimers (triabodies) and tetramers (tetrabodies), Fab dimers (conjugates by adhesive polypeptide or protein domains) and Fab trimers (chemically conjugated), are described by Hudson and Souriau, 2003, Nature Medicine 9:129-134; incorporated herein by reference.

As used herein, the term “percent(%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (ONASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “primatized antibody” refers to an antibody comprising framework regions from primate-derived antibodies and other regions, such as CDRs and constant regions, from antibodies of a non-primate source. Methods for producing primatized antibodies are known in the art. See e.g., U.S. Pat. Nos. 5,658,570; 5,681,722; and 5,693,780; incorporated herein by reference.

As used herein, the term “operatively linked” in the context of a polynucleotide fragment is intended to mean that the two polynucleotide fragments are joined such that the amino acid sequences encoded by the two polynucleotide fragments remain in-frame.

As used herein, the terms “regulatory element” and the like refer to promoters, enhancers, and other expression control elements (e.g., polyadenylation signals) that control the transcription or translation of the antibody chain genes. Such regulatory sequences are described, for example, in Goeddel, Gene Expression Technology: Methods in Enzymology 185 (Academic Press, San Diego, Calif., 1990); incorporated herein by reference. As used herein, the terms “subject” and “patient” refer to an organism that receives treatment for a particular disease or condition as described herein (such as cancer or an infectious disease). Examples of subjects and patients include mammals, such as humans, receiving treatment for diseases or conditions, for example, cell proliferation disorders, such as cancer.

As used herein, the term “scFv” refers to a single-chain Fv antibody in which the variable domains of the heavy chain and the light chain from an antibody have been joined to form one chain. scFv fragments contain a single polypeptide chain that includes the variable region of an antibody light chain (VL) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the VL and VH regions of a scFv fragment can be a peptide linker composed of proteinogenic amino acids. Alternative linkers can be used to so as to increase the resistance of the scFv fragment to proteolytic degradation (e.g., linkers containing D-amino acids), in order to enhance the solubility of the scFv fragment (e.g., hydrophilic linkers such as polyethylene glycol-containing linkers or polypeptides containing repeating glycine and serine residues), to improve the biophysical stability of the molecule (e.g., a linker containing cysteine residues that form intramolecular or intermolecular disulfide bonds), or to attenuate the immunogenicity of the scFv fragment (e.g., linkers containing glycosylation sites). scFv molecules are known in the art and are described, e.g., in U.S. Pat. No. 5,892,019, Flo et al., (Gene 77:51, 1989); Bird et al., (Science 242:423, 1988); Pantoliano et al., (Biochemistry 30:10117, 1991); Milenic et al., (Cancer Research 51:6363, 1991); and Takkinen et al., (Protein Engineering 4:837, 1991). The VL and VH domains of a scFv molecule can be derived from one or more antibody molecules. It will also be understood by one of ordinary skill in the art that the variable regions of the scFv molecules of the invention can be modified such that they vary in amino acid sequence from the antibody molecule from which they were derived. For example, in some embodiments, nucleotide or amino acid substitutions leading to conservative substitutions or changes at amino acid residues can be made (e.g., in CDR and/or framework residues). Alternatively or in addition, mutations are made to CDR amino acid residues to optimize antigen binding using art recognized techniques. scFv fragments are described, for example, in WO 2011/084714; incorporated herein by reference.

As used herein, the phrase “specifically binds” refers to a binding reaction which is determinative of the presence of an antigen in a heterogeneous population of proteins and other biological molecules that is recognized, e.g., by an antibody or antigen-binding fragment thereof, with particularity. An antibody or antigen-binding fragment thereof that specifically binds to an antigen may bind to the antigen with a KD of less than 100 nM. For example, an antibody or antigen-binding fragment thereof that specifically binds to an antigen may bind to the antigen with a KD of up to 100 nM (e.g., between 1 pM and 100 nM). An antibody or antigen-binding fragment thereof that does not exhibit specific binding to a particular antigen or epitope thereof may exhibit a KD of greater than 100 nM (e.g., greater than 500 nm, 1 μM, 100 μM, 500 μM, or 1 mM) for that particular antigen or epitope thereof. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection and the like.

As used herein, the terms “treat” or “treatment” refer to therapeutic treatment, in which the object is to prevent or slow down (lessen) an undesired physiological change or disorder, such as the progression of a cell proliferation disorder, such as cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

As used herein, the term “vector” refers to a nucleic acid vector, e.g., a DNA vector, such as a plasmid, a RNA vector, virus or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/1 1026; incorporated herein by reference. Expression vectors of the invention may contain one or more additional sequence elements used for the expression of proteins and/or the integration of these polynucleotide sequences into the genome of a host cell, such as a mammalian cell (e.g., a human cell). Exemplary vectors that can be used for the expression of antibodies and antibody fragments described herein include plasmids that contain regulatory sequences, such as promoter and enhancer regions, which direct gene transcription. Vectors may contain nucleic acids that modulate the rate of translation of a target gene or that improve the stability or nuclear export of the mRNA that results from gene transcription. These sequence elements may include, e.g., 5′ and 3′ untranslated regions, an internal ribosomal entry site (IRES), and polyadenylation signal site in order to direct efficient transcription of the gene carried on the expression vector. The vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin. As used herein, the term “VH” refers to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 Daltons, composed of two identical light (L) chains and two identical heavy (H) chains. Each heavy chain of a native antibody has at the amino terminus a variable domain (V_{H}) followed by a number of constant domains. Each light chain of a native antibody has a variable domain at the amino terminus (VL) and a constant domain at the carboxy terminus.

### 3.2. Gene Definitions

As used herein, “B8R” refers to an orthopoxvirus (e.g., vaccinia, e.g., Copenhagen) gene, such as a gene that encodes a secreted protein with homology to the gamma interferon (IFN-γ) receptor. A nonlimiting example of a protein sequence encoded by an exemplary B8R gene in a Copenhagen strain of the vaccinia virus is given in UniProtKB database entry P21004 and is reproduced below:

(SEQ ID NO: 1)
MRYIIILAVLFINSIHAKITSYKFESVNFDSKIEWTGDGLYNISLKNYGIK
TWQTMYTNVPEGTYDISAFPKNDFVSFWVKFEQGDYKVEEYCTGLCVEVKI
GPPTVTLTEYDDHINLYIEHPYATRGSKKIPIYKRGDMCDIYLLYTANFTF
GDSEEPVTYDIDDYDCTSTGCSIDFATTEKVCVTAQGATEGFLEKITPWSS
EVCLTPKKNVYTCAIRSKEDVPNFKDKMARVIKRKFNKQSQSYLTKFLGST
SNDVTTFLSMLNLTKYS.

The term “B8R” may also include fragments or variants of the protein listed above, or of homologous genes from another vaccinia virus strain. Variants include, without limitation, those sequences having 85 percent or greater identity to the sequences disclosed herein.

As used herein, “B14R” refers to an orthopoxvirus (e.g., vaccinia, e.g., Copenhagen) gene. An example of a protein sequence encoded by an exemplary B14R gene in a Copenhagen strain of the vaccinia virus is given in UniProtKB database entry P20842 and is reproduced below:

(SEQ ID NO: 2)
MNHCLLAISAVYFKAKWLTPFEKEFTSDYPFYVSPTEMVDVSMMSMYGELF
NHASVKESFGNFSIIELPYVGDTSMMVILPDKIDGLESIEQNLTDTNFKKW
CNSLDAMFIDVHIPKFKVTGSYNLVDTLVKSGLTEVFGSTGDYSNMCNLDV
SVDAMIHKTYIDVNEEYTEAAAATCALVSDCASTITNEFCVDHPFIYVIRH
VDGKILFVGRYCSPTTNC.

As used herein, “B15R” refers to an orthopoxvirus (e.g., vaccinia, e.g., Copenhagen) gene. An example of a protein sequence encoded by an exemplary B15R gene in a Copenhagen strain of the vaccinia virus is given in UniProtKB database entry P21089 and is reproduced below:

(SEQ ID NO: 3)
MTANFSTHVFSPQHCGCDRLTSIDDVKQCLTEYIYWSSYAYRNRQCAGQLY
STLLSFRDDAELVFIDIRELVKNMPWDDVKDCTEIIRCYIPDEQKTIREIS
AIIGLCAYAATYWGGEDHPTSNSLNALFVMLEMLNYVDYNIIFRRMN.

As used herein, “B16R” refers to an orthopoxvirus (e.g., vaccinia, e.g., Copenhagen) gene, such as a gene that encodes a IL-1-beta inhibitor. An example of a protein sequence encoded by an exemplary B16R gene in a Copenhagen strain of the vaccinia virus is given in UniProtKB database entry P21116 and is reproduced below: MSILPVIFLP IFFYSSFVQT FNASECIDKG (SEQ ID NO: 4).

As used herein, “B17L” refers to an orthopoxvirus (e.g., vaccinia, e.g., Copenhagen) gene. An example of a protein sequence encoded by an exemplary B17L gene in a Copenhagen strain of the vaccinia virus is given in UniProtKB database entry P21075 and is reproduced below:

(SEQ ID NO: 5)
MSRKFMQVYEYDREQYLDEFIEDRYNDSFITSPEYYSAEKYMCRYTTLNHN
CVNVRRCALDSKLLHDIITNCKIYNNIELVRATKFVYYLDLIKCNWVSKVG
DSVLYPVIFITHTSTRNLDKVSVKTYKGVKVKKLNRCADHAIVINPFVKFK
LTLPNKTSHAKVLVTFCKLRTDITPVEAPLPGNVLVYTFPDINKRIPGYIH
VNIEGCIDGMIYINSSKFACVLKLHRSMYRIPPFPIDICSCCSQYTNDDIE
IPIHDLIKDVAIFKNKETVYYLKLNNKTIARFTYFNNIDTAITQEHEYVKI
ALGIVCKLMINNMHSIVGVNHSNTFVNCLLEDNV.

As used herein, “B 18R” refers to an orthopoxvirus (e.g., vaccinia, e.g., Copenhagen) gene, such as a gene that encodes an Ankyrin repeat protein. An example of a protein sequence encoded by an exemplary B 18R gene in a Copenhagen strain of the vaccinia virus is given in UniProtKB database entry P21076 and is reproduced below:

(SEQ ID NO: 6)
MSRRLIYVLNINRKSTHKIQENEIYTYFSHCNIDHTSTELDFVVKNYDLNR
RQHVTGYTALHCYLYNNYFTNDVLKILLNHDVNVTMKTSSGRMPVYILLTR
CCNISHDVVIDMIDKDKNHLSHRDYSNLLLEYIKSRYMLLKEEDIDENIVS
TLLDKGIDPNFKQDGYTALHYYYLCLAHVYKPGECRKPITIKKAKRIISLF
IQHGANLNALDNCGNTPFHLYLSIEMCNNIHMTKMLLTFNPNFKICNNHGL
TPILCYITSDYIQHDILVMLIHHYETNVGEMPIDERRMIVFEFIKTYSTRP
ADSITYLMNRFKNINIYTRYEGKTLLHVACEYNNTQVIDYLIRINGDINAL
TDNNKHATQLIIDNKENSPYTINCLLYILRYIVDKNVIRSLVDQLPSLPIF
DIKSFEKFISYCILLDDTFYDRHVKNRDSKTYRYAFSKYMSFDKYDGIITK
CHDETMLLKLSTVLDTTLYAVLRCHNSRKLRRYLTELKKYNNDKSFKIYSN
IMNERYLNVYYKDMYVSKVYDKLFPVFTDKNCLLTLLPSEIIYEILYMLTI
NDLYNISYPPTKV.

As used herein, “B19R” refers to an orthopoxvirus (e.g., vaccinia, e.g., Copenhagen) gene, such as a gene that encodes a IFN-alpha-beta-receptor-like secreted glycoprotein. An example of a protein sequence encoded by an exemplary B19R gene in a Copenhagen strain of the vaccinia virus is given in UniProtKB database entry P21077 and is reproduced below:

(SEQ ID NO: 7)
MTMKMMVHIYFVSLSLLLLLFHSYAIDIENEITEFFNKMRDTLPAKDSKWL
NPACMFGGTMNDMATLGEPFSAKCPPIEDSLLSHRYKDYVVKWERLEKNRR
RQVSNKRVKHGDLWIANYTSKFSNRRYLCTVTTKNGDCVQGIVRSHIKKPP
SCIPKTYELGTHDKYGIDLYCGILYAKHYNNITWYKDNKEINIDDIKYSQT
GKELIIHNPELEDSGRYDCYVHYDDVRIKNDIVVSRCKILTVIPSQDHRFK
LILDPKINVTIGEPANITCTAVSTSLLIDDVLIEWENPSGWLIGFDFDVYS
VLTSRGGITEATLYFENVTEEYIGNTYKCRGHNYYFEKTLTTTVVLE.

As used herein, “B20R” refers to an orthopoxvirus (e.g., vaccinia, e.g., Copenhagen) gene, such as a gene that encodes an Ankyrin repeat protein. An example of a protein sequence encoded by an exemplary B20R gene in a Copenhagen strain of the vaccinia virus is given in UniProtKB database entry P21078 and is reproduced below:

(SEQ ID NO: 8)
MDEDTRLSRYLYLTDREHINVDSIKQLCKISDPNACYRCGCTALHEYFYNY
RSVNGKYKYRYNGYYQYYSSSDYENYNEYYYDDYDRTGMNSESDSESDNIS
IKTEYENEYEFYDETQDQSTQHNDL.

As used herein, “C1L” refers to an orthopoxvirus (e.g., vaccinia, e.g., Copenhagen) gene. An example of a protein sequence encoded by an exemplary C1L gene in a Copenhagen strain of the vaccinia virus is given in UniProtKB database entry P21036 and is reproduced below:

(SEQ ID NO: 9)
MVKNNKISNSCRMIMSTNPNNILMRHLKNLTDDEFKCIIHRSSDFLYLSDS
DYTSITKETLVSEIVEEYPDDCNKILAIIFLVLDKDIDVDIETKLKPKPAV
RFAILDKMTEDIKLTDLVRHYFRYIEQDIPLGPLFKKIDSYRTRAINKYSK
ELGLATEYFNKYGHLMFYTLPIPYNRFFCRNSIGFLAVLSPTIGHVKAFYK
FIEYVSIDDRRKFKKELMSK.

As used herein, “C2L” refers to an orthopoxvirus (e.g., vaccinia, e.g., Copenhagen) gene, such as a gene that encodes a kelch-like protein that affects calcium-independent adhesion to the extracellular matrix. An example of a protein sequence encoded by an exemplary C2L gene in a Copenhagen strain of the vaccinia virus is given in UniProtKB database entry P21037 and is reproduced below:

(SEQ ID NO: 10)
MESVIFSINGEIIQVNKEIITASPYNFFKRIQDHHLKDEATILNGINYHAF
ESLLDYMRWKKINITINNVEMILVAAVIIDVPPVVDLCVKTMIHNINSTNC
IRMFNFSKRYGIKKLYNASMSEIINNITAVTSDPEFGKLSKDELTTILSHE
DVNVNHEDVTAMILLKWIHKNPNDVDIINILHPKFMTNTMRNAISLLGLTI
SKSTKPVTRNGIKHNIVVIKNSDYISTITHYSPRTEYWTIVGNTDRQFYNA
NVLHNCLYIIGGMINNRHVYSVSRVDLETKKWKTVTNMSSLKSEVSTCVND
GKLYVIGGLEFSISTGVAEYLKHGTSKWIRLPNLITPRYSGASVFVNDDIY
VMGGVYTTYEKYVVLNDVECFTKNRWIKKSPMPRHHSIVYAVEYDGDIYVI
TGITHETRNYLYKYIVKEDKWIELYMYFNHVGKMFVCSCGDYILIIADAKY
EYYPKSNTWNLFDMSTRNIEYYDMFTKDETPKCNVTHKSLPSFLSNCEKQF
LQ.

As used herein, “F1L” refers to a orthopoxvirus (e.g., vaccinia, e.g., Copenhagen) gene, such as a gene that encodes a caspase-9 inhibitor. An example of a protein sequence encoded by an exemplary F1L gene in a Copenhagen strain of the vaccinia virus is given in UniProtKB database entry P68450 and is reproduced below:

(SEQ ID NO: 11)
MLSMFMCNNIVDYVDDIDNGIVQDIEDEASNNVDHDYVYPLPENMVYRFDK
STNILDYLSTERDHVMMAVRYYMSKQRLDDLYRQLPTKTRSYIDIINIYCD
KVSNDYNRDMNIMYDMASTKSFTVYDINNEVNTILMDNKGLGVRLATISFI
TELGRRCMNPVKTIKMFTLLSHTICDDCFVDYITDISPPDNTIPNTSTREY
LKLIGITAIMFATYKTLKYMIG.

As used herein, “F2L” refers to an orthopoxvirus (e.g., vaccinia, e.g., Copenhagen) gene, such as a gene that encodes a deoxyuridine triphosphatase (dUTPase). An example of a protein sequence encoded by an exemplary F2L gene in a Copenhagen strain of the vaccinia virus is given in UniProtKB database entry P68634 and is reproduced below:

(SEQ ID NO: 12)
MFNMNINSPVRFVKETNRAKSPTRQSPGAAGYDLYSAYDYTIPPGERQLIK
TDISMSMPKICYGRIAPRSGLSLKGIDIGGGVIDEDYRGNIGVILINNGKC
TFNVNTGDRIAQLIYQRIYYPELEEVQSLDSTNRGDQGFGSTGLR.

As used herein, “F3L” refers to an orthopoxvirus (e.g., vaccinia, e.g., Copenhagen) gene, such as a gene that encodes a kelch-like protein that is an innate immune response modifier and a virulence factor. An example of a protein sequence encoded by an exemplary F3L gene in a Copenhagen strain of the vaccinia virus is given in UniProtKB database entry P21013 and is reproduced below:

(SEQ ID NO: 13)
MPIFVNTVYCKNILALSMTKKFKTIIDAIGGNIIVNSTILKKLSPYFRTHL
RQKYTKNKDPVTRVCLDLDIHSLTSIVIYSYTGKVYIDSHNVVNLLRASIL
TSVEFIIYTCINFILRDFRKEYCVECYMMGIEYGLSNLLCHTKNFIAKHFL
ELEDDIIDNFDYLSMKLILESDELNVPDEDYVVDFVIKWYIKRRNKLGNLL
LLIKNVIRSNYLSPRGINNVKWILDCTKIFHCDKQPRKSYKYPFIEYPMNM
DQIIDIFHMCTSTHVGEVVYLIGGWMNNEIHNNAIAVNYISNNWIPIPPMN
SPRLYATGIPANNKLYVVGGLPNPTSVERWFHGDAAWVNMPSLLKPRCNPA
VASINNVIYVMGGHSETDTTTEYLLPNHDQWQFGPSTYYPHYKSCALVFGR
RLFLVGRNAEFYCESSNTWTLIDDPIYPRDNPELIIVDNKLLLIGGFYRGS
YIDTIEVYNHHTYSWNIWDGK.

As used herein, “K1L” refers to an orthopoxvirus (e.g., vaccinia, e.g., Copenhagen) gene, such as a gene that encodes an NF-κB inhibitor. An example of a protein sequence encoded by an exemplary K1L gene in a Copenhagen strain of the vaccinia virus is given in UniProtKB database entry P20632 and is reproduced below:

(SEQ ID NO: 14)
MDLSRINTWKSKQLKSFLSSKDTFKADVHGHSALYYAIADNNVRLVCTLL
NAGALKNLLENEFPLHQAATLEDTKIVKILLFSGMDDSQFDDKGNTALYY
AVDSGNMQTVKLFVKKNWRLMFYGKTGWKTSFYHAVMLNDVSIVSYFLSE
IPSTFDLAILLSCIHTTIKNGHVDMMILLLDYMTSTNTNNSLLFIPDIKL
AIDNKDIEMLQALFKYDINIYSVNLENVLLDDAEITKMIIEKHVEYKSDS
YTKDLDIVKNNKLDEIISKNKELRLMYVNCVKKN.

As used herein, “K2L” refers to an orthopoxvirus (e.g., vaccinia, e.g., Copenhagen) gene, such as a gene that encodes a serine protease inhibitor that prevents cell fusion. An example of a protein sequence encoded by an exemplary K2L gene in a Copenhagen strain of the vaccinia virus is given in UniProtKB database entry P20532 and is reproduced below:

(SEQ ID NO: 15)
MIALLILSLTCSVSTYRLQGFTNAGIVAYKNIQDDNIVFSPFGYSFSMFM
SLLPASGNTRIELLKTMDLRKRDLGPAFTELISGLAKLKTSKYTYTDLTY
QSFVDNTVCIKPLYYQQYHRFGLYRLNFRRDAVNKINSIVERRSGMSNVV
DSNMLDNNTLWAIINTIYFKGTWQYPFDITKTRNASFTNKYGTKTVPMMN
VVTKLQGNTITIDDEEYDMVRLPYKDANISMYLAIGDNMTHFTDSITAAK
LDYWSFQLGNKVYNLKLPKFSIENKRDIKSIAEMMAPSMFNPDNASFKHM
TRDPLYIYKMFQNAKIDVDEQGTVAEASTIMVATARSSPEKLEFNTPFVF
IIRHDITGFILFMGKVESP.

As used herein, “K3L” refers to an orthopoxvirus (e.g., vaccinia, e.g., Copenhagen) gene, such as a gene that encodes a PKR inhibitor. An example of a protein sequence encoded by an exemplary K3L gene in a Copenhagen strain of the vaccinia virus is given in UniProtKB database entry P20639 and is reproduced below:

(SEQ ID NO: 16)
MLAFCYSLPNAGDVIKGRVYEKDYALYIYLFDYPHSEAILAESVKMHMDR
YVEYRDKLVGKTVKVKVIRVDYTKGYIDVNYKRMCRHQ.

As used herein, “K4L” refers to an orthopoxvirus (e.g., vaccinia, e.g., Copenhagen) gene, such as a gene that encodes a DNA modifying nuclease (e.g., DNA nicking enzyme). An example of a protein sequence encoded by an exemplary K4L gene in a Copenhagen strain of the vaccinia virus is given in UniProtKB database entry P20537 and is reproduced below:

(SEQ ID NO: 17)
MNPDNTIAVITETIPIGMQFDKVYLSTFNMWREILSNTTKTLDISSFYWS
LSDEVGTNFGTIILNEIVQLPKRGVRVRVAVNKSNKPLKDVERLQMAGVE
VRYIDITNILGGVLHTKFWISDNTHIYLGSANMDWRSLTQVKELGIAIFN
NRNLAADLTQIFEVYWYLGVNNLPYNWKNFYPSYYNTDHPLSINVSGVPH
SVFIASAPQQLCTMERTNDLTALLSCIRNASKFVYVSVMNFIPIIYSKAG
KILFWPYIEDELRRSAIDRQVSVKLLISCWQRSSFIMRNFLRSIAMLKSK
NIDIEVKLFIVPDADPPIPYSRVNHAKYMVTDKTAYIGTSNWTGNYFTDT
CGASINITPDDGLGLRQQLEDIFMRDWNSKYSYELYDTSPTKRCKLLKNM
KQCTNDIYCDEIQPEKEIPEYSLE.

As used herein, “K5L” refers to an orthopoxvirus (e.g., vaccinia, e.g., Copenhagen) gene, such as a gene that encodes a putative monoglyceride lipase. An example of a protein sequence encoded by an exemplary K5L gene in a Copenhagen strain of the vaccinia virus is given in UniProtKB database entry P21084 and is reproduced below:

(SEQ ID NO: 18)
MGATISILASYDNPNLFTAMILMSPLVNADAVSRLNLLAAKLMGTITPNA
PVGKLCPESVSRDMDKVYKYQYDPLINHEKIKAGFASQVLKATNKVRKII
SKINTPRLSYSREQTMRLVMFQVHIISCNMQIVIEK.

As used herein, “K6L” refers to an orthopoxvirus (e.g., vaccinia, e.g., Copenhagen) gene, such as a gene that encodes a putative monoglyceride lipase. An example of a protein sequence encoded by an exemplary K6L gene in a Copenhagen strain of the vaccinia virus is given in UniProtKB database entry P68465 and is reproduced below:

(SEQ ID NO: 19)
MSANCMFNLDNDYIYWKPITYPKALVFISHGAGKHSGRYDELAENISSLG
ILVFSHDHIGHGRSNGEKMMIDDFGTARGNY.

As used herein, “K7R” refers to an orthopoxvirus (e.g., vaccinia, e.g., Copenhagen) gene, such as a gene that encodes an inhibitor of NF-κB and IRF3. An example of a protein sequence encoded by an exemplary K7R gene in a Copenhagen strain of the vaccinia virus is given in UniProtKB database entry P68467 and is reproduced below:

(SEQ ID NO: 20)
MATKLDYEDAVFYFVDDDKICSRDSIIDLIDEYITWRNHVIVFNKDITSC
GRLYKELMKFDDVAIRYYGIDKINEIVEAMSEGDHYINFTKVHDQESLFA
TIGICAKITEHWGYKKISESRFQSLGNITDLMTDDNINILILFLEKKLN.

As used herein, “M1L” refers to an orthopoxvirus (e.g., vaccinia, e.g., Copenhagen) gene, such as a gene that encodes an Ankyrin repeat protein. An example of a protein sequence encoded by an exemplary M1L gene in a Copenhagen strain of the vaccinia virus is given in UniProtKB database entry P20640 and is reproduced below:

(SEQ ID NO: 21)
MIFVIESKLLQIYRNRNRNINFYTTMDNIMSAEYYLSLYAKYNSKNLDVF
RNMLQAIEPSGNNYHILHAYCGIKGLDERFVEELLHRGYSPNETDDDGNY
PLHIASKINNNRIVAMLLTHGADPNACDKHNKTPLYYLSGTDDEVIERIN
LLVQYGAKINNSVDEEGCGPLLACTDPSERVFKKIMSIGFEARIVDKFGK
NHIHRHLMSDNPKASTISWMMKLGISPSKPDHDGNTPLHIVCSKTVKNVD
IIDLLLPSTDVNKQNKFGDSPLTLLIKTLSPAHLINKLLSTSNVITDQTV
NICIFYDRDDVLEIINDKGKQYDSTDFKMAVEVGSIRCVKYLLDNDIICE
DAMYYAVLSEYETMVDYLLFNHFSVDSVVNGHTCMSECVRLNNPVILSKL
MLHNPTSETMYLTMKAIEKDKLDKSIIIPFIAYFVLMHPDFCKNRRYFTS
YKRFVTDYVHEGVSYEVFDDYF.

As used herein, “M2L” refers to an orthopoxvirus (e.g., vaccinia, e.g., Copenhagen) gene, such as a gene that encodes an inhibitor of NF-κB and apoptosis. An example of a protein sequence encoded by an exemplary M2L gene in a Copenhagen strain of the vaccinia virus is given in UniProtKB database entry Q1PJ18 and is reproduced below:

(SEQ ID NO: 22)
MVYKLVLLFCIASLGYSVEYKNTICPPRQDYRYWYFAAELTIGVNYDINS
TIIGECHMSESYIDRNANIVLTGYGLEINMTIMDTDQRFVAAAEGVGKDN
KLSVLLFTTQRLDKVHHNISVTITCMEMNCGTTKYDSDLPESIHKSSSCD
ITINGSCVTCVNLETDPTKINPHYLHPKDKYLYHNSEYGMRGSYGVTFID
ELNQCLLDIKELSYDICYRE.

As used herein, “N1L” refers to an orthopoxvirus (e.g., vaccinia, e.g., Copenhagen) gene, such as a gene that encodes a BCL-2-like protein that inhibits NF-κB and apoptosis. An example of a protein sequence encoded by an exemplary N1L gene in a Copenhagen strain of the vaccinia virus is given in UniProtKB database entry P21054 and is reproduced below:

(SEQ ID NO: 23)
MRTLLIRYILWRNDNDQTYYNDDFKKLMLLDELVDDGDVCTLIKNMRMTL
SDGPLLDRLNQPVNNIEDAKRMIAISAKVARDIGERSEIRWEESFTILFR
MIETYFDDLMIDLYGEK.

As used herein, “N2L” refers to an orthopoxvirus (e.g., vaccinia, e.g., Copenhagen) gene, such as a gene that encodes an inhibitor of IRF3. An example of a protein sequence encoded by an exemplary N2L gene in a Copenhagen strain of the vaccinia virus is given in UniProtKB database entry P20641 and is reproduced below:

(SEQ ID NO: 26)
MTSSAMDNNEPKVLEMVYDATILPEGSSMDPNIMDCINRHINMCIQRTYS
SSIIAILNRFLTMNKDELNNTQCHIIKEFMTYEQMAIDHYGEYVNAILYQ
IRKRPNQHHTIDLFKKIKRTPYDTFKVDPVEFVKKVIGFVSILNKYKPVY
SYVLYENVLYDEFKCFINYVETKYF.

Exemplary Copenhagen strain nucleotide sequences of the coding sequences (CDSs) of the genes described herein are provided in Table 42 below. The nucleotide sequence of an exemplary wild-type Copenhagen strain vaccinia virus genome is also provided in Table 42 below. Another exemplary wild-type Copenhagen strain vaccinia virus genome is SEQ ID NO: 590 (as provided in Table 42) but with 1, 2, 3, 4, 5, 6, 7, 8, 9, 10, 11, 12, 13, 14, 15, 16, 17, 18, 19, 20, or all of the nucleotide polymorphisms identified in Table 46. In certain embodiments, the CDS of the genes described herein have nucleotide sequences that are identical to the nucleotide sequences provided in Table 42 except for 1, 2, 3, or more of the nucleotide polymorphisms identified in Table 46.

### 4. BRIEF DESCRIPTION OF THE FIGURES

FIG. 1 shows a phylogenetic analysis of 59 poxvirus strains, including the Orthopoxvirus virus strains.

FIG. 2 shows the abundances of different viral strains after passaging 5 Vaccinia viruses in different tumor types.

FIG. 3 shows the abilities of Vaccinia wild-type strains to replicate in various different patient tumor cores.

FIG. 4 shows plaque size measurements of different Vaccinia wild-type strains.

FIG. 5 shows the genomic structure of a 5p deletion (CopMD5p) and a 3p deletion (CopMD3p). Both CopMD5p and CopMD3p were crossed to generate CopMD5p3p.

FIG. 6 shows a heatmap showing cancer cell death following infection with either Copenhagen or CopMD5p3p at various doses.

FIG. 7 shows the growth curves of Copenhagen and CopMD5p3p replication in 4 different cancer cell lines.

FIG. 8 shows the ability of Copenhagen and CopMD5p3p to replicate in patient ex vivo samples as shown by titering.

FIG. 9 shows that the modified CopMD5p3p virus forms different plaques than the parental virus. CopMD5p3p plaques are much clearer in the middle, with visible syncytia (cell fusion).

FIG. 10 shows CopMD5p3p induces syncytia (cell fusion) in 786-O cells.

FIG. 11 shows that CopMD5p3p is able to control tumor growth similarly to Copenhagen wild-type but does not cause weight loss.

FIG. 12 shows that CopMD5p3p does not cause pox lesion formation when compared to two other Vaccinia strains (Copenhagen and Wyeth) harboring the oncolytic knockout of thymidine kinase.

FIG. 13 shows the IVIS bio-distribution of Vaccinia after systemic administration in nude CD-1 mice. Luciferase encoding CopMD5p3p (TK KO) is tumor specific and does not replicate in off target tissues.

FIG. 14 shows the bio-distribution of Vaccinia after systemic administration. CopMD5p3p replicates similarly to other oncolytic Vaccinia in the tumour but replicates less in off target tissues/organs.

FIG. 15 shows the immunogenicity of Vaccinia in Human PBMCs. The ability of CopMD5p3p to induce human innate immune cell activation is stronger than that of wild-type Copenhagen.

FIG. 16 shows the immunogenicity of Vaccinia in Mouse Splenocytes. The ability of CopMD5p3p to induce mouse innate immune cell activation is stronger than that of Copenhagen.

FIG. 17 shows the immunogenicity of Vaccinia in Human cells. The ability of CopMD5p3p to activate NF-kB immune transcription factor is stronger than that of Copenhagen or VVdd but similar to that of MG-1.

FIG. 18 shows the synergy with immune checkpoint inhibitor Anti-CTLA-4 antibody in an aggressive melanoma model (B16-F10 syngeneic melanoma model in C57BL6 mice). In vivo efficacy measured by survival in an immune competent murine model treated with Vaccinia and Immune Checkpoint Inhibitors Anti-CTLA-4 antibody.

FIG. 19 shows the synergy with immune checkpoint inhibitor Anti-CTLA-4 antibody. In vivo efficacy measured by tumor growth (top row) and survival (bottom row) in an immune competent murine model treated with Vaccinia and Immune Checkpoint Inhibitor Anti-CTLA-4 antibody. CopMD5p3p (left column) is compared to oncolytic Copenhagen TK KO (right column).

FIG. 20 shows the synergy with immune checkpoint inhibitor Anti-PD1 antibody. In vivo efficacy measured by tumor growth (top row) and survival (bottom row) in an immune competent murine model treated with Vaccinia and Immune Checkpoint Inhibitor Anti-PD1 antibody. CopMD5p3p (left column) is compared to oncolytic Copenhagen TK KO (right column).

FIG. 21 shows the synergy with immune checkpoint inhibitor Anti-PD1 antibody and Anti-CTLA-4 antibody. In vivo efficacy measured by tumor growth (top row) and survival (bottom row) in an immune competent murine model treated with Vaccinia and Immune Checkpoint Inhibitors Anti-PD1 antibody and Anti-CTLA-4 antibody. CopMD5p3p (left column) is compared to oncolytic Copenhagen TK KO (right column).

FIG. 22 shows a scheme for the production of modified poxvirus vectors (e.g., modified vaccinia virus vectors, such as modified Copenhagen vaccinia virus vectors) harboring a 5′ (“5p”) major deletion locus (left) and a 3′ (“3p”) major deletion locus (right). 5p targeting construct is composed of 1 kb homologous region to C2L, followed by an eGFP expressing transgene, and 1 kb homologous region to F3L. 3p targeting construct is composed of 729bp homologous region to B14R, followed by the mCherry expressing transgene, and a 415bp homologous region to B29R.

FIG. 23 shows the ability of wild-type Copenhagen vaccinia virus and several modified Copenhagen vaccinia virions to proliferate in various cell lines.

FIG. 24 shows the cytotoxic effects of wild-type Copenhagen vaccinia virus and several modified Copenhagen vaccinia virions on various cell lines, as assessed by crystal violet (upper panels) and an Alamar Blue assay (lower panel). The order of strains listed for each cell line along the x-axis of the chart shown in the lower panel is as follows: from left to right, CopMD5p, CopMD5p3p, CopMD3p, and CopWT (wildtype Copenhagen vaccinia strain).

FIG. 25 shows the distribution of wild-type Copenhagen vaccinia virus and several modified Copenhagen vaccinia virions upon administration to mice.

FIG. 26 shows the ability of wild-type Copenhagen vaccinia virus and several modified Copenhagen vaccinia virions to activate Natural Killer (NK) cells and promote anti-tumor immunity.

FIG. 27 shows the ability of wild-type Copenhagen vaccinia virus and several modified Copenhagen vaccinia virions to enhance NK cell-mediated degranulation against HT29 cells, a measure of NK cell activity and anti-tumor immunity.

FIG. 28 shows the ability of wild-type Copenhagen vaccinia virus and several modified Copenhagen vaccinia virions to prime T-cells to initiate an anti-tumor immune response.

FIG. 29 shows the ability of wild-type Copenhagen vaccinia virus and several modified Copenhagen vaccinia virions to spread to distant locations from the initial point of infection.

FIG. 30 shows the ability of wild-type Copenhagen vaccinia virus and several modified Copenhagen vaccinia virions to form plaques, a measure of viral proliferation.

FIG. 31 shows the ability of wild-type Copenhagen vaccinia virus and several modified Copenhagen vaccinia virions to form plaques in U2OS cells.

FIG. 32 shows the ability of wild-type Copenhagen vaccinia virus and several modified Copenhagen vaccinia virions to form plaques in 786-O cells.

FIG. 33 shows the percentage of genes deleted in CopMD5p3p in various poxvirus genomes.

FIG. 34 shows infection of normal versus cancer cell lines of SKV-B8R+ virus.

FIG. 35 shows SKV-B8R+ does not impair interferon signaling.

FIG. 36 shows B8R recombination targeting strategy for FLt3-LG and IL-12-TM transgenes.

FIG. 37 shows SKV (CopMD5p3p-B8R−) has similar efficacy in tumour control compared to SKV-B8R+.

FIG. 38 shows a linear cartoon schematic depicting the genomic organization of the SKV-123v2 oncolytic platform compared to the base wildtype Copenhagen vaccinia virus genome. FRT is a recognition site for the Flippase enzyme.

FIG. 39 shows SKV engineered to express 2 immunotherapeutic transgenes and an antibody.

FIG. 40 shows SKV engineered to express 2 immunotherapeutic transgenes and an antibody.

FIG. 41 shows hIL-12 production quantified for various SKV viruses expressing transgenes.

FIG. 42 shows IL-12p35 (IL-12) cell surface immunostaining on live Vero cells infected with SKV-123, SKV-3 and control SKV-eGFP viruses (MOI 0.1, 24 hrs post infection).

FIG. 43 shows SKV expressing murine IL-12 p35 membrane bound has greater efficacy in controlling murine tumors.

FIG. 44 shows major double deletions engineered in various vaccinia strains enhance cancer cell killing in vitro.

FIG. 45 shows the phenotypic characterization of HeLa cells infected with various vaccinia strains.

FIG. 46 shows 5p3p vaccinia strains do not induce weight loss compared to wildtype strains. Mouse body mass measurements are shown. CD-1 nude mice were treated with 1×10⁷ pfu (particle forming units) via intravenously tail vein injection and measured at the indicated time points.

FIG. 47 shows 5p3p vaccinia strains do not induce pox lesions compared to wildtype strains. Assessment of the presence of pox lesions is shown. CD-1 nude mice were treated with 1×10⁷ pfu with indicated vaccinia virus strains via intravenously tail vein injection. Mice were examined for pox lesions 6 days post-injection.

FIGS. 48A-48H show tumor volume over time and survival curves in eight xenograft mouse models treated with 0.05 ml of SKV (vaccinia virus) (dose: 1e7 pfu). FIG. 48A shows results from the MiaPaca-2 xenograft mouse model. FIG. 48B shows results from the PC-3 xenograft mouse model. FIG. 48C shows results from the U87MG xenograft model. FIG. 49D shows results from the UACC-62 xenograft model. FIG. 48E shows results from the UM-UC-3 xenograft mouse model. FIG. 48F shows results from the COLO-205 xenograft mouse model. FIG. 48G shows results from the NCI-H460 xenograft mouse model. FIG. 48H shows results from the HT29 xenograft model.

FIG. 49 shows average tumor volumes over time and survival curves in a transgenic C57/BL6 mouse model expressing human CTLA-4, with MC-38 tumors treated with SKV encoding active transgenes. Animals were then randomized into 5 treatment groups and then treated with PBS, PBS plus Ipilimumab, SKV, anti-PD-1 antibody, SKV-12m3v2-eGFP or SKV-12m3v2-eGFP plus anti-PD-1 antibody. SKV-12m3v2-eGFP is SKV expressing the human anti-CTLA-4 antibody, human Flt3 ligand and mouse IL-12 TM p35.

FIG. 50 shows individual tumor volumes of the experiment shown in FIG. 49.

FIG. 51 shows average tumor volumes over time in MC-38 mouse models treated with either membrane-bound mouse IL-12 p35 or membrane-bound mouse IL-12 p70.

FIG. 52 shows results from a heterologous prime:boost oncolytic vaccine regimen using a virus (SKVB^{8R+TK-} encoding OVA antigen).

FIGS. 53A-53F show the biodistribution of FLT3-L and Anti-CTLA-4 Antibody in serum and tissue of BALB/c mice engrafted with CT26 tumor cells and administered SKV-123v2 either IT or IV.

FIGS. 54A-54D show the biodistribution of IL-12-TM in serum and tissue of BALB/c mice engrafted with CT26 tumor cells and administered SKV-123v2 either IT or IV.

FIG. 55 shows tumor volume in NGS mice either untreated or treated with SKV-123v2.

FIG. 56 shows Alamar Blue viability kinetics of SKV-123v2 virus-infected cancer cells (top panels) and normal cells (bottom panels).

FIG. 57 shows virus replication growth curves in SKV-123v2 virus-infected cancer cells (786-O, HeLa) and normal cells (PBMC, PrEC).

FIG. 58 shows anti-CTLA-4 antibody expression levels in SKV-123v2 virus-infected cancer cells (786-O, HeLa) and normal cells (PBMC, PrEC).

FIG. 59 shows FLT3L expression levels in SKV-123v2 virus-infected cancer cells (786-O, HeLa) and normal cells (PBMC, PrEC).

FIG. 60 shows the design of a targeting construct for insertion of a transgene in vaccinia virus genome. Construct can either be a PCR product of amplification or part of a bacterial plasmid. Number of transgenes as well as their orientation are flexible. Order of transgenes and Fluorescent marker are flexible.

### 5. DETAILED DESCRIPTION

The present invention features genetically modified orthopoxviruses, such as vaccinia viruses (e.g., Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1 viruses), as well as the use of the same for the treatment of various cancers. The invention is based in part on the discovery that orthopoxviruses, such as Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1 viruses, exhibit markedly improved oncolytic activity, replication in tumors, infectivity, immune evasion, tumor persistence, capacity for incorporation of exogenous DNA sequences, and amenability for large scale manufacturing when the viruses are engineered to contain deletions in one or more, or all, of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, K ORF A, K ORF B, B ORF E, B ORF F, and B ORF G genes and copies of the B21R, B22R, B23R, B24R, B25R, B26R, B27R, B28R, and B29R ITRs. In various embodiments of the invention, the modified orthopoxviruses contain a deletion of the B8R gene. While inactive in mice, the B8R gene neutralizes antiviral activity of human IFN-γ. In various embodiments, at least one transgene is subsequently inserted into locus of the B8R gene (now deleted) through a homologous recombination targeting strategy. In various embodiments, the modified orthopoxvirus expresses at least one of three transgenes: IL-12-TM, FLT3-L and anti-CLTA4 antibody. As used herein, FLT3L, Flt-3 ligand, FLT3LG, FLT3-LG, FLT3-L are synonyms and all refer to FMS-like tyrosine kinase 3 ligand.

The orthopoxviruses described herein can be administered to a patient, such as a mammalian patient (e.g., a human patient) to treat a variety of cell proliferation disorders, including a wide range of cancers. The sections that follow describe orthopoxviruses and genetic modifications thereto, as well as methods of producing and propagating genetically modified orthopoxviruses and techniques for administering the same to a patient.

### 5.1. Poxvirus

Generally, a poxvirus viral particle is oval or brick-shaped, measuring some 200-400 nm long. The external surface is ridged in parallel rows, sometimes arranged helically. Such particles are extremely complex, containing over 100 distinct proteins. The extracellular forms contain two membranes (EEV: extracellular enveloped virions), whereas intracellular particles only have an inner membrane (IMV: intracellular mature virions). The outer surface is composed of lipid and protein that surrounds the core, which is composed of a tightly compressed nucleoprotein. Antigenically, poxviruses are also very complex, inducing both specific and cross-reacting antibodies. There are at least ten enzymes present in the particle, mostly concerned with nucleic acid metabolism/genome replication.

The genome of the wild-type poxvirus is linear double-stranded DNA of 130-300 Kbp. The ends of the genome have a terminal hairpin loop with several tandem repeat sequences. Several poxvirus genomes have been sequenced, with most of the essential genes being located in the central part of the genome, while non-essential genes are located at the ends. There are about 250 genes in the poxvirus genome. Replication takes place in the cytoplasm, as the virus is sufficiently complex to have acquired all the functions necessary for genome replication. There is some contribution by the cell, but the nature of this contribution is not clear. However, even though poxvirus gene expression and genome replication occur in enucleated cells, maturation is blocked, indicating some role by the cell.

Once into the cell cytoplasm, gene expression is carried out by viral enzymes associated with the core. Expression is divided into 2 phases: early genes, which represent about of 50% genome, and are expressed before genome replication, and late genes, which are expressed after genome replication. The temporal control of expression is provided by the late promoters, which are dependent on DNA replication for activity. Genome replication is believed to involve self-priming, leading to the formation of high molecular weight concatemers, which are subsequently cleaved and repaired to make virus genomes. Viral assembly occurs in the cytoskeleton and probably involves interactions with the cytoskeletal proteins (e.g., actin-binding proteins). Inclusions form in the cytoplasm that mature into virus particles. Cell to cell spread may provide an alternative mechanism for spread of infection. Overall, replication of this large, complex virus is rather quick, taking just 12 hours on average. At least nine different poxviruses cause disease in humans, but variola virus and vaccinia are the best known. Variola strains are divided into variola major (25-30% fatalities) and variola minor (same symptoms but less than 1% death rate). Infection with both viruses occurs naturally by the respiratory route and is systemic, producing a variety of symptoms, but most notably with variola characteristic pustules and scarring of the skin.

### 5.2. Orthopoxvirus

### 5.2.1. Vaccinia Virus

Vaccinia virus is a member of the poxvirus or Poxviridae family, the Chordopoxyirinae subfamily, and the Orthopoxvirus genus. Orthopoxvirus is relatively more homogeneous than other members of the Chordopoxyirinae subfamily and includes 11 distinct but closely related species, which includes vaccinia virus, variola virus (causative agent of smallpox), cowpox virus, buffalopox virus, monkeypox virus, mousepox virus and horsepox virus species as well as others (see Moss, 1996).

Vaccinia virus is a large, complex enveloped virus having a linear double-stranded DNA genome of about 190 kb and encoding approximately 250 genes. Vaccinia is well-known for its role as a vaccine that eradicated smallpox. Post-eradication of smallpox, scientists have been exploring the use of vaccinia as a tool for delivering genes into biological tissues (gene therapy and genetic engineering). Vaccinia virus is unique among DNA viruses as it replicates only in the cytoplasm of the host cell. Therefore, a large genome is required to encode various enzymes and proteins needed for viral DNA replication. During replication, vaccinia produces several infectious forms, which differ in their outer membranes: the intracellular mature virion (IMV), the intracellular enveloped virion (IEV), the cell-associated enveloped virion (CEV), and the extracellular enveloped virion (EEV). IMV is the most abundant infectious form and is thought to be responsible for spread between hosts. On the other hand, the CEV is believed to play a role in cell-to-cell spread, and the EEV is thought to be important for long range dissemination within the host organism.

Vaccinia virus is closely related to the virus that causes cowpox. The precise origin of vaccinia is unknown, but the most common view is that vaccinia virus, cowpox virus, and variola virus (the causative agent for smallpox) were all derived from a common ancestral virus. There is also speculation that vaccinia virus was originally isolated from horses. A vaccinia virus infection is mild and typically asymptomatic in healthy individuals, but it may cause a mild rash and fever, with an extremely low rate of fatality. An immune response generated against a vaccinia virus infection protects that person against a lethal smallpox infection. For this reason, vaccinia virus was used as a live-virus vaccine against smallpox. The vaccinia virus vaccine is safe because it does not contain the smallpox virus, but occasionally certain complications and/or vaccine adverse effects may arise, especially if the vaccine is immunocompromised.

Exemplary strains of the vaccinia virus include, but are not limited to, Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1.

### 5.2.2. Thymidine Kinase Mutants and Hemagglutinin Mutants

Several current clinical studies testing vaccinia virus as an oncolytic virus harbor deletions in the viral Thymidine Kinase (TK) gene. This deletion attenuates the virus, rendering the virus dependent upon the activity of cellular thymidine kinase for DNA replication and, thus, viral propagation. Cellular thymidine kinase is expressed at a low level in most normal tissues and at elevated levels in many cancer cells. Through metabolic targeting, TK-viruses can grow in cells that have a high metabolic rate (e.g., healthy cells or tumor cells) and will not grow well in cells that have low levels of thymidine kinase. Since there exist quiescent tumor cells (e.g., cancer stem cells), TK-iruses are likely compromised in their ability to kill this population of cancer cells just as chemotherapy is largely ineffective. In some embodiments, the modified viral vectors described in this disclosure retains virus synthetic machinery (including TK) and may propagate in quiescent cancer cells. In such embodiments, the viral modifications of this disclosure may allow the virus to be highly selective without deleting TK or other DNA metabolizing enzymes (e.g., ribonucleotide reductase) and could be more effective in tumors with a low metabolic rate. In some embodiments, the modified viral vectors described in this disclosure comprise a functional TK gene (for example, a wild-type TK gene). In other embodiments, the modified viral vectors described in this disclosure comprise a deletion(s) or loss-of-function mutation(s) in the TK gene.

Similarly, inactivation of the hemagglutinin (HA) gene of the vaccinia virus can result in attenuation of the virus. In some embodiments, the modified viral vectors described in this disclosure comprise a functional HA gene (for example, a wild-type HA gene). In other embodiments, the modified viral vectors described in this disclosure comprise a deletion(s) or loss-of-function mutation(s) in the HA gene.

In a specific embodiment, the modified viral vectors described in this disclosure comprise a functional TK gene (for example, a wild-type TK gene) and a functional HA gene (for example, a wild-type HA gene). In another specific embodiment, the modified viral vectors described in this disclosure comprise a functional TK gene (for example, a wild-type TK gene) and a deletion(s) or loss-of-function mutation(s) in the HA gene. In another specific embodiment, the modified viral vectors described in this disclosure comprise a deletion(s) or loss-of-function mutation(s) in the TK gene and a functional HA gene (for example, a wild-type HA gene). In another specific embodiment, the modified viral vectors described in this disclosure comprise a deletion(s) or loss-of-function mutation(s) in the TK gene and a deletion(s) or loss-of-function mutation(s) in the HA gene.

### 5.2.3. Recombinant Orthopoxvirus Genome

In one aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ inverted terminal repeat (ITR): B21R, B22R, B23R, B24R, B25R, B26R, B27R, B28R, and B29R; and (c) a first transgene comprising a first nucleotide sequence encoding an antibody or antigen-binding fragment thereof that specifically binds to Cytotoxic T-lymphocyte Associated Protein 4 (CTLA-4); wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter (e.g., an early H5R promoter, a late H5R promoter, or an early H5R promoter and a late H5R promoter).

In some embodiments, when the flanking endogenous vaccinia virus genes have the same orientation, the first nucleotide sequence is in the same orientation as the flanking endogenous vaccinia virus genes. In other embodiments, when the flanking endogenous vaccinia virus genes have the same orientation, the first nucleotide sequence is in the reverse orientation relative to the flanking endogenous vaccinia virus genes. In other embodiments, when the flanking endogenous vaccinia virus genes have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene.

In preferred embodiments, endogenous genes that flank a nucleotide sequence (i.e., the flanking endogenous genes of a nucleotide sequence) in this disclosure are the two endogenous genes closest to the nucleotide sequence (with one upstream and the other downstream of the nucleotide sequence). The endogenous genes can be partial genes or full-length genes.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); and (d) a nucleotide sequence comprising at least one promoter operably linked to the first nucleotide sequence, wherein the at least one promoter operably linked to the first nucleotide sequence is an H5R promoter, a pS promoter, or a LEO promoter; wherein the deletions in the C2L, F3L, B14R, and B29R vaccinia genes are partial deletions. In specific embodiments, the nucleic acid further comprises a deletion in the B8R gene.

In certain embodiments, the at least one promoter operably linked to the first nucleotide sequence is an H5R promoter (e.g., an early H5R promoter, a late H5R promoter, or an early H5R promoter and a late H5R promoter).

In some embodiments, when the flanking endogenous vaccinia virus genes have the same orientation, the first nucleotide sequence is in the same orientation as the flanking endogenous vaccinia virus genes. In other embodiments, when the flanking endogenous vaccinia virus genes have the same orientation, the first nucleotide sequence is in the reverse orientation relative to the flanking endogenous vaccinia virus genes. In other embodiments, when the flanking endogenous vaccinia virus genes have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; and (c) a second transgene comprising a second nucleotide sequence encoding an Interleukin 12 (IL-12) polypeptide; wherein the deletions in the C2L, F3L, B14R, and B29R vaccinia genes are partial deletions.

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, when the flanking endogenous vaccinia virus genes have the same orientation, the second nucleotide sequence is in the same orientation as the flanking endogenous vaccinia virus genes. In other embodiments, when the flanking endogenous vaccinia virus genes have the same orientation, the second nucleotide sequence is in the reverse orientation relative to the flanking endogenous vaccinia virus genes. In other embodiments, when the flanking endogenous vaccinia virus genes have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B 16R, B17L, B18R, B 19R, and B20R; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a nucleotide sequence comprising at least one promoter operably linked to the second nucleotide sequence, wherein the at least one promoter operably linked to the second nucleotide sequence is a late promoter; wherein the deletions in the C2L, F3L, B14R, and B29R vaccinia genes are partial deletions. In specific embodiments, the nucleic acid further comprises a deletion in the B8R gene.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, when the flanking endogenous vaccinia virus genes have the same orientation, the second nucleotide sequence is in the same orientation as the flanking endogenous vaccinia virus genes. In other embodiments, when the flanking endogenous vaccinia virus genes have the same orientation, the second nucleotide sequence is in the reverse orientation relative to the flanking endogenous vaccinia virus genes. In other embodiments, when the flanking endogenous vaccinia virus genes have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; and (c) a third transgene comprising a third nucleotide sequence encoding FMS-like tyrosine kinase 3 ligand (FLT3L); wherein the deletions in the C2L, F3L, B14R, and B29R vaccinia genes are partial deletions.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, and/or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, when the flanking endogenous vaccinia virus genes have the same orientation, the third nucleotide sequence is in the same orientation as the flanking endogenous vaccinia virus genes. In other embodiments, when the flanking endogenous vaccinia virus genes have the same orientation, the third nucleotide sequence is in the reverse orientation relative to the flanking endogenous vaccinia virus genes. In other embodiments, when the flanking endogenous vaccinia virus genes have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a third transgene comprising a third nucleotide sequence encoding FLT3L; and (d) a nucleotide sequence comprising at least one promoter operably linked to the third nucleotide sequence, wherein the at least one promoter operably linked to the third nucleotide sequence is a B8R promoter, a B 19R promoter, a E3L promoter, an F11L promoter, and/or a B2R promoter; wherein the deletions in the C2L, F3L, B14R, and B29R vaccinia genes are partial deletions. In specific embodiments, the nucleic acid further comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, when the flanking endogenous vaccinia virus genes have the same orientation, the third nucleotide sequence is in the same orientation as the flanking endogenous vaccinia virus genes. In other embodiments, when the flanking endogenous vaccinia virus genes have the same orientation, the third nucleotide sequence is in the reverse orientation relative to the flanking endogenous vaccinia virus genes. In other embodiments, when the flanking endogenous vaccinia virus genes have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes are the C2L and F3L genes. In another specific embodiment, the flanking endogenous vaccinia virus genes are the C3L and F4L genes. In another specific embodiment, the flanking endogenous vaccinia virus genes are the B14R and B29R genes. In another specific embodiment, the flanking endogenous vaccinia virus genes are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); and (d) a second transgene comprising a second nucleotide sequence encoding an IL-12 polypeptide; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter (e.g., an early H5R promoter, a late H5R promoter, or an early H5R promoter and a late H5R promoter).

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene.

In specific embodiments, the anti-CTLA-4 antibody or antigen-binding fragment thereof encoded by the first nucleotide sequence comprises the 6 complementarily-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); and (d) a second transgene comprising a second nucleotide sequence encoding an IL-12 polypeptide; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or (ii) a nucleotide sequence comprising at least one promoter operably linked to the second nucleotide sequence, wherein the at least one promoter operably linked to the second nucleotide sequence is a late promoter. In specific embodiments, the nucleic acid further comprises a deletion in the B8R gene.

In certain embodiments, the at least one promoter operably linked to the first nucleotide sequence is an H5R promoter (e.g., an early H5R promoter, a late H5R promoter, or an early H5R promoter and a late H5R promoter). In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); and (d) a third transgene comprising a third nucleotide sequence encoding FLT3L; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter (e.g., an early H5R promoter, a late H5R promoter, or an early H5R promoter and a late H5R promoter).

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, and/or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B 16R, B17L, B18R, B 19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); and (d) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, and/or a B2R promoter. In specific embodiments, the nucleic acid further comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the first nucleotide sequence is an H5R promoter (e.g., an early H5R promoter, a late H5R promoter, or an early H5R promoter and a late H5R promoter).

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a third transgene comprising a third nucleotide sequence encoding FLT3L; wherein the deletions in the C2L, F3L, B14R, and B29R vaccinia genes are partial deletions. In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, and/or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided here is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B 16R, B17L, B18R, B 19R, and B20R; (b) deletions in the following genes in the 3′ ITR: B21R, B22R, B23R, B24R, B25R, B26R, B27R, B28R, and B29R; (c) a second transgene comprising a second nucleotide sequence encoding an IL-12 polypeptide; and (d) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the second nucleotide sequence, wherein the at least one promoter operably linked to the second nucleotide sequence is a late promoter; and/or (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, and/or a B2R promoter. In specific embodiments, the nucleic acid further comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene. In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions.

In certain embodiments, the nucleic acid further comprises a nucleotide sequence comprising at least one promoter operably linked to the first nucleotide sequence. In a specific embodiment, the at least one promoter operably linked to the first nucleotide sequence is an H5R promoter, a pS promoter, or a LEO promoter. In another specific embodiment, the at least one promoter operably linked to the first nucleotide sequence is an H5R promoter (e.g., an early H5R promoter, a late H5R promoter, or an early H5R promoter and a late H5R promoter).

In certain embodiments, the nucleic acid further comprises a nucleotide sequence comprising at least one promoter operably linked to the second nucleotide sequence. In a specific embodiment, the at least one promoter operably linked to the second nucleotide sequence is a late promoter. In a further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence. In a specific embodiment, the at least one promoter operably linked to the third nucleotide sequence is a B8R promoter, a B19R promoter, a E3L promoter, an F11L promoter, and/or a B2R promoter. In another specific embodiment, the at least one promoter operably linked to the third nucleotide sequence is a B8R promoter. In another specific embodiment, the at least one promoter operably linked to the third nucleotide sequence is a B19R promoter. In another specific embodiment, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70), and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-0, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B 16R, B17L, B18R, B 19R, and B20R; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter, a pS promoter, or a LEO promoter; (ii) a nucleotide sequence comprising at least one promoter operably linked to the second nucleotide sequence, wherein the at least one promoter operably linked to the second nucleotide sequence is a late promoter; and/or (iii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, and/or a B2R promoter. In specific embodiments, the nucleic acid further comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In certain embodiments, the at least one promoter operably linked to the first nucleotide sequence is an H5R promoter (e.g., an early H5R promoter, a late H5R promoter, or an early H5R promoter and a late H5R promoter).

In certain embodiments, the late promoter comprises the nucleotide sequence of SEQ ID NO: 561, an F17R promoter, or a D13L promoter. In a specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the at least one promoter operably linked to the third nucleotide sequence is a B8R promoter. In certain embodiments, the at least one promoter operably linked to the third nucleotide sequence is a B19R promoter. In certain embodiments, the at least one promoter operably linked to the third nucleotide sequence is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene.

In specific embodiments, the anti-CTLA-4 antibody or antigen-binding fragment thereof encoded by the first nucleotide sequence comprises the 6 complementarity-determining regions (CDRs) of ipilimumab. In specific embodiments, the first nucleotide sequence encodes an amino acid sequence comprising the amino acid sequence set forth in SEQ ID NO: 211. In specific embodiments, the first nucleotide sequence comprises the sequence set forth in SEQ ID NO: 214. In specific embodiments, the first nucleotide sequence is set forth in SEQ ID NO: 214.

In specific embodiments, the IL-12 polypeptide is membrane-bound. In specific embodiments, the IL-12 polypeptide comprises IL-12 p35 (e.g., human IL-12 p35), IL-12 p40 (e.g., human IL-12 p40) or IL-12 p70 (e.g., human IL-12 p70). In specific embodiments, the IL-12 polypeptide is membrane-bound and comprises IL-12 p35 (e.g., human IL-12 p35), or IL-12 p70 (e.g., human IL-12 p70)0, and a transmembrane domain and a cytoplasmic domain (e.g., the transmembrane and cytoplasmic domains of B7-1, TNFα, or FLT3L). In specific embodiments, the IL-12 polypeptide comprises the amino acid sequence set forth in SEQ ID NO: 212. In specific embodiments, the second nucleotide sequence comprises the sequence set forth in SEQ ID NO: 215. In specific embodiments, the second nucleotide sequence is set forth in SEQ ID NO: 215.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In a specific embodiment, the first transgene is inserted between the partial C2L and F3L vaccinia genes, and the second transgene and the third transgene are inserted into the locus of the deletion in the B8R gene. In a further specific embodiment, the third transgene is upstream of the second transgene.

In some embodiments of the various embodiments and aspects described herein, the deletion in the B8R gene is a deletion of at least 30% of the B8R gene sequence. In other embodiments, the deletion in the B8R gene is a deletion of at least 40% of the B8R gene sequence. In other embodiments, the deletion in the B8R gene is a deletion of at least 50% of the B8R gene sequence. In other embodiments, the deletion in the B8R gene is a deletion of at least 60% of the B8R gene sequence. In other embodiments, the deletion in the B8R gene is a deletion of at least 70% of the B8R gene sequence. In other embodiments, the deletion in the B8R gene is a deletion of at least 80% of the B8R gene sequence. In other embodiments, the deletion in the B8R gene is a deletion of 30% -90%, 30%-85%, 40%-90%, 40%-85%, 50%-90%, 50%-85%, 60%-90%, 60%-85%, 70%-90%, 70%-85%, 75%-90%, 75%-85%, or 80%-85% of the B8R gene sequence. In a specific embodiment, the deletion in the B8R gene is a deletion of about 75% of the B8R gene sequence. In another specific embodiment, the deletion in the B8R gene is a deletion of about 80% of the B8R gene sequence. In another specific embodiment, the deletion in the B8R gene is a deletion of about 82% of the B8R gene sequence.

For example, in some embodiments, the deletion in the B8R gene is a deletion of at least 30% of the nucleotide sequence of SEQ ID NO: 591. In other embodiments, the deletion in the B8R gene is a deletion of at least 40% of the nucleotide sequence of SEQ ID NO: 591. In other embodiments, the deletion in the B8R gene is a deletion of at least 50% of the nucleotide sequence of SEQ ID NO: 591. In other embodiments, the deletion in the B8R gene is a deletion of at least 60% of the nucleotide sequence of SEQ ID NO: 591. In other embodiments, the deletion in the B8R gene is a deletion of at least 70% of the nucleotide sequence of SEQ ID NO: 591. In other embodiments, the deletion in the B8R gene is a deletion of at least 80% of the nucleotide sequence of SEQ ID NO: 591. In other embodiments, the deletion in the B8R gene is a deletion of 30% -90%, 30%-85%, 40%-90%, 40%-85%, 50%-90%, 50%-85%, 60%-90%, 60%-85%, 70%-90%, 70%-85%, 75%-90%, 75%-85%, or 80%-85% of the nucleotide sequence of SEQ ID NO: 591. In a specific embodiment, the deletion in the B8R gene is a deletion of about 75% of the nucleotide sequence of SEQ ID NO: 591. In another specific embodiment, the deletion in the B8R gene is a deletion of about 80% of the nucleotide sequence of SEQ ID NO: 591. In another specific embodiment, the deletion in the B8R gene is a deletion of about 82% of the nucleotide sequence of SEQ ID NO: 591.

In another example, in some embodiments, the deletion in the B8R gene is a deletion of at least 30% of the nucleotide sequence of ACAACACCATGAGATATATTATA ATTCTCGCAGTTTTGTTCATTAATAGTATACACGCTAAAATAACTAGTTATAAGTT TGAATCCGTCAATTTTGATTCCAAAATTGAATGGACTGGGGATGGTCTATACAAT ATATCCCTTAAAAATTATGGCATCAAGACGTGGCAAACAATGTATACAAATGTAC CAGAAGGAACATACGACATATCCGCATTTCCAAAGAATGATTTCGTATCTTTCTG GGTTAAATTTGAACAAGGCGATTATAAAGTGGAAGAGTATTGTACGGGACTATG CGTCGAAGTAAAAATTGGACCACCGACTGTAACATTGACTGAATACGACGACCA TATCAATTTGTACATCGAGCATCCGTATGCTACTAGAGGTAGCAAAAAGATTCCT ATTTACAAACGCGGTGACATGTGTGATATCTACTTGTTGTATACGGCTAACTTCA CATTCGGAGATTCTGAAGAACCAGTAACATATGATATCGATGACTACGATTGCAC GTCTACAGGTTGCAGCATAGACTTTGCCACAACAGAAAAAGTGTGCGTGACAGC ACAGGGAGCCACAGAAGGGTTTCTCGAAAAAATTACTCCATGGAGTTCGGAAGT ATGTCTGACACCTAAAAAGAATGTATATACATGTGCAATTAGATCCAAAGAAGA TGTTCCCAATTTCAAGGACAAAATGGCCAGAGTTATCAAGAGAAAATTTAATAA ACAGTCTCAATCTTATTTAACTAAATTTCTCGGTAGCACATCAAATGATGTTACC ACTTTTCTTAGCATGCTTAACTTGACTAAATATTCATAA (SEQ ID NO: 550. In other embodiments, the deletion in the B8R gene is a deletion of at least 40% of the nucleotide sequence of SEQ ID NO: 550. In other embodiments, the deletion in the B8R gene is a deletion of at least 50% of the nucleotide sequence of SEQ ID NO: 550. In other embodiments, the deletion in the B8R gene is a deletion of at least 60% of the nucleotide sequence of SEQ ID NO: 550. In other embodiments, the deletion in the B8R gene is a deletion of at least 70% of the nucleotide sequence of SEQ ID NO: 550. In other embodiments, the deletion in the B8R gene is a deletion of at least 80% of the nucleotide sequence of SEQ ID NO: 550. In other embodiments, the deletion in the B8R gene is a deletion of 30% -90%, 30%-85%, 40%-90%, 40%-85%, 50%-90%, 50%-85%, 60%-90%, 60%-85%, 70%-90%, 70%-85%, 75%-90%, 75%-85%, or 80%-85% of the nucleotide sequence of SEQ ID NO: 550. In a specific embodiment, the deletion in the B8R gene is a deletion of about 75% of the nucleotide sequence of SEQ ID NO: 550. In another specific embodiment, the deletion in the B8R gene is a deletion of about 80% of the nucleotide sequence of SEQ ID NO: 550. In another specific embodiment, the deletion in the B8R gene is a deletion of about 82% of the nucleotide sequence of SEQ ID NO: 550.

In certain embodiments, the deletion in the B8R gene does not disturb the function of the B9R gene of the vaccinia genome. In certain embodiments, the deletion in the B8R gene does not disturb the expression of the B9R gene. In certain embodiments, the deletion in the B8R gene does not remove the promoter(s) of the B9R gene. In certain embodiments, the deletion in the B8R gene does not remove the transcriptional regulatory sequences of the B9R gene. In a specific embodiment, the only sequence of the B8R gene that remains after deletion is the sequence necessary for proper B9R function and/or expression. In a specific embodiment, the deletion in the B8R gene does not remove a nucleotide sequence comprising AAAATTTAATAAACA (SEQ ID NO: 551). In another specific embodiment, the deletion in the B8R gene does not remove the nucleotide sequence AAAATTTAATAAACA (SEQ ID NO: 551). In a specific embodiment, the only sequence of the B8R gene that remains is the nucleotide sequence of

(SEQ ID NO: 552)
GATGTTCCCAATTTCAAGGACAAAATGGCCAGAGTTATCAAGAGAAAAT
TTAATAAACAGTCTCAATCTTATTTAACTAAATTTCTCGGTAGCACATC
AAATGATGTTACCACTTTTCTTAGCATGCTTAACTTGACTAAATATTCA
TAA.

In certain embodiments of the various embodiments and aspects described herein, the recombinant vaccinia virus genome is derived from the genome of a Copenhagen strain vaccinia virus. In certain embodiments of the various embodiments and aspects described herein, the recombinant vaccinia virus genome is derived from the nucleotide sequence of GenBank Accession No. M35027.1 (SEQ ID NO: 590). In certain embodiments of the various embodiments and aspects described herein, the recombinant vaccinia virus genome comprises the nucleotide sequence of GenBank Accession No. M35027.1 (SEQ ID NO: 590). In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1, 2, 3, 4, 5, 6, 7, 8, 9, 10 of the nucleotide polymorphisms identified in Table 46. In some embodiments of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1-3, 1-5, 2-4, 2-5, 1-9, 2-8, 4-8, 6-8, 1-9, 2-9, 4-9, 6-9, 7-9, 1-10, 2-10, 5-10, or 8-10 of the nucleotide polymorphisms identified in Table 46. In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 11, 12, 13, 14, 15, 16, 17, 18, 19 or 20 of the nucleotide polymorphisms identified in Table 46. In some embodiments of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 11-20, 12-15, 15-20, or 18-20 of the nucleotide polymorphisms identified in Table 46. In certain embodiments of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1-20, 1-15, 5-20, or 10-20 of the nucleotide polymorphisms identified in Table 46. In some of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises all of the nucleotide polymorphisms identified in Table 46. In certain of such embodiments, the recombinant vaccinia virus genome may be engineered to comprise 1, 2, 3, 4, 5, 6, 7, 8, 9, or 10 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise 10, 11, 12, 13, 14, 15, 16, 17, 18, 19, or 20 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In certain of such embodiments, the recombinant vaccinia virus genome may be engineered to comprise 21, 22, 23, 24, 25, 26, 27, 28, 29, 30, 31, or 32 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise deletions in C2L, C1L, N1L, N2L, M1L, M2L, K1L, K2L, K3L, K4L, K5L, K6L, K7R, F1L, F2L, F3L, B8R, B14R, B15R, B16R, B17L, B18R, and the following genes in the 3′ inverted terminal repeat (ITR): B19R, and B20R, B21R, B22R, B23R, B24R, B25R, B26R, B27R, B28R, and B29R, and insertions of one, two, or three of the transgene(s) described herein.

In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1, 2, 3, or 4 of the nucleotide polymorphisms identified in Table 46 that are synonymous variants. In some embodiments of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1, 2, 3, 4, 5, 6 or 7 of the nucleotide polymorphisms identified in Table 46 that are not in a protein coding region. In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1, 2, 3, 4, 5, 6 or 7 of the nucleotide polymorphisms identified in Table 46 that result in change in the amino acid sequence. In some embodiments of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises 1 or 2 of the nucleotide polymorphisms identified in Table 46 that result in a frameshift. In certain of such embodiments, the recombinant vaccinia virus genome may be engineered to comprise 1, 2, 3, 4, 5, 6, 7, 8, 9, or 10 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise 10, 11, 12, 13, 14, 15, 16, 17, 18, 19, or 20 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In certain of such embodiments, the recombinant vaccinia virus genome may be engineered to comprise 21, 22, 23, 24, 25, 26, 27, 28, 29, 30, 31, or 32 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise deletions in C2L, C1L, N1L, N2L, M1L, M2L, K1L, K2L, K3L, K4L, K5L, K6L, K7R, F1L, F2L, F3L, B8R, B14R, B15R, B16R, B17L, B18R, and the following genes in the 3′ inverted terminal repeat (ITR): B19R, and B20R, B21R, B22R, B23R, B24R, B25R, B26R, B27R, B28R, and B29R, and insertions of one, two, or three of the transgene(s) described herein.

In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises the nucleotide polymorphisms found in 1, 2, 3, 4, 5, 6 or 7 of the genes identified in Table 46. In some of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises of the nucleotide polymorphisms found in 8, 9, 10, 11, 12, 12 or 13 of the genes identified in Table 46. In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises the nucleotide polymorphisms nucleotide polymorphisms found in all of the genes identified in Table 46. In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises the nucleotide polymorphisms found in 1-5, 5-10, 1-13, 5-13, or 10-13 of the genes identified in Table 46. In certain of such embodiments, the recombinant vaccinia virus genome may be engineered to comprise 1, 2, 3, 4, 5, 6, 7, 8, 9, or 10 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise 10, 11, 12, 13, 14, 15, 16, 17, 18, 19, or 20 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In certain of such embodiments, the recombinant vaccinia virus genome may be engineered to comprise 21, 22, 23, 24, 25, 26, 27, 28, 29, 30, 31, or 32 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise deletions in C2L, C1L, N1L, N2L, M1L, M2L, K1L, K2L, K3L, K4L, K5L, K6L, K7R, F1L, F2L, F3L, B8R, B14R, B15R, B16R, B17L, B18R, and the following genes in the 3′ inverted terminal repeat (ITR): B19R, and B20R, B21R, B22R, B23R, B24R, B25R, B26R, B27R, B28R, and B29R, and insertions of one, two, or three of the transgene(s) described herein.

In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises the nucleotide polymorphism(s) identified in Table 46 for vaccinia gene C14L, C2L, C1L, N2L, F3L, F13L, F16L, G7L, L3L, J3R, D6R, A41L, or A46R. In some of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises of the nucleotide polymorphisms identified in Table 46 for vaccinia gene C14L, C2L, C1L, N2L, F3L, F13L, F16L, G7L, L3L, J3R, D6R, A41L, and A46R. In certain of the embodiments and aspects provided herein, the recombinant vaccinia virus genome comprises a nucleotide sequence identical to the nucleotide sequence of GenBank Accession No. M35027.1 (SEQ ID NO: 590),except that the nucleotide sequence comprises the nucleotide polymorphisms identified in Table 46 for 1, 2, 3, 4, 5, 6, or 7 of the following vaccinia genes: C14L, C2L, C1L, N2L, F3L, F13L, F16L, G7L, L3L, J3R, D6R, A41L, and A46R. In certain of the embodiments and aspects provided herein, the recombinant vaccinia virus genome comprises a nucleotide sequence identical to the nucleotide sequence of GenBank Accession No. M35027.1 (SEQ ID NO: 590) except that the nucleotide sequence comprises the nucleotide polymorphisms identified in Table 46 for 8, 9, 10, 11, 12, or 13 of the following vaccinia genes: C14L, C2L, C1L, N2L, F3L, F13L, F16L, G7L, L3L, J3R, D6R, A41L, and A46R. In certain of such embodiments, the recombinant vaccinia virus genome may be engineered to comprise 1, 2, 3, 4, 5, 6, 7, 8, 9, or 10 of the deletions in the vaccinia virus genes identified herein (e.g., C2L, C1L, N1L, N2L, M1L, M2L, K1L, K2L, K3L, K4L, K5 L, K6L, K7R, F1L, F2L, F3L, B8R, B14R, B15R, B16R, B17L, B18R, B19R, B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise 10, 11, 12, 13, 14, 15, 16, 17, 18, 19, or 20 of the deletions in the vaccinia virus genes identified herein (e.g., C2L, C1L, N1L, N2L, M1L, M2L, K1L, K2L, K3L, K4L, K5L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In certain of such embodiments, the recombinant vaccinia virus genome may be engineered to comprise 21, 22, 23, 24, 25, 26, 27, 28, 29, 30, 31, or 32 of the deletions in the vaccinia virus genes identified herein (e.g., C2L, C1L, N1L, N2L, M1L, M2L, K1L, K2L, K3L, K4L, K5 L, K6L, K7R, F1L, F2L, F3L, B8R, B14R, B15R, B16R, B17L, B18R, B19R, and B20R, B21R, B22R, B23R, B24R, B25R, B26R, B27R, B28R, and B29R), and insertions of one, two, or three of the transgene(s) described herein. In some of such embodiments, the recombinant virus genome may be engineered to comprise deletions in C2L, C1L, N1L, N2L, M1L, M2L, K1L, K2L, K3L, K4L, K5L, K6L, K7R, F1L, F2L, F3L, B8R, B14R, B15R, B16R, B17L, B18R, and the following genes in the 3′ inverted terminal repeat (ITR): B19R, and B20R, B21R, B22R, B23R, B24R, B25R, B26R, B27R, B28R, and B29R, and insertions of one, two, or three of the transgene(s) described herein.

In certain embodiments of the various embodiments and aspects described herein, the recombinant vaccinia virus genome comprises the nucleotide sequence of SEQ ID NO: 210. In certain embodiments of the various embodiments and aspects described herein, the recombinant vaccinia virus genome comprises a nucleotide sequence that is identical to the nucleotide sequence of SEQ ID NO: 210 except for 1, 2, 3, 4, 5, 7, 8, 9, 10, 11, 12, 13, 14, 15, 16, 17, 18, 19, or 20 of the nucleotide polymorphisms identified in Table 46.

In certain embodiments, provided herein is a nucleic acid comprising a recombinant vaccinia virus genome, comprising: (a) a vaccinia virus nucleotide sequence of SEQ ID NO: 210; and (b) one, two, or three of the following: (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216).

In certain embodiments, provided herein is a nucleic acid comprising a recombinant vaccinia virus genome, comprising: (a) a vaccinia virus nucleotide sequence of SEQ ID NO: 210; and (b) two or three of the following: (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216).

In certain embodiments, provided herein is a nucleic acid comprising a recombinant vaccinia virus genome, comprising: (a) a vaccinia virus nucleotide sequence of SEQ ID NO: 210; and (b): (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216)

In specific embodiments of the above wherein the nucleic acid comprises the first transgene, the nucleic acid further comprises a nucleotide sequence comprising an H5R promoter operably linked to the first nucleotide sequence encoding the anti-CTLA-4 antibody. In specific embodiments of the above wherein the nucleic acid comprises the second transgene, the nucleic acid further comprises a nucleotide sequence comprising a late promoter operably linked to the second nucleotide sequence encoding the IL-12 polypeptide, wherein the late promoter comprises the nucleotide sequence of SEQ ID NO: 561. In specific embodiments of the above wherein the nucleic acid comprises the third transgene, the nucleic acid further comprises a nucleotide sequence comprising a B8R promoter operably linked to the third nucleotide sequence encoding FLT3L. In specific embodiments of the above wherein the nucleic acid comprises the third transgene, the nucleic acid further comprises a nucleotide sequence comprising a B19R promoter operably linked to the third nucleotide sequence encoding FLT3L. In specific embodiments of the above wherein the nucleic acid comprises the third transgene, the nucleic acid further comprises a nucleotide sequence comprising a B8R promoter and a B19R promoter operably linked to the third nucleotide sequence encoding FLT3L. In specific embodiments of the above, the endogenous vaccinia virus genes that flank the first nucleotide sequence have the same orientation, and the first nucleotide sequence is in the same orientation as the endogenous vaccinia virus genes that flank the first nucleotide sequence. In specific embodiments of the above, the endogenous vaccinia virus genes that flank the second nucleotide sequence have the same orientation, and the second nucleotide sequence is in the same orientation as the endogenous vaccinia virus genes that flank the second nucleotide sequence. In specific embodiments of the above, the endogenous vaccinia virus genes that flank the third nucleotide sequence have the same orientation, and the third nucleotide sequence is in the same orientation as the endogenous vaccinia virus genes that flank the third nucleotide sequence. In a specific embodiment of the above, the nucleic acid comprises the first transgene, the second transgene, and the third transgene, and the first transgene is inserted between the partial C2L and F3L vaccinia genes, and the second transgene and the third transgene are inserted into the locus of the deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In certain embodiments of the various embodiments and aspects described herein, the recombinant vaccinia virus genome comprises the nucleotide sequence of SEQ ID NO: 624. In certain embodiments of the various embodiments and aspects described herein, the recombinant vaccinia virus genome comprises a nucleotide sequence that is identical to the nucleotide sequence of SEQ ID NO: 624 except for 1, 2, 3, 4, 5, 7, 8, 9, 10, 11, 12, 13, 14, 15, 16, 17, 18, 19, or 20 of the nucleotide polymorphisms identified in Table 46.

In certain embodiments, provided herein is a nucleic acid comprising a recombinant vaccinia virus genome, comprising: (a) a vaccinia virus nucleotide sequence of SEQ ID NO: 624; and (b) one, two, or three of the following: (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216). In specific embodiments, the nucleic acid further comprises a promoter, such as described herein, operably linked to the first nucleotide sequence, a promoter, such as described herein, operably linked to the second nucleotide sequence, or a promoter, such as described herein, operably linked to the third promoter. In specific embodiments, the nucleic acid further comprises a promoter, such as described herein, operably linked to the first nucleotide sequence, a promoter, such as described herein, operably linked to the second nucleotide sequence, and a promoter, such as described herein, operably linked to the third promoter. In specific embodiments, the first transgene, second transgene and/or third transgene are inserted into a locus or loci described herein.

In certain embodiments, provided herein is a nucleic acid comprising a recombinant vaccinia virus genome, comprising: (a) a vaccinia virus nucleotide sequence of SEQ ID NO: 624; and (b) two or three of the following: (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216). In specific embodiments, the nucleic acid further comprises a promoter, such as described herein, operably linked to the first nucleotide sequence, a promoter, such as described herein, operably linked to the second nucleotide sequence, or a promoter, such as described herein, operably linked to the third promoter. In specific embodiments, the two or three transgenes are inserted into a locus or loci described herein.

In certain embodiments, provided herein is a nucleic acid comprising a recombinant vaccinia virus genome, comprising: (a) a vaccinia virus nucleotide sequence of SEQ ID NO: 624; and (b): (i) a first transgene comprising a first nucleotide sequence encoding an antibody or antigen-binding fragment thereof that specifically binds to CTLA-4 (e.g., human CTLA-4) (for example, wherein the first nucleotide sequence comprises the nucleotide sequence set forth in SEQ ID NO: 214); (ii) a second transgene comprising a second nucleotide sequence encoding an IL-12 polypeptide (for example, wherein the second nucleotide sequence comprises the nucleotide sequence set forth in SEQ ID NO: 215); and (iii) a third transgene comprising a third nucleotide sequence encoding FLT3L (for example, wherein the third nucleotide sequence comprises the nucleotide sequence set forth in SEQ ID NO: 216). In specific embodiments, the nucleic acid further comprises a promoter, such as described herein, operably linked to the first nucleotide sequence, a promoter, such as described herein, operably linked to the second nucleotide sequence, or a promoter, such as described herein, operably linked to the third promoter. In specific embodiments, the nucleic acid further comprises a promoter, such as described herein, operably linked to the first nucleotide sequence, a promoter, such as described herein, operably linked to the second nucleotide sequence, and a promoter, such as described herein, operably linked to the third promoter. In specific embodiments, the first transgene, second transgene and third transgene are inserted into a locus or loci described herein.

In specific embodiments of the above wherein the nucleic acid comprises the first transgene, the nucleic acid further comprises a nucleotide sequence comprising an H5R promoter operably linked to the first nucleotide sequence encoding the anti-CTLA-4 antibody. In specific embodiments of the above wherein the nucleic acid comprises the second transgene, the nucleic acid further comprises a nucleotide sequence comprising a late promoter operably linked to the second nucleotide sequence encoding the IL-12 polypeptide, wherein the late promoter comprises the nucleotide sequence of SEQ ID NO: 561. In specific embodiments of the above wherein the nucleic acid comprises the third transgene, the nucleic acid further comprises a nucleotide sequence comprising a B8R promoter operably linked to the third nucleotide sequence encoding FLT3L. In specific embodiments of the above wherein the nucleic acid comprises the third transgene, the nucleic acid further comprises a nucleotide sequence comprising a B19R promoter operably linked to the third nucleotide sequence encoding FLT3L. In specific embodiments of the above wherein the nucleic acid comprises the third transgene, the nucleic acid further comprises a nucleotide sequence comprising a B8R promoter and a B19R promoter operably linked to the third nucleotide sequence encoding FLT3L. In specific embodiments of the above, the endogenous vaccinia virus genes that flank the first nucleotide sequence have the same orientation, and the first nucleotide sequence is in the same orientation as the endogenous vaccinia virus genes that flank the first nucleotide sequence. In specific embodiments of the above, the endogenous vaccinia virus genes that flank the second nucleotide sequence have the same orientation, and the second nucleotide sequence is in the same orientation as the endogenous vaccinia virus genes that flank the second nucleotide sequence. In specific embodiments of the above, the endogenous vaccinia virus genes that flank the third nucleotide sequence have the same orientation, and the third nucleotide sequence is in the same orientation as the endogenous vaccinia virus genes that flank the third nucleotide sequence. In specific embodiments, the nucleic acid further comprises a deletion in the B8R gene. In a specific embodiment of the above, the nucleic acid comprises the first transgene, the second transgene, and the third transgene, and further comprises a deletion in the B8R gene, and the first transgene is inserted between the partial C2L and F3L vaccinia genes, and the second transgene and the third transgene are inserted into the locus of the deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4); (d) a second transgene comprising a second nucleotide sequence encoding an IL-12 polypeptide; and (e) a third transgene comprising a third nucleotide sequence encoding FLT3L; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence. In specific embodiments, the first transgene is inserted between the partial C2L and F3L vaccinia genes, and the second transgene and the third transgene are inserted into the locus of the deletion in the B8R gene. In specific embodiments, the first transgene is inserted between the partial B14R and B29R vaccinia genes, and the second transgene and the third transgene are inserted into the locus of the deletion in the B8R gene. In specific embodiments, the third transgene is upstream of the second transgene. In specific embodiments, the third transgene is downstream of the second transgene. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4), wherein the first nucleotide sequence is in the same orientation as endogenous vaccinia virus genes that flank the first nucleotide sequence, and wherein the first transgene is inserted between the partial C2L and F3L vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L, wherein the third nucleotide sequence is in the same orientation as endogenous vaccinia virus genes that flank the third nucleotide sequence, wherein the third transgene is inserted into the locus of the deletion in the B8R gene, and wherein the third transgene is upstream of the second transgene; wherein the deletions in the C2L, F3L, B14R, and B29R vaccinia genes are partial deletions; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4), wherein the first nucleotide sequence is in the same orientation as endogenous vaccinia virus genes that flank the first nucleotide sequence, and wherein the first transgene is inserted between the partial C2L and F3L vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is downstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4), wherein the first nucleotide sequence is in the same orientation as endogenous vaccinia virus genes that flank the first nucleotide sequence, and wherein the first transgene is inserted between the partial B14R and B29R vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is upstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene; (b) deletions in the following genes in the 3′ ITR: B21R, B22R, B23R, B24R, B25R, B26R, B27R, B28R, and B29R; (c) a first transgene comprising a first nucleotide sequence encoding an antibody or antigen-binding fragment thereof that specifically binds to CTLA-4 (e.g., human CTLA-4), wherein the first nucleotide sequence is in the same orientation as endogenous vaccinia virus genes that flank the first nucleotide sequence, and wherein the first transgene is inserted between the partial B14R and B29R vaccinia genes; (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is inserted into the locus of the deletion in the B8R gene; and (e) a third transgene comprising a third nucleotide sequence encoding FLT3L; wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is inserted into the locus of the deletion in the B8R gene, and wherein the third transgene is downstream of the second transgene; and wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In a particular embodiment, the nucleic acid comprises a recombinant vaccinia virus genome that comprises a deletion in the B8R gene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to Cytotoxic T-lymphocyte Associated Protein 4 (CTLA-4), wherein the first nucleotide sequence is set forth in SEQ ID NO: 214; (c) a second transgene comprising a second nucleotide sequence encoding an Interleukin 12 (IL-12) polypeptide, wherein the second nucleotide sequence is set forth in SEQ ID NO: 215; and (d) a third transgene comprising a third nucleotide sequence encoding FMS-like tyrosine kinase 3 ligand (FLT3L), wherein the third nucleotide sequence is set forth in SEQ ID NO: 216.

In some embodiments, when the flanking endogenous vaccinia virus genes of the first nucleotide sequence have the same orientation, the first nucleotide sequence is in the same orientation as the flanking endogenous vaccinia virus genes. In other embodiments, when the flanking endogenous vaccinia virus genes of the first nucleotide sequence have the same orientation, the first nucleotide sequence is in the reverse orientation relative to the flanking endogenous vaccinia virus genes. In other embodiments, when the flanking endogenous vaccinia virus genes of the first nucleotide sequence have opposite orientations, the first nucleotide sequence is in the same orientation as the flanking endogenous vaccinia virus gene that is closer to the 5′ end of the recombinant vaccinia virus genome. In other embodiments, when the flanking endogenous vaccinia virus genes of the first nucleotide sequence have opposite orientations, the first nucleotide sequence is in the same orientation as the flanking endogenous vaccinia virus gene that is closer to the 3′ end of the recombinant vaccinia virus genome. In a specific embodiment, the flanking endogenous vaccinia virus genes of the first nucleotide sequence are the C2L and F3L genes. In another specific embodiment, the flanking endogenous vaccinia virus genes of the first nucleotide sequence are the C3L and F4L genes. In another specific embodiment, the flanking endogenous vaccinia virus genes of the first nucleotide sequence are the B14R and B29R genes. In another specific embodiment, the flanking endogenous vaccinia virus genes of the first nucleotide sequence are the B13R and B29R genes. In yet another embodiment, the first nucleotide sequence is in the same orientation as an endogenous vaccinia gene adjacent to the first nucleotide sequence. In a specific embodiment, the endogenous vaccinia gene adjacent to the first nucleotide sequence is the B13R gene. In some embodiments, when the flanking endogenous vaccinia virus genes of the second nucleotide sequence have the same orientation, the second nucleotide sequence is in the same orientation as the flanking endogenous vaccinia virus genes. In other embodiments, when the flanking endogenous vaccinia virus genes of the second nucleotide sequence have the same orientation, the second nucleotide sequence is in the reverse orientation relative to the flanking endogenous vaccinia virus genes. In other embodiments, when the flanking endogenous vaccinia virus genes of the second nucleotide sequence have opposite orientations, the second nucleotide sequence is in the same orientation as the flanking endogenous vaccinia virus gene that is closer to the 5′ end of the recombinant vaccinia virus genome. In other embodiments, when the flanking endogenous vaccinia virus genes of the second nucleotide sequence have opposite orientations, the second nucleotide sequence is in the same orientation as the flanking endogenous vaccinia virus gene that is closer to the 3′ end of the recombinant vaccinia virus genome. In a specific embodiment, the flanking endogenous vaccinia virus genes of the second nucleotide sequence are the C2L and F3L genes. In another specific embodiment, the flanking endogenous vaccinia virus genes of the second nucleotide sequence are the C3L and F4L genes. In another specific embodiment, the flanking endogenous vaccinia virus genes of the second nucleotide sequence are the B14R and B29R genes. In another specific embodiment, the flanking endogenous vaccinia virus genes of the second nucleotide sequence are the B13R and B29R genes. In yet another embodiment, the second nucleotide sequence is in the same orientation as an endogenous vaccinia gene adjacent to the second nucleotide sequence. In a specific embodiment, the endogenous vaccinia gene adjacent to the second nucleotide sequence is the B13R gene. In some embodiments, when the flanking endogenous vaccinia virus genes of the third nucleotide sequence have the same orientation, the third nucleotide sequence is in the same orientation as the flanking endogenous vaccinia virus genes. In other embodiments, when the flanking endogenous vaccinia virus genes of the third nucleotide sequence have the same orientation, the third nucleotide sequence is in the reverse orientation relative to the flanking endogenous vaccinia virus genes. In other embodiments, when the flanking endogenous vaccinia virus genes of the third nucleotide sequence have opposite orientations, the third nucleotide sequence is in the same orientation as the flanking endogenous vaccinia virus gene that is closer to the 5′ end of the recombinant vaccinia virus genome. In other embodiments, when the flanking endogenous vaccinia virus genes of the third nucleotide sequence have opposite orientations, the third nucleotide sequence is in the same orientation as the flanking endogenous vaccinia virus gene that is closer to the 3′ end of the recombinant vaccinia virus genome. In a specific embodiment, the flanking endogenous vaccinia virus genes of the third nucleotide sequence are the C2L and F3L genes. In another specific embodiment, the flanking endogenous vaccinia virus genes of the third nucleotide sequence are the C3L and F4L genes. In another specific embodiment, the flanking endogenous vaccinia virus genes of the third nucleotide sequence are the B14R and B29R genes. In another specific embodiment, the flanking endogenous vaccinia virus genes of the third nucleotide sequence are the B13R and B29R genes. In yet another embodiment, the third nucleotide sequence is in the same orientation as an endogenous vaccinia gene adjacent to the third nucleotide sequence. In a specific embodiment, the endogenous vaccinia gene adjacent to the third nucleotide sequence is the B13R gene. In certain embodiments, the nucleic acid further comprises a nucleotide sequence comprising at least one promoter operably linked to the first nucleotide sequence encoding the anti-CTLA-4 antibody. In a specific embodiment, the at least one promoter operably linked to the first nucleotide sequence encoding the anti-CTLA-4 antibody is an H5R promoter, a pS promoter, or a LEO promoter. In another specific embodiment, the at least one promoter operably linked to the first nucleotide sequence encoding the anti-CTLA-4 antibody is an H5R promoter (e.g., an early H5R promoter, a late H5R promoter, or an early H5R promoter and a late H5R promoter).

In certain embodiments, the nucleic acid further comprises a nucleotide sequence comprising at least one promoter operably linked to the second nucleotide sequence encoding the IL-12 polypeptide. In a specific embodiment, the at least one promoter operably linked to the second nucleotide sequence encoding the polypoptide is a late promoter. In further specific embodiment, the late promoter comprises the nucleotide sequence of SEQ ID NO: 561, an F17R promoter, or a D13L promoter. In another further specific embodiment, the late promoter comprises the nucleotide sequence of SEQ ID NO: 561. In another embodiment, the F17R promoter comprises the nucleotide sequence of SEQ ID NO:563. In yet another embodiment, the D13L promoter comprises the nucleotide sequence of SEQ ID NO:562.

In certain embodiments, the nucleic acid further comprises a nucleotide sequence comprising at least one promoter operably linked to the third nucleotide sequence encoding FLT3L. In a specific embodiment, the at least one promoter operably linked to the third nucleotide sequence encoding FLT3L is a B8R promoter, a B19R promoter, a E3L promoter, an F11L promoter, and/or a B2R promoter. In another specific embodiment, the at least one promoter operably linked to the third nucleotide sequence encoding FLT3L is a B8R promoter. In another specific embodiment, the at least one promoter operably linked to the third nucleotide sequence encoding FLT3L is a B19R promoter. In another specific embodiment, the at least one promoter operably linked to the third nucleotide sequence encoding FLT3L is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565. In a particular embodiment, the E3L promoter comprises the nucleotide sequence of SEQ ID NO: 567. In a particular embodiment, the F11L promoter comprises the nucleotide sequence of SEQ ID NO: 568. In a particular embodiment, the B2R promoter comprises the nucleotide sequence of SEQ ID NO: 569.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214; (c) a second transgene comprising a second nucleotide sequence encoding an IL-12 polypeptide, wherein the second nucleotide sequence is set forth in SEQ ID NO: 215; and (d) a third transgene comprising a third nucleotide sequence encoding FLT3L, wherein the third nucleotide sequence is set forth in SEQ ID NO: 216; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence. In specific embodiments, the first transgene is inserted between the partial C2L and F3L vaccinia genes in SEQ ID NO: 210, and the second transgene and the third transgene are inserted into the locus of the deletion in the B8R gene. In specific embodiments, the first transgene is inserted between the partial B14R and B29R vaccinia genes in SEQ ID NO: 210, and the second transgene and the third transgene are inserted into the locus of the deletion in the B8R gene. In specific embodiments, the third transgene is upstream of the second transgene. In specific embodiments, the third transgene is downstream of the second transgene. In a particular embodiment, the B8R promoter comprises the nucleotide sequence of SEQ ID NO: 564. In a particular embodiment, the B19R promoter comprises the nucleotide sequence of SEQ ID NO: 565.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the nucleotide sequence of the pS comprises the nucleotide sequence of SEQ ID NO: 555, SEQ ID NO: 556, or SEQ ID NO: 557.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter or a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a B8R promoter and a B19R promoter. In specific embodiments, the at least one promoter operably linked to the third nucleotide sequence is a B8R promoter and a B19R promoter. In specific embodiments, the B8R promoter comprises the nucleotide sequence of SEQ ID NO: 564 and the B19R promoter comprises the nucleotide sequence of SEQ ID NO: 565. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the F17R promoter comprises the nucleotide sequence of SEQ ID NO: 563.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is an H5R promoter; (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and (iii) a nucleotide sequence comprising at least one promoter operably linked to the third nucleotide sequence, wherein the at least one promoter operably linked to the third nucleotide sequence is a E3L promoter. In specific embodiments, the at least one promoter operatively linked to the first nucleotide sequence is an H5R early promoter or an H5R late promoter. In specific embodiments, the at least one promoter operatively linked to the first nucleotide sequence is an H5R early promoter and an H5R late promoter. In specific embodiments, the H5R early promoter comprises the nucleotide sequence of SEQ ID NO: 553 and the H5R late promoter comprises the nucleotide sequence of SEQ ID NO: 554. In specific embodiments, nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial C2L and F3L vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is upstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In another aspect, provided herein is a nucleic acid comprising a recombinant vaccinia virus genome, comprising: (a) a vaccinia virus nucleotide sequence of SEQ ID NO: 210, which comprises partial C2L, F3L, B14R, and B29R vaccinia genes and which comprises a deletion in the B8R gene; (b) a first transgene comprising a first nucleotide sequence encoding an antibody that specifically binds to CTLA-4 (e.g., human CTLA-4), wherein the first nucleotide sequence is set forth in SEQ ID NO: 214, and wherein the first nucleotide sequence is in the same orientation as endogenous vaccinia virus genes that flank the first nucleotide sequence and the first transgene is inserted between the partial B14R and B29R vaccinia genes in SEQ ID NO: 210; (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is inserted into the locus of the deletion in the B8R gene; and (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is inserted into the locus of the deletion in the B8R gene, and wherein the third transgene is downstream of the second transgene; wherein the nucleic acid further comprises: (i) a nucleotide sequence comprising at least one promoter operably linked to the first nucleotide sequence, wherein the at least one promoter operably linked to the first nucleotide sequence is a pS promoter; (ii) a nucleotide sequence comprising at least one promoter operably linked to the second nucleotide sequence, wherein the at least one promoter operably linked to the second nucleotide sequence is an F17R promoter; and (iii) a nucleotide sequence comprising at least one promoter operably linked to the third nucleotide sequence, wherein the at least one promoter operably linked to the third nucleotide sequence is a E3L promoter. In specific embodiments, the nucleotide sequence of the pS promoter comprises the nucleotide sequence of SEQ ID NO: 555, SEQ ID NO: 556, or SEQ ID NO: 557. In specific embodiments, the nucleotide sequence of the F17R promoter comprises the nucleotide sequence of SEQ ID NO: 563. In specific embodiments, the nucleotide sequence of the E3L promoter comprises the nucleotide sequence of SEQ ID NO: 567.

In some embodiments of the various embodiments and aspects described herein wherein the nucleic acid comprises the first transgene, the first transgene is inserted between the partial C2L and F3L vaccinia genes. In another embodiment, the first transgene is inserted adjacent to the partial vaccinia C2L gene. In another embodiment, the first transgene is inserted adjacent to the partial vaccinia F3L gene. In another embodiment, the first transgene is inserted between vaccinia genes C3L and F4L. In other embodiments of the various embodiments and aspects described herein wherein the nucleic acid comprises the first transgene, the first transgene is inserted into the locus of the deletion in the B8R gene. In other embodiments of the various embodiments and aspects described herein wherein the nucleic acid comprises the first transgene, the first transgene is inserted between the partial B14R and B29R vaccinia genes. In another embodiment, the first transgene is inserted adjacent to the partial vaccinia B14R gene. In another embodiment, the first transgene is inserted adjacent to the partial vaccinia B29R gene. In particular embodiments wherein the B14R to B29R genes are deleted, the first transgene is inserted adjacent to the B13R gene.

In some embodiments of the various embodiments and aspects described herein wherein the nucleic acid comprises the second transgene, the second transgene is inserted between the partial C2L and F3L vaccinia genes. In another embodiment, the second transgene is inserted adjacent to the partial vaccinia C2L gene. In another embodiment, the second transgene is inserted adjacent to the partial vaccinia F3L gene. In another embodiment, the second transgene is inserted between vaccinia genes C3L and F4L. In other embodiments of the various embodiments and aspects described herein wherein the nucleic acid comprises the second transgene, the second transgene is inserted into the locus of the deletion in the B8R gene. In other embodiments of the various embodiments and aspects described herein wherein the nucleic acid comprises the second transgene, the second transgene is inserted between the partial B14R and B29R vaccinia genes. In another embodiment, the second transgene is inserted adjacent to the partial vaccinia B14R gene. In another embodiment, the second transgene is inserted adjacent to the partial vaccinia B29R gene. In particular embodiments wherein the B14R to B29R genes are deleted, the second transgene is inserted adjacent to the B13R gene.

In some embodiments of the various embodiments and aspects described herein wherein the nucleic acid comprises the third transgene, the third transgene is inserted between the partial C2L and F3L vaccinia genes. In another embodiment, the third transgene is inserted adjacent to the partial vaccinia C2L gene. In another embodiment, the third transgene is inserted adjacent to the partial vaccinia F3L gene. In another embodiment, the third transgene is inserted between vaccinia genes C3L and F4L. In other embodiments of the various embodiments and aspects described herein wherein the nucleic acid comprises the third transgene, the third transgene is inserted into the locus of the deletion in the B8R gene. In other embodiments of the various embodiments and aspects described herein wherein the nucleic acid comprises the third transgene, the third transgene is inserted between the partial B14R and B29R vaccinia genes. In another embodiment, the third transgene is inserted adjacent to the partial vaccinia B14R gene. In another embodiment, the third transgene is inserted adjacent to the partial vaccinia B29R gene. In particular embodiments wherein the B14R to B29R genes are deleted, the third transgene is inserted adjacent to the B13R gene.

In some embodiments of the various embodiments and aspects described herein wherein the nucleic acid comprises the first transgene and the second transgene, the first transgene and the second transgene are inserted between the partial C2L and F3L vaccinia genes. In another embodiment, the first transgene and the second transgene are inserted adjacent to the partial vaccinia C2L gene. In another embodiment, the first transgene and the second transgene are inserted adjacent to the partial vaccinia F3L gene. In another embodiment, the first transgene and the second transgene are inserted between vaccinia genes C3L and F4L. In other embodiments of the various embodiments and aspects described herein wherein the nucleic acid comprises the first transgene and the second transgene, the first transgene and the second transgene are inserted into the locus of the deletion in the B8R gene. In other embodiments of the various embodiments and aspects described herein wherein the nucleic acid comprises the first transgene and the second transgene, the first transgene and the second transgene are inserted between the partial B14R and B29R vaccinia genes. In another embodiment, the first transgene and the second transgene are inserted adjacent to the partial vaccinia B14R gene. In another embodiment, the first transgene and the second transgene are inserted adjacent to the partial vaccinia B29R gene. In particular embodiments wherein the B14R to B29R genes are deleted, the first transgene and the second transgene are inserted adjacent to the B13R gene.

In some embodiments of the various embodiments and aspects described herein wherein the nucleic acid comprises the first transgene and the third transgene, the first transgene and the third transgene are inserted between the partial C2L and F3L vaccinia genes. In another embodiment, the first transgene and the third transgene are inserted adjacent to the partial vaccinia C2L gene. In another embodiment, the first transgene and the third transgene are inserted adjacent to the partial vaccinia F3L gene. In another embodiment, the first transgene and the third transgene are inserted between vaccinia genes C3L and F4L. In other embodiments of the various embodiments and aspects described herein wherein the nucleic acid comprises the first transgene and the third transgene, the first transgene and the third transgene are inserted into the locus of the deletion in the B8R gene. In other embodiments of the various embodiments and aspects described herein wherein the nucleic acid comprises the first transgene and the third transgene, the first transgene and the third transgene are inserted between the partial B14R and B29R vaccinia genes. In another embodiment, the first transgene and the third transgene are inserted adjacent to the partial vaccinia B14R gene. In another embodiment, the first transgene and the third transgene are inserted adjacent to the partial vaccinia B29R gene. In particular embodiments wherein the B14R to B29R genes are deleted, the first transgene and the third transgene are inserted adjacent to the B13R gene.

In some embodiments of the various embodiments and aspects described herein wherein the nucleic acid comprises the second transgene and the third transgene, the second transgene and the third transgene are inserted between the partial C2L and F3L vaccinia genes. In another embodiment, the second transgene and the third transgene are inserted adjacent to the partial vaccinia C2L gene. In another embodiment, the second transgene and the third transgene are inserted adjacent to the partial vaccinia F3L gene. In another embodiment, the second transgene and the third transgene are inserted between vaccinia genes C3L and F4L. In other embodiments of the various embodiments and aspects described herein wherein the nucleic acid comprises the second transgene and the third transgene, the second transgene and the third transgene are inserted into the locus of the deletion in the B8R gene. In other embodiments of the various embodiments and aspects described herein wherein the nucleic acid comprises the second transgene and the third transgene, the second transgene and the third transgene are inserted between the partial B14R and B29R vaccinia genes. In another embodiment, the second transgene and the third transgene are inserted adjacent to the partial vaccinia B14R gene. In another embodiment, the second transgene and the third transgene are inserted adjacent to the partial vaccinia B29R gene. In particular embodiments wherein the B14R to B29R genes are deleted, the second transgene and the third transgene are inserted adjacent to the B13R gene.

In some embodiments of the various embodiments and aspects described herein wherein the nucleic acid comprises the first transgene and the second transgene, the first transgene is inserted between the partial C2L and F3L vaccinia genes, and the second transgene is inserted into the locus of the deletion in the B8R gene. In another embodiment, the first transgene is inserted adjacent to the partial vaccinia C2L gene, and the second transgene is inserted into the locus of the deletion in the B8R gene. In another embodiment, the first transgene is inserted adjacent to the partial vaccinia F3L gene, and the second transgene is inserted into the locus of the deletion in the B8R gene. In another embodiment, the first transgene is inserted between vaccinia genes C3L and F4L, and the second transgene is inserted into the locus of the deletion in the B8R gene. In other embodiments of the various embodiments and aspects described herein wherein the nucleic acid comprises the first transgene and the second transgene, the second transgene is inserted between the partial C2L and F3L vaccinia genes, and the first transgene is inserted into the locus of the deletion in the B8R gene. In another embodiment, the second transgene is inserted adjacent to the partial vaccinia C2L gene, and the first transgene is inserted into the locus of the deletion in the B8R gene. In another embodiment, the second transgene is inserted adjacent to the partial vaccinia F3L gene, and the first transgene is inserted into the locus of the deletion in the B8R gene. In another embodiment, the second transgene is inserted between vaccinia genes C3L and F4L, and the first transgene is inserted into the locus of the deletion in the B8R gene. In other embodiments of the various embodiments and aspects described herein wherein the nucleic acid comprises the first transgene and the second transgene, the first transgene is inserted between the partial C2L and F3L vaccinia genes, and the second transgene is inserted between the partial B14R and B29R vaccinia genes. In another embodiment, the first transgene is inserted adjacent to the partial vaccinia C2L gene, inserted adjacent to the partial vaccinia F3L gene, or inserted between vaccinia genes C3L and F4L, and the second transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene and the second transgene, the second transgene is inserted between the partial C2L and F3L vaccinia genes, and the first transgene is inserted between the partial B14R and B29R vaccinia genes. In another embodiment, the second transgene is inserted adjacent to the partial vaccinia C2L gene, inserted adjacent to the partial vaccinia F3L gene, or inserted between vaccinia genes C3L and F4L, and the first transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene and the second transgene, the first transgene is inserted into the locus of the deletion in the B8R gene, and the second transgene is inserted between the partial B14R and B29R vaccinia genes. In another embodiment, the first transgene is inserted into the locus of the deletion in the B8R gene, and the second transgene is inserted adjacent to the partial vaccinia B14R gene. In another embodiment, the first transgene is inserted into the locus of the deletion in the B8R gene, and the second transgene is inserted adjacent to the partial vaccinia B29R gene. In particular embodiments wherein the B14R to B29R genes are deleted, the first transgene is inserted into the locus of the deletion in the B8R gene, and the second transgene is inserted adjacent to the B13R gene. In other embodiments of the various embodiments and aspects described herein wherein the nucleic acid comprises the first transgene and the second transgene, the second transgene is inserted into the locus of the deletion in the B8R gene, and the first transgene is inserted between the partial B14R and B29R vaccinia genes. In another embodiment, the second transgene is inserted into the locus of the deletion in the B8R gene, and the first transgene is inserted adjacent to the partial vaccinia B14R gene. In another embodiment, the second transgene is inserted into the locus of the deletion in the B8R gene, and the first transgene is inserted adjacent to the partial vaccinia B29R gene. In particular embodiments wherein the B14R to B29R genes are deleted, the second transgene is inserted into the locus of the deletion in the B8R gene, and the first transgene is inserted adjacent to the B13R gene.

In some embodiments of the various embodiments and aspects described herein wherein the nucleic acid comprises the first transgene and the third transgene, the first transgene is inserted between the partial C2L and F3L vaccinia genes, and the third transgene is inserted into the locus of the deletion in the B8R gene. In another embodiment, the first transgene is inserted adjacent to the partial vaccinia C2L gene, and the third transgene is inserted into the locus of the deletion in the B8R gene. In another embodiment, the first transgene is inserted adjacent to the partial vaccinia F3L gene, and the third transgene is inserted into the locus of the deletion in the B8R gene. In another embodiment, the first transgene is inserted between vaccinia genes C3L and F4L, and the third transgene is inserted into the locus of the deletion in the B8R gene. In other embodiments of the various embodiments and aspects described herein wherein the nucleic acid comprises the first transgene and the third transgene, the third transgene is inserted between the partial C2L and F3L vaccinia genes, and the first transgene is inserted into the locus of the deletion in the B8R gene. In another embodiment, the third transgene is inserted adjacent to the partial vaccinia C2L gene, and the first transgene is inserted into the locus of the deletion in the B8R gene. In another embodiment, the third transgene is inserted adjacent to the partial vaccinia F3L gene, and the first transgene is inserted into the locus of the deletion in the B8R gene. In another embodiment, the third transgene is inserted between vaccinia genes C3L and F4L, and the first transgene is inserted into the locus of the deletion in the B8R gene. In other embodiments of the various embodiments and aspects described herein wherein the nucleic acid comprises the first transgene and the third transgene, the first transgene is inserted between the partial C2L and F3L vaccinia genes, and the third transgene is inserted between the partial B14R and B29R vaccinia genes. In another embodiment, the first transgene is inserted adjacent to the partial vaccinia C2L gene, inserted adjacent to the partial vaccinia F3L gene, or inserted between vaccinia genes C3L and F4L, and the third transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene and the third transgene, the third transgene is inserted between the partial C2L and F3L vaccinia genes, and the first transgene is inserted between the partial B14R and B29R vaccinia genes. In another embodiment, the third transgene is inserted adjacent to the partial vaccinia C2L gene, inserted adjacent to the partial vaccinia F3L gene, or inserted between vaccinia genes C3L and F4L, and the first transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene and the third transgene, the first transgene is inserted into the locus of the deletion in the B8R gene, and the third transgene is inserted between the partial B14R and B29R vaccinia genes. In another embodiment, the first transgene is inserted into the locus of the deletion in the B8R gene, and the third transgene is inserted adjacent to the partial vaccinia B14R gene. In another embodiment, the first transgene is inserted into the locus of the deletion in the B8R gene, and the third transgene is inserted adjacent to the partial vaccinia B29R gene. In particular embodiments wherein the B14R to B29R genes are deleted, the first transgene is inserted into the locus of the deletion in the B8R gene, and the third transgene is inserted adjacent to the B13R gene. In other embodiments of the various embodiments and aspects described herein wherein the nucleic acid comprises the first transgene and the third transgene, the third transgene is inserted into the locus of the deletion in the B8R gene, and the first transgene is inserted between the partial B14R and B29R vaccinia genes. In another embodiment, the third transgene is inserted into the locus of the deletion in the B8R gene, and the first transgene is inserted adjacent to the partial vaccinia B14R gene. In another embodiment, the third transgene is inserted into the locus of the deletion in the B8R gene, and the first transgene is inserted adjacent to the partial vaccinia B29R gene. In particular embodiments wherein the B14R to B29R genes are deleted, the third transgene is inserted into the locus of the deletion in the B8R gene, and the first transgene is inserted adjacent to the B13R gene.

In some embodiments of the various embodiments and aspects described herein wherein the nucleic acid comprises the second transgene and the third transgene, the second transgene is inserted between the partial C2L and F3L vaccinia genes, and the third transgene is inserted into the locus of the deletion in the B8R gene. In another embodiment, the second transgene is inserted adjacent to the partial vaccinia C2L gene, and the third transgene is inserted into the locus of the deletion in the B8R gene. In another embodiment, the second transgene is inserted adjacent to the partial vaccinia F3L gene, and the third transgene is inserted into the locus of the deletion in the B8R gene. In another embodiment, the second transgene is inserted between vaccinia genes C3L and F4L, and the third transgene is inserted into the locus of the deletion in the B8R gene. In other embodiments of the various embodiments and aspects described herein wherein the nucleic acid comprises the second transgene and the third transgene, the third transgene is inserted between the partial C2L and F3L vaccinia genes, and the second transgene is inserted into the locus of the deletion in the B8R gene. In another embodiment, the third transgene is inserted adjacent to the partial vaccinia C2L gene, and the second transgene is inserted into the locus of the deletion in the B8R gene. In another embodiment, the third transgene is inserted adjacent to the partial vaccinia F3L gene, and the second transgene is inserted into the locus of the deletion in the B8R gene. In another embodiment, the third transgene is inserted between vaccinia genes C3L and F4L, and the second transgene is inserted into the locus of the deletion in the B8R gene. In other embodiments of the various embodiments and aspects described herein wherein the nucleic acid comprises the second transgene and the third transgene, the second transgene is inserted between the partial C2L and F3L vaccinia genes, and the third transgene is inserted between the partial B14R and B29R vaccinia genes. In another embodiment, the second transgene is inserted adjacent to the partial vaccinia C2L gene, inserted adjacent to the partial vaccinia F3L gene, or inserted between vaccinia genes C3L and F4L, and the third transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the second transgene and the third transgene, the third transgene is inserted between the partial C2L and F3L vaccinia genes, and the second transgene is inserted between the partial B14R and B29R vaccinia genes. In another embodiment, the third transgene is inserted adjacent to the partial vaccinia C2L gene, inserted adjacent to the partial vaccinia F3L gene, or inserted between vaccinia genes C3L and F4L, and the second transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the second transgene and the third transgene, the second transgene is inserted into the locus of the deletion in the B8R gene, and the third transgene is inserted between the partial B14R and B29R vaccinia genes. In another embodiment, the second transgene is inserted into the locus of the deletion in the B8R gene, and the third transgene is inserted adjacent to the partial vaccinia B14R gene. In another embodiment, the second transgene is inserted into the locus of the deletion in the B8R gene, and the third transgene is inserted adjacent to the partial vaccinia B29R gene. In particular embodiments wherein the B14R to B29R genes are deleted, the second transgene is inserted into the locus of the deletion in the B8R gene, and the third transgene is inserted adjacent to the B13R gene. In other embodiments of the various embodiments and aspects described herein wherein the nucleic acid comprises the second transgene and the third transgene, the third transgene is inserted into the locus of the deletion in the B8R gene, and the second transgene is inserted between the partial B14R and B29R vaccinia genes. In another embodiment, the third transgene is inserted into the locus of the deletion in the B8R gene, and the second transgene is inserted adjacent to the partial vaccinia B14R gene. In another embodiment, the third transgene is inserted into the locus of the deletion in the B8R gene, and the second transgene is inserted adjacent to the partial vaccinia B29R gene. In particular embodiments wherein the B14R to B29R genes are deleted, the third transgene is inserted into the locus of the deletion in the B8R gene, and the second transgene is inserted adjacent to the B13R gene.

In some embodiments of the various embodiments and aspects described herein wherein the nucleic acid comprises the first transgene, the second transgene and the third transgene, the first transgene, the second transgene, and the third transgene are inserted between the partial C2L and F3L vaccinia genes. In another embodiment, the first transgene, the second transgene, and the third transgene are inserted adjacent to the partial vaccinia C2L gene. In another embodiment, the first transgene, the second transgene, and the third transgene are inserted adjacent to the partial vaccinia F3L gene. In another embodiment, the first transgene, the second transgene, and the third transgene are inserted between vaccinia genes C3L and F4L. In other embodiments of the various embodiments and aspects described herein wherein the nucleic acid comprises the first transgene, the second transgene and the third transgene, the first transgene, the second transgene, and the third transgene are inserted into the locus of the deletion in the B8R gene. In other embodiments of the various embodiments and aspects described herein wherein the nucleic acid comprises the first transgene, the second transgene and the third transgene, the first transgene, the second transgene, and the third transgene are inserted between the partial B14R and B29R vaccinia genes. In another embodiment, the first transgene, the second transgene, and the third transgene are inserted adjacent to the partial vaccinia B14R gene. In another embodiment, the first transgene, the second transgene, and the third transgene are inserted adjacent to the partial vaccinia B29R gene. In particular embodiments wherein the B14R to B29R genes are deleted, the first transgene, the second transgene, and the third transgene are inserted adjacent to the B13R gene.

In other embodiments of the various embodiments and aspects described herein wherein the nucleic acid comprises the first transgene, the second transgene and the third transgene, the first transgene is inserted between the partial C2L and F3L vaccinia genes, and the second transgene and the third transgene are inserted into the locus of the deletion in the B8R gene. In another embodiment, the first transgene is inserted adjacent to the partial vaccinia C2L gene, and the second transgene and the third transgene are inserted into the locus of the deletion in the B8R gene. In another embodiment, the first transgene is inserted adjacent to the partial vaccinia F3L gene, and the second transgene and the third transgene are inserted into the locus of the deletion in the B8R gene. In another embodiment, the first transgene is inserted between vaccinia genes C3L and F4L, and the second transgene and the third transgene are inserted into the locus of the deletion in the B8R gene. In other embodiments of the various embodiments and aspects described herein wherein the nucleic acid comprises the first transgene, the second transgene and the third transgene, the second transgene is inserted between the partial C2L and F3L vaccinia genes, and the first transgene and the third transgene are inserted into the locus of the deletion in the B8R gene. In another embodiment, the second transgene is inserted adjacent to the partial vaccinia C2L gene, and the first transgene and the third transgene are inserted into the locus of the deletion in the B8R gene. In another embodiment, the second transgene is inserted adjacent to the partial vaccinia F3L gene, and the first transgene and the third transgene are inserted into the locus of the deletion in the B8R gene. In another embodiment, the second transgene is inserted between vaccinia genes C3L and F4L, and the first transgene and the third transgene are inserted into the locus of the deletion in the B8R gene. In other embodiments of the various embodiments and aspects described herein wherein the nucleic acid comprises the first transgene, the second transgene and the third transgene, the third transgene is inserted between the partial C2L and F3L vaccinia genes, and the first transgene and the second transgene are inserted into the locus of the deletion in the B8R gene. In another embodiment, the third transgene is inserted adjacent to the partial vaccinia C2L gene, and the first transgene and the second transgene are inserted into the locus of the deletion in the B8R gene. In another embodiment, the third transgene is inserted adjacent to the partial vaccinia F3L gene, and the first transgene and the second transgene are inserted into the locus of the deletion in the B8R gene. In another embodiment, the third transgene is inserted between vaccinia genes C3L and F4L, and the first transgene and the second transgene are inserted into the locus of the deletion in the B8R gene. In other embodiments of the various embodiments and aspects described herein wherein the nucleic acid comprises the first transgene, the second transgene and the third transgene, the first transgene and the second transgene are inserted between the partial C2L and F3L vaccinia genes, and the third transgene is inserted into the locus of the deletion in the B8R gene. In another embodiment, the first transgene and the second transgene are inserted adjacent to the partial vaccinia C2L gene, and the third transgene is inserted into the locus of the deletion in the B8R gene. In another embodiment, the first transgene and the second transgene are inserted adjacent to the partial vaccinia F3L gene, and the third transgene is inserted into the locus of the deletion in the B8R gene. In another embodiment, the first transgene and the second transgene are inserted between vaccinia genes C3L and F4L, and the third transgene is inserted into the locus of the deletion in the B8R gene. In other embodiments of the various embodiments and aspects described herein wherein the nucleic acid comprises the first transgene, the second transgene and the third transgene, the first transgene and the third transgene are inserted between the partial C2L and F3L vaccinia genes, and the second transgene is inserted into the locus of the deletion in the B8R gene. In another embodiment, the first transgene and the third transgene are inserted adjacent to the partial vaccinia C2L gene, and the second transgene is inserted into the locus of the deletion in the B8R gene. In another embodiment, the first transgene and the third transgene are inserted adjacent to the partial vaccinia F3L gene, and the second transgene is inserted into the locus of the deletion in the B8R gene. In another embodiment, the first transgene and the third transgene are inserted between vaccinia genes C3L and F4L, and the second transgene is inserted into the locus of the deletion in the B8R gene. In other embodiments of the various embodiments and aspects described herein wherein the nucleic acid comprises the first transgene, the second transgene and the third transgene, the second transgene and the third transgene are inserted between the partial C2L and F3L vaccinia genes, and the first transgene is inserted into the locus of the deletion in the B8R gene. In another embodiment, the second transgene and the third transgene are is inserted adjacent to the partial vaccinia C2L gene, and the first transgene is inserted into the locus of the deletion in the B8R gene. In another embodiment, the second transgene and the third transgene are inserted adjacent to the partial vaccinia F3L gene, and the first transgene is inserted into the locus of the deletion in the B8R gene. In another embodiment, the second transgene and the third transgene are inserted between vaccinia genes C3L and F4L, and the first transgene is inserted into the locus of the deletion in the B8R gene. In other embodiments of the various embodiments and aspects described herein wherein the nucleic acid comprises the first transgene, the second transgene and the third transgene, the first transgene is inserted within between the partial C2L and F3L vaccinia genes, and the second transgene and the third transgene are inserted between the partial B14R and B29R vaccinia genes. In another embodiment, the first transgene is inserted adjacent to the partial vaccinia C2L gene, inserted adjacent to the partial vaccinia F3L gene, or inserted between vaccinia genes C3L and F4L; and the second transgene and the third transgene are inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the second transgene is inserted between the partial C2L and F3L vaccinia genes, and the first transgene and the third transgene are inserted between the partial B14R and B29R vaccinia genes. In another embodiment, the second transgene is inserted adjacent to the partial vaccinia C2L gene, inserted adjacent to the partial vaccinia F3L gene, or inserted between vaccinia genes C3L and F4L; and the first transgene and the third transgene are inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the third transgene is inserted between the partial C2L and F3L vaccinia genes, and the first transgene and the second transgene are inserted between the partial B14R and B29R vaccinia genes. In another embodiment, the third transgene is inserted adjacent to the partial vaccinia C2L gene, inserted adjacent to the partial vaccinia F3L gene, or inserted between vaccinia genes C3L and F4L; and the first transgene and the second transgene are inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the first transgene and the second transgene are inserted between the partial C2L and F3L vaccinia genes, and the third transgene is inserted between the partial B14R and B29R vaccinia genes. In another embodiment, the first transgene and the second transgene are inserted adjacent to the partial vaccinia C2L gene, inserted adjacent to the partial vaccinia F3L gene, or inserted between vaccinia genes C3L and F4L; and the third transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the first transgene and the third transgene are inserted between the partial C2L and F3L vaccinia genes, and the second transgene is inserted between the partial B14R and B29R vaccinia genes. In another embodiment, the first transgene and the third transgene are inserted adjacent to the partial vaccinia C2L gene, inserted adjacent to the partial vaccinia F3L gene, or inserted between vaccinia genes C3L and F4L; and the second transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the second transgene and the third transgene are inserted between the partial C2L and F3L vaccinia genes, and the first transgene is inserted between the partial B14R and B29R vaccinia genes. In another embodiment, the second transgene and the third transgene are inserted adjacent to the partial vaccinia C2L gene, inserted adjacent to the partial vaccinia F3L gene, or inserted between vaccinia genes C3L and F4L; and the first transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the first transgene is inserted into the locus of the deletion in the B8R gene, and the second transgene and the third transgene are inserted between the partial B14R and B29R vaccinia genes. In another embodiment, the first transgene is inserted into the locus of the deletion in the B8R gene, and the second transgene and the third transgene are inserted adjacent to the partial vaccinia B14R gene. In another embodiment, the first transgene is inserted into the locus of the deletion in the B8R gene, and the second transgene and the third transgene are inserted adjacent to the partial vaccinia B29R gene. In particular embodiments wherein the B14R to B29R genes are deleted, the first transgene is inserted into the locus of the deletion in the B8R gene, and the second transgene and the third transgene are inserted adjacent to the B13R gene. In other embodiments of the various embodiments and aspects described herein wherein the nucleic acid comprises the first transgene, the second transgene and the third transgene, the second transgene is inserted into the locus of the deletion in the B8R gene, and the first transgene and the third transgene are inserted between the partial B14R and B29R vaccinia genes. In another embodiment, the second transgene is inserted into the locus of the deletion in the B8R gene, and the first transgene and the third transgene are inserted adjacent to the partial vaccinia B14R gene. In another embodiment, the second transgene is inserted into the locus of the deletion in the B8R gene, and the first transgene and the third transgene are inserted adjacent to the partial vaccinia B29R gene. In particular embodiments wherein the B14R to B29R genes are deleted, the second transgene is inserted into the locus of the deletion in the B8R gene, and the first transgene and the third transgene are inserted adjacent to the B13R gene. In other embodiments of the various embodiments and aspects described herein wherein the nucleic acid comprises the first transgene, the second transgene and the third transgene, the third transgene is inserted into the locus of the deletion in the B8R gene, and the first transgene and the second transgene are inserted between the partial B14R and B29R vaccinia genes. In another embodiment, the third transgene is inserted into the locus of the deletion in the B8R gene, and the first transgene and the second transgene are inserted adjacent to the partial vaccinia B14R gene. In another embodiment, the third transgene is inserted into the locus of the deletion in the B8R gene, and the first transgene and the second transgene are inserted adjacent to the partial vaccinia B29R gene. In particular embodiments wherein the B14R to B29R genes are deleted, the third transgene is inserted into the locus of the deletion in the B8R gene, and the first transgene and the second transgene are inserted adjacent to the B13R gene. In other embodiments of the various embodiments and aspects described herein wherein the nucleic acid comprises the first transgene, the second transgene and the third transgene, the first transgene and the second transgene are inserted into the locus of the deletion in the B8R gene, and the third transgene is inserted between the partial B14R and B29R vaccinia genes. In another embodiment, the first transgene and the second transgene are inserted into the locus of the deletion in the B8R gene, and the third transgene is inserted adjacent to the partial vaccinia B14R gene. In another embodiment, the first transgene and the second transgene are inserted into the locus of the deletion in the B8R gene, and the third transgene is inserted adjacent to the partial vaccinia B29R gene. In particular embodiments wherein the B14R to B29R genes are deleted, the first transgene and the second transgene are inserted into the locus of the deletion in the B8R gene, and the third transgene is inserted adjacent to the B13R gene. In other embodiments of the various embodiments and aspects described herein wherein the nucleic acid comprises the first transgene, the second transgene and the third transgene, the first transgene and the third transgene are inserted into the locus of the deletion in the B8R gene, and the second transgene is inserted between the partial B14R and B29R vaccinia genes. In another embodiment, the first transgene and the third transgene are inserted into the locus of the deletion in the B8R gene, and the second transgene is inserted adjacent to the partial vaccinia B14R gene. In another embodiment, the first transgene and the third transgene are inserted into the locus of the deletion in the B8R gene, and the second transgene is inserted adjacent to the partial vaccinia B29R gene. In particular embodiments wherein the B14R to B29R genes are deleted, the first transgene and the third transgene are inserted into the locus of the deletion in the B8R gene, and the second transgene is inserted adjacent to the B13R gene. In other embodiments of the various embodiments and aspects described herein wherein the nucleic acid comprises the first transgene, the second transgene and the third transgene, the second transgene and the third transgene are inserted into the locus of the deletion in the B8R gene, and the first transgene is inserted between the partial B14R and B29R vaccinia genes. In another embodiment, the second transgene and the third transgene are inserted into the locus of the deletion in the B8R gene, and the first transgene is inserted adjacent to the partial vaccinia B14R gene. In another embodiment, the second transgene and the third transgene are inserted into the locus of the deletion in the B8R gene, and the first transgene is inserted adjacent to the partial vaccinia B29R gene. In particular embodiments wherein the B14R to B29R genes are deleted, the second transgene and the third transgene are inserted into the locus of the deletion in the B8R gene, and the first transgene is inserted adjacent to the B13R gene.

In other embodiments of the various embodiments and aspects described herein wherein the nucleic acid comprises the first transgene, the second transgene and the third transgene, the first transgene is inserted between the partial C2L and F3L vaccinia genes, the second transgene is inserted into the locus of the deletion in the B8R gene, and the third transgene is inserted between the partial B14R and B29R vaccinia genes. In another embodiment, the first transgene is inserted adjacent to the partial vaccinia C2L gene, inserted adjacent to the partial vaccinia F3L gene, or inserted between vaccinia genes C3L and F4L, the second transgene is inserted into the locus of the deletion in the B8R gene, and the third transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the first transgene is inserted between the partial C2L and F3L vaccinia genes, the third transgene is inserted into the locus of the deletion in the B8R gene, and the second transgene is inserted between the partial B14R and B29R vaccinia genes. In another embodiment, the first transgene is inserted adjacent to the partial vaccinia C2L gene, inserted adjacent to the partial vaccinia F3L gene, or inserted between vaccinia genes C3L and F4L, the third transgene is inserted into the locus of the deletion in the B8R gene, and the second transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the second transgene is inserted between the partial C2L and F3L vaccinia genes, the first transgene is inserted into the locus of the deletion in the B8R gene, and the third transgene is inserted between the partial B14R and B29R vaccinia genes. In another embodiment, the second transgene is inserted adjacent to the partial vaccinia C2L gene, inserted adjacent to the partial vaccinia F3L gene, or inserted between vaccinia genes C3L and F4L, the first transgene is inserted into the locus of the deletion in the B8R gene, and the third transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the second transgene is inserted between the partial C2L and F3L vaccinia genes, the third transgene is inserted into the locus of the deletion in the B8R gene, and the first transgene is inserted between the partial B14R and B29R vaccinia genes. In another embodiment, the second transgene is inserted adjacent to the partial vaccinia C2L gene, inserted adjacent to the partial vaccinia F3L gene, or inserted between vaccinia genes C3L and F4L, the third transgene is inserted into the locus of the deletion in the B8R gene, and the first transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the third transgene is inserted between the partial C2L and F3L vaccinia genes, the first transgene is inserted into the locus of the deletion in the B8R gene, and the second transgene is inserted between the partial B14R and B29R vaccinia genes. In another embodiment, the third transgene is inserted adjacent to the partial vaccinia C2L gene, inserted adjacent to the partial vaccinia F3L gene, or inserted between vaccinia genes C3L and F4L, the first transgene is inserted into the locus of the deletion in the B8R gene, and the second transgene is inserted adjacent to the partial vaccinia B14R gene, inserted adjacent to the partial vaccinia B29R gene, or, when the B14R to B29R genes are deleted, inserted adjacent to the B13R gene. In other embodiments of the various embodiments and aspects described herein wherein the nucleic acid comprises the first transgene, the second transgene and the third transgene, the third transgene is inserted between the partial C2L and F3L vaccinia genes, the second transgene is inserted into the locus of the deletion in the B8R gene, and the first transgene is inserted between the partial B14R and B29R vaccinia genes. In another embodiment, the third transgene is inserted adjacent to the partial vaccinia C2L gene, inserted adjacent to the partial vaccinia F3L gene, or inserted between vaccinia genes C3L and F4L, the second transgene is inserted into the locus of the deletion in the B8R gene, and the first transgene is inserted adjacent to the partial vaccinia B14R gene, inserted adjacent to the partial vaccinia B29R gene, or, when the B14R to B29R genes are deleted, inserted adjacent to the B13R gene.

In various embodiments and aspects described herein wherein the nucleic acid comprises the nucleotide sequence of SEQ ID NO: 210, the partial C2L and F3L vaccinia genes are partial C2L and F3L vaccinia genes in SEQ ID NO: 210. In various embodiments and aspects described herein wherein the nucleic acid comprises the nucleotide sequence of SEQ ID NO: 210, the partial B14R and B29R vaccinia genes are partial B14R and B29R vaccinia genes in SEQ ID NO: 210.

In various embodiments and aspects described herein wherein the nucleic acid comprises the nucleotide sequence of SEQ ID NO: 624, the partial C2L and F3L vaccinia genes are partial C2L and F3L vaccinia genes in SEQ ID NO: 624. In various embodiments and aspects described herein wherein the nucleic acid comprises the nucleotide sequence of SEQ ID NO: 624, the partial B14R and B29R vaccinia genes are partial B14R and B29R vaccinia genes in SEQ ID NO: 624.

In a specific embodiment of the various embodiments and aspects described herein, insertion into the partial C2L and F3L vaccinia genes is insertion within the boundaries of a 5p deletion present in the recombinant vaccinia virus genome. In a specific embodiment of the various embodiments and aspects described herein, insertion between the partial B14R and B29R vaccinia genes is insertion within the boundaries of a 3p deletion present in the recombinant vaccinia virus genome.

In some embodiments of the various embodiments and aspects described herein, the anti-CTLA-4 antibody or antigen-binding fragment thereof encoded by the first nucleotide sequence is a full-length antibody (for example, a full-length human antibody, a full-length humanized antibody, or a full-length mouse antibody). In a specific embodiment, the first nucleotide sequence encodes a polypeptide that comprises the heavy chain and light chains of ipilimumab linked by a cleavage peptide, for example, a self-cleavage peptide, such as a 2A self-cleaving peptide (e.g., a T2A peptide). In another specific embodiment, the first nucleotide sequence encodes a polypeptide that comprises the heavy chain signal peptide and heavy chain, and light chain signal peptide and light chain of ipilimumab, linked by a cleavage peptide, for example, a self-cleavage peptide, such as a 2A self-cleaving peptide (e.g., a T2A peptide). In other embodiments of the various embodiments and aspects described herein, the anti-CTLA-4 antibody or antigen-binding fragment thereof encoded by the first nucleotide sequence is a single chain antibody (for example, a single chain human antibody, single chain humanized antibody, or a single chain mouse antibody, such as, for example, 9D9).

In a specific embodiment of the various embodiments and aspects described herein, the at least one promoter operably linked to the first nucleotide sequence encoding the anti-CTLA-4 antibody or antigen-binding fragment thereof is a B8R promoter. In another specific embodiment of the various embodiments and aspects described herein, the at least one promoter operably linked to the first nucleotide sequence encoding the anti-CTLA-4 antibody or antigen-binding fragment thereof is an H5R promoter. In a particular embodiment, the B8R promoter comprises the nucleotide sequence of SEQ ID NO: 564.

In some embodiments of the various embodiments and aspects described herein, the IL-12 peptide encoded by the second nucleotide sequence is a membrane-bound version of the cytokine. In specific embodiments, the IL-12 polypeptide comprises IL-12 p35 (e.g., human IL-12 p35) and a transmembrane domain. In a specific embodiment, the IL-12 polypeptide consists of IL-12 p35 (e.g., human IL-12 p35) and a transmembrane domain (IL12-TMp35). In specific embodiments, the IL-12 polypeptide comprises IL-12 p35 (e.g., human IL-12 p35), a transmembrane domain and a cytoplasmic domain. In a specific embodiment, the IL-12 polypeptide consists of IL-12 p35 (e.g., human IL-12 p35), a transmembrane domain and a cytoplasmic domain. The transmembrane domain can be derived from any membrane-bound protein (e.g., B7-1, membrane-bound TNFα, or membrane-bound FLT3L). The cytoplasmic domain can be derived from any protein that contains a cytoplasmic domain (e.g., B7-1, TNFα, or FLT3L). In a specific embodiment, the IL-12 polypeptide comprises IL-12 p35 (e.g., human IL-12 p35) and a B7 cytoplasmic and membrane domain from the B7-1 antigen, a commonly used element for mammalian surface display. In a specific embodiment, the IL-12 polypeptide consists of IL-12 p35 (e.g., human IL-12 p35) and a B7 cytoplasmic and membrane domain from the B7-1 antigen. In specific embodiments, the IL-12 polypeptide comprises IL-12 p70 (e.g., human IL-12 p70), which comprises a p40 subunit (e.g., human IL-12 p40) and a p35 subunit (e.g., human IL-12 p35), and a transmembrane domain. In a specific embodiment, the IL-12 polypeptide consists of IL-12 p70 (e.g., human IL-12 p70), which comprises a p40 subunit (e.g., human IL-12 p40) and a p35 subunit (e.g., human IL-12 p35), and a transmembrane domain (IL12-TMp70, or p40-p35-TM). In specific embodiments, the IL-12 polypeptide comprises IL-12 p70 (e.g., human IL-12 p70), which comprises a p40 subunit (e.g., human IL-12 p40) and a p35 subunit (e.g., human IL-12 p35), and a transmembrane domain and a cytoplasmic domain. In a specific embodiment, the IL-12 polypeptide comprises IL-12 p70 (e.g., human IL-12 p70), which comprises a p40 subunit (e.g., human IL-12 p40) and a p35 subunit (e.g., human IL-12 p35), and a transmembrane domain and a cytoplasmic domain. The transmembrane domain can be derived from any membrane-bound protein (e.g., B7-1, membrane-bound TNFα, or membrane-bound FLT3L). The cytoplasmic domain can be derived from any protein that contains a cytoplasmic domain (e.g., B7-1, TNFα, or FLT3L). In a specific embodiment, the IL-12 polypeptide comprises IL-12 p70 (e.g., human IL-12 p70), which comprises a p40 subunit (e.g., human IL-12 p40) and a p35 subunit (e.g., human IL-12 p35), and a B7 cytoplasmic and membrane domain from the B7-1 antigen. In a specific embodiment, the IL-12 polypeptide consists of IL-12 p70 (e.g., human IL-12 p70), which comprises a p40 subunit (e.g., human IL-12 p40) and a p35 subunit (e.g., human IL-12 p35), and a B7 cytoplasmic and membrane domain from the B7-1 antigen. In certain embodiments, the IL-12 polypeptide is a human IL-12 polypeptide (for example, human IL12-TMp35 or human IL12-TMp70). In certain embodiments, the IL-12 polypeptide is a mouse IL-12 polypeptide (for example, mouse IL12-TMp35 or mouse IL12-TMp70).

In a specific embodiment of the various embodiments and aspects described herein, the at least one promoter operably linked to the second nucleotide sequence encoding the IL-12 polypeptide is a late promoter having the nucleotide sequence of SEQ ID NO: 561. In a specific embodiment of the various embodiments and aspects described herein, the at least one promoter operably linked to the second nucleotide sequence encoding the IL-12 polypeptide is a B8R promoter. In a specific embodiment of the various embodiments and aspects described herein, the at least one promoter operably linked to the second nucleotide sequence encoding the IL-12 polypeptide is a late promoter having the nucleotide sequence of SEQ ID NO: 561 and a B8R promoter. In a particular embodiment, the B8R promoter comprises the nucleotide sequence of SEQ ID NO: 564.

In specific embodiments, the FLT3L encoded by the third nucleotide sequence is a soluble form of FLT3L. In particular embodiments, the FLT3L encoded by the third nucleotide sequence is a soluble form of human FLT3L. In certain embodiments, the FLT3L encoded by the third nucleotide sequence is a soluble form of the human FLT3L set forth in GenBank Accession No. U03858.1. For example, in specific embodiments, the FLT3L encoded by the third nucleotide sequence lacks the entire FLT3L transmembrane (e.g., the transmembrane domain of the human FLT3L set forth in GenBank Accession No. U03858.1). In other examples, the FLT3L encoded by the third nucleotide sequence lacks at least 80%, at least 85%, at least 90%, or at least 95% of the FLT3L transmembrane domain (e.g., the transmembrane domain of the human FLT3L set forth in GenBank Accession No. U03858.1). In one embodiment, the FLT3L encoded by the third nucleotide sequence lacks the entire FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and the entire FLT3L cytoplasmic domain. In another embodiment, the FLT3L encoded by the third nucleotide sequence lacks at least 80%, at least 85%, at least 90%, or at least 95% of the FLT3L transmembrane domain and at least 25%, at least 30%, at least 40%, at least 50%, at least 60%, at least 70%, at least 80%, at least 85%, at least 90%, or at least 95% of the FLT3L cytoplasmic domain. In another embodiment, the FLT3L encoded by the third nucleotide sequence lacks the entire FLT3L transmembrane domain and 5, 6, 7, 8, 9, 10, 12, 13, 14, or 15 of the N-terminal amino acid residues of the FLT3L cytoplasmic domain. In another embodiment, the FLT3L encoded by the third nucleotide sequence lacks the entire FLT3L transmembrane domain and 1, 2, 3, or 4 of the N-terminal amino acid residues of the FLT3L cytoplasmic domain. In certain of the embodiments and aspects, the transmembrane and cytoplasmic domains are of the FLT3L sequence set forth in GenBank Accession No. U03858.1.

In another embodiment, the FLT3L encoded by the third nucleotide sequence lacks the entire FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane and extracellular domains are of the FLT3L sequence set forth in GenBank Accession No. U03858.1.

In another embodiment, the FLT3L encoded by the third nucleotide sequence lacks the entire FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 25%, at least 30%, at least 40%, at least 50%, at least 60%, at least 70%, at least 80%, at least 85%, at least 90%, or at least 95% of the FLT3L transmembrane domain, the entire cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, at least 25%, at least 30%, at least 40%, at least 50%, at least 60%, at least 70%, at least 80%, at least 85%, at least 90%, or at least 95% of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks the entire FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1, 2, 3, 4, 5, 6, 7, 8, 9 or 10 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 11, 12, 13, 14, 15, 16, 17, 18, 19 or 20 amino acids from the carboxy-terminus of the FLT3L extracellular domain. In another embodiment, the FLT3L encoded by the third nucleotide sequence lacks at least 80%, at least 85%, at least 90%, or at least 95% of the FLT3L transmembrane domain, 1, 2, 3, 4, 5, 6, 7, 8, 9, 10, 12, 13, 14, or 15 of the N-terminal amino acid residues of the cytoplasmic domain, and 1-5, 1-10, 5-10, 10-20, 15-20, 1-20, 5-20, or 10-20 amino acids from the carboxy-terminus of the FLT3L extracellular domain. In certain of the embodiments and aspects, the transmembrane, cytoplasmic and extracellular domains are of the FLT3L sequence set forth in GenBank Accession No. U03858.1.

In a specific embodiment, the FLT3L encoded by the third nucleotide sequence is an X7 isoform and the third nucleotide sequence lacks a 179-nucleotide sequence as described in Lyman et al., 1994, Blood 83:2795-2801. In specific embodiments, the FLT3L comprises the amino acid sequence set forth in SEQ ID NO: 213. In specific embodiments, the third nucleotide sequence comprises the sequence set forth in SEQ ID NO: 216. In specific embodiments, the third nucleotide sequence is set forth in SEQ ID NO: 216.

In a specific embodiment of the various embodiments and aspects described herein, the at least one promoter operably linked to the third nucleotide sequence encoding FLT3L is a B8R promoter. In a specific embodiment of the various embodiments and aspects described herein, the at least one promoter operably linked to the third nucleotide sequence encoding FLT3L is a B19R promoter. In a specific embodiment of the various embodiments and aspects described herein, the at least one promoter operably linked to the third nucleotide sequence encoding FLT3L is a B8R promoter and a B19R promoter. In a particular embodiment, the B8R promoter comprises the nucleotide sequence of SEQ ID NO: 564. In a particular embodiment, the B19R promoter comprises the nucleotide sequence of SEQ ID NO: 565.

The invention also contemplates nucleic acids as described herein which further comprise a fourth transgene comprising a fourth nucleotide sequence encoding a detectable marker, e.g., a fluorescent marker (for example, a green fluorescent protein (GFP) such as an enhanced GFP (eGFP)). In certain embodiments, the nucleic acid further comprises a nucleotide sequence comprising at least one promoter operably linked to the fourth nucleotide sequence encoding the fluorescent marker. In certain embodiments, the fourth nucleotide sequence encoding the fluorescent marker is linked and downstream of one of the first, second, and third nucleotide sequences.

In a specific embodiment, the at least one promoter operably linked to the fourth nucleotide sequence encoding the fluorescent marker is an E3L promoter. In another specific embodiment, the at least one promoter operably linked to the fourth nucleotide sequence encoding the fluorescent marker is a p7.5 promoter.

In specific embodiments, provided herein is a vector comprising a nucleotide sequence of SEQ ID NO: 210 or SEQ ID NO: 624 with transgene(s) identified in Table 45, inserted into the locus (loci) identified in Table 45, and operably linked to the promoter(s) identified in Table 45. In specific embodiments, provided herein is a vector as described in Table 45.

It is also contemplated that the first transgene, the second transgene, the third transgene, and/or the fourth transgene can be inserted into the TK gene locus. The other transgenes (if any) can be inserted at other loci, for example, between the partial C2L and F3L vaccinia genes, the locus of the deletion in the B8R gene, between the partial B14R and B29R vaccinia genes, and/or the HA gene locus. In some embodiments, the recombinant vaccinia virus genome comprises a deletion in the TK gene. In a specific embodiment, the first transgene, the second transgene, the third transgene, and/or the fourth transgene is inserted into the locus of the deletion in the TK gene. In other embodiments, the TK gene is not deleted but the first transgene, the second transgene, the third transgene, and/or the fourth transgene is inserted into the TK gene and disrupts the function of the TK gene.

In other embodiments, the recombinant vaccinia virus genome comprises a functional, e.g., wild-type, TK gene and none of the transgene(s) is inserted into the TK gene locus. A wild-type TK gene includes a TK gene naturally found in a vaccinia virus genome.

It is also contemplated that the first transgene, the second transgene, the third transgene, and/or the fourth transgene can be inserted into the HA gene locus. The other transgenes (if any) can be inserted at other loci, for example, between the partial C2L and F3L vaccinia genes, the locus of the deletion in the B8R gene, between the partial B14R and B29R vaccinia genes, and/or the TK gene locus. In some embodiments, the recombinant vaccinia virus genome comprises a deletion in the HA gene. In a specific embodiment, the first transgene, the second transgene, the third transgene, and/or the fourth transgene is inserted into the locus of the deletion in the HA gene. In other embodiments, the HA gene is not deleted but the first transgene, the second transgene, the third transgene, and/or the fourth transgene is inserted into the HA gene and disrupts the function of the HA gene.

In other embodiments, the recombinant vaccinia virus genome comprises a functional, e.g., wild-type, HA gene and none of the transgene(s) is inserted into the HA gene locus. A wild-type HA gene includes a HA gene naturally found in a vaccinia virus genome.

In certain embodiments of the various embodiments and aspects described herein, at least one promoter is operably linked to the first nucleotide sequence, the second nucleotide sequence and/or the third nucleotide sequence, wherein the at least one promoter is an early promoter, a late promoter, or an early/late promoter. In particular embodiments, the at least one promoter is an early promoter and a late promoter. In specific embodiments, a late promoter may comprise a TAAAT nucleotide sequence (SEQ ID NO. 631).

In certain embodiments of the various embodiments and aspects described herein, the at least one promoter operably linked to the first nucleotide sequence encoding the anti-CTLA-4 antibody is an early promoter, a late promoter, or an early/late promoter. In particular embodiments, the at least one promoter is an early promoter and a late promoter. In specific embodiments, a late promoter may comprise a TAAAT nucleotide sequence (SEQ ID NO. 631). In a specific embodiment, the at least one promoter operably linked to the first nucleotide sequence is an H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence). In another specific embodiment, the at least one promoter operably linked to the first nucleotide sequence is an H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). In a specific embodiment, the at least one promoter operably linked to the first nucleotide sequence is an H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence) and an H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). When the H5R late promoter comprises the nucleotide sequence of TAAAT (SEQ ID NO. 631), in one embodiment, the nucleic acid comprises an intervening sequence between TAAAT and the ATG translation initiation codon (for example, an intervening sequence that is about 10, 20, 30, or 40-nucleotide in length); in another embodiment, there is no intervening sequence between TAAAT and the ATG translation initiation codon (for example, the last two nucleotides of TAAAT are the first two nucleotides of the ATG translation initiation codon). In another specific embodiment, the at least one promoter operably linked to the first nucleotide sequence is a pS early promoter (e.g., comprising the nucleotide sequence of AAAATTGAAATTTTA (SEQ ID NO. 555)). In another specific embodiment, the at least one promoter operably linked to the first nucleotide sequence is a pS late promoter (e.g., comprising the nucleotide sequence of TTTTATTTTTTTTTTTTGGAATATAAATA (SEQ ID NO. 556)). In another specific embodiment, the at least one promoter operably linked to the first nucleotide sequence is a pS early/late promoter (e.g., comprising the nucleotide sequence of AAAATTGAAATTTTATTTTTTTTTTTTGGAATATAAATA (SEQ ID NO. 557)). In another specific embodiment, the at least one promoter operably linked to the first nucleotide sequence is a LEO early promoter (e.g., comprising the nucleotide sequence of TTTTATTTTTTTTTTTTGGAATATAAATA (SEQ ID NO. 556)). In another specific embodiment, the at least one promoter operably linked to the first nucleotide sequence is a LEO late promoter (e.g., comprising the nucleotide sequence of AAAATTGAAAAAATA (SEQ ID NO. 558)). In another specific embodiment, the at least one promoter operably linked to the first nucleotide sequence is a LEO early/late promoter (e.g., comprising the nucleotide sequence of TTTTATTTTTTTTTTTTGGAATATAAATATCCGGTAAAATTGAAAAAATA (SEQ ID NO. 559)).

In certain embodiments of the various embodiments and aspects described herein, the at least one promoter operably linked to the second nucleotide sequence encoding the IL-12 polypeptide is a late promoter. In a specific embodiment, the late promoter comprises the nucleotide sequence of TTNTTTTTTNTTTTTTTNNNNTATAAAT (SEQ ID NO: 560, wherein N is any nucleotide). In another specific embodiment, the late promoter comprises the nucleotide sequence of TTGTATTTTCTTTTGTTGGCATATAAAT (SEQ ID NO: 561). In another specific embodiment, the late promoter is a D13L promoter (e.g., comprising the nucleotide sequence of TTTATTGTAAGCTTTTTCCATTTTAAAT (SEQ ID NO. 562)). In another specific embodiment, the late promoter is a F17R promoter (e.g., comprising the nucleotide sequence of TCATTTTGTTTTTTTCTATGCTATAAAT (SEQ ID NO. 563)).

In certain embodiments of the various embodiments and aspects described herein, the at least one promoter operably linked to the third nucleotide sequence encoding FLT3L is an early promoter, a late promoter, or an early/late promoter. In a specific embodiment, the at least one promoter operably linked to the third nucleotide sequence is the B8R promoter (e.g., comprising the nucleotide sequence of TAAAAATTTAAAATATATTATCACTTCAGT (SEQ ID NO. 564)). In another specific embodiment, the at least one promoter operably linked to the third nucleotide sequence is the B19R promoter (e.g., comprising the nucleotide sequence of AAAAAACTGATATTATATAAATATTTTAGT (SEQ ID NO. 565)). In another specific embodiment, the at least one promoter operably linked to the third nucleotide sequence comprises the nucleotide sequence of NNAAAANTGAAAANATA (SEQ ID NO. 566, wherein N is any nucleotide). In another specific embodiment, the at least one promoter operably linked to the third nucleotide sequence is the E3L promoter (e.g., comprising the nucleotide sequence of AAAAAAATGATAAAGTAGGTTCAGTTTTAT (SEQ ID NO. 567)). In another specific embodiment, the at least one promoter operably linked to the third nucleotide sequence is the F11L promoter (e.g., comprising the nucleotide sequence of TAAAAAGTGAAAAACAATATTATTTTTATC (SEQ ID NO. 568)). In another specific embodiment, the at least one promoter operably linked to the third nucleotide sequence is the B2R promoter (e.g., comprising the nucleotide sequence of AAAATTAAAAAATAACTTAATTTATTATTG (SEQ ID NO. 569)).

In certain embodiments of the various embodiments and aspects described herein, the promoter sequence overlaps with or is within about 100 nucleotide of the translation initiation codon of transgene to which the promoter is operatively linked. In a specific embodiment, the promoter sequence is within about 80 nucleotides of the translation initiation codon of the transgene to which the promoter is operatively linked. In a specific embodiment, the promoter sequence is within about 70 nucleotides of the translation initiation codon of the transgene to which the promoter is operatively linked. In a specific embodiment, the promoter sequence is within about 60 nucleotides of the translation initiation codon of the transgene to which the promoter is operatively linked. In a specific embodiment, the promoter sequence is within about 50 nucleotides of the translation initiation codon of the transgene to which the promoter is operatively linked. In another specific embodiment, the promoter sequence is within about 40 nucleotides of the translation initiation codon of the transgene to which the promoter is operatively linked. In another specific embodiment, the promoter sequence is within about 30 nucleotides of the translation initiation codon of the transgene to which the promoter is operatively linked. In another specific embodiment, the promoter sequence is within about 20 nucleotides of the translation initiation codon of the transgene to which the promoter is operatively linked. In another specific embodiment, the promoter sequence is within about 10 nucleotides of the translation initiation codon of the transgene to which the promoter is operatively linked. In another specific embodiment, the promoter sequence is within about 5 nucleotides of the translation initiation codon of the transgene to which the promoter is operatively linked. In another specific embodiment, the promoter sequence is within 2 nucleotides of the translation initiation codon of the transgene to which the promoter is operatively linked. In another specific embodiment, the promoter sequence overlaps with the translation initiation codon of the transgene to which the promoter is operatively linked.

In certain embodiments of the various embodiments and aspects described herein wherein the nucleic acid comprises a transgene comprising a nucleotide sequence the nucleic acid may further comprises a nucleotide sequence comprising an untranslated region (UTR), for example, a H5R UTR or a portion thereof, e.g., at least 80%, at least 85%, at least 90%, or at least 95% of the H5R UTR, operably linked to the nucleotide sequence. In a specific embodiment, the H5R UTR or a portion thereof comprises a H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence). In another specific embodiment, the H5R UTR or a portion thereof comprises a H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). In another specific embodiment, the H5R UTR or a portion thereof comprises the H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence) and the H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). In another specific embodiment, the H5R UTR comprises the nucleotide sequence of TTAAAGTTACAAACAACTAGGAAATTGGTTTATGATGTATAATTTTTTTAGTTTTT ATAGATTCTTTATTCTATACTTAAAAAATGAAAATAAATACAAAGGTTCTTGAGG GTTGTGTTAAATTGAAAGCGAGAAATAATCATAAATTATTTCATTATCGCGATAT CCGTTAAGTTTGTATCGTA (SEQ ID NO. 626).

In specific embodiments of the various embodiments and aspects described herein wherein the nucleic acid comprises a first transgene comprising a first nucleotide sequence encoding an antibody or antigen-binding fragment thereof that specifically binds to CTLA-4, the nucleic acid may further comprise a nucleotide sequence encoding an untranslated region (UTR). For example, the UTR can comprise an H5R UTR or a portion thereof (e.g., at least 80%, at least 85%, at least 90%, or at least 95% of the H5R UTR) operably linked to the first nucleotide sequence. In a specific embodiment, the H5R UTR comprises a H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence). In another specific embodiment, the H5R UTR comprises a H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). In another specific embodiment, the H5R UTR comprises a H5R early promoter (e.g., comprising the nucleotide sequence of AAAAATGAAAATAAA (SEQ ID NO. 630) or TAAAAAATGAAAATAAATACAAAGGTTCTT (SEQ ID NO. 553), optionally with one, two, three, four, five, or more nucleotides upstream and/or downstream of the sequence) and a H5R late promoter (e.g., comprising the nucleotide sequence of TAAAT (SEQ ID NO. 631), TCTTGAGGGTTGTGTTAAATTGAAAGCGAGAAATAATCATAAAT (SEQ ID NO. 632), or AAATTGAAAGCGAGAAATAATCATAAAT (SEQ ID NO. 554), optionally with one, two, three, four, five, or more nucleotides upstream and/or downstream of the sequence). In another specific embodiment, the H5R UTR comprises the nucleotide sequence of TTAAAGTTACAAACAACTAGGAAATTGGTTTATGATGTATAATTTTTTTAGTTTTT ATAGATTCTTTATTCTATACTTAAAAAATGAAAATAAATACAAAGGTTCTTGAGG GTTGTGTTAAATTGAAAGCGAGAAATAATCATAAATTATTTCATTATCGCGATAT CCGTTAAGTTTGTATCGTA (SEQ ID NO. 626).

In certain embodiments of the various embodiments and aspects described herein, at least one, two, three, four, five, six, seven, eight, nine, ten, twenty, thirty, forty, fifty, sixty, seventy, eighty, ninety, or a hundred of, or all of the following genes are not deleted from the recombinant vaccinia virus genome: C3L, C4L, CSL, C6L, C7L, CBL, C9L, C10L, C11R, C12L, C13L, C14L, C15L (in the 5′ ITR), C16L (in the 5′ ITR), C17L (in the 5′ ITR), C18L (in the 5′ ITR), C19L (in the 5′ ITR), C2OL (in the 5′ ITR), C21L (in the 5′ ITR), C22L (in the 5′ ITR), C23L (in the 5′ ITR), F4L, FSL, F6L, F7L, F8L, F9L, F10L, F11L, F12L, F13L, F14L, F15L, F16L, F17R, E1L, E2L, E3L, E4L, ESR, E6R, E7R, EBR, E9L, E10R, E11L, O1L, O2L, I1L, I2L, I3L, I4L, I5L, I6L, I7L, I8R, G1L, G2R, G3L, G4L, G5R, G6R, G7L, G8R, G9R, L1R, L2R, L3L, L4R, LSR, J1R, J2R, J3R, J4R, J5L, J6R, H1L, H2R, H3L, H4L, H5R, H6R, H7R, D1R, D2L, D3R, D4R, D5R, D6R, D7R, D8L, D9R, D10R, D11L, D12L, D13L, A1L, A2L, A3L, A4L, ASR, A6L, A7L, A8R, A9L, A10L, A11R, A12L, A13L, A14L, A15L, A16L, A17L, A18R, A19L, A20R, A21L, A22R, A23R, A24R, A25L, A26L, A27L, A28L, A29L, A30L, A31R, A32L, A33R, A34R, A35R, A36R, A37R, A38L, A39R, A4OR, A41L, A42R, A43R, A44L, A45R, A46R, A47L, A48R, A49R, ASOR, A51R, A52R, A53R, A54L, A55R, A56R, A57R, B1R, B2R, B3R, B4R, B5R, B6R, B7R, B8R, B9R, B10R, B11R, B12R, and B13R (see, e.g., Goebel et al., 1990, Virology 179(1):247-266 for a description of the genes, which is incorporated herein by reference for such description).

In another aspect, provided herein is a nucleic acid comprising the nucleic acid sequence described in Table 43.

In another aspect, provided herein is a nucleic acid described in an example in Section 6.

In one aspect, provided are nucleic acids comprising a recombinant orthopoxvirus genome, wherein said recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 16 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 17 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 18 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 19 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 20 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 21 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of at least 22 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In some embodiments, said recombinant orthopoxvirus genome comprises a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.

In one aspect, provided are nucleic acids comprising a recombinant orthopoxvirus genome, wherein said recombinant orthopoxvirus genome comprises a deletion of at least 1 gene selected from the group consisting of the B14R, B15R, B16R, B17L, B18R, B19R, and B20R genes.

In some embodiments, said recombinant orthopoxvirus genome comprises a deletion of at least 2 genes, each gene selected from the group consisting of the B14R, B15R, B16R, B17L, B18R, B19R, and B20R genes.

In some embodiments, said recombinant orthopoxvirus genome comprises a deletion of at least 3 genes, each gene selected from the group consisting of the B14R, B15R, B16R, B17L, B18R, B19R, and B20R genes.

In some embodiments, said recombinant orthopoxvirus genome comprises a deletion of at least 4 genes, each gene selected from the group consisting of the B14R, B15R, B16R, B17L, B18R, B19R, and B20R genes.

In some embodiments, said recombinant orthopoxvirus genome comprises a deletion of at least 5 genes, each gene selected from the group consisting of the B14R, B15R, B16R, B17L, B18R, B19R, and B20R genes.

In some embodiments, said recombinant orthopoxvirus genome comprises a deletion of at least 6 genes, each gene selected from the group consisting of the B14R, B15R, B16R, B17L, B18R, B19R, and B20R genes.

In some embodiments, said recombinant orthopoxvirus genome comprises a deletion of each of the B14R, B15R, B16R, B17L, B18R, B19R, and B20R genes.

In one aspect, provided are nucleic acids comprising a recombinant orthopoxvirus genome, wherein said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 2 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of each of the C2L, C1L, N1L, N2L, M1L, M2L, K1L, K2L, K3L, K4L, K5L, K6L, K7R, F1L, F2L, and F3L genes.

In some embodiments, said recombinant orthopoxvirus genome comprises a deletion of at least 1 gene that encodes a protein involved in host interaction. For example, in some embodiments, said protein affects calcium-independent adhesion to the extracellular matrix. In some embodiments, said protein is an NF-κB inhibitor, e.g., an NF-κB inhibitor encoded by a gene selected from the group consisting of the C2L, N1L, M2L, K1L, and K7R genes. In some embodiments, said protein is an apoptosis inhibitor, e.g., a caspase-9 inhibitor (such as one encoded by the F1L gene), a BCL-2-like protein (such as one encoded by N1L). In some embodiments, said protein is an interferon regulatory factor 3 (IRF3) inhibitor (such as one encoded by N2L or K7R), a serine protease inhibitor, a protein that prevents cell fusion (such as one encoded by K2L), an RNA-activated protein kinase (PKR) inhibitor (such as one encoded by K1L or K3L), a virulence factor (such as one encoded by F3L), an IL-1-beta inhibitor (such as one encoded by B16R), or a secreted IFNα sequestor (such as one encoded by B19R).

In some embodiments, said recombinant orthopoxvirus genome comprises a deletion of at least 1 gene encoding a protein involved in DNA replication. For example, in some embodiments, said protein is a DNA modifying nuclease (e.g., a protein encoded by K4L) or a deoxyuridine triphosphatase (dUTPase) (e.g., a protein encoded by is F2L). In some embodiments, at least one deleted gene's entire nucleotide sequence is deleted. In some embodiments, at least one deleted gene is only partially deleted, and the partial deletion is sufficient to render said partially deleted gene nonfunctional upon introduction into a host cell.

In some embodiments, said recombinant orthopoxvirus genome comprises at least two copies of inverted terminal repeats (ITRs).

In some embodiments, said recombinant orthopoxvirus genome lacks any copies of ITRs.

In some embodiments, said recombinant orthopoxvirus genome comprises a deletion in at least one copy of an ITR selected from the group consisting of B21R-ITR, B22R-ITR, B23R-ITR, B24R-ITR, B25R-ITR, B26R-ITR, B27R-ITR, B28R-ITR, and B29R-ITR.

In some embodiments, said recombinant orthopoxvirus genome comprises a deletion in at least all of the following copies of ITRs: B21R-ITR, B22R-ITR, B23R-ITR, B24R-ITR, B25R-ITR, B26R-ITR, B27R-ITR, B28R-ITR, and B29R-ITR.

In some embodiments, said recombinant orthopoxvirus genome comprises a deletion in the B8R gene.

In some embodiments, said recombinant orthopoxvirus genome comprises an intact B8R gene.

In one aspect, provided are nucleic acids comprising a recombinant orthopoxvirus genome, wherein said recombinant orthopoxvirus genome comprises (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes; and (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR.

In one aspect, provided are nucleic acids comprising a recombinant orthopoxvirus genome, wherein said recombinant orthopoxvirus genome comprises (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes; and (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR, wherein said recombinant orthopoxvirus genome comprises an intact B8R gene.

In some embodiments, provided nucleic acids further comprise at least one transgene selected from the group consisting of a transgene encoding an immune checkpoint inhibitor, a transgene encoding an interleukin (IL), and a transgene encoding a cytokine. In some embodiments, provided nucleic acids further comprise at least two transgenes selected from the group consisting of a transgene encoding an immune checkpoint inhibitor, a transgene encoding an interleukin (IL), and a transgene encoding a cytokine. In some embodiments, provided nucleic acids further comprise a transgene encoding an immune checkpoint inhibitor, a transgene encoding an interleukin (IL), and a transgene encoding a cytokine.

For example, in some embodiments, provided nucleic acids comprise a transgene encoding an immune checkpoint inhibitor. In some embodiments, said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In some embodiments, said immune checkpoint inhibitor is an anti-PD-L1 antibody or antigen-binding fragment thereof or an anti-CTLA-4 antibody or antigen-binding fragment thereof. In some embodiments, said immune checkpoint inhibitor is an anti-PD1 antibody or antigen-binding fragment thereof. In some embodiments, said immune checkpoint inhibitor is an anti-CTLA-4 antibody or antigen-binding fragment thereof.

For example, in some embodiments, provided nucleic acids comprise a transgene encoding an interleukin (IL). In some embodiments, said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In some embodiments, said interleukin is selected from the group consisting of IL-12 p35, IL-12 p40, and IL-12 p70. In some embodiments, said interleukin is membrane-bound. In some embodiments, said interleukin is membrane-bound IL-12 p70. In some embodiments, said interleukin is membrane-bound IL-12 p35.

For example, in some embodiments, provided nucleic acids comprise a transgene encoding a cytokine. In some embodiments, said cytokine is an interferon (IFN). In some embodiments, the interferon is selected from the group consisting of IFN-alpha, IFN-beta, IFN-delta, IFN-epsilon, IFN-tau, IFN-omega, IFN-zeta, and IFN-gamma.

In some embodiments, the cytokine is a TNF superfamily member protein. In some embodiments, the TNF superfamily member protein is selected from the group consisting of TRAIL, Fas ligand, LIGHT (TNFSF-14), TNF-alpha, and 4-1BB ligand.

In some embodiments, the cytokine is selected from the group consisting of GM-CSF, Flt3 ligand, CD40 ligand, TGF-beta, VEGF-R2, and c-kit. In some embodiments, the cytokine is Flt3 ligand.

In some embodiments, said recombinant orthopoxvirus genome comprises a deletion in the B8R gene and at least one transgene is inserted into the deletion in the B8R gene. In some embodiments, at least two transgenes are inserted into the deletion in the B8R gene. In some embodiments, at least three transgenes are inserted into the deletion in the B8R gene. In some embodiments, at least one transgene is inserted in a locus that is not at the deletion in the B8R gene, for example, a locus at the boundary of a deletion at the 5′ end of the orthopoxvirus genome or at a locus at the boundary of a deletion at the 3′ end of the orthopoxvirus genome.

In one aspect, provided are nucleic acids comprising a recombinant orthopoxvirus genome, wherein said recombinant orthopoxvirus genome comprises (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes; (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR; (iii) an IL-12-TM transgene inserted into the deletion in the B8R gene; (iv) an Flt3 ligand transgene inserted into the deletion in the B8R gene; and (v) one of: (a) a transgene encoding a single chain anti-CTLA-4 antibody or antigen-binding fragment thereof, or (b) (i) a transgene encoding a heavy chain of an anti-CTLA-4 antibody or antigen-binding fragment thereof, and (ii) a transgene encoding a light chain of an anti-CTLA-4 antibody or antigen-binding fragment thereof, wherein the transgene(s) in part (v) is/are inserted within the boundaries of a 5p deletion present in the recombinant orthopoxvirus genome, an wherein the anti-CTLA-4 antibody or antigen-binding fragment thereof is capable of binding CTLA-4.

In some embodiments, the orthopoxvirus genome is derived from a sequence of SEQ ID NO: 210, wherein (a) said derived sequence comprises a deletion of the B8R gene, and the IL-12-TM transgene, the Flt3 ligand transgene, and the transgene(s) encoding the single or double-chain anti-CTLA-4 antibody; (b) the IL-12-TM transgene encodes a protein comprising an amino acid sequence of is SEQ ID NO: 212; (c) the Flt3 ligand transgene encodes a protein comprising an amino acid sequence of SEQ ID NO: 213; and (d) the anti-CTLA-4 antibody comprises an amino acid sequence of SEQ ID NO: 211.

In some embodiments of provided nucleic acids, the nucleic acid further comprises a transgene encoding a tumor-associated antigen, for example, a tumor-associated antigen listed in any one of Tables 3-30. In some embodiments, the tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.

In some embodiments, the tumor-associated antigen comprises MAGE-A3, or one or more fragments thereof.

In some embodiments, the tumor-associated antigen comprises NY-ESO-1, or one or more fragments thereof.

In some embodiments, the tumor-associated antigen comprises one or more human papillomavirus (HPV) proteins, or fragments thereof. In some embodiments, the HPV proteins or fragments thereof comprise one or more of (i) E6 and E7 proteins, or fragments thereof, of HPV16 and (ii) E6 and E7 proteins, or fragments thereof, of HPV18. In some embodiments, the sequences of said HPV proteins or fragments are disclosed in International Patent Publication WO/2014/127478, the contents of which are incorporated herein by reference.

In some embodiments, the tumor-associated antigen comprises brachyury or one or more fragments thereof.

In some embodiments, the tumor-associated antigen comprises prostatic acid phosphatase, or one or more fragments thereof.

When more than one transgene described herein is inserted into a recombinant orthopoxvirus genome (e.g., a recombinant vaccinia virus genome), the transgenes can be inserted into one locus or multiple loci (e.g., two loci or three loci). When two or more transgenes described above are inserted into the same loci, the transgenes can be inserted with the same orientation or different orientations relative to one of or both of the flanking endogenous orthopoxvirus genes (e.g., vaccinia virus genes), and also relative to each other. It is also contemplated that, when two or more transgenes are inserted into the same locus, the order of the transgenes inserted into the same locus of the recombinant orthopoxvirus genome (e.g., a recombinant vaccinia virus genome) can be different.

In certain embodiments of the various embodiments and aspects described herein, the nucleotide sequence encoding the antibody or antigen-binding fragment thereof that specifically binds to CTLA-4 encodes the heavy and light chains of the anti-CTLA-4 antibody (e.g., ipilimumab) separated by cleavage peptide, for example a self-cleavage peptide, e.g., a 2A self-cleaving peptide. In a specific embodiment, the 2A self-cleavage peptide is a T2A peptide. In a particular embodiment, the T2A peptide comprises the amino acid sequence of GSGEGRGSLLTCGDVEENPGP (SEQ ID NO: 570). In a particular embodiment, the T2A peptide comprises the amino acid sequence of PRGSGEGRGSLLTCGDVEENPGP (SEQ ID NO: 571). In another particular embodiment, the T2A peptide comprises the amino acid sequence of EGRGSLLTCGDVEENPGP (SEQ ID NO: 572). In another specific embodiment, the 2A self-cleavage peptide is a P2A peptide. In a particular embodiment, the P2A peptide comprises the amino acid sequence of GSGATNFSLLKQAGDVEENPGP (SEQ ID NO: 573). In another particular embodiment, the P2A peptide comprises the amino acid sequence of ATNFSLLKQAGDVEENPGP (SEQ ID NO: 574). In another specific embodiment, the 2A self-cleavage peptide is a E2A peptide. In a particular embodiment, the E2A peptide comprises the amino acid sequence of GSGQCTNYALLKLAGDVESNPGP (SEQ ID NO: 575). In another particular embodiment, the E2A peptide comprises the amino acid sequence of QCTNYALLKLAGDVESNPGP (SEQ ID NO: 576). In another specific embodiment, the 2A self-cleavage peptide is a F2A peptide. In a particular embodiment, the F2A peptide comprises the amino acid sequence of GSGVKQTLNFDLLKLAGDVESNPGP (SEQ ID NO: 577). In another particular embodiment, the F2A peptide comprises the amino acid sequence of VKQTLNFDLLKLAGDVESNPGP (SEQ ID NO: 578). Linking of the heavy and light chains of the antibody by a 2A self-cleavage peptide enables the antibody transgene to be translated in one open reading frame and self-cleavage to occur co-translationally, resulting in equal amounts of the co-expressed heavy and light chains. In a specific embodiment, the anti-CTLA-4 antibody encoded by a nucleotide sequence described herein comprises the amino acid sequence of SEQ ID NO: 211.

In various embodiments, the nucleic acid provided herein is a recombinant nucleic acid.

### 5.2.4. Modified Orthopoxviruses

In one aspect, provided herein is a virus comprising the nucleic acid described in Section 5.2.3. In a specific embodiment, provided herein is a virus comprising the nucleic acid described in Section 5.2.3, wherein the nucleic acid comprises a recombinant vaccinia virus genome that comprises a second transgene comprising a second nucleotide sequence encoding a membrane-bound IL-12 polypeptide.

In another aspect, provided herein is a virus described in an example in Section 6.

In another aspect, provided are viruses comprising the nucleic acid comprising the recombinant orthopoxvirus genome described herein. In some embodiments, a) the recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes; b) said recombinant orthopoxvirus genome comprises a deletion of at least 1 gene selected from the group consisting of the B14R, B15R, B16R, B17L, B18R, B19R, and B20R genes; or c) said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.

In some embodiments, said virus is derived from a vaccinia virus. In some embodiments, said vaccinia virus is derived from a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16mO, Tashkent, Tian Tan, and WAU86/88-1. In some embodiments, said vaccinia virus is derived from a strain selected from the group consisting of Copenhagen, Western Reserve, Tian Tan, Wyeth, and Lister. In some embodiments, said vaccinia virus is derived from a Copenhagen strain vaccinia virus.

In some embodiments, said recombinant orthopoxvirus genome further comprises a Thymidine Kinase (TK) gene. In some embodiments, said recombinant orthopoxvirus genome further comprises a ribonucleotide reductase gene.

In some embodiments of provided viruses, upon contacting a population of cells (e.g., mammalian cells) with said virus, the population of cells (e.g., mammalian cells) exhibit increased syncytia formation relative to a population of cells (e.g., mammalian cells) of the same type contacted with a form of the virus that does not comprise said deletion.

In some embodiments of provided viruses, upon contacting a population of cells (e.g., mammalian cells) with said virus, the population of cells (e.g., mammalian cells) exhibit increased spreading of the virus relative to a population of cells (e.g., mammalian cells) of the same type contacted with a form of the virus that does not comprise said deletion.

In some embodiments of provided viruses, said recombinant orthopoxvirus vector exerts an increased cytotoxic effect on a population of cells (e.g., mammalian cells) relative to that of a form of the virus that does not comprise said deletion.

In some embodiments, said mammalian cells are human cells.

In some embodiments, said human cells are cancer cells.

In some embodiments, said mammalian cells are from a cell line selected from the group consisting of U2OS, 293, 293T, Vero, HeLa, A549, BHK, BSC40, CHO, OVCAR-8, 786-0, NCI-H23, U251, SF-295, T-47D, SKMEL2, BT-549, SK-MEL-28, MDA-MB-231, SK-OV-3, MCF7, M14, SF-268, CAKI-1, HPAV, OVCAR-4, HCT15, K-562, and HCT-116.

In some embodiments of provided viruses, the virus further comprises a transgene encoding a tumor-associated antigen, for example, a tumor-associated antigen listed in any one of Tables 3-30. In some embodiments, the tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.

In some embodiments, the tumor-associated antigen comprises MAGE-A3, or one or more fragments thereof.

In some embodiments, the tumor-associated antigen comprises NY-ESO-1, or one or more fragments thereof.

In some embodiments, the tumor-associated antigen comprises one or more human papillomavirus (HPV) proteins, or fragments thereof. In some embodiments, the HPV proteins or fragments thereof comprise one or more of (i) E6 and E7 proteins, or fragments thereof, of HPV16 and (ii) E6 and E7 proteins, or fragments thereof, of HPV18. In some embodiments, the sequences of said HPV proteins or fragments are disclosed in International Patent Publication WO/2014/127478, the contents of which are incorporated herein by reference.

In some embodiments, the tumor-associated antigen comprises brachyury or one or more fragments thereof.

In some embodiments, the tumor-associated antigen comprises prostatic acid phosphatase, or one or more fragments thereof.

In certain embodiments, the virus provided herein is isolated. In certain embodiments, the virus provided herein is purified.

In various embodiments, the virus provided herein is a recombinant virus.

In certain embodiments, the virus provided herein does not cause pox lesion formation when administered to a patient (e.g., a mammalian patient). In certain embodiments, the virus provided herein is able to replicate in vitro and/or when administered to a patient (e.g., a mammalian patient). In certain embodiments, the virus provided herein is able to express the transgene(s) described herein in vitro and/or when administered to a patient (e.g., a mammalian patient). In certain embodiments, the virus provided herein is able to kill target tumor cells (e.g., exhibits cancer cytotoxicity) in vitro and/or when administered to a patient (e.g., a mammalian patient). See the examples in Section 6 for exemplary assays that may be used to determine pox lesion formation, replication, transgene expression, or killing of target tumor cells (e.g., cancer cytotoxicity).

### 5.2.5. Assays for Measuring Virus Characteristics

In certain embodiments, the viruses described herein have been tested for their ability to replicate/spread, viability, transgene expression, and/or ability to kill target tumor cells (e.g., cancer cytotoxicity), using a method known in the art. See the examples in Section 6 for exemplary assays that may be used to determine replication/spreading, viability, transgene expression, or killing of target tumor cells (e.g., cancer cytotoxicity).

Assays known in the art to measure the tumor spreading and virulence of a virus include but are not limited to measuring plaque size, syncytia formation, and/or comet assays (EEVs). Assays known in the art to measure the immunostimulatory activity of a virus include but are not limited to NK activation (measured in % CD69 expression), NK degranulation (measured in fold increase of CD107a), and/or T-cell priming assays. Assays known in the art to measure the selectivity of a virus include, but are not limited to, tail pox lesions, biodistribution, and/or body mass measurements.

### 5.2.6. Cells, Cell Lines and Packaging Cell Lines

In one aspect, provided herein is a cell comprising the nucleic acid described in Section 5.2.3. In another aspect, provided herein is a cell comprising the virus described in Section 5.2.4. In certain embodiments, the cell provided herein is a mammalian cell (e.g., a human cell). In certain embodiments, the cell provided herein is a host cell (e.g., a host cell described in Section 5.4).

In one aspect, provided herein is a cell line comprising the nucleic acid described in Section 5.2.3. In another aspect, provided herein is a cell line comprising the virus described in Section 5.2.4. In certain embodiments, the cell line provided herein is a mammalian cell line (e.g., a human cell line).

In one aspect, provided herein is a packaging cell line comprising the nucleic acid described in Section 5.2.3. In another aspect, provided herein is a packaging cell line comprising the virus described in Section 5.2.4. The packaging cell line can be any cell line suitable for packaging orthopoxvirus viruses (e.g., vaccinia viruses). In certain embodiments, the packaging cell line provided herein is a mammalian packaging cell line (e.g., a human packaging cell line).

Exemplary cells that can be used to culture a virus described herein include, for example, the HeLa cells, U2OS cells, 293T cells, NIH3T3 cells, Jurkat cells, 293 cells, COS cells, CHO cells, Saos cells, PC12 cells, and chicken embryo fibroblasts (CEF). Exemplary packaging cell lines that can be used to package a virus described herein include, for example, the HeLa cell line, the U2-OS cell line, the HEK293T cell line, the 786-O cell line, the A549 cell line or an adherent human cancer cell line. In certain embodiments, the cells also express or are engineered to express one or more factors necessary for the replication and/or packaging of the vaccinia virus.

In certain embodiments, the cell, cell line, or the packaging cell line provided herein is a cell, cell line or packaging cell line described in an example in Section 6.

### 5.2.7 Examples of Proteins Encoded by Orthopoxvirus Genes

Exemplary proteins encoded by orthopoxvirus genes described in this disclosure are reproduced in Tables 31-40 below. As used below, the term “location” refers to the location of the gene with respect to the deleted nucleic acids in exemplary orthopoxvirus vectors described herein. For various genes, amino acid sequence information and protein accession ID numbers are provided.

### 5.3. Methods of Genetic Modification

Methods for the insertion or deletion of nucleic acids from a target genome include those described herein and known in the art. Methods for nucleic acid delivery to effect expression of compositions of the present invention are believed to include virtually any method by which a nucleic acid (e.g., DNA, including viral and non-viral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Depicted below are clusters of deleted genes and their function in CopMD5p, CopMD3p, and CopMD5p3p virus. ITR genes (designated in Table 2 by “-ITR” and “*”) are deleted in one copy, the right ITR of the genome. However, these genes have a second copy in the left ITR, which remains intact in these virus. Deletions were confirmed by whole genome sequencing. Most of the deleted genes are either involved in blocking host response to viral infection or have an unknown function.

TABLE 2
Deleted genes in Vaccinia viruses

| Name | Category | Function | Virus Deletions | |
|---|---|---|---|---|
| C2L | Host | Inhibits NFkB | CopMD5p | CopMD5p3p |
| | interaction | | | |
| C1L | Unknown | Unknown | | |
| N1L | Host | Inhibits NFkB and | | |
| | interaction | Apoptosis | | |
| N2L | Host | Inhibits IRF3 | | |
| | interaction | | | |
| M1L | Unknown | Unknown | | |
| M2L | Host | Inhibits NFkB and | | |
| | interaction | Apoptosis | | |
| K1L | Host | Inhibits PKR and | | |
| | interaction | NF-kB | | |
| K2L | Host | Prevents cell fusion | | |
| | interaction | | | |
| K3L | Host | Inhibits PKR | | |
| | interaction | | | |
| K4L | DNA | DNA modifying | | |
| | replication | nuclease | | |
| K5L | Pseudogene | Pseudogene | | |
| K6L | Pseudogene | Pseudogene | | |
| K7R | Host | Inhibits NFkB and | | |
| | interaction | IRF3 | | |
| F1L | Host | Inhibits Apoptosis | | |
| | interaction | | | |
| F2L | DNA | Deoxyuridine | | |
| | replication | triphosphatase | | |
| F3L | Host | Virulence factor | | |
| | interaction | | | |
| B14R | Pseudogene | Pseudogene | CopMD3p | |
| B15R | Unknown | Unknown | | |
| B16R | Host | IL-1-beta-inhibitor | | |
| | interaction | | | |
| B17L | Unknown | Unknown | | |
| B18R | Unknown | Ankyrin-like | | |
| B19R | Host | Secreted IFNα | | |
| | interaction | sequestor | | |
| B20R | Unknown | Ankyrin-like | | |
| B21R- | Unknown | Unknown | | |
| ITR* | | | | |
| B22R- | Unknown | Unknown | | |
| ITR* | | | | |
| B23R- | Unknown | Unknown | | |
| ITR* | | | | |
| B24R- | Unknown | Unknown | | |
| ITR* | | | | |
| B25R- | Unknown | Unknown | | |
| ITR* | | | | |
| B26R- | Unknown | Unknown | | |
| ITR* | | | | |
| B27R- | Unknown | Unknown | | |
| ITR* | | | | |
| B28R- | Pseudogene | TNF-a receptor | | |
| ITR* | | | | |
| B29R- | Host | Secreted CC- | | |
| ITR* | interaction | chemokine | | |
| | | sequestor | | |

In various embodiments, the orthopox viruses are further genetically modified to contain deletions in the B8R gene. The vaccinia virus B8R gene encodes a secreted protein with homology to gamma interferon receptor (IFN-γ). In vitro, the B8R protein binds to and neutralizes the antiviral activity of several species of gamma interferon including human and rat gamma interferon; it does not, however, bind significantly to murine IFN-γ. Deleting the B8R gene prevents the impairment of IFN-γ in humans. In various embodiments, one, two or three transgenes are inserted into the locus of the deleted B8R gene. In some strains, in addition to the transgene(s) present at the site of the B8R deletion, the strain also has, at least one transgene is inserted into an additional locus on the orthopox virus that is not the locus of the deleted B8R gene. In various embodiments, at least one transgene is inserted into boundaries of the 5p deletions, at least one transgene is inserted into the boundaries of the 3p deletions or both. In various, embodiments at least three, four, five or more transgenes are inserted into the modified orthopox virus genome.

In various embodiments, the sequence of the modified orthopoxvirus vector is the sequence depicted below in Table 43 as SEQ ID NO: 210. In some embodiments, the sequence of the modified orthopoxvirus vector is a derivative of SEQ ID NO: 210. For example, as noted herein, the modified orthopoxvirus vector may be modified to express one or more transgenes as discussed herein.

In various embodiments, the sequence of the modified orthopoxvirus vector is the sequence depicted below in Table 43 as SEQ ID NO: 624. In some embodiments, the sequence of the modified orthopoxvirus vector is a derivative of SEQ ID NO: 624. For example, as noted herein, the modified orthopoxvirus vector may contain a deletion of the B8R sequence and/or may be modified to express one or more transgenes as discussed herein.

In various embodiments, the modified orthopoxvirus expresses at least one of three transgenes: IL-12-TM, FLT3-L and anti-CLTA4 antibody. Non-limiting examples of sequences of these transgenes and/or of amino acid sequences encoded by them are described below:

Full-length anti-human CTLA-4 antibody (full-length
antibody comprising ipilimumab heavy and light chains
with T2A intervening the heavy and light chains

| Nucleotide | ATGGAAACAGACACCCTATTATTATGGGTTTTGCTTCTATGGGTGCCAGGATCT |
|---|---|
| SEQ ID | ACGGGTCAGGTTCAGCTAGTCGAATCGGGTGGAGGAGTCGTGCAGCCGGGACGT |
| NO: 214 | TCCTTACGTTTATCTTGCGCAGCGTCTGGTTTTACTTTCTCGTCCTACACTATG |
| | CATTGGGTTCGTCAGGCTCCGGGAAAGGGATTGGAGTGGGTAACATTTATAAGT |
| | TACGACGGTAATAATAAATACTATGCAGACAGTGTGAAGGGACGTTTCACTATA |
| | TCTCGAGATAATAGTAAGAACACTTTGTATTTGCAGATGAATTCATTGAGAGCG |
| | GAAGATACAGCAATTTACTACTGCGCCAGAACCGGATGGTTGGGTCCCTTTGAC |
| | TATTGGGGACAGGGTACTCTTGTTACGGTGTCTTCTGCTTCAACAAAGGGTCCC |
| | TCTGTCTTCCCGCTTGCGCCCTCATCAAAATCGACGTCGGGTGGAACCGCTGCC |
| | TTGGGATGCTTGGTTAAGGACTATTTCCCCGAACCTGTCACCGTGTCTTGGAAT |
| | TCCGGTGCTCTAACGTCTGGTGTGCACACTTTCCCTGCCGTTTTACAAAGTTCC |
| | GGACTATATTCACTTTCGTCCGTAGTAACTGTTCCAAGTTCGTCCCTTGGAACA |
| | CAGACCTATATATGCAACGTAAACCACAAACCCTCCAATACCAAAGTCGATAAA |
| | AGAGTTGAGCCTAAATCCTGCGACAAAACACACACCTGCCCACCTTGCCCGGCC |
| | CCTGAACTTCTTGGAGGACCATCTGTATTCCTTTTCCCACCGAAGCCTAAGGAC |
| | ACCTTGATGATATCCAGAACTCCCGAAGTCACGTGCGTAGTAGTCGATGTGAGT |
| | CACGAAGATCCGGAAGTCAAGTTTAACTGGTATGTAGACGGAGTAGAGGTTCAT |
| | AACGCCAAGACCAAGCCAAGAGAAGAACAATATAACTCGACTTACAGAGTCGTG |
| | TCTGTATTAACCGTCTTGCATCAGGACTGGTTAAACGGTAAAGAGTACAAGTGC |
| | AAGGTCTCCAATAAAGCCCTACCTGCCCCCATCGAAAAAACCATATCCAAGGCT |
| | AAGGGTCAGCCTAGAGAACCTCAAGTTTACACATTACCGCCCAGTAGAGATGAA |
| | CTTACGAAGAATCAAGTGAGTCTAACCTGCCTTGTTAAAGGATTCTACCCCAGT |
| | GACATAGCGGTGGAGTGGGAGTCCAACGGTCAACCCGAGAACAATTATAAGACG |
| | ACACCGCCCGTTCTTGACAGTGACGGATCGTTCTTTCTATACTCTAAGTTGACT |
| | GTGGATAAATCCCGATGGCAGCAGGGAAACGTATTCTCTTGCTCAGTGATGCAT |
| | GAGGCGTTGCACAATCATTACACCCAAAAGTCTTTGTCGCTAAGTCCAGGTAAA |
| | CCGCGGGGCAGCGGAGAGGGCAGAGGAAGCCTGCTGACTTGTGGCGATGTGGAA |
| | GAGAACCCTGGCCCTAAGCTTATGGCTTGGACACCAGGAATCTTCATGGTACTT |
| | AGTTACTTGACAGGATCTTTCTCGGAAATAGTCTTAACTCAGTCACCGGGTACA |
| | CTTTCCCTTTCGCCCGGAGAGCGTGCGACCCTATCGTGTCGAGCTTCCCAGTCG |
| | GTTGGTTCTTCGTATCTTGCTTGGTATCAGCAGAAGCCCGGACAAGCTCCTCGT |
| | CTTTTGATCTACGGTGCGTTTTCGAGAGCGACTGGTATCCCGGATAGATTTTCT |
| | GGATCGGGTTCTGGTACTGATTTCACTTTAACGATTTCGAGACTAGAGCCCGAA |
| | GATTTTGCTGTGTATTATTGCCAGCAATATGGATCTAGTCCGTGGACGTTCGGT |
| | CAGGGTACCAAGGTCGAGATAAAAAGAACTGTGGCCGCACCCTCCGTGTTTATC |
| | TTTCCCCCTTCCGACGAACAGCTAAAGTCGGGTACTGCATCGGTGGTATGTTTA |
| | CTTAACAACTTTTACCCACGAGAGGCCAAGGTACAATGGAAGGTGGATAACGCC |
| | TTACAATCAGGAAACTCACAAGAGTCCGTCACCGAGCAAGATTCCAAGGACAGT |
| | ACATACTCGTTATCCTCGACATTAACATTGAGTAAGGCGGATTATGAGAAGCAT |
| | AAGGTTTACGCATGCGAAGTGACGCACCAAGGACTTTCATCCCCCGTCACCAAG |
| | TCTTTCAATCGTGGTGAGTGCTGA |
| Amino | METDTLLLWVLLLWVPGSTGQVQLVESGGGVVQPGRSLRLSCAASGFTFSSYTM |
| acid | HWVRQAPGKGLEWVTFISYDGNNKYYADSVKGRFTISRDNSKNTLYLQMNSLRA |
| SEQ ID | EDTAIYYCARTGWLGPFDYWGQGTLVTVSSASTKGPSVFPLAPSSKSTSGGTAA |
| NO: 211 | LGCLVKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPKPKD |
| | TLMISRTPEVTCVVVDVSHEDPEVKFNWYVDGVEVHNAKTKPREEQYNSTYRVV |
| | SVLTVLHQDWLNGKEYKCKVSNKALPAPIEKTISKAKGQPREPQVYTLPPSRDE |
| | LTKNQVSLTCLVKGFYPSDIAVEWESNGQPENNYKTTPPVLDSDGSFFLYSKLT |
| | VDKSRWQQGNVFSCSVMHEALHNHYTQKSLSLSPGKPRGSGEGRGSLLTCGDVE |
| | ****************** |
| | ENPGPKLMAWTPGIFMVLSYLTGSFSEIVLTQSPGTLSLSPGERATLSCRASQS |
| | ***** |
| | VGSSYLAWYQQKPGQAPRLLIYGAFSRATGIPDRFSGSGSGTDFTLTISRLEPE |
| | DFAVYYCQQYGSSPWTFGQGTKVEIKRTVAAPSVFIFPPSDEQLKSGTASVVCL |
| | LNNFYPREAKVQWKVDNALQSGNSQESVTEQDSKDSTYSLSSTLTLSKADYEKH |
| | KVYACEVTHQGLSSPVTKSFNRGEC |
| | (italicized: signal peptide sequence; sequence having |
| | * beneath the letters: T2A sequence (includinGa PRGSG |
| | (SEQ ID NO: 589) linker); bold: heavy chain sequence; |
| | bold and underlined: lighTchain sequence) |
| Heavy | QVQLVESGGGVVQPGRSLRLSCAASGFTFSSYTMHWVRQAPGKGLEWVTFISYD |
| chain | GNNKYYADSVKGRFTISRDNSKNTLYLQMNSLRAEDTAIYYCARTGWLGPFDYW |
| (amino | GQGTLVTVSSASTKGPSVFPLAPSSKSTSGGTAALGCLVKDYFPEPVTVSWNSG |
| acid) | ALTSGVHTFPAVLQSSGLYSLSSVVTVPSSSLGTQTYICNVNHKPSNTKVDKRV |
| (SEQ ID | EPKSCDKTHTCPPCPAPELLGGPSVFLFPPKPKDTLMISRTPEVTCVVVDVSHE |
| NO: 217) | DPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEA |
| | LHNHYTQKSLSLSPGKPRGSGEGRGSLLTCGDVEENPG |
| Light chain | EIVLTQSPGTLSLSPGERATLSCRASQSVGSSYLAWYQQKPGQAPRLLIYGAFS |
| (amino | RATGIPDRFSGSGSGTDFTLTISRLEPEDFAVYYCQQYGSSPWTFGQGTKVEIK |
| acid) (SEQ | RTVAAPSVFIFPPSDEQLKSGTASVVCLLNNFYPREAKVQWKVDNALQSGNSQE |
| ID NO: | SVTEQDSKDSTYSLSSTLTLSKADYEKHKVYACEVTHQGLSSPVTKSFNRGEC |
| 218) | |

Anti-CTLA-4 (human single chain)

| | |
|---|---|
| Nucleotide | ATGGAGACAGATACTCTATTGCTATGGGTTTTGTTATTATGGGTGCCGGGTTCC |
| SEQ ID | ACGGGAATTCGTCGTGCTGACATTGTTTTAACACAGTCTCCAGGTACCCTATCA |
| NO: 579 | TTGTCCCCTGGTGAGCGTGCCACGCTTAGTTGCAGAGCCTCTCAGTCTGTGGGA |
| | TCAAGTTATTTAGCTTGGTATCAACAAAAACCTGGACAAGCACCTCGTTTGTTA |
| | ATATATGGTGCATTCAGTAGAGCAACTGGAATTCCTGATCGATTCTCAGGATCT |
| | GGATCTGGAACAGACTTCACACTTACCATCTCACGTCTTGAACCCGAGGACTTT |
| | GCGGTCTACTACTGCCAGCAGTATGGTTCGTCGCCTTGGACTTTCGGACAAGGA |
| | ACCAAAGTTGAAATCAAGCGAGGAGGAGGTGGATCGGGTGGTGGAGGATCCGGT |
| | GGAGGTGGAAGTGAAGCGAAGCTAGTCGAATCTGGAGGTGGTGTAGTGCAGCCT |
| | GGACGTTCGCTTCGACTATCCTGTGCTGCTTCAGGATTTACCTTTTCATCTTAC |
| | ACGATGCACTGGGTGCGTCAAGCGCCTGGTAAAGGACTAGAGTGGGTCACGTTT |
| | ATCTCATACGACGGTAATAACAAGTATTATGCTGATTCCGTAAAGGGACGTTTC |
| | ACCATATCGCGTGACAATTCTAAGAATACCTTGTATCTTCAAATGAATAGTCTT |
| | AGAGCTGAAGACACTGCCATTTACTATTGTGCACGAACGGGATGGCTTGGACCT |
| | TTTGATTATTGGGGTCAGGGTACTTTGGTCACCGTCTCCACAGCGAAGACAACA |
| | CCTCCCTCAGTCTATCCACTTGCTCCGCGTAGTTAA |
| Amino | METDTLLLWVLLLWVPGSTGIRRADIVLTQSPGTLSLSPGERATLSCRASQSVG |
| acid | SSYLAWYQQKPGQAPRLLIYGAFSRATGIPDRFSGSGSGTDFTLTISRLEPEDF |
| SEQ ID | AVYYCQQYGSSPWTFGQGTKVEIKRGGGGSGGGGSGGGGSEAKLVESGGGVVQP |
| NO: 580 | GRSLRLSCAASGFTFSSYTMHWVRQAPGKGLEWVTFISYDGNNKYYADSVKGRF |
| | TISRDNSKNTLYLQMNSLRAEDTAIYYCARTGWLGPFDYWGQGTLVTVSTAKTT |
| | PPSVYPLAPRS |

Anti-CTLA-4 (mouse single chain)

| | |
|---|---|
| Nucleotide | ATGGAAACCGATACTTTGCTTCTATGGGTCCTTTTGCTATGGGTGCCCGGATCA |
| SEQ ID | ACCGGAATAAGACGTGCTGATATCGTTATGACCCAGACCACGCTATCATTACCA |
| NO: 581 | GTTAGTCTAGGTGACCAGGCCAGTATCAGTTGCCGTTCATCTCAGTCCATTGTA |
| | CACTCAAACGGAAACACCTACTTGGAGTGGTATCTTCAGAAACCTGGTCAATCT |
| | CCCAAGCTTCTAATTTACAAAGTGTCTAACCGATTTTCTGGTGTGCCGGATCGA |
| | TTTTCGGGTTCTGGTAGTGGAACGGATTTCACGCTAAAAATATCCCGAGTCGAA |
| | GCTGAAGACCTAGGAGTATATTATTGCTTTCAAGGATCTCACGTCCCGTACACC |
| | TTTGGTGGAGGAACCAAGCTTGAAATAAAGCGAGGAGGTGGAGGATCAGGAGGA |
| | GGTGGTTCGGGTGGTGGTGGTTCCGAGGCGAAGTTACAGGAGTCGGGACCCGTA |
| | TTAGTGAAGCCTGGTGCGAGTGTCAAAATGAGTTGCAAAGCCAGTGGTTACACC |
| | TTCACAGACTATTACATGAACTGGGTGAAGCAGTCTCACGGAAAATCTTTAGAG |
| | TGGATAGGAGTAATTAACCCGTACAATGGTGATACGAGTTACAACCAGAAGTTC |
| | AAAGGTAAGGCGACCTTGACGGTCGATAAATCCTCTAGTACTGCGTACATGGAA |
| | CTAAACTCTTTAACCTCTGAGGATTCTGCCGTATATTATTGTGCCAGATATTAT |
| | GGTTCGTGGTTCGCATATTGGGGACAGGGAACTTTAATTACGGTCTCGACAGCC |
| | AAAACGACTCCCCCCTCAGTTTATCCCCTTGCTCCTAGAAGTTAA |
| Amino | METDTLLLWVLLLWVPGSTGIRRADIVMTQTTLSLPVSLGDQASISCRSSQSIV |
| acid | HSNGNTYLEWYLQKPGQSPKLLIYKVSNRFSGVPDRFSGSGSGTDFTLKISRVE |
| SEQ ID | AEDLGVYYCFQGSHVPYTFGGGTKLEIKRGGGGSGGGGSGGGGSEAKLQESGPV |
| NO: 582 | LVKPGASVKMSCKASGYTFTDYYMNWVKQSHGKSLEWIGVINPYNGDTSYNQKF |
| | KGKATLTVDKSSSTAYMELNSLTSEDSAVYYCARYYGSWFAYWGQGTLITVSTA |
| | KTTPPSVYPLAPRS |

Human IL-12 TM p35

| | |
|---|---|
| Nucleotide | ATGTGTCCCGCGCGATCGTTATTGTTAGTTGCGACGTTGGTCCTACTTGACCAT |
| SEQ ID | CTATCACTAGCGCGTAATTTGCCCGTTGCCACACCAGATCCCGGAATGTTTCCT |
| NO: 215 | TGCTTACATCATAGTCAGAACTTACTTCGTGCAGTCTCCAACATGTTACAGAAA |
| | GCCCGACAGACCTTAGAGTTCTATCCCTGTACTTCCGAAGAGATAGATCACGAG |
| | GACATAACGAAAGACAAAACATCGACCGTTGAAGCGTGCTTACCCTTAGAACTA |
| | ACCAAAAATGAAAGTTGTCTAAACTCTAGAGAAACGAGTTTTATCACCAATGGA |
| | AGTTGCTTGGCGTCTAGAAAAACATCATTTATGATGGCCTTGTGTCTTTCCTCC |
| | ATATACGAGGACTTGAAGATGTATCAGGTCGAGTTCAAGACAATGAACGCGAAA |
| | TTGCTTATGGACCCCAAACGACAGATATTTTTGGACCAAAACATGTTAGCTGTT |
| | ATAGACGAATTGATGCAGGCGCTAAACTTCAATTCGGAAACTGTGCCACAGAAG |
| | TCATCCTTAGAGGAGCCCGATTTTTACAAGACAAAAATCAAGTTATGCATTCTT |
| | CTTCACGCGTTTAGAATTCGTGCCGTTACGATTGATAGAGTAATGTCGTACTTG |
| | AATGCGTCGGGTGGAGGAGGTTCCGGAGGAGGAGGATCCGGAGGAGGTGGATCC |
| | TTACTTCCTTCGTGGGCTATAACATTAATCTCCGTTAATGGTATCTTCGTGATT |
| | TGCTGTCTAACATACTGCTTTGCATGA |
| | (Underlined and bold: B7-1 transmembrane domain; |
| | Bold: B7-1 cytoplasmic domain.) |
| Amino | MCPARSLLLVATLVLLDHLSLARNLPVATPDPGMFPCLHHSQNLLRAVSNMLQK |
| acid: SEQ | ARQTLEFYPCTSEEIDHEDITKDKTSTVEACLPLELTKNESCLNSRETSFITNG |
| ID NO: | SCLASRKTSFMMALCLSSIYEDLKMYQVEFKTMNAKLLMDPKRQIFLDQNMLAV |
| 212 | IDELMQALNFNSETVPQKSSLEEPDFYKTKIKLCILLHAFRIRAVTIDRVMSYL |
| | NASGGGGSGGGGSGGGGSLLPSWAITLISVNGIFVICCLTYCFA |
| | (Underlined and bold: B7-1 transmembrane domain; |
| | Bold: B7-1 cytoplasmic domain.) |

Human IL-12 TM p70

| | |
|---|---|
| Nucleotide | ATGTGTCACCAGCAGTTGGTCATCTCTTGGTTTTCCCTGGTTTTTCTGGCATCT |
| SEQ ID | CCCCTCGTGGCCATATGGGAACTGAAGAAAGATGTTTATGTCGTAGAATTGGAT |
| NO: 583 | TGGTATCCGGATGCCCCTGGAGAAATGGTGGTCCTCACCTGTGACACCCCTGAA |
| | GAAGATGGTATCACCTGGACCTTGGACCAGAGCAGTGAGGTCTTAGGCTCTGGC |
| | AAAACCCTGACCATCCAAGTCAAAGAGTTTGGAGATGCTGGCCAGTACACCTGT |
| | CACAAAGGAGGCGAGGTTCTAAGCCATTCGCTCCTGCTGCTTCACAAAAAGGAA |
| | GATGGAATTTGGTCCACTGATATTTTAAAGGACCAGAAAGAACCCAAAAATAAG |
| | ACCTTTCTAAGATGCGAGGCCAAGAATTATTCTGGACGTTTCACCTGCTGGTGG |
| | CTGACGACAATCAGTACTGATTTGACATTCAGTGTCAAAAGCAGCAGAGGCTCT |
| | TCTGACCCCCAAGGGGTGACGTGCGGAGCTGCTACACTCTCTGCAGAGAGAGTC |
| | AGAGGGGACAACAAGGAGTATGAGTACTCAGTGGAGTGCCAGGAGGACAGTGCC |
| | TGCCCAGCTGCTGAGGAGAGTCTGCCCATTGAGGTCATGGTGGATGCCGTTCAC |
| | AAGCTCAAGTATGAAAACTACACCAGCAGCTTCTTCATCAGGGACATCATCAAA |
| | CCTGACCCACCCAAGAACTTGCAGCTGAAGCCATTAAAGAATTCTCGGCAGGTG |
| | GAGGTCAGCTGGGAGTACCCTGACACCTGGAGTACTCCACATTCCTACTTCTCC |
| | CTGACATTCTGCGTTCAGGTCCAGGGCAAGAGCAAGAGAGAAAAGAAAGATAGA |
| | GTCTTCACGGACAAGACCTCAGCCACGGTCATCTGCCGCAAAAATGCCAGCATT |
| | AGCGTGCGGGCCCAGGACCGCTACTATAGCTCATCTTGGAGCGAATGGGCATCT |
| | GTGCCCTGCAGTGTTCCTGGAGTAGGGGTACCTGGGGTGGGCGCCAGAAACCTC |
| | CCCGTGGCCACTCCAGACCCAGGAATGTTCCCATGCCTTCACCACTCCCAAAAC |
| | CTGCTGAGGGCCGTCAGCAACATGCTCCAGAAGGCCAGACAAACTCTAGAATTT |
| | TACCCTTGCACTTCTGAAGAGATTGATCATGAAGATATCACAAAAGATAAAACC |
| | AGCACAGTGGAGGCCTGTTTACCATTGGAATTAACCAAGAATGAGAGTTGCCTA |
| | AATTCCAGAGAGACCTCTTTCATAACTAATGGGAGTTGCCTGGCCTCCAGAAAG |
| | ACCTCTTTTATGATGGCCCTGTGCCTTAGTAGTATTTATGAAGACTTGAAGATG |
| | TACCAGGTGGAGTTCAAGACCATGAATGCAAAGCTGCTGATGGACCCTAAGAGG |
| | CAGATCTTTCTAGATCAAAACATGCTGGCAGTTATTGATGAGCTGATGCAGGCC |
| | CTGAATTTCAACAGTGAGACTGTGCCACAAAAATCCTCCCTTGAAGAACCGGAT |
| | TTTTATAAAACTAAAATCAAGCTCTGCATACTTCTTCATGCTTTCAGAATTCGG |
| | GCAGTGACTATTGATAGAGTGATGAGCTATCTGAATGCTTCCGGAGGAGGTGGA |
| | TCGGGTGGTGGAGGATCTGGTGGAGGTGGAAGTCTGCTCCCATCCTGGGCCATT |
| | ACCTTAATCTCAGTAAATGGAATTTTTGTGATATGCTGCCTGACCTACTGCTTT |
| | GCCTAA |
| | (Underlined and bold: B7-1 transmembrane domain; |
| | Bold: B7-1 cytoplasmic domain.) |
| Amino | MCHQQLVISWFSLVFLASPLVAIWELKKDVYVVELDWYPDAPGEMVVLTCDTPE |
| acid: SEQ | EDGITWTLDQSSEVLGSGKTLTIQVKEFGDAGQYTCHKGGEVLSHSLLLLHKKE |
| ID NO: | DGIWSTDILKDQKEPKNKTFLRCEAKNYSGRFTCWWLTTISTDLTFSVKSSRGS |
| 584 | SDPQGVTCGAATLSAERVRGDNKEYEYSVECQEDSACPAAEESLPIEVMVDAVH |
| | KLKYENYTSSFFIRDIIKPDPPKNLQLKPLKNSRQVEVSWEYPDTWSTPHSYFS |
| | LTFCVQVQGKSKREKKDRVFTDKTSATVICRKNASISVRAQDRYYSSSWSEWAS |
| | VPCSVPGVGVPGVGARNLPVATPDPGMFPCLHHSQNLLRAVSNMLQKARQTLEF |
| | YPCTSEEIDHEDITKDKTSTVEACLPLELTKNESCLNSRETSFITNGSCLASRK |
| | TSFMMALCLSSIYEDLKMYQVEFKTMNAKLLMDPKRQIFLDQNMLAVIDELMQA |
| | LNFNSETVPQKSSLEEPDFYKTKIKLCILLHAFRIRAVTIDRVMSYLNASGGGG |
| | SGGGGSGGGGSLLPSWAITLISVNGIFVICCLTYCFA |
| | (Underlined and bold: B7-1 transmembrane domain; |
| | Bold: B7-1 cytoplasmic domain.) |

Mouse IL-12 TM p35

| | |
|---|---|
| Nucleotide | ATGTGTCAGTCTCGATACCTTCTTTTCCTAGCAACCTTAGCGTTATTGAATCAT |
| SEQ ID | CTTTCATTAGCGCGTGTCATTCCGGTCTCCGGTCCCGCCCGTTGCCTTTCGCAA |
| NO: 585 | AGTCGTAACTTGCTTAAGACTACGGATGATATGGTCAAAACTGCTAGAGAAAAG |
| | TTAAAACACTACTCGTGTACGGCTGAAGACATAGACCACGAAGACATCACGCGA |
| | GATCAAACAAGTACCCTAAAGACTTGCTTACCGCTAGAGCTTCATAAAAACGAG |
| | AGTTGCCTAGCGACCCGAGAGACATCTTCAACAACCAGAGGATCTTGTCTACCC |
| | CCTCAGAAGACTTCTCTAATGATGACCTTGTGCCTTGGTAGTATATATGAAGAT |
| | TTAAAGATGTACCAGACTGAATTTCAGGCAATCAATGCGGCATTACAAAACCAC |
| | AACCATCAGCAGATAATATTAGATAAAGGAATGCTTGTAGCCATTGACGAACTT |
| | ATGCAGTCTCTAAACCACAACGGTGAGACACTTCGTCAGAAACCTCCGGTTGGT |
| | GAGGCAGATCCTTACCGTGTGAAAATGAAGTTGTGCATTCTATTGCATGCATTT |
| | TCGACGCGTGTGGTCACCATCAACAGAGTCATGGGTTATCTAAGTTCTGCTGGT |
| | GGAGGTGGAAGTGGAGGTGGAGGAAGTGGTGGAGGAGGAAGTACCTTAGTGTTG |
| | TTTGGAGCAGGTTTCGGTGCAGTCATAACGGTTGTCGTTATCGTGGTGATCATA |
| | AAATGTTTCTGCAAGTGA |
| | (Underlined and bold: transmembrane and cytoplasmic |
| | domain.) |
| Amino | MCQSRYLLFLATLALLNHLSLARVIPVSGPARCLSQSRNLLKTTDDMVKTAREK |
| acid: SEQ | LKHYSCTAEDIDHEDITRDQTSTLKTCLPLELHKNESCLATRETSSTTRGSCLP |
| ID NO: | PQKTSLMMTLCLGSIYEDLKMYQTEFQAINAALQNHNHQQIILDKGMLVAIDEL |
| 586 | MQSLNHNGETLRQKPPVGEADPYRVKMKLCILLHAFSTRVVTINRVMGYLSSAG |
| | GGGSGGGGSGGGGSTLVLFGAGFGAVITVVVIVVIIKCFCK |
| | (Underlined and bold: transmembrane and cytoplasmic |
| | domain.) |

Mouse IL-12 TM p70

| | |
|---|---|
| Nucleotide | ATGTGCCCCCAAAAGTTGACCATCTCATGGTTCGCAATTGTACTACTAGTGAGT |
| SEQ ID | CCCTTGATGGCAATGTGGGAGCTTGAAAAGGACGTCTATGTGGTGGAGGTTGAC |
| NO: 587 | TGGACGCCCGATGCGCCAGGAGAAACAGTGAATCTAACTTGCGACACACCTGAA |
| | GAGGATGACATAACGTGGACATCTGATCAAAGACATGGTGTGATAGGTTCTGGT |
| | AAGACACTTACGATTACCGTCAAGGAATTTTTGGACGCTGGACAATACACTTGT |
| | CACAAAGGAGGTGAAACACTATCGCATTCACACCTACTTTTACACAAGAAGGAA |
| | AATGGTATTTGGAGTACGGAGATCCTAAAGAATTTTAAAAATAAGACCTTTTTA |
| | AAGTGCGAAGCACCAAATTATTCCGGACGATTTACATGTTCATGGTTAGTTCAA |
| | CGAAATATGGATTTAAAATTCAATATCAAGTCAAGTTCTAGTTCCCCGGATTCC |
| | CGAGCGGTTACTTGCGGTATGGCCAGTTTGAGTGCCGAGAAAGTCACACTAGAC |
| | CAGCGAGATTATGAGAAATATTCCGTTTCCTGCCAGGAGGACGTAACTTGTCCG |
| | ACTGCCGAGGAGACTTTGCCGATAGAGCTTGCCTTAGAGGCCCGACAGCAGAAT |
| | AAGTACGAGAATTACTCTACCTCTTTTTTCATCCGAGACATCATCAAACCTGAC |
| | CCACCAAAGAATTTGCAAATGAAACCCTTAAAAAACTCACAGGTGGAGGTGTCC |
| | TGGGAATATCCTGACTCTTGGTCTACCCCCCATTCTTACTTTTCCCTTAAGTTC |
| | TTCGTTAGAATACAACGTAAAAAAGAGAAGATGAAAGAAACGGAAGAAGGTTGC |
| | AACCAGAAAGGAGCATTTCTAGTTGAAAAGACCTCTACGGAAGTCCAGTGTAAA |
| | GGTGGAAATGTGTGTGTGCAAGCCCAGGACCGATATTACAACAGTTCGTGTTCG |
| | AAGTGGGCTTGCGTGCCGTGTCGTGTCCGATCTGTACCAGGAGTCGGAGTTCCT |
| | GGAGTAGGTCGTGTAATACCGGTATCCGGACCAGCTCGTTGCTTATCTCAATCG |
| | CGAAACCTACTTAAAACAACCGATGACATGGTGAAGACAGCGAGAGAAAAGCTT |
| | AAACATTATTCCTGTACCGCCGAAGACATCGATCATGAGGATATCACGAGAGAC |
| | CAGACCTCGACACTTAAGACATGCTTGCCACTAGAACTTCATAAAAATGAGTCT |
| | TGCTTAGCAACGCGAGAAACGTCGTCTACGACGCGTGGTTCATGTTTGCCGCCC |
| | CAAAAGACATCCTTGATGATGACGTTGTGTCTTGGATCCATCTATGAGGATTTA |
| | AAAATGTACCAGACAGAATTTCAGGCTATCAATGCAGCTCTACAGAATCATAAT |
| | CACCAGCAGATTATCTTAGACAAGGGAATGTTAGTAGCAATCGATGAGCTTATG |
| | CAATCCTTGAATCATAACGGAGAAACATTACGACAAAAGCCTCCGGTCGGTGAA |
| | GCGGATCCATATCGTGTGAAGATGAAATTATGTATATTGTTGCACGCCTTTAGT |
| | ACGCGTGTGGTCACCATAAACCGAGTAATGGGATACTTGTCCTCGGCGGGAGGT |
| | GGTGGTTCTGGTGGAGGTGGATCAGGTGGTGGTGGTTCAACCTTGGTTCTTTTT |
| | GGTGCGGGTTTCGGTGCCGTGATTACTGTCGTCGTCATAGTCGTCATTATTAAA |
| | TGTTTTTGTAAGTAA |
| | (Underlined and bold: transmembrane and cytoplasmic |
| | domain.) |
| Amino | MCPQKLTISWFAIVLLVSPLMAMWELEKDVYVVEVDWTPDAPGETVNLTCDTPE |
| acid: SEQ | EDDITWTSDQRHGVIGSGKTLTITVKEFLDAGQYTCHKGGETLSHSHLLLHKKE |
| ID NO: | NGIWSTEILKNFKNKTFLKCEAPNYSGRFTCSWLVQRNMDLKFNIKSSSSSPDS |
| 588 | RAVTCGMASLSAEKVTLDQRDYEKYSVSCQEDVTCPTAEETLPIELALEARQQN |
| | KYENYSTSFFIRDIIKPDPPKNLQMKPLKNSQVEVSWEYPDSWSTPHSYFSLKF |
| | FVRIQRKKEKMKETEEGCNQKGAFLVEKTSTEVQCKGGNVCVQAQDRYYNSSCS |
| | KWACVPCRVRSVPGVGVPGVGRVIPVSGPARCLSQSRNLLKTTDDMVKTAREKL |
| | KHYSCTAEDIDHEDITRDQTSTLKTCLPLELHKNESCLATRETSSTTRGSCLPP |
| | QKTSLMMTLCLGSIYEDLKMYQTEFQAINAALQNHNHQQIILDKGMLVAIDELM |
| | QSLNHNGETLRQKPPVGEADPYRVKMKLCILLHAFSTRVVTINRVMGYLSSAGG |
| | GGSGGGGSGGGGSTLVLFGAGFGAVITVVVIVVIIKCFCK |
| | (Underlined and bold: transmembrane and cytoplasmic |
| | domain.) |

Human FLT3-L

| | |
|---|---|
| Nucleotide | ATGACAGTCTTAGCGCCAGCTTGGAGTCCTACCACATATTTACTACTATTATTG |
| SEQ ID | CTTTTATCTAGTGGTTTATCAGGAACGCAAGACTGCTCATTCCAACATTCACCG |
| NO: 216 | ATCAGTTCCGACTTTGCAGTTAAGATTCGAGAACTATCAGACTACCTATTGCAA |
| | GACTATCCGGTGACGGTAGCATCGAATCTTCAAGACGAAGAGCTTTGTGGTGGA |
| | CTATGGCGTCTTGTACTTGCCCAAAGATGGATGGAGCGACTAAAAACCGTTGCC |
| | GGTTCAAAGATGCAGGGTTTACTAGAGCGTGTGAATACGGAAATTCATTTTGTT |
| | ACGAAATGTGCGTTCCAACCCCCACCCAGTTGCTTGCGTTTCGTGCAGACTAAT |
| | ATCTCTCGTTTACTACAAGAAACATCCGAGCAACTAGTGGCGCTAAAGCCCTGG |
| | ATAACACGTCAAAACTTTAGTCGTTGCTTGGAGTTACAGTGCCAACCCGGTGCC |
| | CCACGACCTCAAAGTCCTGGTCCAGCGGCTTGCGGAGCCCTTACCTGGCCTCGA |
| | CCGCATCCGGCCGAACCATGA |
| Amino | MTVLAPAWSPTTYLLLLLLLSSGLSGTQDCSFQHSPISSDFAVKIRELSDYLLQ |
| acid | DYPVTVASNLQDEELCGGLWRLVLAQRWMERLKTVAGSKMQGLLERVNTEIHFV |
| SEQ ID | TKCAFQPPPSCLRFVQTNISRLLQETSEQLVALKPWITRQNFSRCLELQCQPGA |
| NO: 213 | PRPQSPGPAACGALTWPRPHPAEP |

See Example 32 in Section 6.32 for exemplary methods of generating a recombinant vaccinia virus described herein.

### 5.4. Virus Propagation

The present invention features recombinant orthopoxviruses, including those constructed with one or more gene deletions compared to wild-type, such that the virus exhibits desirable properties for use against cancer cells, while being less toxic or non-toxic to non-cancer cells. This section summarizes various protocols, by way of example, for producing recombinant orthopoxviruses described herein, such as methods for generating mutated viruses through the use of recombinant DNA technology.

For example, to generate mutations in the orthopoxvirus genome, native and modified polypeptides may be encoded by a nucleic acid molecule comprised in a vector. Vectors may include, for example, plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al., 1994, both incorporated herein by reference in their entirety. In addition to encoding a modified polypeptide, a vector may encode non-modified polypeptide sequences such as a tag or targeting molecule.

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors or viruses (which qualify as a vector if they express an exogenous polypeptide). A host cell may be “transfected” or “transformed,” which refers to a process by which exogenous nucleic acid, such as a modified protein-encoding sequence, is transferred or introduced into the host cell. A transformed cell includes the primary subject cell and its progeny. Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KCB, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris. Examples of eukaryotic host cells for replication and/or expression of a vector include HeLa, NIH3T3, Jurkat, 293, COS, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector. Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Also provided herein are methods of propagating a virus described in Section 5.2.4 using a cell, a cell line, or a packaging cell line described in Sections 5.2.6 and 5.4. In one aspect, provided herein is a method of propagating a virus, comprising culturing a cell, a cell line, or a packaging cell line infected with a virus described herein. In some embodiments, the virus is isolated or purified after propagation. See examples in Section 6 for exemplary methods and techniques for propagating viruses.

### 5.5. Methods of Treatment

### 5.5.1. Pharmaceutical Composition, Administration, and Doses

Also provided herein are pharmaceutical composition comprising a virus described in Section 5.2.4 and a physiologically acceptable carrier. In certain embodiments, the pharmaceutical composition provided herein comprises a therapeutically effective amount of the virus. In certain embodiments, the pharmaceutical composition provided herein is to be used in a method of treatment described herein.

Therapeutic compositions containing recombinant orthopoxvirus vectors of the invention can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

To induce oncolysis, kill cells, inhibit growth, inhibit metastases, decrease tumor size and otherwise reverse or reduce the malignant phenotype of tumor cells, using the methods and compositions of the present invention, one may contact a tumor with the modified orthopoxvirus, e.g., by administration of the orthopoxvirus to a patient having cancer by way of, for instance, one or more of the routes of administration described herein. The route of administration may vary with the location and nature of the cancer, and may include, e.g., intradermal, transdermal, parenteral, intravenous, intramuscular, intranasal, subcutaneous, regional (e.g., in the proximity of a tumor, particularly with the vasculature or adjacent vasculature of a tumor), percutaneous, intratracheal, intraperitoneal, intraarterial, intravesical, intratumoral, inhalation, perfusion, lavage, and oral administration and formulation. In specific embodiments, the pharmaceutical composition provided herein is formulated so that it is suitable for the route of administration to be employed.

The term “intravascular” is understood to refer to delivery into the vasculature of a patient, meaning into, within, or in a vessel or vessels of the patient. In certain embodiments, the administration is into a vessel considered to be a vein (intravenous), while in others administration is into a vessel considered to be an artery. Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Intratumoral injection, or injection directly into the tumor vasculature is specifically contemplated for discrete, solid, accessible tumors. Local, regional or systemic administration also may be appropriate. The viral particles may advantageously be contacted by administering multiple injections to the tumor, spaced, for example, at approximately 1 cm intervals. In the case of surgical intervention, the present invention may be used preoperatively, such as to render an inoperable tumor subject to resection. Continuous administration also may be applied where appropriate, for example, by implanting a catheter into a tumor or into tumor vasculature. Such continuous perfusion may take place, for example, for a period of from about 1-2 hours, to about 2-6 hours, to about 6-12 hours, or about 12-24 hours following the initiation of treatment. Generally, the dose of the therapeutic composition via continuous perfusion may be equivalent to that given by a single or multiple injections, adjusted over a period of time during which the perfusion occurs. It is further contemplated that limb perfusion may be used to administer therapeutic compositions of the present invention, particularly in the treatment of melanomas and sarcomas.

Treatment regimens may vary, and often depend on tumor type, tumor location, disease progression, and health and age of the patient. Certain types of tumor will require more aggressive treatment, while at the same time, certain patients cannot tolerate more taxing protocols. The clinician will be best suited to make such decisions based on the known efficacy and toxicity (if any) of the therapeutic formulations. In certain embodiments, the tumor being treated may not, at least initially, be resectable. Treatments with the therapeutic agent of the disclosure may increase the resectability of the tumor due to shrinkage at the margins or by elimination of certain particularly invasive portions. Following treatments, resection may be possible. Additional treatments subsequent to resection will serve to eliminate microscopic residual disease at the tumor site.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) for a viral construct. Unit doses may range from 10³, 10⁴, 10⁵, 10⁶, 10⁷, 10⁸, 10⁹, 10¹⁰, 10¹¹, 10¹², to 10¹³ pfu and higher. Additionally or alternatively, depending on the kind of virus and the titer attainable, one may deliver 1 to 100, 10 to 50, 100-1000, or up to about or at least about 1×10⁴, 1×10⁵, 1×10⁶, 1×10⁷, 1×10⁸, 1×10⁹, 1×10¹¹, 1×10¹², 1×10¹³, 1×10¹⁴, or 1×10¹⁵ or higher infectious viral particles (vp), including all values and ranges there between, to the tumor or tumor site.

Another method of delivery of the recombinant orthopoxvirus genome disclosed herein to cancer or tumor cells may be via intratumoral injection. However, the pharmaceutical compositions disclosed herein may alternatively be administered parenterally, intravenously, intradermally, intramuscularly, transdermally or even intraperitoneally as described in U.S. Pat. Nos. 5,543,158; 5,641,515 and 5,399,363 (each specifically incorporated herein by reference in its entirety). Injection of nucleic acid constructs may be delivered by syringe or any other method used for injection of a solution, as long as the expression construct can pass through the particular gauge of needle required for injection. An exemplary needleless injection system that may be used for the administration of recombinant orthopoxviruses described herein is exemplified in U.S. Pat. No. 5,846,233. This system features a nozzle defining an ampule chamber for holding the solution and an energy device for pushing the solution out of the nozzle to the site of delivery. Another exemplary syringe system is one that permits multiple injections of predetermined quantities of a solution precisely at any depth (U.S. Pat. No. 5,846,225).

Mixtures of the viral particles or nucleic acids described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form may be sterile and may be fluid to the extent that easy syringability exists. It may be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

### 5.5.2. Methods of Treatment

Also provided herein are methods of treating a cell proliferation disorder, such as cancer in a patient (e.g., a mammalian patient, such as a human patient).

In one aspect, provided herein is a method of treating a cell proliferation disorder, such as cancer in a patient (e.g., a mammalian patient, such as a human patient), the method comprising administering to the patient (e.g., a mammalian patient, such as a human patient) a therapeutically effective amount of a virus described in Section 5.2.4.

In another aspect, provided herein is a method of treating a cell proliferation disorder, such as cancer in a patient (e.g., a mammalian patient, such as a human patient), the method comprising administering to the patient (e.g., a mammalian patient, such as a human patient) a therapeutically effective amount of a pharmaceutical composition described in Section 5.5.1.

In a specific embodiment of the method of treating described herein, the mammalian patient is a human patient.

In certain embodiments of the method of treating described herein, the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

In certain embodiments of the method of treating described herein, the cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin's lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi's sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

In some embodiments of the method of treating described herein, the virus and the pharmaceutical composition are not administered in combination with another agent for treating the cell proliferation disorder (such as cancer).

In other embodiments of the method of treating described herein, the virus or the pharmaceutical composition is administered in combination with one or more additional agents for treating the cell proliferation disorder (such as cancer), for example, the one or more additional agents described in Section 5.5.3.

The recombinant orthopoxvirus and the pharmaceutical composition disclosed herein can be administered to a subject, e.g., a mammalian subject, such as a human, suffering from a cell proliferation disorder, such as cancer, e.g., to kill cancer cells directly by oncolysis and/or to enhance the effectiveness of the adaptive immune response against the target cancer cells. In some embodiments, the cell proliferation disorder is a cancer, such as leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer. In particular cases, the cell proliferation disorder may be a cancer selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

A physician having ordinary skill in the art can readily determine an effective amount of the recombinant orthopoxvirus vector for administration to a subject, e.g., a mammalian subject (e.g., a human) in need thereof. For example, a physician may start prescribing doses of recombinant orthopoxvirus vector at levels lower than that required in order to achieve the desired therapeutic effect and gradually increase the dosage until the desired effect is achieved. Alternatively, a physician may begin a treatment regimen by administering a dose of recombinant orthopoxvirus vector and subsequently administer progressively lower doses until a therapeutic effect is achieved (e.g., a reduction in the volume of one or more tumors). In general, a suitable daily dose of a recombinant orthopoxvirus vector of the invention will be an amount of the recombinant orthopoxvirus vector which is the lowest dose effective to produce a therapeutic effect. A daily dose of a therapeutic composition of the recombinant orthopoxvirus vector of the invention may be administered as a single dose or as two, three, four, five, six or more doses administered separately at appropriate intervals throughout the day, week, month, or year, optionally, in unit dosage forms. While it is possible for the recombinant orthopoxvirus vector of the invention to be administered alone, it may also be administered as a pharmaceutical formulation in combination with excipients, carriers, and optionally, additional therapeutic agents.

Recombinant orthopoxvirus vectors of the invention can be monitored for their ability to attenuate the progression of a cell proliferation disease, such as cancer, by any of a variety of methods known in the art. For instance, a physician may monitor the response of a subject, e.g., a mammalian subject (e.g., a human) to treatment with recombinant orthopoxvirus vector of the invention by analyzing the volume of one or more tumors in the patient. Alternatively, a physician may monitor the responsiveness of a subject (e.g., a human) t to treatment with recombinant orthopoxvirus vector of the invention by analyzing the T-reg cell population in the lymph of a particular subject. For instance, a physician may withdraw a sample from a subject, e.g., a mammalian subject (e.g., a human) and determine the quantity or density of cancer cells using established procedures, such as fluorescence activated cell sorting. A finding that the quantity of cancer cells in the sample has decreased (e.g., by 1%, 2%, 3%, 4%, 5%, 6%, 7%, 8%, 9%, 10%, 20%, 30%, 40%, 50%, 60%, 70%, 80%, 90%, 95%, 96%, 97%, 98%, 99%, or more) relative to the quantity of cancer cells in a sample obtained from the subject prior to administration of the recombinant orthopoxvirus may be an indication that the orthopoxvirus administration is effectively treating the cancer.

### 5.5.3. Combination Therapy

The recombinant orthopoxvirus vectors described herein may be administered with one or more additional agents, such as an immune checkpoint inhibitor. For instance, the recombinant orthopoxvirus vector can be administered simultaneously with, admixed with, or administered separately from an immune checkpoint inhibitor. Exemplary immune checkpoint inhibitors for use in conjunction with the compositions and methods of the invention include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. Additionally or alternatively, a vector of the invention can be administered simultaneously with, admixed with, or administered separately from an interleukin (IL). For instance, the recombinant orthopoxvirus vector can be administered simultaneously with, admixed with, or administered separately from an interleukin. Exemplary interleukins for use in conjunction with the compositions and methods of the invention include but are not limited to IL-1 alpha, IL-1 beta, IL-2, IL-4, IL-7, IL-10, IL-12 p35, IL-12 p40, IL-12 p70, IL-15, IL-18, IL-21, and IL-23. Additionally or alternatively, a vector of the invention can be administered simultaneously with, admixed with, or administered separately from an interferon. For instance, the recombinant orthopoxvirus vector can be administered simultaneously with, admixed with, or administered separately from an interferon. Exemplary interferons for use in conjunction with the compositions and methods of the invention include but are not limited to IFN-alpha, IFN-beta, IFN-delta, IFN-epsilon, IFN-tau, IFN-omega, IFN-zeta, and IFN-gamma. Additionally or alternatively, a vector of the invention can be administered simultaneously with, admixed with, or administered separately from a TNF superfamily member protein. For instance, the recombinant orthopoxvirus vector can be administered simultaneously with, admixed with, or administered separately from a TNF superfamily member protein. Exemplary TNF superfamily member proteins for use in conjunction with the compositions and methods of the invention include but are not limited to TRAIL, Fas ligand, LIGHT (TNFSF-14), TNF-alpha, and 4-1BB ligand. Additionally or alternatively, a vector of the invention can be administered simultaneously with, admixed with, or administered separately from a cytokine. For instance, the recombinant orthopoxvirus vector can be administered simultaneously with, admixed with, or administered separately from a cytokine. Exemplary cytokines for use in conjunction with the compositions and methods of the invention includes but are not limited to GM-CSF, Flt3 ligand, CD40 ligand, anti-TGF-beta, anti-VEGF-R2, and cGAS (guanyl adenylate cyclase).

Additionally or alternatively, immune checkpoint inhibitors may be expressed in the orthopoxvirus itself. For instance, the recombinant orthopoxvirus vector can include a transgene encoding an immune checkpoint inhibitor. Exemplary immune checkpoint inhibitors for expression by the orthopoxvirus of the compositions and methods of the invention include but are not limited to OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. Additionally or alternatively, interleukins may be expressed in the orthopoxvirus itself. For instance, the recombinant orthopoxvirus vector can include a transgene encoding an interleukin. Exemplary immune checkpoint inhibitors for expression by the orthopoxvirus of the compositions and methods of the invention include but are not limited to IL-1 alpha, IL-1 beta, IL-2, IL-4, IL-7, IL-10, IL-12 p35, IL-12 p40, IL-12 p70, IL-15, IL-18, IL-21, and IL-23. Additionally or alternatively, interferons may be expressed in the orthopoxvirus itself. For instance, the recombinant orthopoxvirus vector can include a transgene encoding an interferon. Exemplary interferons for expression by the orthopoxvirus of the compositions and methods of the invention include but are not limited to IFN-alpha, IFN-beta, IFN-delta, IFN-epsilon, IFN-tau, IFN-omega, IFN-zeta, and IFN-gamma. Additionally or alternatively, TNF superfamily member proteins may be expressed in the orthopoxvirus itself. For instance, the recombinant orthopoxvirus vector can include a transgene encoding a TNF superfamily member protein. Exemplary TNF superfamily member proteins for expression by the orthopoxvirus of the compositions and methods of the invention include but are not limited to TRAIL, Fas ligand, LIGHT (TNFSF-14), TNF-alpha, and 4-1BB ligand. Additionally or alternatively, cytokines may be expressed in the orthopoxvirus itself. For instance, the recombinant orthopoxvirus vector can include a transgene encoding a cytokine. Exemplary cytokines for expression by the orthopoxvirus of the compositions and methods of the invention include but are not limited to GM-CSF, fms-related tyrosine kinase 3 (Flt3) ligand, CD40 ligand, TGF-beta, VEGF-R2, and c-KIT.

Additionally or alternatively, tumor-associated antigens may be expressed in the orthopoxvirus itself. For instance, the recombinant orthopoxvirus vector can include a transgene encoding a tumor-associated antigen. Exemplary tumor-associated antigens for expression by the orthopoxvirus of the compositions and methods of the invention include but are not limited to CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, NTRK, MAGE-A3, NY-ESO-1, one or more human papillomavirus (HPV) proteins, E6 and E7 proteins of HPV16, E6 and E7 proteins of HPV18, brachyury, or prostatic acid phosphatase, or one or more fragments thereof. Additional examples of tumor-associated antigens for use in conjunction with the compositions and methods described herein include, but are not limited to, those listed in Tables 3-30

In certain embodiments of the method of treating described herein, the method further comprises administering to the patient (e.g., a mammalian patient, such as a human patient) an immune checkpoint inhibitor. In specific embodiments, the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof. In a specific embodiment, the immune checkpoint inhibitor is an anti-PD1 antibody or antigen-binding fragment thereof or an anti-CTLA-4 antibody or antigen-binding fragment thereof. In another specific embodiment, the immune checkpoint inhibitor is an anti-PD1 antibody or antigen-binding fragment thereof. In another specific embodiment, the immune checkpoint inhibitor is an anti-CTLA-4 antibody or antigen-binding fragment thereof. In another specific embodiment, the immune checkpoint inhibitor is an anti-PD-L1 antibody or antigen-binding fragment thereof. In a specific embodiment, the immune checkpoint inhibitor is ipilimumab. In another specific embodiment, the immune checkpoint inhibitor is tremelimumab. In another specific embodiment, the immune checkpoint inhibitor is nivolumab. In another specific embodiment, the immune checkpoint inhibitor is pembrolizumab. In another specific embodiment, the immune checkpoint inhibitor is cemiplimab. In another specific embodiment, the immune checkpoint inhibitor is atezolizumab. In another specific embodiment, the immune checkpoint inhibitor is avelumab. In another specific embodiment, the immune checkpoint inhibitor is durvalumab.

In certain embodiments of the method of treating described herein, the method further comprises administering to the patient (e.g., a mammalian patient, such as a human patient) an interleukin. In specific embodiments, the interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23. In specific embodiments, the interleukin is selected from the group consisting of IL-12 p35, IL-12 p40, and IL-12 p70. In specific embodiments, the interleukin is membrane-bound.

In certain embodiments of the method of treating described herein, the method further comprises administering to the patient (e.g., a mammalian patient, such as a human patient) an interferon. In specific embodiments, the interferon is selected from the group consisting of IFN-alpha, IFN-beta, IFN-delta, IFN-epsilon, IFN-tau, IFN-omega, IFN-zeta, and IFN-gamma.

In certain embodiments of the method of treating described herein, the method further comprises administering to the patient (e.g., a mammalian patient, such as a human patient) a cytokine. In specific embodiments, the cytokine is a TNF superfamily member protein. In a specific embodiment, the TNF superfamily member protein is selected from the group consisting of TRAIL, Fas ligand, LIGHT (TNFSF-14), TNF-alpha, and 4-1BB ligand. In specific embodiments, the cytokine is selected from the group consisting of GM-CSF, Flt3 ligand, CD40 ligand, TGF-beta, VEGF-R2, and cKit. In a specific embodiment, the cytokine is Flt3 ligand.

### 5.6. Kits

Also provided herein are kits that can be used in accordance with the invention.

In one aspect, provided herein is a kit comprising a nucleic acid described in Section 5.2.3 and a package insert instructing a user of the kit to express the nucleic acid in a host cell.

In another aspect, provided herein is a kit comprising a virus described in Section 5.2.4 and a package insert instructing a user of the kit to express the virus in a host cell.

In another aspect, provided herein is a kit comprising a virus described in Section 5.2.4 and a package insert instructing a user to administer a therapeutically effective amount of the virus to a patient (e.g., a mammalian patient, such as a human patient) having cancer, thereby treating the cancer. In certain embodiments, the mammalian patient is a human patient. The cancer to be treated can be a cancer described in Section 5.5.

In preferred embodiments, the nucleic acid or the virus is stored in one or more containers suitable for storing the nucleic acid or the virus. In certain embodiments, the kits provided herein further comprise controls suitable for their intended use.

### 5.7. Illustrative Embodiments

### 5.7.1. Set 1

1. A nucleic acid comprising a recombinant orthopoxvirus genome, wherein said recombinant orthopoxvirus genome comprises a deletion of at least 2 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
2. The nucleic acid of embodiment 1, wherein said deletion comprises at least 3 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
3. The nucleic acid of embodiment 2, wherein said deletion comprises at least 4 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
4. The nucleic acid of embodiment 3, wherein said deletion comprises at least 5 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
5. The nucleic acid of embodiment 4, wherein said deletion comprises at least 6 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
6. The nucleic acid of embodiment 5, wherein said deletion comprises at least 7 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
7. The nucleic acid of embodiment 6, wherein said deletion comprises at least 8 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
8. The nucleic acid of embodiment 7, wherein said deletion comprises at least 9 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
9. The nucleic acid of embodiment 8, wherein said deletion comprises at least 10 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
10. The nucleic acid of embodiment 9, wherein said deletion comprises at least 11 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
11. The nucleic acid of embodiment 10, wherein said deletion comprises at least 12 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
12. The nucleic acid of embodiment 11, wherein said deletion comprises at least 13 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
13. The nucleic acid of embodiment 12, wherein said deletion comprises at least 14 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
14. The nucleic acid of embodiment 13, wherein said deletion comprises at least 15 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
15. The nucleic acid of embodiment 14, wherein said deletion comprises at least 16 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
16. The nucleic acid of embodiment 15, wherein said deletion comprises at least 17 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
17. The nucleic acid of embodiment 16, wherein said deletion comprises at least 18 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
18. The nucleic acid of embodiment 17, wherein said deletion comprises at least 19 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
19. The nucleic acid of embodiment 18, wherein said deletion comprises at least 20 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
20. The nucleic acid of embodiment 19, wherein said deletion comprises at least 21 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
21. The nucleic acid of embodiment 20, wherein said deletion comprises each of said F1L, N1L, B14R, M2L, K1L, K7R, C2L, N2L, M1L, K2L, K3L, F3L, B16R, B19R, K4L, K5L, K6L, F2L, B15R, B17L, B18R, and B20R genes.
22. A nucleic acid comprising a recombinant orthopoxvirus genome, wherein said recombinant orthopoxvirus genome comprises a deletion of at least 1 gene selected from the group consisting of B14R, B16R, B17L, B18R, B19R, and B20R.
23. The nucleic acid of any one of embodiments 1-22, wherein said recombinant orthopoxvirus genome comprises a deletion of at least 1 gene selected from the group consisting of B14R, B16R, B17L, B18R, B19R, and B20R.
24. The nucleic acid of embodiment 23, wherein said deletion comprises at least 2 genes, each independently selected from the group consisting of B14R, B16R, B17L, B18R, B19R, and B20R.
25. The nucleic acid of embodiment 24, wherein said deletion comprises at least 3 genes, each independently selected from the group consisting of B14R, B16R, B17L, B18R, B19R, and B20R.
26. The nucleic acid of embodiment 25, wherein said deletion comprises at least 4 genes, each independently selected from the group consisting of B14R, B16R, B17L, B18R, B19R, and B20R.
27. The nucleic acid of embodiment 26, wherein said deletion comprises at least 5 genes, each independently selected from the group consisting of B14R, B16R, B17L, B18R, B19R, and B20R.
28. The nucleic acid of embodiment 27, wherein said deletion comprises each of said B14R, B16R, B17L, B18R, B19R, and B20R genes.
29. A nucleic acid comprising a recombinant orthopoxvirus genome, wherein said recombinant orthopoxvirus genome comprises a deletion of at least 1 gene selected from the group consisting of C2L, C1L, N1L, N2L, M1L, K1L, K2L, K3L, K4L, K7R, and F2L.
30. The nucleic acid of any one of embodiments 1-29, wherein said recombinant orthopoxvirus genome comprises a deletion of at least 1 gene selected from the group consisting of C2L, C1L, N1L, N2L, M1L, K1L, K2L, K3L, K4L, K7R, and F2L.
31. The nucleic acid of embodiment 30, wherein said deletion comprises at least 2 genes, each independently selected from the group consisting of C2L, C1L, N1L, N2L, M1L, K1L, K2L, K3L, K4L, K7R, and F2L.
32. The nucleic acid of embodiment 31, wherein said deletion comprises at least 3 genes, each independently selected from the group consisting of C2L, C1L, N1L, N2L, M1L, K1L, K2L, K3L, K4L, K7R, and F2L.
33. The nucleic acid of embodiment 32, wherein said deletion comprises at least 4 genes, each independently selected from the group consisting of C2L, C1L, N1L, N2L, M1L, K1L, K2L, K3L, K4L, K7R, and F2L.
34. The nucleic acid of embodiment 33, wherein said deletion comprises at least 5 genes, each independently selected from the group consisting of C2L, C1L, N1L, N2L, M1L, K1L, K2L, K3L, K4L, K7R, and F2L.
35. The nucleic acid of embodiment 34, wherein said deletion comprises at least 6 genes, each independently selected from the group consisting of C2L, C1L, N1L, N2L, M1L, K1L, K2L, K3L, K4L, K7R, and F2L.
36. The nucleic acid of embodiment 35, wherein said deletion comprises at least 7 genes, each independently selected from the group consisting of C2L, C1L, N1L, N2L, M1L, K1L, K2L, K3L, K4L, K7R, and F2L.
37. The nucleic acid of embodiment 36, wherein said deletion comprises at least 8 genes, each independently selected from the group consisting of C2L, C1L, N1L, N2L, M1L, K1L, K2L, K3L, K4L, K7R, and F2L.
38. The nucleic acid of embodiment 37, wherein said deletion comprises at least 9 genes, each independently selected from the group consisting of C2L, C1L, N1L, N2L, M1L, K1L, K2L, K3L, K4L, K7R, and F2L.
39. The nucleic acid of embodiment 38, wherein said deletion comprises at least 10 genes, each independently selected from the group consisting of C2L, C1L, N1L, N2L, M1L, K1L, K2L, K3L, K4L, K7R, and F2L.
40. The nucleic acid of embodiment 39, wherein said deletion comprises each of said C2L, C1L, N1L, N2L, M1L, K1L, K2L, K3L, K4L, K7R, and F2L genes.
41. A nucleic acid comprising a recombinant orthopoxvirus genome, wherein said recombinant orthopoxvirus genome comprises a deletion of at least 1 gene that encodes a caspase-9 inhibitor.
42. The nucleic acid of any one of embodiments 1-41, wherein said recombinant orthopoxvirus genome comprises a deletion of at least 1 gene that encodes a caspase-9 inhibitor.
43. The nucleic acid of embodiment 41 or 42, wherein said gene that encodes a caspase-9 inhibitor is F1L.
44. A nucleic acid comprising a recombinant orthopoxvirus genome, wherein said recombinant orthopoxvirus genome comprises a deletion of at least 1 gene that encodes a BCL-2 inhibitor.
45. The nucleic acid of any one of embodiments 1-44, wherein said recombinant orthopoxvirus genome comprises a deletion of at least 1 gene that encodes a BCL-2 inhibitor.
46. The nucleic acid of embodiment 44 or 45, wherein said gene that encodes a BCL-2 inhibitor is N1L.
47. A nucleic acid comprising a recombinant orthopoxvirus genome, wherein said recombinant orthopoxvirus genome comprises a deletion of at least 1 gene that encodes a dUTPase.
48. The nucleic acid of any one of embodiments 1-47, wherein said recombinant orthopoxvirus genome comprises a deletion of at least 1 gene that encodes a dUTPase.
49. The nucleic acid of embodiment 46 or 47, wherein said gene that encodes a dUTPase is F2L.
50. A nucleic acid comprising a recombinant orthopoxvirus genome, wherein said recombinant orthopoxvirus genome comprises a deletion of at least 1 gene that encodes a IFN-alpha/beta-receptor-like secreted glycoprotein.
51. The nucleic acid of any one of embodiments 1-50, wherein said recombinant orthopoxvirus genome comprises a deletion of at least 1 gene that encodes a IFN-alpha/beta-receptor-like secreted glycoprotein.
52. The nucleic acid of embodiment 50 or 51, wherein said gene that encodes a IFN-alpha/beta-receptor-like secreted glycoprotein is B19R.
53. A nucleic acid comprising a recombinant orthopoxvirus genome, wherein said recombinant orthopoxvirus genome comprises a deletion of at least 1 gene that encodes an IL-1-beta-inhibitor.
54. The nucleic acid of any one of embodiments 1-53, wherein said recombinant orthopoxvirus genome comprises a deletion of at least 1 gene that encodes an IL-1-beta-inhibitor.
55. The nucleic acid of embodiment 53 or 54, wherein said gene that encodes an IL-1-beta-inhibitor is B16R.
56. A nucleic acid comprising a recombinant orthopoxvirus genome, wherein said recombinant orthopoxvirus genome comprises a deletion of at least 1 gene that encodes a phospholipase-D.
57. The nucleic acid of any one of embodiments 1-56, wherein said recombinant orthopoxvirus genome comprises a deletion of at least 1 gene that encodes a phospholipase-D.
58. The nucleic acid of embodiment 56 or 57, wherein said gene that encodes a phospholipase-D is K4L.
59. A nucleic acid comprising a recombinant orthopoxvirus genome, wherein said recombinant orthopoxvirus genome comprises a deletion of at least 1 gene that encodes a PKR inhibitor.
60. The nucleic acid of any one of embodiments 1-59, wherein said recombinant orthopoxvirus genome comprises a deletion of at least 1 gene that encodes a PKR inhibitor.
61. The nucleic acid of embodiment 59 or 60, wherein said gene that encodes a PKR inhibitor is K3L.
62. A nucleic acid comprising a recombinant orthopoxvirus genome, wherein said recombinant orthopoxvirus genome comprises a deletion of at least 1 gene that encodes a serine protease inhibitor.
63. The nucleic acid of any one of embodiments 1-62, wherein said recombinant orthopoxvirus genome comprises a deletion of at least 1 gene that encodes a serine protease inhibitor.
64. The nucleic acid of embodiment 62 or 63, wherein said gene that encodes a serine protease inhibitor is K2L.
65. A nucleic acid comprising a recombinant orthopoxvirus genome, wherein said recombinant orthopoxvirus genome comprises a deletion of at least 1 gene that encodes a TLR signaling inhibitor.
66. The nucleic acid of any one of embodiments 1-65, wherein said recombinant orthopoxvirus genome comprises a deletion of at least 1 gene that encodes a TLR signaling inhibitor.
67. The nucleic acid of embodiment 65 or 66, wherein said gene that encodes a TLR signaling inhibitor is N2L.
68. A nucleic acid comprising a recombinant orthopoxvirus genome, wherein said recombinant orthopoxvirus genome comprises a deletion of at least 1 gene that encodes a kelch-like protein.
69. The nucleic acid of any one of embodiments 1-68, wherein said recombinant orthopoxvirus genome comprises a deletion of at least 1 gene that encodes a kelch-like protein.
70. The nucleic acid of embodiment 69, wherein said recombinant orthopoxvirus genome comprises a deletion of at least 2 genes that each encodes a kelch-like protein.
71. The nucleic acid of any one of embodiments 68-70, wherein said genes that encode a kelch-like protein are, independently, selected from the group consisting of F3L and C2L.
72. A nucleic acid comprising a recombinant orthopoxvirus genome, wherein said recombinant orthopoxvirus genome comprises a deletion of at least 1 gene that encodes a monoglyceride lipase.
73. The nucleic acid of any one of embodiments 1-72, wherein said recombinant orthopoxvirus genome comprises a deletion of at least 1 gene that encodes a monoglyceride lipase.
74. The nucleic acid of embodiment 73, wherein said recombinant orthopoxvirus genome comprises a deletion of at least 2 genes that each encodes a monoglyceride lipase.
75. The nucleic acid of any one of embodiments 72-74, wherein said genes that encode a monoglyceride lipase are, independently, selected from the group consisting of K5L and K6L.
76. A nucleic acid comprising a recombinant orthopoxvirus genome, wherein said recombinant orthopoxvirus genome comprises a deletion of at least 1 gene that encodes an NF-κB inhibitor.
77. The nucleic acid of any one of embodiments 1-76, wherein said recombinant orthopoxvirus genome comprises a deletion of at least 1 gene that encodes an NF-κB inhibitor.
78. The nucleic acid of embodiment 77, wherein said recombinant orthopoxvirus genome comprises a deletion of at least 2 genes that each encodes an NF-κB inhibitor.
79. The nuclei acid of embodiment 78, wherein said recombinant orthopoxvirus genome comprises a deletion of at least 3 genes that each encodes an NF-κB inhibitor.
80. The nucleic acid of any one of embodiments 76-79, wherein said genes that encode an NF-κB inhibitor are, independently, selected from the group consisting of K7R, K1L, and M2L.
81. A nucleic acid comprising a recombinant orthopoxvirus genome, wherein said recombinant orthopoxvirus genome comprises a deletion of at least 1 gene that encodes an Ankyrin repeat protein.
82. The nucleic acid of any one of embodiments 1-81, wherein said recombinant orthopoxvirus genome comprises a deletion of at least 1 gene that encodes an Ankyrin repeat protein.
83. The nucleic acid of embodiment 82, wherein said recombinant orthopoxvirus genome comprises a deletion of at least 2 genes that each encodes an Ankyrin repeat protein.
84. The nuclei acid of embodiment 83, wherein said recombinant orthopoxvirus genome comprises a deletion of at least 3 genes that each encodes an Ankyrin repeat protein.
85. The nucleic acid of any one of embodiments 81-84, wherein said genes that encode an Ankyrin repeat protein are, independently, selected from the group consisting of B 18R, B20R, and M1L.
86. A nucleic acid comprising a recombinant orthopoxvirus genome, wherein said recombinant orthopoxvirus genome comprises a deletion of at least 1 gene selected from the group consisting of B15R, B17R, and B14R.
87. The nucleic acid of any one of embodiments 1-86, wherein said recombinant orthopoxvirus genome comprises a deletion of at least 1 gene selected from the group consisting of B15R, B17R, and B14R.
88. The nucleic acid of embodiment 87, wherein said deletion comprises at least 2 genes, each independently selected from the group consisting of B15R, B17R, and B14R.
89. The nucleic acid of embodiment 88, wherein said deletion comprises each of said B15R, B17R, and B14R genes.
90. The nucleic acid of any one of embodiments 1-89, wherein said recombinant orthopoxvirus genome comprises a deletion of at least 1 gene selected from the group of ITR genes consisting of B21R, B22R, B23R, B24R, B25R, B26R, B27R, B28R, and B29R.
91. The nucleic acid of embodiment 90, wherein said deletion comprises at least 2 genes, each independently selected from said group of ITR genes consisting of B21R, B22R, B23R, B24R, B25R, B26R, B27R, B28R, and B29R.
92. The nucleic acid of embodiment 91, wherein said deletion comprises at least 3 genes, each independently selected from said group of ITR genes consisting of B21R, B22R, B23R, B24R, B25R, B26R, B27R, B28R, and B29R.
93. The nucleic acid of embodiment 92, wherein said deletion comprises at least 4 genes, each independently selected from said group of ITR genes consisting of B21R, B22R, B23R, B24R, B25R, B26R, B27R, B28R, and B29R.
94. The nucleic acid of embodiment 93, wherein said deletion comprises at least 5 genes, each independently selected from said group of ITR genes consisting of B21R, B22R, B23R, B24R, B25R, B26R, B27R, B28R, and B29R.
95. The nucleic acid of embodiment 94, wherein said deletion comprises at least 6 genes, each independently selected from said group of ITR genes consisting of B21R, B22R, B23R, B24R, B25R, B26R, B27R, B28R, and B29R.
96. The nucleic acid of embodiment 95, wherein said deletion comprises at least 7 genes, each independently selected from said group of ITR genes consisting of B21R, B22R, B23R, B24R, B25R, B26R, B27R, B28R, and B29R.
97. The nucleic acid of embodiment 96, wherein said deletion comprises at least 8 genes, each independently selected from said group of ITR genes consisting of B21R, B22R, B23R, B24R, B25R, B26R, B27R, B28R, and B29R.
98. The nucleic acid of embodiment 97, wherein said deletion comprises each of said B21R, B22R, B23R, B24R, B25R, B26R, B27R, B28R, and B29R genes.
99.
100.
102. The nucleic acid of embodiment 101, wherein said vaccinia virus is a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1.
103. The nucleic acid of embodiment 101, wherein said vaccinia virus is a strain selected from the group consisting of Copenhagen, Western Reserve, Tian Tan, Wyeth, and Lister.
104. The nucleic acid of embodiment 101, wherein said vaccinia virus is a Copenhagen strain vaccinia virus.
105. The nucleic acid of any one of embodiments 1-104, wherein each of said deletions is a deletion of the entire polynucleotide encoding the corresponding gene.
106. The nucleic acid of any one of embodiments 1-104, wherein each of said deletions is a deletion of a portion of the polynucleotide encoding the corresponding gene, and wherein said deletion is sufficient to render said gene nonfunctional upon introduction into a host cell.
107. The nucleic acid of any one of embodiments 1-106, wherein said nucleic acid further comprises a transgene encoding a tumor-associated antigen.
108. The nucleic acid of embodiment 107, wherein said tumor-associated antigen is a tumor-associated antigen listed in any one of Tables 3-30.
109. The nucleic acid of embodiment 107, wherein said tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.
110. The nucleic acid of embodiment 107, wherein said tumor-associated antigen comprises MAGE-A3, or one or more fragments thereof.
111. The nucleic acid of embodiment 107, wherein said tumor-associated antigen comprises NY-ESO-1, or one or more fragments thereof.
112. The nucleic acid of embodiment 107, wherein said tumor-associated antigen comprises one or more human papillomavirus (HPV) proteins, or fragments thereof.
113. The nucleic acid of embodiment 107, wherein said tumor-associated antigen comprises (i) E6 and E7 proteins, or fragments thereof, of HPV16 and (ii) E6 and E7 proteins, or fragments thereof, of HPV18.
114. The nucleic acid of embodiment 107, wherein said tumor-associated antigen comprises brachyury or one or more fragments thereof.
115. The nucleic acid of embodiment 107, wherein said tumor-associated antigen comprises prostatic acid phosphatase, or one or more fragments thereof.
116. A method of producing the nucleic acid of any one of embodiments 107-115, said method comprising:
   a. introducing a transposon insertion site into the nucleic acid of any one of embodiments 1-106;
   b. contacting the nucleic acid formed in (a) with a transposable element comprising a gene encoding said tumor-associated antigen, thereby introducing said gene into said nucleic acid; and
   c. recovering the nucleic acid formed in (b).
117. The nucleic acid of any one of embodiments 1-115, wherein said nucleic acid further comprises a transgene encoding an immune checkpoint inhibitor.
118. The nucleic acid of embodiment 117, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
119. The nucleic acid of embodiment 117, wherein said immune checkpoint inhibitor is an anti-PD-L1 antibody or antigen-binding fragment thereof or an anti-CTLA-4 antibody or antigen-binding fragment thereof.
120. The nucleic acid of embodiment 117, wherein said immune checkpoint inhibitor is an anti-PD1 antibody or antigen-binding fragment thereof.
121. The nucleic acid of embodiment 117, wherein said immune checkpoint inhibitor is an anti-CTLA-4 antibody or antigen-binding fragment thereof.
122. A method of producing the nucleic acid of any one of embodiments 117-121, said method comprising:
   a. introducing a transposon insertion site into the nucleic acid of any one of embodiments 1-116;
   b. contacting the nucleic acid formed in (a) with a transposable element comprising a gene encoding said immune checkpoint inhibitor, thereby introducing said gene into said nucleic acid; and
   c. recovering the nucleic acid formed in (b).
123. The nucleic acid of any one of embodiments 1-115 and 117-121, wherein said nucleic acid further comprises a transgene encoding an interleukin (IL).
124. The nucleic acid of embodiment 123, wherein said interleukin is selected from the group consisting of
IL-1 alpha, IL-1 beta, IL-2, IL-4, IL-7, IL-10, IL-12 p35, IL-12 p40, IL-12 p70, IL-15, IL-18, IL-21, and IL-23.
125. The nucleic acid of embodiment 123, wherein said interleukin is selected from the group consisting of IL-12 p35, IL-12 p70.
126. The nucleic acid of embodiment 125, wherein said interleukin is membrane-bound.
127. A method of producing the nucleic acid of one of embodiments 123-126, said method comprising:
   a. introducing a transposon insertion site into the nucleic acid of any one of embodiments 1-115 and 117-121;
   b. contacting the nucleic acid formed in (a) with a transposable element comprising a gene encoding said interleukin, thereby introducing said gene into said nucleic acid; and
   c. recovering the nucleic acid formed in (b).
128. The nucleic acid of any one of embodiments 1-115, 117-121, and 123-126, wherein said nucleic acid further comprises a transgene encoding an interferon (IFN).
129. The nucleic acid of embodiment 128, wherein said interferon is selected from the group consisting of IFN-alpha, IFN-beta, IFN-delta, IFN-epsilon, IFN-tau, IFN-omega, IFN-zeta, and IFN-gamma.
130. A method of producing the nucleic acid of embodiment 128 or 129, said method comprising:
   a. introducing a transposon insertion site into the nucleic acid of any one of embodiments 1-115, 117-121, and 123-126;
   b. contacting the nucleic acid formed in (a) with a transposable element comprising a gene encoding said interferon, thereby introducing said gene into said nucleic acid; and
   c. recovering the nucleic acid formed in (b).
131. The nucleic acid of any one of embodiments 1-115, 117-121, 123-126, 128, and 129, wherein said nucleic acid further comprises a transgene encoding a TNF superfamily member protein.
132. The nucleic acid of embodiment 131, wherein said TNF superfamily member protein is selected from the group consisting of TRAIL, Fas ligand, LIGHT (TNFSF-14), TNF-alpha, and 4-1BB ligand.
133. A method of producing the nucleic acid of embodiment 131 or 132, said method comprising:
   a. introducing a transposon insertion site into the nucleic acid of any one of embodiments 1-115, 117-121, 123-126, 128, and 129;
   b. contacting the nucleic acid formed in (a) with a transposable element comprising a gene encoding said TNF superfamily member protein, thereby introducing said gene into said nucleic acid; and
   c. recovering the nucleic acid formed in (b).
134. The nucleic acid of any one of embodiments 1-115, 117-121, 123-126, 128, 129, 131, and 132 wherein said nucleic acid further comprises a transgene encoding a cytokine.
135. The nucleic acid of embodiment 134, wherein said cytokine is selected from the group consisting of GM-CSF, Flt3 ligand, CD40 ligand, anti-TGF-beta, anti-VEGF-R2, and cGAS (guanyl adenylate cyclase).
136. The nucleic acid of embodiment 134, wherein said cytokine is Flt3 ligand.
137. A method of producing the nucleic acid of embodiment 135 or 136, said method comprising:
   a. introducing a transposon insertion site into the nucleic acid of any one of embodiments 1-115, 117-121, 123-126, 128, 129, 131, and 132;
   b. contacting the nucleic acid formed in (a) with a transposable element comprising a gene encoding said cytokine, thereby introducing said gene into said nucleic acid; and
   c. recovering the nucleic acid formed in (b).
138. The nucleic acid of any one of embodiments 1-115, 117-121, 123-126, 128, 129, 131, 132, and 134-136, wherein said nucleic acid further comprises a transgene encoding microRNA (miRNA).
139. The nucleic acid of embodiment 138, wherein said miRNA is mir-6.
140. A method of producing the nucleic acid of embodiment 138 or 139, said method comprising:
   a. introducing a transposon insertion site into the nucleic acid of any one of embodiments 1-115, 117-121, 123-126, 128, 129, 131, 132, and 134-136;
   b. contacting the nucleic acid formed in (a) with a transposable element comprising a gene encoding said miRNA, thereby introducing said gene into said nucleic acid; and
   c. recovering the nucleic acid formed in (b).
141. A recombinant orthopoxvirus vector comprising a deletion of at least 2 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
142. The recombinant orthopoxvirus vector of embodiment 141, wherein said deletion comprises at least 3 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
143. The recombinant orthopoxvirus vector of embodiment 142, wherein said deletion comprises at least 4 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
144. The recombinant orthopoxvirus vector of embodiment 143, wherein said deletion comprises at least 5 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
145. The recombinant orthopoxvirus vector of embodiment 144, wherein said deletion comprises at least 6 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
146. The recombinant orthopoxvirus vector of embodiment 145, wherein said deletion comprises at least 7 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
147. The recombinant orthopoxvirus vector of embodiment 146, wherein said deletion comprises at least 8 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
148. The recombinant orthopoxvirus vector of embodiment 147, wherein said deletion comprises at least 9 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
149. The recombinant orthopoxvirus vector of embodiment 148, wherein said deletion comprises at least 10 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
150. The recombinant orthopoxvirus vector of embodiment 149, wherein said deletion comprises at least 11 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
151. The recombinant orthopoxvirus vector of embodiment 150, wherein said deletion comprises at least 12 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
152. The recombinant orthopoxvirus vector of embodiment 151, wherein said deletion comprises at least 13 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
153. The recombinant orthopoxvirus vector of embodiment 152, wherein said deletion comprises at least 14 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
154. The recombinant orthopoxvirus vector of embodiment 153, wherein said deletion comprises at least 15 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
155. The recombinant orthopoxvirus vector of embodiment 154, wherein said deletion comprises at least 16 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
156. The recombinant orthopoxvirus vector of embodiment 155, wherein said deletion comprises at least 17 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
157. The recombinant orthopoxvirus vector of embodiment 156, wherein said deletion comprises at least 18 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
158. The recombinant orthopoxvirus vector of embodiment 157, wherein said deletion comprises at least 19 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
159. The recombinant orthopoxvirus vector of embodiment 158, wherein said deletion comprises at least 20 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
160. The recombinant orthopoxvirus vector of embodiment 159, wherein said deletion comprises at least 21 genes, each independently selected from the group consisting of F1L, N1L, B14R, M2L, K1L, K7R, C2L, N2L, M1L, K2L, K3L, F3L, B16R, B19R, K4L, K5L, K6L, F2L, B15R, B17L, B18R, and B20R.
161. The recombinant orthopoxvirus vector of embodiment 160, wherein said deletion comprises each of said F1L, N1L, B14R, M2L, K1L, K7R, C2L, N2L, M1L, K2L, K3L, F3L, B16R, B19R, K4L, K5L, K6L, F2L, B15R, B17L, B18R, and B20R genes.
162. A recombinant orthopoxvirus vector comprising a deletion of at least 1 gene selected from the group consisting of B14R, B16R, B17L, B18R, B19R, and B20R.
163. The recombinant orthopoxvirus vector of any one of embodiments 141-162, wherein said vector comprises a deletion of at least 1 gene selected from the group consisting of B14R, B16R, B17L, B18R, B19R, and B20R.
164. The vector of embodiment 163, wherein said deletion comprises at least 2 genes, each independently selected from the group consisting of B14R, B16R, B17L, B18R, B19R, and B20R.
165. The vector of embodiment 164, wherein said deletion comprises at least 3 genes, each independently selected from the group consisting of B14R, B16R, B17L, B18R, B19R, and B20R.
166. The vector of embodiment 165, wherein said deletion comprises at least 4 genes, each independently selected from the group consisting of B14R, B16R, B17L, B18R, B19R, and B20R.
167. The vector of embodiment 166, wherein said deletion comprises at least 5 genes, each independently selected from the group consisting of B14R, B16R, B17L, B18R, B19R, and B20R.
168. The vector of embodiment 167, wherein said deletion comprises each of B14R, B16R, B17L, B18R, B19R, and B20R.
169. A recombinant orthopoxvirus vector comprising a deletion of at least 1 gene selected from the group consisting of C2L, C1L, N1L, N2L, M1L, K1L, K2L, K3L, K4L, K7R, and F2L.
170. The recombinant orthopoxvirus vector of any one of embodiments 141-169, wherein said vector comprises a deletion of at least 1 gene selected from the group consisting of C2L, C1L, N1L, N2L, M1L, K1L, K2L, K3L, K4L, K7R, and F2L.
171. The vector of embodiment 170, wherein said deletion comprises at least 2 genes, each independently selected from the group consisting of C2L, C1L, N1L, N2L, M1L, K1L, K2L, K3L, K4L, K7R, and F2L.
172. The vector of embodiment 171, wherein said deletion comprises at least 3 genes, each independently selected from the group consisting of C2L, C1L, N1L, N2L, M1L, K1L, K2L, K3L, K4L, K7R, and F2L.
173. The vector of embodiment 172, wherein said deletion comprises at least 4 genes, each independently selected from the group consisting of C2L, C1L, N1L, N2L, M1L, K1L, K2L, K3L, K4L, K7R, and F2L.
174. The vector of embodiment 173, wherein said deletion comprises at least 5 genes, each independently selected from the group consisting of C2L, C1L, N1L, N2L, M1L, K1L, K2L, K3L, K4L, K7R, and F2L.
175. The vector of embodiment 174, wherein said deletion comprises at least 6 genes, each independently selected from the group consisting of C2L, C1L, N1L, N2L, M1L, K1L, K2L, K3L, K4L, K7R, and F2L.
176. The vector of embodiment 175, wherein said deletion comprises at least 7 genes, each independently selected from the group consisting of C2L, C1L, N1L, N2L, M1L, K1L, K2L, K3L, K4L, K7R, and F2L.
177. The vector of embodiment 176, wherein said deletion comprises at least 8 genes, each independently selected from the group consisting of C2L, C1L, N1L, N2L, M1L, K1L, K2L, K3L, K4L, K7R, and F2L.
178. The vector of embodiment 177, wherein said deletion comprises at least 9 genes, each independently selected from the group consisting of C2L, C1L, N1L, N2L, M1L, K1L, K2L, K3L, K4L, K7R, and F2L.
179. The vector of embodiment 178, wherein said deletion comprises at least 10 genes, each independently selected from the group consisting of C2L, C1L, N1L, N2L, M1L, K1L, K2L, K3L, K4L, K7R, and F2L.
180. The vector of embodiment 179, wherein said deletion comprises each of C2L, C1L, N1L, N2L, M1L, K1L, K2L, K3L, K4L, K7R, and F2L.
181. A recombinant orthopoxvirus vector comprising a deletion of at least 1 gene that encodes a caspase-9 inhibitor.
182. The recombinant orthopoxvirus vector of any one of embodiments 141-181, wherein said vector comprises a deletion of at least 1 gene that encodes a caspase-9 inhibitor.
183. The vector of embodiment 181 or 182, wherein said gene that encodes a caspase-9 inhibitor is F1L.
184. A recombinant orthopoxvirus vector, wherein said vector comprises a deletion of at least 1 gene that encodes a BCL-2 inhibitor.
185. The recombinant orthopoxvirus vector of any one of embodiments 141-184, wherein said vector comprises a deletion of at least 1 gene that encodes a BCL-2 inhibitor.
186. The vector of embodiment 184 or 185, wherein said gene that encodes a BCL-2 inhibitor is N1L.
187. A recombinant orthopoxvirus vector, wherein said vector comprises a deletion of at least 1 gene that encodes a dUTPase.
188. The recombinant orthopoxvirus vector of any one of embodiments 141-187, wherein said vector comprises a deletion of at least 1 gene that encodes a dUTPase.
189. The vector of embodiment 187 or 188, wherein said gene that encodes a dUTPase is F2L.
190. A recombinant orthopoxvirus vector, wherein said vector comprises a deletion of at least 1 gene that encodes a IFN-alpha/beta-receptor-like secreted glycoprotein.
191. The recombinant orthopoxvirus vector of any one of embodiments 141-190, wherein said vector comprises a deletion of at least 1 gene that encodes a IFN-alpha/beta-receptor-like secreted glycoprotein.
192. The vector of embodiment 190 or 191, wherein said gene that encodes a IFN-alpha/beta-receptor-like secreted glycoprotein is B19R.
193. A recombinant orthopoxvirus vector, wherein said vector comprises a deletion of at least 1 gene that encodes an IL-1-beta-inhibitor.
194. The recombinant orthopoxvirus vector of any one of embodiments 141-193, wherein said vector comprises a deletion of at least 1 gene that encodes an IL-1-beta-inhibitor.
195. The vector of embodiment 193 or 194, wherein said gene that encodes an IL-1-beta-inhibitor is B16R.
196. A recombinant orthopoxvirus vector, wherein said vector comprises a deletion of at least 1 gene that encodes a phospholipase-D.
197. The recombinant orthopoxvirus vector of any one of embodiments 141-196, wherein said vector comprises a deletion of at least 1 gene that encodes a phospholipase-D.
198. The vector of embodiment 196 or 197, wherein said gene that encodes a phospholipase-D is K4L.
199. A recombinant orthopoxvirus vector, wherein said vector comprises a deletion of at least 1 gene that encodes a PKR inhibitor.
200. The recombinant orthopoxvirus vector of any one of embodiments 141-199, wherein said vector comprises a deletion of at least 1 gene that encodes a PKR inhibitor.
201. The vector of embodiment 199 or 200, wherein said gene that encodes a PKR inhibitor is K3L.
202. A recombinant orthopoxvirus vector, wherein said vector comprises a deletion of at least 1 gene that encodes a serine protease inhibitor.
203. The recombinant orthopoxvirus vector of any one of embodiments 141-202, wherein said vector comprises a deletion of at least 1 gene that encodes a serine protease inhibitor.
204. The vector of embodiment 202 or 203, wherein said gene that encodes a serine protease inhibitor is K2L.
205. A recombinant orthopoxvirus vector, wherein said vector comprises a deletion of at least 1 gene that encodes a TLR signaling inhibitor.
206. The recombinant orthopoxvirus vector of any one of embodiments 141-205, wherein said vector comprises a deletion of at least 1 gene that encodes a TLR signaling inhibitor.
207. The vector of embodiment 205 or 206, wherein said gene that encodes a TLR signaling inhibitor is N2L.
208. A recombinant orthopoxvirus vector, wherein said vector comprises a deletion of at least 1 gene that encodes a kelch-like protein.
209. The recombinant orthopoxvirus vector of any one of embodiments 141-208, wherein said vector comprises a deletion of at least 1 gene that encodes a kelch-like protein.
210. The vector of embodiment 209, wherein said vector comprises a deletion of at least 2 genes that each encodes a kelch-like protein.
211. The vector of any one of embodiments 208-210, wherein said genes that encode a kelch-like protein are, independently, selected from the group consisting of F3L and C2L.
212. A recombinant orthopoxvirus vector, wherein said vector comprises a deletion of at least 1 gene that encodes a monoglyceride lipase.
213. The recombinant orthopoxvirus vector of any one of embodiments 141-212, wherein said vector comprises a deletion of at least 1 gene that encodes a monoglyceride lipase.
214. The vector of embodiment 213, wherein said vector comprises a deletion of at least 2 genes that each encodes a monoglyceride lipase.
215. The vector of any one of embodiments 212-214, wherein said genes that encode a monoglyceride lipase are, independently, selected from the group consisting of K5L and K6L.
216. A recombinant orthopoxvirus vector, wherein said vector comprises a deletion of at least 1 gene that encodes an NF-κB inhibitor.
217. The recombinant orthopoxvirus vector of any one of embodiments 141-216, wherein said vector comprises a deletion of at least 1 gene that encodes an NF-κB inhibitor.
218. The vector of embodiment 217, wherein said deletion comprises at least 2 genes that each encodes an NF-κB inhibitor.
219. The vector of embodiment 218, wherein said deletion comprises at least 3 genes that each encodes an NF-κB inhibitor.
220. The vector of any one of embodiments 216-219, wherein said genes that encode an NF-κB inhibitor are, independently, selected from the group consisting of K7R, K1L, and M2L.
221. A recombinant orthopoxvirus vector, wherein said vector comprises a deletion of at least 1 gene that encodes an Ankyrin repeat protein.
222. The recombinant orthopoxvirus vector of any one of embodiments 141-221, wherein said vector comprises a deletion of at least 1 gene that encodes an Ankyrin repeat protein.
223. The vector of embodiment 222, wherein said deletion comprises at least 2 genes that each encodes an Ankyrin repeat protein.
224. The vector of embodiment 223, wherein said deletion comprises at least 3 genes that each encodes an Ankyrin repeat protein.
225. The recombinant orthopoxvirus vector of any one of embodiments 221-224, wherein said genes that encode an Ankyrin repeat protein are, independently, selected from the group consisting of B18R, B20R, and M1L.
226. A recombinant orthopoxvirus vector, wherein said vector comprises a deletion of at least 1 gene selected from the group consisting of B15R, B17R, and B14R.
227. The recombinant orthopoxvirus vector of any one of embodiments 1-226, wherein said vector comprises a deletion of at least 1 gene selected from the group consisting of B15R, B17R, and B14R.
228. The vector of embodiment 227, wherein said deletion comprises at least 2 genes, each independently selected from the group consisting of B15R, B17R, and B14R.
229. The vector of embodiment 228, wherein said deletion comprises each of said B15R, B17R, and B14R genes.
230. The recombinant orthopoxvirus vector of any one of embodiments 141-229, wherein said vector comprises a deletion of at least 1 gene selected from the group of ITR genes consisting of B21R, B22R, B23R, B24R, B25R, B26R, B27R, B28R, and B29R.
231. The vector of embodiment 230, wherein said deletion comprises at least 2 genes, each independently selected from said group of ITR genes consisting of B21R, B22R, B23R, B24R, B25R, B26R, B27R, B28R, and B29R.
232. The vector of embodiment 231, wherein said deletion comprises at least 3 genes, each independently selected from said group of ITR genes consisting of B21R, B22R, B23R, B24R, B25R, B26R, B27R, B28R, and B29R.
233. The vector of embodiment 232, wherein said deletion comprises at least 4 genes, each independently selected from said group of ITR genes consisting of B21R, B22R, B23R, B24R, B25R, B26R, B27R, B28R, and B29R.
234. The vector of embodiment 233, wherein said deletion comprises at least 5 genes, each independently selected from said group of ITR genes consisting of B21R, B22R, B23R, B24R, B25R, B26R, B27R, B28R, and B29R.
235. The vector of embodiment 234, wherein said deletion comprises at least 6 genes, each independently selected from said group of ITR genes consisting of B21R, B22R, B23R, B24R, B25R, B26R, B27R, B28R, and B29R.
236. The vector of embodiment 235, wherein said deletion comprises at least 7 genes, each independently selected from said group of ITR genes consisting of B21R, B22R, B23R, B24R, B25R, B26R, B27R, B28R, and B29R.
237. The vector of embodiment 236, wherein said deletion comprises at least 8 genes, each independently selected from said group of ITR genes consisting of B21R, B22R, B23R, B24R, B25R, B26R, B27R, B28R, and B29R.
238. The vector of embodiment 237, wherein said deletion comprises each of B21R, B22R, B23R, B24R, B25R, B26R, B27R, B28R, and B29R.
239.
242. The recombinant orthopoxvirus vector of embodiment 241, wherein said vaccinia virus is a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16m8, LC16mO, Tashkent, Tian Tan, and WAU86/88-1.
243. The recombinant orthopoxvirus vector of embodiment 241, wherein said vaccinia virus is a strain selected from the group consisting of Copenhagen, Western Reserve, Tian Tan, Wyeth, and Lister.
244. The recombinant orthopoxvirus vector of embodiment 241, wherein said vaccinia virus is a Copenhagen strain vaccinia virus.
245. The recombinant orthopoxvirus vector of any one of embodiments 141-244, wherein said deletions is a deletion of the entire polynucleotide encoding the corresponding gene.
246. The recombinant orthopoxvirus vector of any one of embodiments 141-244, wherein each of said deletions is a deletion of a portion of the polynucleotide encoding the corresponding gene, and wherein said deletion is sufficient to render said gene nonfunctional upon introduction into a host cell.
247. The recombinant orthopoxvirus vector of any one of embodiments 14141-246, wherein said vector further comprises a transgene encoding a tumor-associated antigen.
248. The recombinant orthopoxvirus vector of any one of embodiments 141-247, wherein said recombinant orthopoxvirus vector further comprises a transgene encoding a tumor-associated antigen.
249. The recombinant orthopoxvirus vector of embodiment 248, wherein said tumor-associated antigen is a tumor-associated antigen listed in any one of Tables 3-30.
250. The recombinant orthopoxvirus vector of embodiment 248, wherein said tumor-associated antigen is a tumor-associated antigen selected from the group consisting of CD19, CD33, EpCAM, CEA, PSMA, EGFRvIII, CD274, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.
251. The recombinant orthopoxvirus vector of embodiment 248, wherein said tumor-associated antigen comprises MAGE-A3, or one or more fragments thereof.
252. The recombinant orthopoxvirus vector of embodiment 248, wherein said tumor-associated antigen comprises NY-ESO-1, or one or more fragments thereof.
253. The recombinant orthopoxvirus vector of embodiment 248, wherein said tumor-associated antigen comprises one or more human papillomavirus (HPV) proteins, or fragments thereof.
254. The recombinant orthopoxvirus vector of embodiment 248, wherein said tumor-associated antigen comprises (i) E6 and E7 proteins, or fragments thereof, of HPV16 and (ii) E6 and E7 proteins, or fragments thereof, of HPV18.
255. The recombinant orthopoxvirus vector of embodiment 248, wherein said tumor-associated antigen comprises brachyury or one or more fragments thereof.
256. The recombinant orthopoxvirus vector of embodiment 248, wherein said tumor-associated antigen comprises prostatic acid phosphatase, or one or more fragments thereof.
257. A method of producing the recombinant orthopoxvirus vector of any one of embodiments 248-256, said method comprising:
   d. introducing a transposon insertion site into the recombinant orthopoxvirus vector of any one of embodiments 141-247;
   e. contacting the recombinant orthopoxvirus vector formed in (a) with a transposable element comprising a gene encoding said tumor-associated antigen, thereby introducing said gene into said recombinant orthopoxvirus vector; and
   f. recovering the recombinant orthopoxvirus vector formed in (b).
258. The recombinant orthopoxvirus vector of any one of embodiments 141-256, wherein said recombinant orthopoxvirus vector further comprises a transgene encoding an immune checkpoint inhibitor.
259. The recombinant orthopoxvirus vector of embodiment 258, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
260. The recombinant orthopoxvirus vector of embodiment 258, wherein said immune checkpoint inhibitor is an anti-PD-L1 antibody or antigen-binding fragment thereof or an anti-CTLA-4 antibody or antigen-binding fragment thereof.
261. The recombinant orthopoxvirus vector of embodiment 258, wherein said immune checkpoint inhibitor is an anti-PD1 antibody or antigen-binding fragment thereof.
262. The recombinant orthopoxvirus vector of embodiment 258, wherein said immune checkpoint inhibitor is an anti-CTLA-4 antibody or antigen-binding fragment thereof.
263. A method of producing the recombinant orthopoxvirus vector of any one of embodiments 258-262, said method comprising:
   d. introducing a transposon insertion site into the recombinant orthopoxvirus vector of any one of embodiments 141-257;
   e. contacting the recombinant orthopoxvirus vector formed in (a) with a transposable element comprising a gene encoding said immune checkpoint inhibitor, thereby introducing said gene into said recombinant orthopoxvirus vector; and
   f. recovering the recombinant orthopoxvirus vector formed in (b).
264. The recombinant orthopoxvirus vector of any one of embodiments 141-256 and 258-262, wherein said recombinant orthopoxvirus vector further comprises a transgene encoding an interleukin (IL).
265. The recombinant orthopoxvirus vector of embodiment 264, wherein said interleukin is selected from the group consisting of
IL-1 alpha, IL-1 beta, IL-2, IL-4, IL-7, IL-10, IL-12 p35, IL-12 p40, IL-12 p70, IL-15, IL-18, IL-21, and IL-23.
266. The recombinant orthopoxvirus vector of embodiment 264, wherein said interleukin is selected from the group consisting of IL-12 p35, IL-12 p40, and IL-12 p70.
267. The recombinant orthopoxvirus vector of embodiment 266, wherein said interleukin is membrane-bound.
268. A method of producing the recombinant orthopoxvirus vector of one of embodiments 264-267, said method comprising:
   d. introducing a transposon insertion site into the recombinant orthopoxvirus vector of any one of embodiments 141-256 and 258-262;
   e. contacting the recombinant orthopoxvirus vector formed in (a) with a transposable element comprising a gene encoding said interleukin, thereby introducing said gene into said recombinant orthopoxvirus vector; and
   f. recovering the recombinant orthopoxvirus vector formed in (b).
269. The recombinant orthopoxvirus vector of any one of embodiments 141-256, 258-262, and 264-267, wherein said recombinant orthopoxvirus vector further comprises a transgene encoding an interferon (IFN).
270. The recombinant orthopoxvirus vector of embodiment 269, wherein said interferon is selected from the group consisting of IFN-alpha, IFN-beta, IFN-delta, IFN-epsilon, IFN-tau, IFN-omega, IFN-zeta, and IFN-gamma.
271. A method of producing the recombinant orthopoxvirus vector of embodiment 269 or 270, said method comprising:
   d. introducing a transposon insertion site into the recombinant orthopoxvirus vector of any one of embodiments 141-256, 258-262, and 264-267;
   e. contacting the recombinant orthopoxvirus vector formed in (a) with a transposable element comprising a gene encoding said interferon, thereby introducing said gene into said recombinant orthopoxvirus vector; and
   f. recovering the recombinant orthopoxvirus vector formed in (b).
272. The recombinant orthopoxvirus vector of any one of embodiments 141-256, 258-262, 264-267, 269, and 270, wherein said recombinant orthopoxvirus vector further comprises a transgene encoding a TNF superfamily member protein.
273. The recombinant orthopoxvirus vector of embodiment 272, wherein said TNF superfamily member protein is selected from the group consisting of TRAIL, Fas ligand, LIGHT (TNFSF-14), TNF-alpha, and 4-1BB ligand.
274. A method of producing the recombinant orthopoxvirus vector of embodiment 272 or 273, said method comprising:
   d. introducing a transposon insertion site into the recombinant orthopoxvirus vector of any one of embodiments 141-256, 258-262, 264-267, 269, and 270;
   e. contacting the recombinant orthopoxvirus vector formed in (a) with a transposable element comprising a gene encoding said TNF superfamily member protein, thereby introducing said gene into said recombinant orthopoxvirus vector; and
   f. recovering the recombinant orthopoxvirus vector formed in (b).
275. The recombinant orthopoxvirus vector of any one of embodiments 141-256, 258-262, 264-267, 269, 270, 272, and 273 wherein said recombinant orthopoxvirus vector further comprises a transgene encoding a cytokine.
276. The recombinant orthopoxvirus vector of embodiment 275, wherein said cytokine is selected from the group consisting of GM-CSF, Flt3 ligand, CD40 ligand, anti-TGF-beta, anti-VEGF-R2, and cGAS (guanyl adenylate cyclase).
277. The recombinant orthopoxvirus vector of embodiment 275, wherein said cytokine is Flt3 ligand.
278. A method of producing the recombinant orthopoxvirus vector of embodiment 276 or 277, said method comprising:
   d. introducing a transposon insertion site into the recombinant orthopoxvirus vector of any one of embodiments 141-256, 258-262, 264-267, 269, 270, 272, and 273;
   e. contacting the recombinant orthopoxvirus vector formed in (a) with a transposable element comprising a gene encoding said cytokine, thereby introducing said gene into said recombinant orthopoxvirus vector; and
   f. recovering the recombinant orthopoxvirus vector formed in (b).
279. The recombinant orthopoxvirus vector of any one of embodiments 141-256, 258-262, 264-267, 269, 270, 272, 273, and 275-277, wherein said recombinant orthopoxvirus vector further comprises a transgene encoding microRNA (miRNA).
280. The recombinant orthopoxvirus vector of embodiment 279, wherein said miRNA is mir-6.
281. A method of producing the recombinant orthopoxvirus vector of embodiment 279 or 280, said method comprising:
   d. introducing a transposon insertion site into the recombinant orthopoxvirus vector of any one of embodiments 141-256, 258-262, 264-267, 269, 270, 272, 273, and 275-277;
   e. contacting the recombinant orthopoxvirus vector formed in (a) with a transposable element comprising a gene encoding said miRNA, thereby introducing said gene into said recombinant orthopoxvirus vector; and
   f. recovering the recombinant orthopoxvirus vector formed in (b).
282. The nucleic acid of any one of embodiments 1-115, 117-121, 123-126, 128, 129, 131, 132, 134-136, 138, and 139, or the recombinant orthopoxvirus vector of any one of embodiments 141-256, 258-262, 264-267, 269, 270, 272, 273, 275-277, 279, and 280, wherein said nucleic acid or said recombinant orthopoxvirus vector comprises the Thymidine Kinase (TK) gene.
283. The nucleic acid of any one of embodiments 1-115, 117-121, 123-126, 128, 129, 131, 132, 134-136, 138, 139, and 282, or the recombinant orthopoxvirus vector of any one of embodiments 141-256, 258-262, 264-267, 269, 270, 272, 273, 275-277, 279, 280, and 282, wherein said nucleic acid or said recombinant orthopoxvirus vector comprises the ribonucleotide reductase gene.
284. The nucleic acid of any one of embodiments 1-115, 117-121, 123-126, 128, 129, 131, 132, 134-136, 138, 139, 282, and 283, or the recombinant orthopoxvirus vector of any one of embodiments 141-256, 258-262, 264-267, 269, 270, 272, 273, 275-277, 279, 280, 282, and 283, wherein upon contacting a population of mammalian cells with said nucleic acid or said recombinant orthopoxvirus vector, the cells exhibit increased syncytia formation relative to a population of mammalian cells of the same type contacted with a form of the orthopoxvirus vector that does not comprise said deletions.
285. The nucleic acid of any one of embodiments 1-115, 117-121, 123-126, 128, 129, 131, 132, 134-136, 138, 139, and 282-284 or the recombinant orthopoxvirus vector of any one of embodiments 141-256, 258-262, 264-267, 269, 270, 272, 273, 275-277, 279, 280, and 282-284, wherein upon contacting a population of mammalian cells with said nucleic acid or said recombinant orthopoxvirus vector, the cells exhibit increased spreading of the orthopoxvirus vector relative to a population of mammalian cells of the same type contacted with a form of the orthopoxvirus vector that does not comprise said deletions.
286. The nucleic acid of any one of embodiments 1-115, 117-121, 123-126, 128, 129, 131, 132, 134-136, 138, 139, and 282-285, or the recombinant orthopoxvirus vector of any one of embodiments 141-256, 258-262, 264-267, 269, 270, 272, 273, 275-277, 279, 280, and 282-285, wherein said nucleic acid or said recombinant orthopoxvirus vector exerts an increased cytotoxic effect on a population of mammalian cells relative to that of a form of the orthopoxvirus vector that does not comprise said deletions.
287. The nucleic acid or the recombinant orthopoxvirus vector of any one of embodiments 284-286, wherein said mammalian cells are human cells.
288. The nucleic acid or the recombinant orthopoxvirus vector of embodiment 287, wherein said human cells are cancer cells.
289. The nucleic acid or the recombinant orthopoxvirus vector of any one of embodiments 284-286, wherein said mammalian cells are from a cell line selected from the group consisting of U2OS, 293, 293T, Vero, HeLa, A549, BHK, BSC40, CHO, OVCAR-8, 786-0, NCI-H23, U251, SF-295, T-47D, SKMEL2, BT-549, SK-MEL-28, MDA-MB-231, SK-OV-3, MCF7, M14, SF-268, CAKI-1, HPAV, OVCAR-4, HCT15, K-562, and HCT-116.
290. A packaging cell line comprising the nucleic acid of any one of embodiments 1-115, 117-121, 123-126, 128, 129, 131, 132, 134-136, 138, 139, and 282-289 or the recombinant orthopoxvirus vector of any one of embodiments 141-256, 258-262, 264-267, 269, 270, 272, 273, 275-277, 279, 280, and 282-289.
291. A method of treating cancer in a mammalian patient, said method comprising administering a therapeutically effective amount of the nucleic acid of any one of embodiments 1-115, 117-121, 123-126, 128, 129, 131, 132, 134-136, 138, 139, and 282-289, or the recombinant orthopoxvirus vector of any one of embodiments 141-256, 258-262, 264-267, 269, 270, 272, 273, 275-277, 279, 280, and 282-289 to said patient.
292. The method of embodiment 291, wherein said mammalian patient is a human patient.
293. The method of embodiment 291 or 292, wherein said cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.
294. The method of embodiment 291 or 292, wherein said cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.
295. The method of any one of embodiments 291-294, wherein said method further comprises administering to said patient an immune checkpoint inhibitor.
296. The method of embodiment 295, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
297. The method of embodiment 295, wherein said immune checkpoint inhibitor is an anti-PD1 antibody or antigen-binding fragment thereof or an anti-CTLA-4 antibody or antigen-binding fragment thereof.
298. The method of embodiment 295, wherein said immune checkpoint inhibitor is an anti-PD1 antibody or antigen-binding fragment thereof.
299. The method of embodiment 295, wherein said immune checkpoint inhibitor is an anti-CTLA-4 antibody or antigen-binding fragment thereof.
300. The method of any one of embodiments 291-299, wherein said method further comprises administering to said patient an interleukin.
301. The method of embodiment 300, wherein said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
302. The method of embodiment 300, wherein said interleukin is selected from the group consisting of IL-12 p35, IL-12 p40, and IL-12 p70.
303. The method of embodiment 301 or 302, wherein said interleukin is membrane-bound.
304. The method of any one of embodiments 291-303, wherein said method further comprises administering to said patient an interferon.
305. The method of embodiment 304, wherein said interferon is selected from the group consisting of
IFN-alpha, IFN-beta, IFN-delta, IFN-epsilon, IFN-tau, IFN-omega, IFN-zeta, and IFN-gamma.
306. The method of any one of embodiments 291-305, wherein said method further comprises administering to said patient a TNF superfamily member protein.
307. The method of embodiment 306, wherein said TNF superfamily member protein is selected from the group consisting of TRAIL, Fas ligand, LIGHT (TNFSF-14), TNF-alpha, and 4-1BB ligand.
308. The method of any one of embodiments 291-307, wherein said method further comprises administering to said patient a cytokine.
309. The method of embodiment 308, wherein said cytokine is selected from the group consisting of GM-CSF, Flt3 ligand, CD40 ligand, anti-TGF-beta, anti-VEGF-R2, and cGAS (guanyl adenylate cyclase).
310. The method of embodiment 308, wherein said cytokine is Flt3 ligand.
311. The method of any one of embodiments 291-310, wherein said method further comprises administering to said patient a miRNA.
312. The method of embodiment 311, wherein said miRNA is mir-6.
313. A kit comprising the nucleic acid of any one of embodiments 1-115, 117-121, 123-126, 128, 129, 131, 132, 134-136, 138, 139, and 282-289 or the recombinant orthopoxvirus vector of any one of embodiments 141-256, 258-262, 264-267, 269, 270, 272, 273, 275-277, 279, 280, and 282-289 and a package insert instructing a user of said kit to express said nucleic acid or said vector in a host cell.
314. A kit comprising the nucleic acid of any one of embodiments 1-115, 117-121, 123-126, 128, 129, 131, 132, 134-136, 138, 139, and 282-289 or the recombinant orthopoxvirus vector of any one of embodiments 141-256, 258-262, 264-267, 269, 270, 272, 273, 275-277, 279, 280, and 282-289 and a package insert instructing a user to administer a therapeutically effective amount of said nucleic acid or recombinant orthopoxvirus vector to a mammalian patient having cancer, thereby treating said cancer.
315. The kit of embodiment 314, wherein said mammalian patient is a human patient.
316. The orthopox virus of any of the preceding embodiments wherein the B8R gene is deleted.
317. The orthopox virus of embodiment 316, wherein at least one transgene is inserted into the locus of the deleted B8R gene. 0

318. The orthopox virus of embodiment 317, wherein at least two transgenes are inserted into the locus of the deleted B8R gene.
319. The orthopox virus of embodiment 318, wherein at least three transgenes are inserted into the locus of the deleted B8R gene.
320. The orthopox virus of any one of embodiments 316-319, wherein at least one additional transgene is inserted at locus that is not the locus of the B8R gene.
321. The orthopox virus of embodiment 320, wherein the locus is the boundary of the 5p deletion.
322. The orthopox virus of embodiment 321, wherein the locus is the boundary of the 3p deletion.
323. The orthopox virus of embodiments 316-322, wherein at least one of the following transgenes is inserted: IL-12TM, FLT3-L or anti-CLTA-4 antibody.
324. The orthopox virus of embodiment 323, wherein the IL-12-TM and FLT3-L genes are inserted into the locus of the deleted B8R gene.
325. The orthopox virus of any one of embodiments 323-324, wherein the anti-CLTA-4 antibody is inserted within the boundary of the 5p deletion.
326. The orthopox virus of anyone of embodiments 323-325, wherein the virus comprises the sequence of SEQ ID. NO: 210.
326. The orthopox virus of any one of embodiments 323-325 wherein the anti-CLTA antibody is SEQ ID NO: 211.
327. The orthopox virus of any one of embodiments 323-326, wherein the IL-12-TM is SEQ ID NO: 212.
328. The orthopox virus of any one of embodiments 323-327 wherein the FLT3-L is SEQ ID NO: 213.
329. An orthopox virus comprising a nucleic acid sequence wherein the nucleic acid sequence is a derivative of SEQ ID NO: 210,
   wherein said derivative comprises a deletion of the B8R gene of SEQ ID NO: 210,
   wherein, an IL-12-TM and an FLT3-L transgene are inserted into the locus of said deleted B8R gene,
   wherein, genes encoding a heavy and a light chain of an anti-CLTA-4 antibody are inserted within the boundaries of the 5p deletion present in SEQ ID NO: 210;
   wherein the IL-12-TM is SEQ ID NO: 212;
   wherein the FLT3-L is SEQ ID NO: 213; and
   wherein the anti-CLTA4 antibody is encoded by SEQ ID NO: 211.

### 5.7.2. Sett

1. A nucleic acid comprising a recombinant orthopoxvirus genome, wherein said recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
2. The nucleic acid of embodiment 1, wherein said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
3. The nucleic acid of embodiment 2, wherein said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
4. The nucleic acid of embodiment 3, wherein said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
5. The nucleic acid of embodiment 4, wherein said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
6. The nucleic acid of embodiment 5, wherein said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
7. The nucleic acid of embodiment 6, wherein said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
8. The nucleic acid of embodiment 7, wherein said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
9. The nucleic acid of embodiment 8, wherein said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
10. The nucleic acid of embodiment 9, wherein said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
11. The nucleic acid of embodiment 10, wherein said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
12. The nucleic acid of embodiment 11, wherein said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
13. The nucleic acid of embodiment 12, wherein said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
14. The nucleic acid of embodiment 13, wherein said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
15. The nucleic acid of embodiment 14, wherein said recombinant orthopoxvirus genome comprises a deletion of at least 16 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
16. The nucleic acid of embodiment 15, wherein said recombinant orthopoxvirus genome comprises a deletion of at least 17 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
17. The nucleic acid of embodiment 16, wherein said recombinant orthopoxvirus genome comprises a deletion of at least 18 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
18. The nucleic acid of embodiment 17, wherein said recombinant orthopoxvirus genome comprises a deletion of at least 19 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
19. The nucleic acid of embodiment 18, wherein said recombinant orthopoxvirus genome comprises a deletion of at least 20 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
20. The nucleic acid of embodiment 19, wherein said recombinant orthopoxvirus genome comprises a deletion of at least 21 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
21. The nucleic acid of embodiment 20, wherein said recombinant orthopoxvirus genome comprises a deletion of at least 22 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
22. The nucleic acid of embodiment 21, wherein said recombinant orthopoxvirus genome comprises a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
23. A nucleic acid comprising a recombinant orthopoxvirus genome, wherein said recombinant orthopoxvirus genome comprises a deletion of at least 1 gene selected from the group consisting of the B14R, B15R, B16R, B17L, B18R, B19R, and B20R genes.
24. The nucleic acid of embodiment 23, wherein said recombinant orthopoxvirus genome comprises a deletion of at least 2 genes, each gene selected from the group consisting of the B14R, B15R, B16R, B17L, B18R, B19R, and B20R genes.
25. The nucleic acid of embodiment 24, wherein said recombinant orthopoxvirus genome comprises a deletion of at least 3 genes, each gene selected from the group consisting of the B14R, B15R, B16R, B17L, B18R, B19R, and B20R genes.
26. The nucleic acid of embodiment 25, wherein said recombinant orthopoxvirus genome comprises a deletion of at least 4 genes, each gene selected from the group consisting of the B14R, B15R, B16R, B17L, B18R, B19R, and B20R genes.
27. The nucleic acid of embodiment 26, wherein said recombinant orthopoxvirus genome comprises a deletion of at least 5 genes, each gene selected from the group consisting of the B14R, B15R, B16R, B17L, B18R, B19R, and B20R genes.
28. The nucleic acid of embodiment 27, wherein said recombinant orthopoxvirus genome comprises a deletion of at least 6 genes, each gene selected from the group consisting of the B14R, B15R, B16R, B17L, B18R, B19R, and B20R genes.
29. The nucleic acid of embodiment 28, wherein said recombinant orthopoxvirus genome comprises a deletion of each of the B14R, B15R, B16R, B17L, B18R, B19R, and B20R genes.
30. A nucleic acid comprising a recombinant orthopoxvirus genome, wherein said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.
31. The nucleic acid of embodiment 30, wherein said recombinant orthopoxvirus genome comprises a deletion of at least 2 genes, each gene selected from the group consisting of the C2L, C1L, N1L, N2L, M1L, M2L, K1L, K2L, K3L, K4L, K5L, K6L, K7R, F1L, F2L, and F3L genes.
32. The nucleic acid of embodiment 31, wherein said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, and F3L genes.
33. The nucleic acid of embodiment 32, wherein said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, and F3L genes.
34. The nucleic acid of embodiment 33, wherein said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, and F3L genes.
35. The nucleic acid of embodiment 34, wherein said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, and F3L genes.
36. The nucleic acid of embodiment 35, wherein said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, and F3L genes.
37. The nucleic acid of embodiment 36, wherein said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, and F3L genes.
38. The nucleic acid of embodiment 37, wherein said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, and F3L genes.
39. The nucleic acid of embodiment 38, wherein said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, and F3L genes.
40. The nucleic acid of embodiment 39, wherein said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, and F3L genes.
41. The nucleic acid of embodiment 40, wherein said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, and F3L genes.
42. The nucleic acid of embodiment 41, wherein said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, and F3L genes.
43. The nucleic acid of embodiment 42, wherein said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, and F3L genes.
44. The nucleic acid of embodiment 43, wherein said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, and F3L genes.
45. The nucleic acid of embodiment 44, wherein said recombinant orthopoxvirus genome comprises a deletion of each of the C2L, C1L, N1L, N2L, M1L, M2L, K1L, K2L, K3L, K4L, K5L, K6L, K7R, F1L, F2L, and F3L genes.
46. The nucleic acid comprising a recombinant orthopoxvirus genome of any one of embodiments 1-45, wherein said recombinant orthopoxvirus genome comprises a deletion of at least 1 gene that encodes a protein involved in host interaction.
47. The nucleic acid of embodiment 46, wherein said protein affects calcium-independent adhesion to the extracellular matrix.
48. The nucleic acid of embodiment 46, wherein said protein is an NF-κB inhibitor.
49. The nucleic acid of embodiment 48, wherein said protein is encoded by a gene selected from the group consisting of the C2L, N1L, M2L, K1L, and K7R genes.
50. The nucleic acid of embodiment 46, wherein said protein is an apoptosis inhibitor.
51. The nucleic acid of embodiment 47, wherein said apoptosis inhibitor is a caspase-9 inhibitor.
52. The nucleic acid of embodiment 51, wherein said caspase-9 inhibitor is encoded by the F1L gene.
53. The nucleic acid of embodiment 50, wherein said apoptosis inhibitor is a BCL-2-like protein.
54. The nucleic acid of embodiment 53, wherein said BCL-2-like protein is encoded by N1L.
55. The nucleic acid of embodiment 46, wherein said protein is an interferon regulatory factor 3 (IRF3) inhibitor.
56. The nucleic acid of embodiment 55, wherein said IRF3 inhibitor is encoded by N2L or K7R.
57. The nucleic acid of embodiment 46, wherein said protein is a serine protease inhibitor.
58. The nucleic acid of embodiment 46, wherein said protein prevents cell fusion.
59. The nucleic acid of embodiment 58, wherein said protein is encoded by K2L.
60. The nucleic acid of embodiment 46, wherein said protein is an RNA-activated protein kinase (PKR) inhibitor.
61. The nucleic acid of embodiment 60, wherein said protein is encoded by K1L or K3L.
62. The nucleic acid of embodiment 46, wherein said protein is a virulence factor.
63. The nucleic acid of embodiment 63, wherein said protein is encoded by F3L.
64. The nucleic acid of embodiment 46, wherein said protein is an IL-1-beta inhibitor.
65. The nucleic acid of embodiment 64, wherein said protein is encoded by B16R.
66. The nucleic acid of embodiment 46, wherein said protein is a secreted IFNα sequestor.
67. The nucleic acid of embodiment 67, wherein said protein is encoded by B19R.
68. The nucleic acid comprising a recombinant orthopoxvirus genome of any one of embodiments 1-67, wherein said recombinant orthopoxvirus genome comprises a deletion of at least 1 gene encoding a protein involved in DNA replication.
69. The nucleic acid of embodiment 68, wherein said protein is a DNA modifying nuclease.
70. The nucleic acid of embodiment 69, wherein said protein is encoded by K4L.
71. The nucleic acid of embodiment 70, wherein said protein is a deoxyuridine triphosphatase (dUTPase).
72. The nucleic acid of embodiment 71, wherein the dUTPase is encoded by F2L.
73. The nucleic acid of any one of embodiments 1-72, wherein at least one deleted gene's entire nucleotide sequence is deleted.
74. The nucleic acid of any one of embodiments 1-72, wherein at least one deleted gene is only partially deleted, and wherein the partial deletion is sufficient to render said partially deleted gene nonfunctional upon introduction into a host cell.
75. The nucleic acid of any one of embodiments 1-74, wherein said recombinant orthopoxvirus genome comprises at least two copies of inverted terminal repeats (ITRs).
76. The nucleic acid of any one of embodiments 1-74, wherein said recombinant orthopoxvirus genome lacks any copies of ITRs.
77. The nucleic acid of any one of embodiments 1-74, wherein said recombinant orthopoxvirus genome comprises a deletion in at least one copy of an ITR selected from the group consisting of B21R-ITR, B22R-ITR, B23R-ITR, B24R-ITR, B25R-ITR, B26R-ITR, B27R-ITR, B28R-ITR, and B29R-ITR.
78. The nucleic acid of any one of embodiments 1-74, wherein said recombinant orthopoxvirus genome comprises a deletion in at least all of the following copies of ITRs: B21R-ITR, B22R-ITR, B23R-ITR, B24R-ITR, B25R-ITR, B26R-ITR, B27R-ITR, B28R-ITR, and B29R-ITR.
79. The nucleic acid of any one of embodiments 1-78, wherein said recombinant orthopoxvirus genome comprises a deletion in the B8R gene.
80. The nucleic acid of any one of embodiments 1-78, wherein said recombinant orthopoxvirus genome comprises an intact B8R gene.
81. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes; and
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR.
82. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes; and
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR,
   wherein said recombinant orthopoxvirus genome comprises an intact B8R gene.
83. The nucleic acid of any one of embodiments 1-82, further comprising at least one transgene selected from the group consisting of a transgene encoding an immune checkpoint inhibitor, a transgene encoding an interleukin (IL), and a transgene encoding a cytokine.
84. The nucleic acid of embodiment 83, wherein the nucleic acid comprises at least two transgenes selected from the group consisting of a transgene encoding an immune checkpoint inhibitor, a transgene encoding an interleukin (IL), and a transgene encoding a cytokine.
85. The nucleic acid of embodiment 84, wherein the nucleic acid comprises a transgene encoding an immune checkpoint inhibitor, a transgene encoding an interleukin (IL), and a transgene encoding a cytokine.
86. The nucleic acid of any one of embodiment 83 or 84, wherein said nucleic acid comprises a transgene encoding an immune checkpoint inhibitor.
87. The nucleic acid of embodiment 85 or 86, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
88. The nucleic acid of embodiment 87, wherein said immune checkpoint inhibitor is an anti-PD-L1 antibody or antigen-binding fragment thereof or an anti-CTLA-4 antibody or antigen-binding fragment thereof.
89. The nucleic acid of embodiment 88, wherein said immune checkpoint inhibitor is an anti-PD1 antibody or antigen-binding fragment thereof.
90. The nucleic acid of embodiment 88, wherein said immune checkpoint inhibitor is an anti-CTLA-4 antibody or antigen-binding fragment thereof.
91. The nucleic acid of embodiment 83 or 84, wherein said nucleic acid comprises a transgene encoding an interleukin (IL).
92. The nucleic acid of embodiment 85 or 91, wherein said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
93. The nucleic acid of embodiment 92, wherein said interleukin is selected from the group consisting of IL-12 p35, IL-12 p40, and IL-12 p70.
94. The nucleic acid of embodiment 93, wherein said interleukin is membrane-bound.
95. The nucleic acid of embodiment 94, wherein said interleukin is membrane-bound IL-12 p70.
96. The nucleic acid of embodiment 83 or 84, wherein said nucleic acid comprises a transgene encoding a cytokine.
97. The nucleic acid of embodiment 96, wherein said cytokine is an interferon (IFN).
98. The nucleic acid of embodiment 97, wherein said interferon is selected from the group consisting of IFN-alpha, IFN-beta, IFN-delta, IFN-epsilon, IFN-tau, IFN-omega, IFN-zeta, and IFN-gamma.
99. The nucleic acid of embodiment 96, wherein said cytokine is a TNF superfamily member protein.
100. The nucleic acid of embodiment 99, wherein said TNF superfamily member protein is selected from the group consisting of TRAIL, Fas ligand, LIGHT (TNFSF-14), TNF-alpha, and 4-1BB ligand.
101. The nucleic acid of embodiment 96, wherein said cytokine is selected from the group consisting of GM-CSF, Flt3 ligand, CD40 ligand, TGF-beta, VEGF-R2, and c-kit.
102. The nucleic acid of embodiment 101, wherein said cytokine is Flt3 ligand.
103. The nucleic acid of embodiment 83-102, wherein said recombinant orthopoxvirus genome comprises a deletion in the B8R gene and at least one transgene is inserted into the deletion in the B8R gene. 0

104. The nucleic acid of embodiment 103, wherein at least two transgenes are inserted into the deletion in the B8R gene.
105. The nucleic acid of embodiment 104, wherein at least three transgenes are inserted into the deletion in the B8R gene.
106. The nucleic acid of any one of embodiments 103-105, wherein at least one transgene is inserted in a locus that is not at the deletion in the B8R gene.
107. The nucleic acid of embodiment 106, wherein the locus is at the boundary of a deletion at the 5′ end of the orthopoxvirus genome.
108. The nucleic acid of embodiment 106, wherein the locus is at the boundary of a deletion at the 3′ end of the orthopoxvirus genome.
109. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes;
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR;
   (iii) an IL-12-TM transgene inserted into the deletion in the B8R gene;
   (iv) an Flt3 ligand transgene inserted into the deletion in the B8R gene; and
   (v) one of:
      (a) a transgene encoding a single chain anti-CTLA-4 antibody or antigen-binding fragment thereof
      or
      (b) (i) a transgene encoding a heavy chain of an anti-CTLA-4 antibody or antigen-binding fragment thereof, and
         (ii) a transgene encoding a light chain of an anti-CTLA-4 antibody or antigen-binding fragment thereof,
   wherein the transgene(s) in part (v) is/are inserted within the boundaries of a 5p deletion present in the recombinant orthopoxvirus genome, and
   wherein the anti-CTLA-4 antibody or antigen-binding fragment thereof is capable of binding CTLA-4.
110. The nucleic acid of embodiment 109, wherein the orthopoxvirus genome is derived from a sequence of SEQ ID NO: 210, wherein
   (a) said derived sequence comprises a deletion of the B8R gene, and the IL-12-TM transgene, the Flt3 ligand transgene, and the transgene(s) encoding the single or double-chain anti-CTLA-4 antibody;
   (b) the IL-12-TM transgene encodes a protein comprising an amino acid sequence of is SEQ ID NO: 212;
   (c) the Flt3 ligand transgene encodes a protein comprising an amino acid sequence of SEQ ID NO: 213; and
   (d) the anti-CTLA-4 antibody comprises an amino acid sequence of SEQ ID NO: 211.
111. A virus comprising the nucleic acid comprising the recombinant orthopoxvirus genome of any one of embodiments 1-110.
112. The virus of embodiment 111, wherein
   a) said recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes;
   b) said recombinant orthopoxvirus genome comprises a deletion of at least 1 gene selected from the group consisting of the B14R, B15R, B16R, B17L, B18R, B19R, and B20R genes; or
   c) said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.
113. The virus of embodiment 111 or 112, wherein said virus is derived from a vaccinia virus.
114. The virus of embodiment 113, wherein said vaccinia virus is derived from a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16mO, Tashkent, Tian Tan, and WAU86/88-1.
115. The virus of embodiment 114, wherein said vaccinia virus is derived from a strain selected from the group consisting of Copenhagen, Western Reserve, Tian Tan, Wyeth, and Lister.
116. The virus of embodiment 115, wherein said vaccinia virus is derived from a Copenhagen strain vaccinia virus.
117. The virus of any one of embodiments 111-116, wherein said recombinant orthopoxvirus genome further comprises a Thymidine Kinase (TK) gene.
118. The virus of any one of embodiments 111-117, wherein said recombinant orthopoxvirus genome further comprises a ribonucleotide reductase gene.
119. The virus of any one of embodiments 111-118, wherein upon contacting a population of mammalian cells with said virus, the population of mammalian cells exhibit increased syncytia formation relative to a population of mammalian cells of the same type contacted with a form of the virus that does not comprise said deletion.
120. The virus of any one of embodiments 111-119, wherein upon contacting a population of mammalian cells with said virus, the population of mammalian cells exhibit increased spreading of the virus relative to a population of mammalian cells of the same type contacted with a form of the virus that does not comprise said deletion.
121. The virus of any one of embodiments 111-120, wherein said virus exerts an increased cytotoxic effect on a population of mammalian cells relative to that of a form of the virus that does not comprise said deletion.
122. The virus of any one of embodiments 119-121, wherein said mammalian cells are human cells.
123. The virus of embodiment 122, wherein said human cells are cancer cells.
124. The virus of any one of embodiments 119-121, wherein said mammalian cells are from a cell line selected from the group consisting of U2OS, 293, 293T, Vero, HeLa, A549, BHK, BSC40, CHO, OVCAR-8, 786-0, NCI-H23, U251, SF-295, T-47D, SKMEL2, BT-549, SK-MEL-28, MDA-MB-231, SK-OV-3, MCF7, M14, SF-268, CAKI-1, HPAV, OVCAR-4, HCT15, K-562, and HCT-116.
125. The nucleic acid of any one of embodiments 1-110 or the virus of any one of embodiments 111-124, wherein said nucleic acid or said virus further comprises a transgene encoding a tumor-associated antigen.
126. The nucleic acid or the virus of embodiment 125, wherein said tumor-associated antigen is a tumor-associated antigen listed in any one of Tables 3-30.
127. The nucleic acid or the virus of embodiment 126, wherein said tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.
128. The nucleic acid or the virus of embodiment 125, wherein said tumor-associated antigen comprises MAGE-A3, or one or more fragments thereof.
129. The nucleic acid or the virus of embodiment 125, wherein said tumor-associated antigen comprises NY-ESO-1, or one or more fragments thereof.
130. The nucleic acid or the virus of embodiment 125, wherein said tumor-associated antigen comprises one or more human papillomavirus (HPV) proteins, or fragments thereof.
131. The nucleic acid or the virus of embodiment 125, wherein said HPV proteins or fragments thereof comprise one or more of (i) E6 and E7 proteins, or fragments thereof, of HPV16 and (ii) E6 and E7 proteins, or fragments thereof, of HPV18.
132. The nucleic acid or the virus of embodiment 131, wherein the sequences of said HPV proteins or fragments are disclosed in International Patent Publication WO/2014/127478, the contents of which are incorporated herein by reference.
133. The nucleic acid or the virus of embodiment 125, wherein said tumor-associated antigen comprises brachyury or one or more fragments thereof.
134. The nucleic acid or the virus of embodiment 125, wherein said tumor-associated antigen comprises prostatic acid phosphatase, or one or more fragments thereof.
135. A packaging cell line comprising the nucleic acid of any one of embodiments 1-110 or the virus of any one of embodiments 111-124.
136. A method of treating cancer in a mammalian patient, said method comprising administering a therapeutically effective amount of the virus of any one of embodiments 111-134 to said patient.
137. The method of embodiment 136, wherein said mammalian patient is a human patient.
138. The method of embodiment 136 or 137, wherein the virus is used as a prime in a prime:boost treatment.
139. The method of embodiment 136 or 137, wherein the virus is used as a boost in a prime:boost treatment.

140. The method of any one of embodiments 136-139, wherein said mammalian patient has cancer.
141. The method of embodiment 140, wherein said cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.
142. The method of embodiment 140, wherein said cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin's lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi's sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia. 0

143. The method of any one of embodiments 136-142, wherein said method further comprises administering to said patient an immune checkpoint inhibitor.
144. The method of embodiment 143, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
145. The method of embodiment 144, wherein said immune checkpoint inhibitor is an anti-PD1 antibody or antigen-binding fragment thereof or an anti-CTLA-4 antibody or antigen-binding fragment thereof.
146. The method of embodiment 145, wherein said immune checkpoint inhibitor is an anti-PD1 antibody or antigen-binding fragment thereof.
147. The method of embodiment 145, wherein said immune checkpoint inhibitor is an anti-CTLA-4 antibody or antigen-binding fragment thereof.
148. The method of any one of embodiments 136-147, wherein said method further comprises administering to said patient an interleukin.
149. The method of embodiment 148, wherein said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
150. The method of embodiment 149, wherein said interleukin is selected from the group consisting of IL-12 p35, IL-12 p40, and IL-12 p70.
151. The method of embodiment 149 or 150, wherein said interleukin is membrane-bound.
152. The method of any one of embodiments 136-151, wherein said method further comprises administering to said patient an interferon.
153. The method of embodiment 152, wherein said interferon is selected from the group consisting of
IFN-alpha, IFN-beta, IFN-delta, IFN-epsilon, IFN-tau, IFN-omega, IFN-zeta, and IFN-gamma.
154. The method of any one of embodiments 136-153, wherein said method further comprises administering to said patient a cytokine.
155. The method of embodiment 154, wherein said cytokine is a TNF superfamily member protein.
156. The method of embodiment 155, wherein said TNF superfamily member protein is selected from the group consisting of TRAIL, Fas ligand, LIGHT (TNFSF-14), TNF-alpha, and 4-1BB ligand.
157. The method of embodiment 154, wherein said cytokine is selected from the group consisting of GM-CSF, Flt3 ligand, CD40 ligand, TGF-beta, VEGF-R2, and cKit.
158. The method of embodiment 157, wherein said cytokine is Flt3 ligand.
159. A kit comprising the nucleic acid of any one of embodiments 1-110 or the virus of any one of embodiments 111-134 and a package insert instructing a user of said kit to express said nucleic acid or said virus in a host cell.
160. A kit comprising the virus of any one of embodiments 111-134 and a package insert instructing a user to administer a therapeutically effective amount of said virus to a mammalian patient having cancer, thereby treating said cancer.
161. The kit of embodiment 160, wherein said mammalian patient is a human patient.
162. The nucleic acid of embodiment 94, wherein said interleukin is membrane-bound IL-12 p35.

### 5.7.3. Set 3

1. A nucleic acid comprising a recombinant orthopoxvirus genome, wherein said recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
2. The nucleic acid of embodiment 1, wherein said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
3. The nucleic acid of embodiment 2, wherein said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
4. The nucleic acid of embodiment 3, wherein said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
5. The nucleic acid of embodiment 4, wherein said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
6. The nucleic acid of embodiment 5, wherein said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
7. The nucleic acid of embodiment 6, wherein said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
8. The nucleic acid of embodiment 7, wherein said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
9. The nucleic acid of embodiment 8, wherein said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes. 0

10. The nucleic acid of embodiment 9, wherein said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
11. The nucleic acid of embodiment 10, wherein said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
12. The nucleic acid of embodiment 11, wherein said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
13. The nucleic acid of embodiment 12, wherein said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
14. The nucleic acid of embodiment 13, wherein said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
15. The nucleic acid of embodiment 14, wherein said recombinant orthopoxvirus genome comprises a deletion of at least 16 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
16. The nucleic acid of embodiment 15, wherein said recombinant orthopoxvirus genome comprises a deletion of at least 17 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
17. The nucleic acid of embodiment 16, wherein said recombinant orthopoxvirus genome comprises a deletion of at least 18 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
18. The nucleic acid of embodiment 17, wherein said recombinant orthopoxvirus genome comprises a deletion of at least 19 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
19. The nucleic acid of embodiment 18, wherein said recombinant orthopoxvirus genome comprises a deletion of at least 20 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
20. The nucleic acid of embodiment 19, wherein said recombinant orthopoxvirus genome comprises a deletion of at least 21 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
21. The nucleic acid of embodiment 20, wherein said recombinant orthopoxvirus genome comprises a deletion of at least 22 genes, each gene selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
22. The nucleic acid of embodiment 21, wherein said recombinant orthopoxvirus genome comprises a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes.
23. A nucleic acid comprising a recombinant orthopoxvirus genome, wherein said recombinant orthopoxvirus genome comprises a deletion of at least 1 gene selected from the group consisting of the B14R, B15R, B16R, B17L, B18R, B19R, and B20R genes.
24. The nucleic acid of embodiment 23, wherein said recombinant orthopoxvirus genome comprises a deletion of at least 2 genes, each gene selected from the group consisting of the B14R, B15R, B16R, B17L, B18R, B19R, and B20R genes.
25. The nucleic acid of embodiment 24, wherein said recombinant orthopoxvirus genome comprises a deletion of at least 3 genes, each gene selected from the group consisting of the B14R, B15R, B16R, B17L, B18R, B19R, and B20R genes.
26. The nucleic acid of embodiment 25, wherein said recombinant orthopoxvirus genome comprises a deletion of at least 4 genes, each gene selected from the group consisting of the B14R, B15R, B16R, B17L, B18R, B19R, and B20R genes.
27. The nucleic acid of embodiment 26, wherein said recombinant orthopoxvirus genome comprises a deletion of at least 5 genes, each gene selected from the group consisting of the B14R, B15R, B16R, B17L, B18R, B19R, and B20R genes.
28. The nucleic acid of embodiment 27, wherein said recombinant orthopoxvirus genome comprises a deletion of at least 6 genes, each gene selected from the group consisting of the B14R, B15R, B16R, B17L, B18R, B19R, and B20R genes.
29. The nucleic acid of embodiment 28, wherein said recombinant orthopoxvirus genome comprises a deletion of each of the B14R, B15R, B16R, B17L, B18R, B19R, and B20R genes.
30. A nucleic acid comprising a recombinant orthopoxvirus genome, wherein said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.
31. The nucleic acid of embodiment 30, wherein said recombinant orthopoxvirus genome comprises a deletion of at least 2 genes, each gene selected from the group consisting of the C2L, C1L, N1L, N2L, M1L, M2L, K1L, K2L, K3L, K4L, K5L, K6L, K7R, F1L, F2L, and F3L genes.
32. The nucleic acid of embodiment 31, wherein said recombinant orthopoxvirus genome comprises a deletion of at least 3 genes, each gene selected from the group consisting of the C2L, C1L, N1L, N2L, M1L, M2L, K1L, K2L, K3L, K4L, K5L, K6L, K7R, F1L, F2L, and F3L genes.
33. The nucleic acid of embodiment 32, wherein said recombinant orthopoxvirus genome comprises a deletion of at least 4 genes, each gene selected from the group consisting of the C2L, C1L, N1L, N2L, M1L, M2L, K1L, K2L, K3L, K4L, K5L, K6L, K7R, F1L, F2L, and F3L genes.
34. The nucleic acid of embodiment 33, wherein said recombinant orthopoxvirus genome comprises a deletion of at least 5 genes, each gene selected from the group consisting of the C2L, C1L, N1L, N2L, M1L, M2L, K1L, K2L, K3L, K4L, K5L, K6L, K7R, F1L, F2L, and F3L genes.
35. The nucleic acid of embodiment 34, wherein said recombinant orthopoxvirus genome comprises a deletion of at least 6 genes, each gene selected from the group consisting of the C2L, C1L, N1L, N2L, M1L, M2L, K1L, K2L, K3L, K4L, K5L, K6L, K7R, F1L, F2L, and F3L genes.
36. The nucleic acid of embodiment 35, wherein said recombinant orthopoxvirus genome comprises a deletion of at least 7 genes, each gene selected from the group consisting of the C2L, C1L, N1L, N2L, M1L, M2L, K1L, K2L, K3L, K4L, K5L, K6L, K7R, F1L, F2L, and F3L genes.
37. The nucleic acid of embodiment 36, wherein said recombinant orthopoxvirus genome comprises a deletion of at least 8 genes, each gene selected from the group consisting of the C2L, C1L, N1L, N2L, M1L, M2L, K1L, K2L, K3L, K4L, K5L, K6L, K7R, F1L, F2L, and F3L genes.
38. The nucleic acid of embodiment 37, wherein said recombinant orthopoxvirus genome comprises a deletion of at least 9 genes, each gene selected from the group consisting of the C2L, C1L, N1L, N2L, M1L, M2L, K1L, K2L, K3L, K4L, K5L, K6L, K7R, F1L, F2L, and F3L genes.
39. The nucleic acid of embodiment 38, wherein said recombinant orthopoxvirus genome comprises a deletion of at least 10 genes, each gene selected from the group consisting of the C2L, C1L, N1L, N2L, M1L, M2L, K1L, K2L, K3L, K4L, K5L, K6L, K7R, F1L, F2L, and F3L genes.
40. The nucleic acid of embodiment 39, wherein said recombinant orthopoxvirus genome comprises a deletion of at least 11 genes, each gene selected from the group consisting of the C2L, C1L, N1L, N2L, M1L, M2L, K1L, K2L, K3L, K4L, K5L, K6L, K7R, F1L, F2L, and F3L genes.
41. The nucleic acid of embodiment 40, wherein said recombinant orthopoxvirus genome comprises a deletion of at least 12 genes, each gene selected from the group consisting of the C2L, C1L, N1L, N2L, M1L, M2L, K1L, K2L, K3L, K4L, K5L, K6L, K7R, F1L, F2L, and F3L genes.
42. The nucleic acid of embodiment 41, wherein said recombinant orthopoxvirus genome comprises a deletion of at least 13 genes, each gene selected from the group consisting of the C2L, C1L, N1L, N2L, M1L, M2L, K1L, K2L, K3L, K4L, K5L, K6L, K7R, F1L, F2L, and F3L genes.
43. The nucleic acid of embodiment 42, wherein said recombinant orthopoxvirus genome comprises a deletion of at least 14 genes, each gene selected from the group consisting of the C2L, C1L, N1L, N2L, M1L, M2L, K1L, K2L, K3L, K4L, K5L, K6L, K7R, F1L, F2L, and F3L genes.
44. The nucleic acid of embodiment 43, wherein said recombinant orthopoxvirus genome comprises a deletion of at least 15 genes, each gene selected from the group consisting of the C2L, C1L, N1L, N2L, M1L, M2L, K1L, K2L, K3L, K4L, K5L, K6L, K7R, F1L, F2L, and F3L genes.
45. The nucleic acid of embodiment 44, wherein said recombinant orthopoxvirus genome comprises a deletion of each of the C2L, C1L, N1L, N2L, M1L, M2L, K1L, K2L, K3L, K4L, K5L, K6L, K7R, F1L, F2L, and F3L genes.
46. The nucleic acid comprising a recombinant orthopoxvirus genome of any one of embodiments 1-45, wherein said recombinant orthopoxvirus genome comprises a deletion of at least 1 gene that encodes a protein involved in host interaction.
47. The nucleic acid of embodiment 46, wherein said protein affects calcium-independent adhesion to the extracellular matrix.
48. The nucleic acid of embodiment 46, wherein said protein is an NF-κB inhibitor.
49. The nucleic acid of embodiment 48, wherein said protein is encoded by a gene selected from the group consisting of the C2L, N1L, M2L, K1L, and K7R genes.
50. The nucleic acid of embodiment 46, wherein said protein is an apoptosis inhibitor.
51. The nucleic acid of embodiment 47, wherein said apoptosis inhibitor is a caspase-9 inhibitor.
52. The nucleic acid of embodiment 51, wherein said caspase-9 inhibitor is encoded by the F1L gene.
53. The nucleic acid of embodiment 50, wherein said apoptosis inhibitor is a BCL-2-like protein.
54. The nucleic acid of embodiment 53, wherein said BCL-2-like protein is encoded by N1L.
55. The nucleic acid of embodiment 46, wherein said protein is an interferon regulatory factor 3 (IRF3) inhibitor.
56. The nucleic acid of embodiment 55, wherein said IRF3 inhibitor is encoded by N2L or K7R.
57. The nucleic acid of embodiment 46, wherein said protein is a serine protease inhibitor.
58. The nucleic acid of embodiment 46, wherein said protein prevents cell fusion.
59. The nucleic acid of embodiment 58, wherein said protein is encoded by K2L.
60. The nucleic acid of embodiment 46, wherein said protein is an RNA-activated protein kinase (PKR) inhibitor.
61. The nucleic acid of embodiment 60, wherein said protein is encoded by K1L or K3L.
62. The nucleic acid of embodiment 46, wherein said protein is a virulence factor.
63. The nucleic acid of embodiment 63, wherein said protein is encoded by F3L.
64. The nucleic acid of embodiment 46, wherein said protein is an IL-1-beta inhibitor.
65. The nucleic acid of embodiment 64, wherein said protein is encoded by B16R.
66. The nucleic acid of embodiment 46, wherein said protein is a secreted IFNα sequestor.
67. The nucleic acid of embodiment 67, wherein said protein is encoded by B19R.
68. The nucleic acid comprising a recombinant orthopoxvirus genome of any one of embodiments 1-67, wherein said recombinant orthopoxvirus genome comprises a deletion of at least 1 gene encoding a protein involved in DNA replication.
69. The nucleic acid of embodiment 68, wherein said protein is a DNA modifying nuclease.
70. The nucleic acid of embodiment 69, wherein said protein is encoded by K4L.
71. The nucleic acid of embodiment 70, wherein said protein is a deoxyuridine triphosphatase (dUTPase).
72. The nucleic acid of embodiment 71, wherein the dUTPase is encoded by F2L.
73. The nucleic acid of any one of embodiments 1-72, wherein at least one deleted gene's entire nucleotide sequence is deleted.
74. The nucleic acid of any one of embodiments 1-72, wherein at least one deleted gene is only partially deleted, and wherein the partial deletion is sufficient to render said partially deleted gene nonfunctional upon introduction into a host cell.
75. The nucleic acid of any one of embodiments 1-74, wherein said recombinant orthopoxvirus genome comprises at least two copies of inverted terminal repeats (ITRs).
76. The nucleic acid of any one of embodiments 1-74, wherein said recombinant orthopoxvirus genome lacks any copies of ITRs.
77. The nucleic acid of any one of embodiments 1-74, wherein said recombinant orthopoxvirus genome comprises a deletion in at least one copy of an ITR selected from the group consisting of B21R-ITR, B22R-ITR, B23R-ITR, B24R-ITR, B25R-ITR, B26R-ITR, B27R-ITR, B28R-ITR, and B29R-ITR.
78. The nucleic acid of any one of embodiments 1-74, wherein said recombinant orthopoxvirus genome comprises a deletion in at least all of the following copies of ITRs: B21R-ITR, B22R-ITR, B23R-ITR, B24R-ITR, B25R-ITR, B26R-ITR, B27R-ITR, B28R-ITR, and B29R-ITR.
79. The nucleic acid of any one of embodiments 1-78, wherein said recombinant orthopoxvirus genome comprises a deletion in the B8R gene.
80. The nucleic acid of any one of embodiments 1-78, wherein said recombinant orthopoxvirus genome comprises an intact B8R gene.
81. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes; and
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR.
82. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes; and
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR,
   wherein said recombinant orthopoxvirus genome comprises an intact B8R gene.
83. The nucleic acid of any one of embodiments 1-82, further comprising at least one transgene selected from the group consisting of a transgene encoding an immune checkpoint inhibitor, a transgene encoding an interleukin (IL), and a transgene encoding a cytokine.
84. The nucleic acid of embodiment 83, wherein the nucleic acid comprises at least two transgenes selected from the group consisting of a transgene encoding an immune checkpoint inhibitor, a transgene encoding an interleukin (IL), and a transgene encoding a cytokine.
85. The nucleic acid of embodiment 84, wherein the nucleic acid comprises a transgene encoding an immune checkpoint inhibitor, a transgene encoding an interleukin (IL), and a transgene encoding a cytokine.
86. The nucleic acid of any one of embodiment 83 or 84, wherein said nucleic acid comprises a transgene encoding an immune checkpoint inhibitor.
87. The nucleic acid of embodiment 85 or 86, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
88. The nucleic acid of embodiment 87, wherein said immune checkpoint inhibitor is an anti-PD-L1 antibody or antigen-binding fragment thereof or an anti-CTLA-4 antibody or antigen-binding fragment thereof.
89. The nucleic acid of embodiment 88, wherein said immune checkpoint inhibitor is an anti-PD1 antibody or antigen-binding fragment thereof.
90. The nucleic acid of embodiment 88, wherein said immune checkpoint inhibitor is an anti-CTLA-4 antibody or antigen-binding fragment thereof.
91. The nucleic acid of embodiment 83 or 84, wherein said nucleic acid comprises a transgene encoding an interleukin (IL).
92. The nucleic acid of embodiment 85 or 91, wherein said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
93. The nucleic acid of embodiment 92, wherein said interleukin is selected from the group consisting of IL-12 p35, IL-12 p40, and IL-12 p70.
94. The nucleic acid of embodiment 93, wherein said interleukin is membrane-bound.
95. The nucleic acid of embodiment 94, wherein said interleukin is membrane-bound IL-12 p70.
96. The nucleic acid of embodiment 83 or 84, wherein said nucleic acid comprises a transgene encoding a cytokine.
97. The nucleic acid of embodiment 96, wherein said cytokine is an interferon (IFN).
98. The nucleic acid of embodiment 97, wherein said interferon is selected from the group consisting of IFN-alpha, IFN-beta, IFN-delta, IFN-epsilon, IFN-tau, IFN-omega, IFN-zeta, and IFN-gamma.
99. The nucleic acid of embodiment 96, wherein said cytokine is a TNF superfamily member protein.
100. The nucleic acid of embodiment 99, wherein said TNF superfamily member protein is selected from the group consisting of TRAIL, Fas ligand, LIGHT (TNFSF-14), TNF-alpha, and 4-1BB ligand.
101. The nucleic acid of embodiment 96, wherein said cytokine is selected from the group consisting of GM-CSF, Flt3 ligand, CD40 ligand, TGF-beta, VEGF-R2, and c-kit.
102. The nucleic acid of embodiment 101, wherein said cytokine is Flt3 ligand.
103. The nucleic acid of embodiment 83-102, wherein said recombinant orthopoxvirus genome comprises a deletion in the B8R gene and at least one transgene is inserted into the deletion in the B8R gene.
104. The nucleic acid of embodiment 103, wherein at least two transgenes are inserted into the deletion in the B8R gene.
105. The nucleic acid of embodiment 104, wherein at least three transgenes are inserted into the deletion in the B8R gene.
106. The nucleic acid of any one of embodiments 103-105, wherein at least one transgene is inserted in a locus that is not at the deletion in the B8R gene.
107. The nucleic acid of embodiment 106, wherein the locus is at the boundary of a deletion at the 5′ end of the orthopoxvirus genome.
108. The nucleic acid of embodiment 106, wherein the locus is at the boundary of a deletion at the 3′ end of the orthopoxvirus genome.
109. A nucleic acid comprising a recombinant orthopoxvirus genome, wherein said recombinant orthopoxvirus genome comprises
   (i) a deletion of each of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, B20R, and B8R genes;
   (ii) a deletion in each copy of the following ITRs: B21R-ITR, B22R-ITR, B23R-ITR, B24R-ITR, B25R-ITR, B26R-ITR, B27R-ITR, B28R-ITR, and B29R-ITR;
   (iii) an IL-12-TM transgene inserted into the deletion in the B8R gene;
   (iv) an Flt3 ligand transgene inserted into the deletion in the B8R gene; and
   (v) one of:
      (a) a transgene encoding a single chain anti-CTLA-4 antibody or antigen-binding fragment thereof
      or
      (b) (i) a transgene encoding a heavy chain of an anti-CTLA-4 antibody or antigen-binding fragment thereof, and
         (ii) a transgene encoding a light chain of an anti-CTLA-4 antibody or antigen-binding fragment thereof,
   wherein the transgene(s) in part (v) is/are inserted within the boundaries of a 5p deletion present in the recombinant orthopoxvirus genome, and
   wherein the anti-CTLA-4 antibody or antigen-binding fragment thereof is capable of binding CTLA-4.
110. The nucleic acid of embodiment 109, wherein the orthopoxvirus genome is derived from a sequence of SEQ ID NO: 210, wherein
   (a) said derived sequence comprises a deletion of the B8R gene, and the IL-12-TM transgene, the Flt3 ligand transgene, and the transgene(s) encoding the single or double-chain anti-CTLA-4 antibody;
   (b) the IL-12-TM transgene encodes a protein comprising an amino acid sequence of is SEQ ID NO: 212;
   (c) the Flt3 ligand transgene encodes a protein comprising an amino acid sequence of SEQ ID NO: 213; and
   (d) the anti-CTLA-4 antibody comprises an amino acid sequence of SEQ ID NO: 211.
111. A virus comprising the nucleic acid comprising the recombinant orthopoxvirus genome of any one of embodiments 1-110.
112. The virus of embodiment 111, wherein
   a) said recombinant orthopoxvirus genome comprises a deletion of at least 2 genes selected from the group consisting of the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes;
   b) said recombinant orthopoxvirus genome comprises a deletion of at least 1 gene selected from the group consisting of the B14R, B15R, B16R, B17L, B18R, B19R, and B20R genes; or
   c) said recombinant orthopoxvirus genome comprises a deletion of at least 1 gene selected from the group consisting of the C2L, C1L, N1L, N2L, M1L, M2L, K1L, K2L, K3L, K4L, K5L, K6L, K7R, F1L, F2L, and F3L genes.
113. The virus of embodiment 111 or 112, wherein said virus is derived from a vaccinia virus.
114. The virus of embodiment 113, wherein said vaccinia virus is derived from a strain selected from the group consisting of Copenhagen, Western Reserve, Wyeth, Lister, EM63, ACAM2000, LC16m8, CV-1, modified vaccinia Ankara (MVA), Dairen I, GLV-1h68, IHD-J, L-IVP, LC16mO, Tashkent, Tian Tan, and WAU86/88-1.
115. The virus of embodiment 114, wherein said vaccinia virus is derived from a strain selected from the group consisting of Copenhagen, Western Reserve, Tian Tan, Wyeth, and Lister.
116. The virus of embodiment 115, wherein said vaccinia virus is derived from a Copenhagen strain vaccinia virus.
117. The virus of any one of embodiments 111-116, wherein said recombinant orthopoxvirus genome further comprises a Thymidine Kinase (TK) gene.
118. The virus of any one of embodiments 111-117, wherein said recombinant orthopoxvirus genome further comprises a ribonucleotide reductase gene.
119. The virus of any one of embodiments 111-118, wherein upon contacting a population of mammalian cells with said virus, the population of mammalian cells exhibit increased syncytia formation relative to a population of mammalian cells of the same type contacted with a form of the virus that does not comprise said deletion.
120. The virus of any one of embodiments 111-119, wherein upon contacting a population of mammalian cells with said virus, the population of mammalian cells exhibit increased spreading of the virus relative to a population of mammalian cells of the same type contacted with a form of the virus that does not comprise said deletion.
121. The virus of any one of embodiments 111-120, wherein said virus exerts an increased cytotoxic effect on a population of mammalian cells relative to that of a form of the virus that does not comprise said deletion.
122. The virus of any one of embodiments 119-121, wherein said mammalian cells are human cells.
123. The virus of embodiment 122, wherein said human cells are cancer cells.
124. The virus of any one of embodiments 119-121, wherein said mammalian cells are from a cell line selected from the group consisting of U2OS, 293, 293T, Vero, HeLa, A549, BHK, BSC40, CHO, OVCAR-8, 786-0, NCI-H23, U251, SF-295, T-47D, SKMEL2, BT-549, SK-MEL-28, MDA-MB-231, SK-OV-3, MCF7, M14, SF-268, CAKI-1, HPAV, OVCAR-4, HCT15, K-562, and HCT-116.
125. The nucleic acid of any one of embodiments 1-110 or the virus of any one of embodiments 111-124, wherein said nucleic acid or said virus further comprises a transgene encoding a tumor-associated antigen.
126. The nucleic acid or the virus of embodiment 125, wherein said tumor-associated antigen is a tumor-associated antigen listed in any one of Tables 3-30.
127. The nucleic acid or the virus of embodiment 126, wherein said tumor-associated antigen is a tumor-associated antigen selected from the group consisting of CD19, CD33, EpCAM, CEA, PSMA, EGFRvIII, CD133, EGFR, CDH19, ENPP3, DLL3, MSLN, ROR1, HER2, HLAA2, EpHA2, EpHA3, MCSP, CSPG4, NG2, RON, FLT3, BCMA, CD20, FAPα, FRα, CA-9, PDGFRα, PDGFRβ, FSP1, S100A4, ADAM12m, RET, MET, FGFR, INSR, and NTRK.
128. The nucleic acid or the virus of embodiment 125, wherein said tumor-associated antigen comprises MAGE-A3, or one or more fragments thereof.
129. The nucleic acid or the virus of embodiment 125, wherein said tumor-associated antigen comprises NY-ESO-1, or one or more fragments thereof.
130. The nucleic acid or the virus of embodiment 125, wherein said tumor-associated antigen comprises one or more human papillomavirus (HPV) proteins, or fragments thereof.
131. The nucleic acid or the virus of embodiment 125, wherein said HPV proteins or fragments thereof comprise one or more of (i) E6 and E7 proteins, or fragments thereof, of HPV16 and (ii) E6 and E7 proteins, or fragments thereof, of HPV18.
132. The nucleic acid or the virus of embodiment 131, wherein the sequences of said HPV proteins or fragments are disclosed in International Patent Publication WO/2014/127478, the contents of which are incorporated herein by reference.
133. The nucleic acid or the virus of embodiment 125, wherein said tumor-associated antigen comprises brachyury or one or more fragments thereof.
134. The nucleic acid or the virus of embodiment 125, wherein said tumor-associated antigen comprises prostatic acid phosphatase, or one or more fragments thereof.
135. A packaging cell line comprising the nucleic acid of any one of embodiments 1-110 or the virus of any one of embodiments 111-124.
136. A method of treating cancer in a mammalian patient, said method comprising administering a therapeutically effective amount of the virus of any one of embodiments 111-134 to said patient.
137. The method of embodiment 136, wherein said mammalian patient is a human patient.
138. The method of embodiment 136 or 137, wherein the virus is used as a prime in a prime:boost treatment.
139. The method of embodiment 136 or 137, wherein the virus is used as a boost in a prime:boost treatment.
140. The method of any one of embodiments 136-139, wherein said mammalian patient has cancer.
141. The method of embodiment 140, wherein said cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.
142. The method of embodiment 140, wherein said cancer is selected from the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, Ewing sarcoma family, osteosarcoma and malignant fibrous histiocytoma, central nervous system embryonal tumors, central nervous system germ cell tumors, craniopharyngioma, ependymoma, bronchial tumors, Burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, Langerhans cell histiocytosis, Hodgkin lymphoma, hypopharyngeal cancer, islet cell tumors, pancreatic neuroendocrine tumors, Wilms tumor and other childhood kidney tumors, Langerhans cell histiocytosis, small cell lung cancer, cutaneous T cell lymphoma, intraocular melanoma, Merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin's lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, Kaposi's sarcoma, rhabdomyosarcoma, Sézary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.
143. The method of any one of embodiments 136-142, wherein said method further comprises administering to said patient an immune checkpoint inhibitor.
144. The method of embodiment 143, wherein said immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
145. The method of embodiment 144, wherein said immune checkpoint inhibitor is an anti-PD1 antibody or antigen-binding fragment thereof or an anti-CTLA-4 antibody or antigen-binding fragment thereof.
146. The method of embodiment 145, wherein said immune checkpoint inhibitor is an anti-PD1 antibody or antigen-binding fragment thereof.
147. The method of embodiment 145, wherein said immune checkpoint inhibitor is an anti-CTLA-4 antibody or antigen-binding fragment thereof.
148. The method of any one of embodiments 136-147, wherein said method further comprises administering to said patient an interleukin.
149. The method of embodiment 148, wherein said interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
150. The method of embodiment 149, wherein said interleukin is selected from the group consisting of IL-12 p35, IL-12 p40, and IL-12 p70.
151. The method of embodiment 149 or 150, wherein said interleukin is membrane-bound.
152. The method of any one of embodiments 136-151, wherein said method further comprises administering to said patient an interferon.
153. The method of embodiment 152, wherein said interferon is selected from the group consisting of
IFN-alpha, IFN-beta, IFN-delta, IFN-epsilon, IFN-tau, IFN-omega, IFN-zeta, and IFN-gamma.
154. The method of any one of embodiments 136-153, wherein said method further comprises administering to said patient a cytokine.
155. The method of embodiment 154, wherein said cytokine is a TNF superfamily member protein.
156. The method of embodiment 155, wherein said TNF superfamily member protein is selected from the group consisting of TRAIL, Fas ligand, LIGHT (TNFSF-14), TNF-alpha, and 4-1BB ligand.
157. The method of embodiment 154, wherein said cytokine is selected from the group consisting of GM-CSF, Flt3 ligand, CD40 ligand, TGF-beta, VEGF-R2, and cKit.
158. The method of embodiment 157, wherein said cytokine is Flt3 ligand.
159. A kit comprising the nucleic acid of any one of embodiments 1-110 or the virus of any one of embodiments 111-134 and a package insert instructing a user of said kit to express said nucleic acid or said virus in a host cell. 0

160. A kit comprising the virus of any one of embodiments 111-134 and a package insert instructing a user to administer a therapeutically effective amount of said virus to a mammalian patient having cancer, thereby treating said cancer.
161. The kit of embodiment 160, wherein said mammalian patient is a human patient.
162. The nucleic acid of embodiment 94, wherein said interleukin is membrane-bound IL-12 p35.

### 5.7.4. Set 4

1. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to Cytotoxic T-lymphocyte Associated Protein 4 (CTLA-4), wherein the first nucleotide sequence is set forth in SEQ ID NO: 214;
   (c) a second transgene comprising a second nucleotide sequence encoding an Interleukin 12 (IL-12) polypeptide, wherein the second nucleotide sequence is set forth in SEQ ID NO: 215; and
   (d) a third transgene comprising a third nucleotide sequence encoding FMS-like tyrosine kinase 3 ligand (FLT3L), wherein the third nucleotide sequence is set forth in SEQ ID NO: 216.
2. The nucleic acid of embodiment 1, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.
3. The nucleic acid of embodiment 1 or 2, further comprising a nucleotide sequence comprising at least one promoter operably linked to the first nucleotide sequence encoding the anti-CTLA-4 antibody.
4. The nucleic acid of embodiment 3, wherein the at least one promoter operably linked to the first nucleotide sequence encoding the anti-CTLA-4 antibody is an H5R promoter, a pS promoter, or a LEO promoter.
5. The nucleic acid of embodiment 3, wherein the at least one promoter operably linked to the first nucleotide sequence encoding the anti-CTLA-4 antibody is an H5R promoter.
6. The nucleic acid of any one of embodiments 1-5, further comprising a nucleotide sequence comprising at least one promoter operably linked to the second nucleotide sequence encoding the IL-12 polypeptide.
7. The nucleic acid of embodiment 6, wherein the at least one promoter operably linked to the second nucleotide sequence encoding the IL-12 polypeptide is a late promoter.
8. The nucleic acid of embodiment 7, wherein the late promoter comprises the nucleotide sequence of SEQ ID NO: 561, an F17R promoter, or a D13L promoter.
9. The nucleic acid of embodiment 7, wherein the late promoter comprises the nucleotide sequence of SEQ ID NO: 561.
10. The nucleic acid of any one of embodiments 1-9, further comprising a nucleotide sequence comprising at least one promoter operably linked to the third nucleotide sequence encoding FLT3L.
11. The nucleic acid of embodiment 10, wherein the at least one promoter operably linked to the third nucleotide sequence encoding FLT3L is a B8R promoter, a B19R promoter, a E3L promoter, an F 11L promoter, or a B2R promoter.
12. The nucleic acid of embodiment 10, wherein the at least one promoter operably linked to the third nucleotide sequence encoding FLT3L is a B8R promoter.
13. The nucleic acid of embodiment 10, wherein the at least one promoter operably linked to the third nucleotide sequence encoding FLT3L is a B19R promoter.
14. The nucleic acid of embodiment 10, wherein the at least one promoter operably linked to the third nucleotide sequence encoding FLT3L is a B8R promoter and a B19R promoter.
15. The nucleic acid of any one of embodiments 1-14, wherein the first transgene is present between the partial C2L and F3L vaccinia genes in SEQ ID NO: 210.
16. The nucleic acid of any one of embodiments 1-14, wherein the second transgene is present between the partial C2L and F3L vaccinia genes in SEQ ID NO: 210.
17. The nucleic acid of any one of embodiments 1-14, wherein the third transgene is present between the partial C2L and F3L vaccinia genes in SEQ ID NO: 210.
18. The nucleic acid of any one of embodiments 1-14, wherein the first transgene is present in the locus of the deletion in the B8R gene.
19. The nucleic acid of any one of embodiments 1-14, wherein the second transgene is present in the locus of the deletion in the B8R gene.
20. The nucleic acid of any one of embodiments 1-14, wherein the third transgene is present in the locus of the deletion in the B8R gene.
21. The nucleic acid of any one of embodiments 1-14, wherein the first transgene is present between the partial B14R and B29R vaccinia genes in SEQ ID NO: 210.
22. The nucleic acid of any one of embodiments 1-14, wherein the second transgene is present between the partial B14R and B29R vaccinia genes in SEQ ID NO: 210.
23. The nucleic acid of any one of embodiments 1-14, wherein the third transgene is present between the partial B14R and B29R vaccinia genes in SEQ ID NO: 210.
24. The nucleic acid of any one of embodiments 1-14, wherein the first transgene and the second transgene are present between the partial C2L and F3L vaccinia genes in SEQ ID NO: 210.
25. The nucleic acid of any one of embodiments 1-14, wherein the first transgene and the second transgene are present in the locus of the deletion in the B8R gene.
26. The nucleic acid of any one of embodiments 1-14, wherein the first transgene and the second transgene are present between the partial B14R and B29R vaccinia genes in SEQ ID NO: 210.
27. The nucleic acid of any one of embodiments 1-14, wherein the first transgene and the third transgene are present between the partial C2L and F3L vaccinia genes in SEQ ID NO: 210.
28. The nucleic acid of any one of embodiments 1-14, wherein the first transgene and the third transgene are present in the locus of the deletion in the B8R gene.
29. The nucleic acid of any one of embodiments 1-14, wherein the first transgene and the third transgene are present between the partial B14R and B29R vaccinia genes in SEQ ID NO: 210.
30. The nucleic acid of any one of embodiments 1-14, wherein the second transgene and the third transgene are present between the partial C2L and F3L vaccinia genes in SEQ ID NO: 210.
31. The nucleic acid of any one of embodiments 1-14, wherein the second transgene and the third transgene are present in the locus of the deletion in the B8R gene.
32. The nucleic acid of any one of embodiments 1-14, wherein the second transgene and the third transgene are present between the partial B14R and B29R vaccinia genes in SEQ ID NO: 210.
33. The nucleic acid of any one of embodiments 1-14, wherein the first transgene is present between the partial C2L and F3L vaccinia genes in SEQ ID NO: 210, and the second transgene is present in the locus of the deletion in the B8R gene.
34. The nucleic acid of any one of embodiments 1-14, wherein the second transgene is present between the partial C2L and F3L vaccinia genes in SEQ ID NO: 210, and the first transgene is present in the locus of the deletion in the B8R gene.
35. The nucleic acid of any one of embodiments 1-14, wherein the first transgene is present between the partial C2L and F3L vaccinia genes in SEQ ID NO: 210, and the third transgene is present in the locus of the deletion in the B8R gene.
36. The nucleic acid of any one of embodiments 1-14, wherein the third transgene is present between the partial C2L and F3L vaccinia genes in SEQ ID NO: 210, and the first transgene is present in the locus of the deletion in the B8R gene.
37. The nucleic acid of any one of embodiments 1-14, wherein the second transgene is present between the partial C2L and F3L vaccinia genes in SEQ ID NO: 210, and the third transgene is present in the locus of the deletion in the B8R gene.
38. The nucleic acid of any one of embodiments 1-14, wherein the third transgene is present between the partial C2L and F3L vaccinia genes in SEQ ID NO: 210, and the second transgene is present in the locus of the deletion in the B8R gene.
39. The nucleic acid of any one of embodiments 1-14, wherein the first transgene is present between the partial C2L and F3L vaccinia genes in SEQ ID NO: 210, and the second transgene is present between the partial B14R and B29R vaccinia genes in SEQ ID NO: 210.
40. The nucleic acid of any one of embodiments 1-14, wherein the second transgene is present between the partial C2L and F3L vaccinia genes in SEQ ID NO: 210, and the first transgene is present between the partial B14R and B29R vaccinia genes in SEQ ID NO: 210.
41. The nucleic acid of any one of embodiments 1-14, wherein the first transgene is present between the partial C2L and F3L vaccinia genes in SEQ ID NO: 210, and the third transgene is present between the partial B14R and B29R vaccinia genes in SEQ ID NO: 210.
42. The nucleic acid of any one of embodiments 1-14, wherein the third transgene is present between the partial C2L and F3L vaccinia genes in SEQ ID NO: 210, and the first transgene is present between the partial B14R and B29R vaccinia genes in SEQ ID NO: 210.
43. The nucleic acid of any one of embodiments 1-14, wherein the second transgene is present between the partial C2L and F3L vaccinia genes in SEQ ID NO: 210, and the third transgene is present between the partial B14R and B29R vaccinia genes in SEQ ID NO: 210.
44. The nucleic acid of any one of embodiments 1-14, wherein the third transgene is present between the partial C2L and F3L vaccinia genes in SEQ ID NO: 210, and the second transgene is present between the partial B14R and B29R vaccinia genes in SEQ ID NO: 210.
45. The nucleic acid of any one of embodiments 1-14, wherein the first transgene is present in the locus of the deletion in the B8R gene, and the second transgene is present between the partial B14R and B29R vaccinia genes in SEQ ID NO: 210.
46. The nucleic acid of any one of embodiments 1-14, wherein the second transgene is present in the locus of the deletion in the B8R gene, and the first transgene is present between the partial B14R and B29R vaccinia genes in SEQ ID NO: 210.
47. The nucleic acid of any one of embodiments 1-14, wherein the first transgene is present in the locus of the deletion in the B8R gene, and the third transgene is present between the partial B14R and B29R vaccinia genes in SEQ ID NO: 210.
48. The nucleic acid of any one of embodiments 1-14, wherein the third transgene is present in the locus of the deletion in the B8R gene, and the first transgene is present between the partial B14R and B29R vaccinia genes in SEQ ID NO: 210.
49. The nucleic acid of any one of embodiments 1-14, wherein the second transgene is present in the locus of the deletion in the B8R gene, and the third transgene is present between the partial B14R and B29R vaccinia genes in SEQ ID NO: 210.
50. The nucleic acid of any one of embodiments 1-14, wherein the third transgene is present in the locus of the deletion in the B8R gene, and the second transgene is present between the partial B14R and B29R vaccinia genes in SEQ ID NO: 210.
51. The nucleic acid of any one of embodiments 1-14, wherein the first transgene, the second transgene, and the third transgene are present between the partial C2L and F3L vaccinia genes in SEQ ID NO: 210.
52. The nucleic acid of any one of embodiments 1-14, wherein the first transgene, the second transgene, and the third transgene are present in the locus of the deletion in the B8R gene.
53. The nucleic acid of any one of embodiments 1-14, wherein the first transgene, the second transgene, and the third transgene are present between the partial B14R and B29R vaccinia genes in SEQ ID NO: 210.
54. The nucleic acid of any one of embodiments 1-14, wherein the first transgene is present between the partial C2L and F3L vaccinia genes in SEQ ID NO: 210, and the second transgene and the third transgene are present in the locus of the deletion in the B8R gene.
55. The nucleic acid of any one of embodiments 1-14, wherein the second transgene is present between the partial C2L and F3L vaccinia genes in SEQ ID NO: 210, and the first transgene and the third transgene are present in the locus of the deletion in the B8R gene.
56. The nucleic acid of any one of embodiments 1-14, wherein the third transgene is present between the partial C2L and F3L vaccinia genes in SEQ ID NO: 210, and the first transgene and the second transgene are present in the locus of the deletion in the B8R gene. 0

57. The nucleic acid of any one of embodiments 1-14, wherein the first transgene and the second transgene are present between the partial C2L and F3L vaccinia genes in SEQ ID NO: 210, and the third transgene is present in the locus of the deletion in the B8R gene.
58. The nucleic acid of any one of embodiments 1-14, wherein the first transgene and the third transgene are present between the partial C2L and F3L vaccinia genes in SEQ ID NO: 210, and the second transgene is present in the locus of the deletion in the B8R gene.
59. The nucleic acid of any one of embodiments 1-14, wherein the second transgene and the third transgene are present between the partial C2L and F3L vaccinia genes in SEQ ID NO: 210, and the first transgene is present in the locus of the deletion in the B8R gene.
60. The nucleic acid of any one of embodiments 1-14, wherein the first transgene is present between the partial C2L and F3L vaccinia genes in SEQ ID NO: 210, and the second transgene and the third transgene are present between the partial B14R and B29R vaccinia genes in SEQ ID NO: 210.
61. The nucleic acid of any one of embodiments 1-14, wherein the second transgene is present between the partial C2L and F3L vaccinia genes in SEQ ID NO: 210, and the first transgene and the third transgene are present between the partial B14R and B29R vaccinia genes in SEQ ID NO: 210.
62. The nucleic acid of any one of embodiments 1-14, wherein the third transgene is present between the partial C2L and F3L vaccinia genes in SEQ ID NO: 210, and the first transgene and the second transgene are present between the partial B14R and B29R vaccinia genes in SEQ ID NO: 210.
63. The nucleic acid of any one of embodiments 1-14, wherein the first transgene and the second transgene are present between the partial C2L and F3L vaccinia genes in SEQ ID NO: 210, and the third transgene is present between the partial B14R and B29R vaccinia genes in SEQ ID NO: 210.
64. The nucleic acid of any one of embodiments 1-14, wherein the first transgene and the third transgene are present between the partial C2L and F3L vaccinia genes in SEQ ID NO: 210, and the second transgene is present between the partial B14R and B29R vaccinia genes in SEQ ID NO: 210.
65. The nucleic acid of any one of embodiments 1-14, wherein the second transgene and the third transgene are present between the partial C2L and F3L vaccinia genes in SEQ ID NO: 210, and the first transgene is present between the partial B14R and B29R vaccinia genes in SEQ ID NO: 210.
66. The nucleic acid of any one of embodiments 1-14, wherein the first transgene is present in the locus of the deletion in the B8R gene, and the second transgene and the third transgene are present between the partial B14R and B29R vaccinia genes in SEQ ID NO: 210.
67. The nucleic acid of any one of embodiments 1-14, wherein the second transgene is present in the locus of the deletion in the B8R gene, and the first transgene and the third transgene are present between the partial B14R and B29R vaccinia genes in SEQ ID NO: 210.
68. The nucleic acid of any one of embodiments 1-14, wherein the third transgene is present in the locus of the deletion in the B8R gene, and the first transgene and the second transgene are present between the partial B14R and B29R vaccinia genes in SEQ ID NO: 210.
69. The nucleic acid of any one of embodiments 1-14, wherein the first transgene and the second transgene are present in the locus of the deletion in the B8R gene, and the third transgene is present between the partial B14R and B29R vaccinia genes in SEQ ID NO: 210.
70. The nucleic acid of any one of embodiments 1-14, wherein the first transgene and the third transgene are present in the locus of the deletion in the B8R gene, and the second transgene is present between the partial B14R and B29R vaccinia genes in SEQ ID NO: 210.
71. The nucleic acid of any one of embodiments 1-14, wherein the second transgene and the third transgene are present in the locus of the deletion in the B8R gene, and the first transgene is present between the partial B14R and B29R vaccinia genes in SEQ ID NO: 210.
72. The nucleic acid of any one of embodiments 1-14, wherein the first transgene is present between the partial C2L and F3L vaccinia genes in SEQ ID NO: 210, the second transgene is present in the locus of the deletion in the B8R gene, and the third transgene is present between the partial B14R and B29R vaccinia genes in SEQ ID NO: 210.
73. The nucleic acid of any one of embodiments 1-14, wherein the first transgene is present between the partial C2L and F3L vaccinia genes in SEQ ID NO: 210, the third transgene is present in the locus of the deletion in the B8R gene, and the second transgene is present between the partial B14R and B29R vaccinia genes in SEQ ID NO: 210.
74. The nucleic acid of any one of embodiments 1-14, wherein the second transgene is present between the partial C2L and F3L vaccinia genes in SEQ ID NO: 210, the first transgene is present in the locus of the deletion in the B8R gene, and the third transgene is present between the partial B14R and B29R vaccinia genes in SEQ ID NO: 210.
75. The nucleic acid of any one of embodiments 1-14, wherein the second transgene is present between the partial C2L and F3L vaccinia genes in SEQ ID NO: 210, the third transgene is present in the locus of the deletion in the B8R gene, and the first transgene is present between the partial B14R and B29R vaccinia genes in SEQ ID NO: 210.
76. The nucleic acid of any one of embodiments 1-14, wherein the third transgene is present between the partial C2L and F3L vaccinia genes in SEQ ID NO: 210, the first transgene is present in the locus of the deletion in the B8R gene, and the second transgene is present between the partial B14R and B29R vaccinia genes in SEQ ID NO: 210.
77. The nucleic acid of any one of embodiments 1-14, wherein the third transgene is present between the partial C2L and F3L vaccinia genes in SEQ ID NO: 210, the second transgene is present in the locus of the deletion in the B8R gene, and the first transgene is present between the partial B14R and B29R vaccinia genes in SEQ ID NO: 210.
78. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214;
   (c) a second transgene comprising a second nucleotide sequence encoding an IL-12 polypeptide, wherein the second nucleotide sequence is set forth in SEQ ID NO: 215; and
   (d) a third transgene comprising a third nucleotide sequence encoding FLT3L, wherein the third nucleotide sequence is set forth in SEQ ID NO: 216; wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter and a B19R promoter.
79. The nucleic acid of embodiment 78, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.
80. The nucleic acid of embodiment 78 or 79, wherein the first transgene is present between the partial C2L and F3L vaccinia genes in SEQ ID NO: 210, and the second transgene and the third transgene are present in the locus of the deletion in the B8R gene.
81. The nucleic acid of embodiment 78 or 79, wherein the first transgene is present between the partial B14R and B29R vaccinia genes in SEQ ID NO: 210, and the second transgene and the third transgene are present in the locus of the deletion in the B8R gene.
82. The nucleic acid of embodiment 80 or 81, wherein the third transgene is upstream of the second transgene.
83. The nucleic acid of embodiment 80 or 81, wherein the third transgene is downstream of the second transgene.
84. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
85. The nucleic acid of embodiment 84, wherein the at least one promoter operably linked to the third nucleotide sequence is a B8R promoter and a B19R promoter.
86. The nucleic acid of embodiment 85, wherein the B8R promoter comprises the nucleotide sequence of SEQ ID NO: 564 and the B19R promoter comprises the nucleotide sequence of SEQ ID NO: 565.
87. The nucleic acid of any one of embodiments 84-86, wherein the at least one promoter operatively linked to the first nucleotide sequence is an H5R early promoter or an H5R late promoter.
88. The nucleic acid of embodiment 87, wherein the at least one promoter operatively linked to the first nucleotide sequence is an H5R early promoter and an H5R late promoter.
89. The nucleic acid of embodiment 88, wherein the H5R early promoter comprises the nucleotide sequence of SEQ ID NO: 553 and the H5R late promoter comprises the nucleotide sequence of SEQ ID NO: 554.
90. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
91. The nucleic acid of embodiment 90, wherein the at least one promoter operably linked to the third nucleotide sequence is a B8R promoter and a B19R promoter.
92. The nucleic acid of embodiment 91, wherein the B8R promoter comprises the nucleotide sequence of SEQ ID NO: 564 and the B19R promoter comprises the nucleotide sequence of SEQ ID NO: 565.
93. The nucleic acid of any one of embodiments 90-92, wherein the at least one promoter operatively linked to the first nucleotide sequence is an H5R early promoter or an H5R late promoter.
94. The nucleic acid of embodiment 93, wherein the at least one promoter operatively linked to the first nucleotide sequence is an H5R early promoter and an H5R late promoter.
95. The nucleic acid of embodiment 94, wherein the H5R early promoter comprises the nucleotide sequence of SEQ ID NO: 553 and the H5R late promoter comprises the nucleotide sequence of SEQ ID NO: 554.
96. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes in SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene; 1

wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.
97. The nucleic acid of embodiment 96, wherein the at least one promoter operably linked to the third nucleotide sequence is a B8R promoter and a B19R promoter.
98. The nucleic acid of embodiment 97, wherein the B8R promoter comprises the nucleotide sequence of SEQ ID NO: 564 and the B19R promoter comprises the nucleotide sequence of SEQ ID NO: 565.
99. The nucleic acid of any one of embodiments 96-98, wherein the at least one promoter operatively linked to the first nucleotide sequence is an H5R early promoter or an H5R late promoter.
100. The nucleic acid of embodiment 99, wherein the at least one promoter operatively linked to the first nucleotide sequence is an H5R early promoter and an H5R late promoter.
101. The nucleic acid of embodiment 100, wherein the H5R early promoter comprises the nucleotide sequence of SEQ ID NO: 553 and the H5R late promoter comprises the nucleotide sequence of SEQ ID NO: 554.
102. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
103. The nucleic acid of embodiment 102, wherein the at least one promoter operably linked to the third nucleotide sequence is a B8R promoter and a B19R promoter.
104. The nucleic acid of embodiment 103, wherein the B8R promoter comprises the nucleotide sequence of SEQ ID NO: 564 and the B19R promoter comprises the nucleotide sequence of SEQ ID NO: 565.
105. The nucleic acid of any one of embodiments 102-104, wherein the at least one promoter operatively linked to the first nucleotide sequence is an H5R early promoter or an H5R late promoter.
106. The nucleic acid of embodiment 105, wherein the at least one promoter operatively linked to the first nucleotide sequence is an H5R early promoter and an H5R late promoter.
107. The nucleic acid of embodiment 106, wherein the H5R early promoter comprises the nucleotide sequence of SEQ ID NO: 553 and the H5R late promoter comprises the nucleotide sequence of SEQ ID NO: 554.
108. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
109. The nucleic acid of embodiment 108, wherein the at least one promoter operably linked to the third nucleotide sequence is a B8R promoter and a B19R promoter.
110. The nucleic acid of embodiment 109, wherein the B8R promoter comprises the nucleotide sequence of SEQ ID NO: 564 and the B19R promoter comprises the nucleotide sequence of SEQ ID NO: 565.
111. The nucleic acid of any one of embodiments 108-110, wherein the nucleotide sequence of the pS comprises the nucleotide sequence of SEQ ID NO: 555, SEQ ID NO: 556, or SEQ ID NO: 557.
112. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
113. The nucleic acid of embodiment 112, wherein the at least one promoter operably linked to the third nucleotide sequence is a B8R promoter and a B19R promoter.
114. The nucleic acid of embodiment 113, wherein the B8R promoter comprises the nucleotide sequence of SEQ ID NO: 564 and the B19R promoter comprises the nucleotide sequence of SEQ ID NO: 565.
115. The nucleic acid of any one of embodiments 112-114, wherein the nucleotide sequence of the pS comprises the nucleotide sequence of SEQ ID NO: 555, SEQ ID NO: 556, or SEQ ID NO: 557.
116. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
117. The nucleic acid of embodiment 116, wherein the at least one promoter operably linked to the third nucleotide sequence is a B8R promoter and a B19R promoter.
118. The nucleic acid of embodiment 117, wherein the B8R promoter comprises the nucleotide sequence of SEQ ID NO: 564 and the B19R promoter comprises the nucleotide sequence of SEQ ID NO: 565.
119. The nucleic acid of any one of embodiments 116-118, wherein the nucleotide sequence of the pS comprises the nucleotide sequence of SEQ ID NO: 555, SEQ ID NO: 556, or SEQ ID NO: 557.
120. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter or a B19R promoter.
121. The nucleic acid of embodiment 120, wherein the at least one promoter operably linked to the third nucleotide sequence is a B8R promoter and a B19R promoter.
122. The nucleic acid of embodiment 121, wherein the B8R promoter comprises the nucleotide sequence of SEQ ID NO: 564 and the B19R promoter comprises the nucleotide sequence of SEQ ID NO: 565.
123. The nucleic acid of any one of embodiments 120-122, wherein the nucleotide sequence of the pS comprises the nucleotide sequence of SEQ ID NO: 555, SEQ ID NO: 556, or SEQ ID NO: 557.
124. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a B8R promoter or a B19R promoter.
125. The nucleic acid of embodiment 124, wherein the at least one promoter operably linked to the third nucleotide sequence is a B8R promoter and a B19R promoter.
126. The nucleic acid of embodiment 125, wherein the B8R promoter comprises the nucleotide sequence of SEQ ID NO: 564 and the B19R promoter comprises the nucleotide sequence of SEQ ID NO: 565.
127. The nucleic acid of any one of embodiments 124-126, wherein the at least one promoter operatively linked to the first nucleotide sequence is an H5R early promoter or an H5R late promoter.
128. The nucleic acid of embodiment 127, wherein the at least one promoter operatively linked to the first nucleotide sequence is an H5R early promoter and an H5R late promoter.
129. The nucleic acid of embodiment 128, wherein the H5R early promoter comprises the nucleotide sequence of SEQ ID NO: 553 and the H5R late promoter comprises the nucleotide sequence of SEQ ID NO: 554.
130. The nucleic acid of any one of embodiments 124-129, nucleotide sequence of the F17R promoter comprises the nucleotide sequence of SEQ ID NO: 563.
131. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene, of the vaccinia virus nucleotide sequence of SEQ ID NO: 210 and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a B8R promoter or a B19R promoter.
132. The nucleic acid of embodiment 131, wherein the at least one promoter operably linked to the third nucleotide sequence is a B8R promoter and a B19R promoter.
133. The nucleic acid of embodiment 132, wherein the B8R promoter comprises the nucleotide sequence of SEQ ID NO: 564 and the B19R promoter comprises the nucleotide sequence of SEQ ID NO: 565.
134. The nucleic acid of any one of embodiments 131-133, wherein the at least one promoter operatively linked to the first nucleotide sequence is an H5R early promoter or an H5R late promoter.
135. The nucleic acid of embodiment 134, wherein the at least one promoter operatively linked to the first nucleotide sequence is an H5R early promoter and an H5R late promoter.
136. The nucleic acid of embodiment 135, wherein the H5R early promoter comprises the nucleotide sequence of SEQ ID NO: 553 and the H5R late promoter comprises the nucleotide sequence of SEQ ID NO: 554.
137. The nucleic acid of any one of embodiments 131-136, nucleotide sequence of the F17R promoter comprises the nucleotide sequence of SEQ ID NO: 563.
138. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a B8R promoter or a B19R promoter.
139. The nucleic acid of embodiment 138, wherein the at least one promoter operably linked to the third nucleotide sequence is a B8R promoter and a B19R promoter.
140. The nucleic acid of embodiment 139, wherein the B8R promoter comprises the nucleotide sequence of SEQ ID NO: 564 and the B19R promoter comprises the nucleotide sequence of SEQ ID NO: 565.
141. The nucleic acid of any one of embodiments 138-140, wherein the at least one promoter operatively linked to the first nucleotide sequence is an H5R early promoter or an H5R late promoter.
142. The nucleic acid of embodiment 141, wherein the at least one promoter operatively linked to the first nucleotide sequence is an H5R early promoter and an H5R late promoter.
143. The nucleic acid of embodiment 142, wherein the H5R early promoter comprises the nucleotide sequence of SEQ ID NO: 553 and the H5R late promoter comprises the nucleotide sequence of SEQ ID NO: 554.
144. The nucleic acid of any one of embodiments 138-143, nucleotide sequence of the F17R promoter comprises the nucleotide sequence of SEQ ID NO: 563.
145. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a B8R promoter and a B19R promoter.
146. The nucleic acid of embodiment 145, wherein the at least one promoter operably linked to the third nucleotide sequence is a B8R promoter and a B19R promoter.
147. The nucleic acid of embodiment 146, wherein the B8R promoter comprises the nucleotide sequence of SEQ ID NO: 564 and the B19R promoter comprises the nucleotide sequence of SEQ ID NO: 565.
148. The nucleic acid of any one of embodiments 145-147, wherein the at least one promoter operatively linked to the first nucleotide sequence is an H5R early promoter or an H5R late promoter.
149. The nucleic acid of embodiment 148, wherein the at least one promoter operatively linked to the first nucleotide sequence is an H5R early promoter and an H5R late promoter.
150. The nucleic acid of embodiment 149, wherein the H5R early promoter comprises the nucleotide sequence of SEQ ID NO: 553 and the H5R late promoter comprises the nucleotide sequence of SEQ ID NO: 554.
151. The nucleic acid of any one of embodiments 145-150, nucleotide sequence of the F17R promoter comprises the nucleotide sequence of SEQ ID NO: 563.
152. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a E3L promoter. 0

153. The nucleic acid of embodiment 152, wherein the at least one promoter operatively linked to the first nucleotide sequence is an H5R early promoter or an H5R late promoter.
154. The nucleic acid of embodiment 153, wherein the at least one promoter operatively linked to the first nucleotide sequence is an H5R early promoter and an H5R late promoter.
155. The nucleic acid of embodiment 154, wherein the H5R early promoter comprises the nucleotide sequence of SEQ ID NO: 553 and the H5R late promoter comprises the nucleotide sequence of SEQ ID NO: 554.
156. The nucleic acid of any one of embodiments 152-155, nucleotide sequence of the E3L promoter comprises the nucleotide sequence of SEQ ID NO: 567.
157. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a E3L promoter.
158. The nucleic acid of embodiment 157, wherein the at least one promoter operatively linked to the first nucleotide sequence is an H5R early promoter or an H5R late promoter.
159. The nucleic acid of embodiment 158, wherein the at least one promoter operatively linked to the first nucleotide sequence is an H5R early promoter and an H5R late promoter.
160. The nucleic acid of embodiment 159, wherein the H5R early promoter comprises the nucleotide sequence of SEQ ID NO: 553 and the H5R late promoter comprises the nucleotide sequence of SEQ ID NO: 554.
161. The nucleic acid of any one of embodiments 157-160, nucleotide sequence of the E3L promoter comprises the nucleotide sequence of SEQ ID NO: 567.
162. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a E3L promoter.
163. The nucleic acid of embodiment 162, wherein the at least one promoter operatively linked to the first nucleotide sequence is an H5R early promoter or an H5R late promoter.
164. The nucleic acid of embodiment 163, wherein the at least one promoter operatively linked to the first nucleotide sequence is an H5R early promoter and an H5R late promoter.
165. The nucleic acid of embodiment 164, wherein the H5R early promoter comprises the nucleotide sequence of SEQ ID NO: 553 and the H5R late promoter comprises the nucleotide sequence of SEQ ID NO: 554.
166. The nucleic acid of any one of embodiments 162-165, nucleotide sequence of the E3L promoter comprises the nucleotide sequence of SEQ ID NO: 567.
167. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene; 1

wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a E3L promoter.
168. The nucleic acid of embodiment 167, wherein the at least one promoter operatively linked to the first nucleotide sequence is an H5R early promoter or an H5R late promoter.
169. The nucleic acid of embodiment 168, wherein the at least one promoter operatively linked to the first nucleotide sequence is an H5R early promoter and an H5R late promoter.
170. The nucleic acid of embodiment 169, wherein the H5R early promoter comprises the nucleotide sequence of SEQ ID NO: 553 and the H5R late promoter comprises the nucleotide sequence of SEQ ID NO: 554.
171. The nucleic acid of any one of embodiments 167-170, nucleotide sequence of the E3L promoter comprises the nucleotide sequence of SEQ ID NO: 567.
172. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence an F17R promoter; and
   (iii) a nucleotide sequence comprising at least one promoter operably linked to the third nucleotide sequence, wherein the at least one promoter operably linked to the third nucleotide sequence is a E3L promoter.
173. The nucleic acid of embodiment 172, wherein the nucleotide sequence of the pS promoter comprises the nucleotide sequence of SEQ ID NO: 555, SEQ ID NO: 556, or SEQ ID NO: 557.
174. The nucleic acid of embodiment 172 or 173, wherein the nucleotide sequence of the F17R promoter comprises the nucleotide sequence of SEQ ID NO: 563.
175. The nucleic acid of any one of embodiments 172-174, wherein the nucleotide sequence of the E3L promoter comprises the nucleotide sequence of SEQ ID NO: 567.
176. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a E3L promoter.
177. The nucleic acid of embodiment 176, wherein the nucleotide sequence of the pS promoter comprises the nucleotide sequence of SEQ ID NO: 555, SEQ ID NO: 556, or SEQ ID NO: 557.
178. The nucleic acid of embodiment 176 or 177, wherein the nucleotide sequence of the F17R promoter comprises the nucleotide sequence of SEQ ID NO: 563.
179. The nucleic acid of any one of embodiments 176-178, wherein the nucleotide sequence of the E3L promoter comprises the nucleotide sequence of SEQ ID NO: 567.
180. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a E3L promoter.
181. The nucleic acid of embodiment 180, wherein the nucleotide sequence of the pS promoter comprises the nucleotide sequence of SEQ ID NO: 555, SEQ ID NO: 556, or SEQ ID NO: 557.
182. The nucleic acid of embodiment 170 or 181, wherein the nucleotide sequence of the F17R promoter comprises the nucleotide sequence of SEQ ID NO: 563.
183. The nucleic acid of any one of embodiments 180-182, wherein the nucleotide sequence of the E3L promoter comprises the nucleotide sequence of SEQ ID NO: 567.
184. A nucleic acid comprising a recombinant vaccinia virus genome, comprising:
   (a) a vaccinia virus nucleotide sequence of SEQ ID NO: 210, which comprises partial C2L, F3L, B14R, and B29R vaccinia genes and which comprises a deletion in the B8R gene;
   (b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
   (c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
   (d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
   wherein the nucleic acid further comprises:
   (i) a nucleotide sequence comprising at least one promoter operably linked to the first nucleotide sequence, wherein the at least one promoter operably linked to the first nucleotide sequence is a pS promoter;
   (ii) a nucleotide sequence comprising at least one promoter operably linked to the second nucleotide sequence, wherein the at least one promoter operably linked to the second nucleotide sequence is an F17R promoter; and
   (iii) a nucleotide sequence comprising at least one promoter operably linked to the third nucleotide sequence, wherein the at least one promoter operably linked to the third nucleotide sequence is a E3L promoter.
185. The nucleic acid of embodiment 184, wherein the nucleotide sequence of the pS promoter comprises the nucleotide sequence of SEQ ID NO: 555, SEQ ID NO: 556, or SEQ ID NO: 557.
186. The nucleic acid of embodiment 174 or 185, wherein the nucleotide sequence of the F17R promoter comprises the nucleotide sequence of SEQ ID NO: 563.
187. The nucleic acid of any one of embodiments 184-186, wherein the nucleotide sequence of the E3L promoter comprises the nucleotide sequence of SEQ ID NO: 567.
188. A virus comprising the nucleic acid comprising a recombinant vaccinia virus genome of any one of embodiments 1-187.
189. A packaging cell line comprising the nucleic acid of any one of embodiments 1-187.
190. A packaging cell line comprising the virus of embodiment 188.
191. A pharmaceutical composition comprising the virus of embodiment 188 and a physiologically acceptable carrier.
192. A kit comprising the nucleic acid of any one of embodiments 1-187 and a package insert instructing a user of the kit to express the nucleic acid in a host cell.
193. A kit comprising the virus of embodiment 188 and a package insert instructing a user of the kit to express the virus in a host cell.
194. A kit comprising the virus of embodiment 188 and a package insert instructing a user to administer a therapeutically effective amount of the virus to a mammalian patient having cancer, thereby treating the cancer.
195. The kit of embodiment 194, wherein the mammalian patient is a human patient.
196. A method of treating cancer in a mammalian patient, the method comprising administering to the mammalian patient a therapeutically effective amount of the virus of embodiment 188.
197. A method of treating cancer in a mammalian patient, the method comprising administering to the mammalian patient a therapeutically effective amount of the pharmaceutical composition of embodiment 191.
198. The method of embodiment 196 or 197, wherein the mammalian patient is a human patient.
199. The method of any one of embodiments 196-198, wherein the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.
200. The method of embodiment 199, wherein the method further comprises administering to the mammalian patient an anti-PD1 antibody or an anti-PD-L1 antibody.

### 5.7.5. Set 5

1. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ inverted terminal repeat (ITR): B21R, B22R, B23R, B24R, B25R, B26R, B27R, B28R, and B29R; and
   (c) a first transgene comprising a first nucleotide sequence encoding an antibody or antigen-binding fragment thereof that specifically binds to Cytotoxic T-lymphocyte Associated Protein 4 (CTLA-4);
   wherein the deletions in the C2L, F3L, B14R, and B29R vaccinia genes are partial deletions.
2. The nucleic acid of embodiment 1, further comprising a nucleotide sequence comprising at least one promoter operably linked to the first nucleotide sequence.
3. The nucleic acid of embodiment 2, wherein the at least one promoter operably linked to the first nucleotide sequence is an H5R promoter, a pS promoter, or a LEO promoter.
4. The nucleic acid of embodiment 2, wherein the at least one promoter operably linked to the first nucleotide sequence is an H5R promoter.
5. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4; and
   (d) a nucleotide sequence comprising at least one promoter operably linked to the first nucleotide sequence, wherein the at least one promoter operably linked to the first nucleotide sequence is an H5R promoter, a pS promoter, or a LEO promoter;
   wherein the deletions in the C2L, F3L, B14R, and B29R vaccinia genes are partial deletions.
6. The nucleic acid of embodiment 5, wherein the at least one promoter operably linked to the first nucleotide sequence is an H5R promoter.
7. The nucleic acid of any one of embodiments 1-6, wherein the first nucleotide sequence is in the same orientation as endogenous vaccinia virus genes that flank the first nucleotide sequence.
8. The nucleic acid of any one of embodiments 1-7, wherein the first nucleotide sequence encodes an amino acid sequence comprising the amino acid sequence set forth in SEQ ID NO: 211.
9. The nucleic acid of any one of embodiments 1-8, wherein the first nucleotide sequence comprises the sequence set forth in SEQ ID NO: 214.
10. The nucleic acid of any one of embodiments 1-8, wherein the first nucleotide sequence is set forth in SEQ ID NO: 214.
11. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R; and
   (c) a second transgene comprising a second nucleotide sequence encoding an Interleukin 12 (IL-12) polypeptide;
   wherein the deletions in the C2L, F3L, B14R, and B29R vaccinia genes are partial deletions.
12. The nucleic acid of embodiment 11, further comprising a nucleotide sequence comprising at least one promoter operably linked to the second nucleotide sequence.
13. The nucleic acid of embodiment 12, wherein the at least one promoter operably linked to the second nucleotide sequence is a late promoter.
14. The nucleic acid of embodiment 13, wherein the late promoter comprises the nucleotide sequence of SEQ ID NO: 561, an F17R promoter, or a D13L promoter.
15. The nucleic acid of embodiment 13, wherein the late promoter comprises the nucleotide sequence of SEQ ID NO: 561.
16. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a second transgene comprising a second nucleotide sequence encoding an IL-12 polypeptide; and
   (d) a nucleotide sequence comprising at least one promoter operably linked to the second nucleotide sequence, wherein the at least one promoter operably linked to the second nucleotide sequence is a late promoter;
   wherein the deletions in the C2L, F3L, B14R, and B29R vaccinia genes are partial deletions.
17. The nucleic acid of embodiment 16, wherein the late promoter comprises the nucleotide sequence of SEQ ID NO: 561, an F17R promoter, or a D13L promoter.
18. The nucleic acid of embodiment 16, wherein the late promoter comprises the nucleotide sequence of SEQ ID NO: 561.
19. The nucleic acid of any one of embodiments 11-18, wherein the second nucleotide sequence is in the same orientation as endogenous vaccinia virus genes that flank the second nucleotide sequence.
20. The nucleic acid of any one of embodiments 11-19, wherein the IL-12 polypeptide is membrane-bound.
21. The nucleic acid of any one of embodiments 11-20, wherein the IL-12 polypeptide comprises IL-12 p35 or IL-12 p70.
22. The nucleic acid of any one of embodiments 11-21, wherein the IL-12 polypeptide comprises the amino acid sequence set forth in SEQ ID NO: 212.
23. The nucleic acid of any one of embodiments 11-22, wherein the second nucleotide sequence comprises the sequence set forth in SEQ ID NO: 215.
24. The nucleic acid of any one of embodiments 11-22, wherein the second nucleotide sequence is set forth in SEQ ID NO: 215.
25. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R; and
   (c) a third transgene comprising a third nucleotide sequence encoding FMS-like tyrosine kinase 3 ligand (FLT3L);
   wherein the deletions in the C2L, F3L, B14R, and B29R vaccinia genes are partial deletions.
26. The nucleic acid of embodiment 25, further comprising a nucleotide sequence comprising at least one promoter operably linked to the third nucleotide sequence.
27. The nucleic acid of embodiment 26, wherein the at least one promoter operably linked to the third nucleotide sequence is a B8R promoter, a B19R promoter, a E3L promoter, an F11L promoter, or a B2R promoter.
28. The nucleic acid of embodiment 26, wherein the at least one promoter operably linked to the third nucleotide sequence is a B8R promoter.
29. The nucleic acid of embodiment 26, wherein the at least one promoter operably linked to the third nucleotide sequence is a B19R promoter.
30. The nucleic acid of embodiment 26, wherein the at least one promoter operably linked to the third nucleotide sequence is a B8R promoter and a B19R promoter.
31. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a third transgene comprising a third nucleotide sequence encoding FLT3L; and
   (d) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter;
   wherein the deletions in the C2L, F3L, B14R, and B29R vaccinia genes are partial deletions.
32. The nucleic acid of embodiment 31, wherein the at least one promoter operably linked to the third nucleotide sequence is a B8R promoter.
33. The nucleic acid of embodiment 31, wherein the at least one promoter operably linked to the third nucleotide sequence is a B19R promoter.
34. The nucleic acid of embodiment 31, wherein the at least one promoter operably linked to the third nucleotide sequence is a B8R promoter and a B19R promoter.
35. The nucleic acid of any one of embodiments 25-34, wherein the third nucleotide sequence is the same orientation as endogenous vaccinia virus genes that flank the third nucleotide sequence.
36. The nucleic acid of any one of embodiments 25-35, wherein the FLT3L comprises the amino acid sequence set forth in SEQ ID NO: 213.
37. The nucleic acid of any one of embodiments 25-36, wherein the third nucleotide sequence comprises the sequence set forth in SEQ ID NO: 216.
38. The nucleic acid of any one of embodiments 25-36, wherein the third nucleotide sequence is set forth in SEQ ID NO: 216.
39. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4; and
   (d) a second transgene comprising a second nucleotide sequence encoding an IL-12 polypeptide;
   wherein the deletions in the C2L, F3L, B14R, and B29R vaccinia genes are partial deletions.
40. The nucleic acid of embodiment 39, further comprising a nucleotide sequence comprising at least one promoter operably linked to the first nucleotide sequence.
41. The nucleic acid of embodiment 40, wherein the at least one promoter operably linked to the first nucleotide sequence is an H5R promoter, a pS promoter, or a LEO promoter.
42. The nucleic acid of embodiment 40, wherein the at least one promoter operably linked to the first nucleotide sequence is an H5R promoter.
43. The nucleic acid of any one of embodiments 39-42, further comprising a nucleotide sequence comprising at least one promoter operably linked to the second nucleotide sequence.
44. The nucleic acid of embodiment 43, wherein the at least one promoter operably linked to the second nucleotide sequence is a late promoter.
45. The nucleic acid of embodiment 44, wherein the late promoter comprises the nucleotide sequence of SEQ ID NO: 561, an F17R promoter, or a D13L promoter.
46. The nucleic acid of embodiment 44, wherein the late promoter comprises the nucleotide sequence of SEQ ID NO: 561.
47. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4; and
   (d) a second transgene comprising a second nucleotide sequence encoding an IL-12 polypeptide;
   wherein the deletions in the C2L, F3L, B14R, and B29R vaccinia genes are partial deletions; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or
   (ii) a nucleotide sequence comprising at least one promoter operably linked to the second nucleotide sequence, wherein the at least one promoter operably linked to the second nucleotide sequence is a late promoter.
48. The nucleic acid comprising embodiment 47, wherein the at least one promoter operably linked to the first nucleotide sequence is an H5R promoter.
49. The nucleic acid comprising embodiment 47 or 48, wherein the late promoter comprises the nucleotide sequence of SEQ ID NO: 561, an F17R promoter, or a D13L promoter.
50. The nucleic acid of embodiment 47 or 48, wherein the late promoter comprises the nucleotide sequence of SEQ ID NO: 561.
51. The nucleic acid of any one of embodiments 39-50, wherein the first nucleotide sequence is in the same orientation as endogenous vaccinia virus genes that flank the first nucleotide sequence, and the second nucleotide sequence is in the same orientation as endogenous vaccinia virus genes that flank the second nucleotide sequence.
52. The nucleic acid of any one of embodiments 39-51, wherein the IL-12 polypeptide is membrane-bound.
53. The nucleic acid of any one of embodiments 39-52, wherein the IL-12 polypeptide comprises IL-12 p35 or IL-12 p70.
54. The nucleic acid of any one of embodiments 39-53, wherein the first nucleotide sequence encodes an amino acid sequence comprising the amino acid sequence set forth in SEQ ID NO: 211.
55. The nucleic acid of any one of embodiments 39-54, wherein the first nucleotide sequence comprises the sequence set forth in SEQ ID NO: 214.
56. The nucleic acid of any one of embodiments 39-54, wherein the first nucleotide sequence is set forth in SEQ ID NO: 214.
57. The nucleic acid of any one of embodiments 39-56, wherein the IL-12 polypeptide comprises the amino acid sequence set forth in SEQ ID NO: 212.
58. The nucleic acid of any one of embodiments 39-57, wherein the second nucleotide sequence comprises the sequence set forth in SEQ ID NO: 215.
59. The nucleic acid of any one of embodiments 39-57, wherein the second nucleotide sequence is set forth in SEQ ID NO: 215.
60. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4; and
   (d) a third transgene comprising a third nucleotide sequence encoding FLT3L; wherein the deletions in the C2L, F3L, B14R, and B29R vaccinia genes are partial deletions.
61. The nucleic acid of embodiment 60, further comprising a nucleotide sequence comprising at least one promoter operably linked to the first nucleotide sequence.
62. The nucleic acid of embodiment 61, wherein the at least one promoter operably linked to the first nucleotide sequence is an H5R promoter, a pS promoter, or a LEO promoter.
63. The nucleic acid of embodiment 61, wherein the at least one promoter operably linked to the first nucleotide sequence is an H5R promoter.
64. The nucleic acid of any one of embodiments 60-63, further comprising a nucleotide sequence comprising at least one promoter operably linked to the third nucleotide sequence.
65. The nucleic acid of embodiment 64, wherein the at least one promoter operably linked to the third nucleotide sequence is a B8R promoter, a B19R promoter, a E3L promoter, an F11L promoter, or a B2R promoter.
66. The nucleic acid of embodiment 64, wherein the at least one promoter operably linked to the third nucleotide sequence is a B8R promoter.
67. The nucleic acid of embodiment 64, wherein the at least one promoter operably linked to the third nucleotide sequence is a B19R promoter.
68. The nucleic acid of embodiment 64, wherein the at least one promoter operably linked to the third nucleotide sequence is a B8R promoter and a B19R promoter.
69. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4; and
   (d) a third transgene comprising a third nucleotide sequence encoding FLT3L; wherein the deletions in the C2L, F3L, B14R, and B29R vaccinia genes are partial deletions; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter, a pS promoter, or a LEO promoter; and/or
   (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter.
70. The nucleic acid of embodiment 69, wherein the at least one promoter operably linked to the first nucleotide sequence is an H5R promoter.
71. The nucleic acid of embodiment 69 or 70, wherein the at least one promoter operably linked to the third nucleotide sequence is a B8R promoter.
72. The nucleic acid of embodiment 69 or 70, wherein the at least one promoter operably linked to the third nucleotide sequence is a B19R promoter.
73. The nucleic acid of embodiment 69 or 70, wherein the at least one promoter operably linked to the third nucleotide sequence is a B8R promoter and a B19R promoter.
74. The nucleic acid of any one of embodiments 60-73, wherein the first nucleotide sequence is in the same orientation as endogenous vaccinia virus genes that flank the first nucleotide sequence and the third nucleotide sequence is in the same orientation as endogenous vaccinia virus genes that flank the third nucleotide sequence.
75. The nucleic acid of any one of embodiments 60-74, wherein the first nucleotide sequence encodes an amino acid sequence comprising the amino acid sequence set forth in SEQ ID NO: 211.
76. The nucleic acid of any one of embodiments 60-75, wherein the first nucleotide sequence comprises the sequence set forth in SEQ ID NO: 214.
77. The nucleic acid of any one of embodiments 60-75, wherein the first nucleotide sequence is set forth in SEQ ID NO: 214.
78. The nucleic acid of any one of embodiments 60-77, wherein the FLT3L comprises the amino acid sequence set forth in SEQ ID NO: 213.
79. The nucleic acid of any one of embodiments 60-78, wherein the third nucleotide sequence comprises the sequence set forth in SEQ ID NO: 216.
80. The nucleic acid of any one of embodiments 60-78, wherein the third nucleotide sequence is set forth in SEQ ID NO: 216.
81. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R;
   (c) a second transgene comprising a second nucleotide sequence encoding an IL-12 polypeptide; and
   (d) a third transgene comprising a third nucleotide sequence encoding FLT3L; wherein the deletions in the C2L, F3L, B14R, and B29R vaccinia genes are partial deletions.
82. The nucleic acid of embodiment 81, further comprising a nucleotide sequence comprising at least one promoter operably linked to the second nucleotide sequence.
83. The nucleic acid of embodiment 82, wherein the at least one promoter operably linked to the second nucleotide sequence is a late promoter.
84. The nucleic acid of embodiment 83, wherein the late promoter comprises the nucleotide sequence of SEQ ID NO: 561, an F17R promoter, or a D13L promoter.
85. The nucleic acid of embodiment 83, wherein the late promoter comprises the nucleotide sequence of SEQ ID NO: 561.
86. The nucleic acid of any one of embodiments 81-85, further comprising a nucleotide sequence comprising at least one promoter operably linked to the third nucleotide sequence.
87. The nucleic acid of embodiment 86, wherein the at least one promoter operably linked to the third nucleotide sequence is a B8R promoter, a B19R promoter, a E3L promoter, an F11L promoter, or a B2R promoter.
88. The nucleic acid of embodiment 86, wherein the at least one promoter operably linked to the third nucleotide sequence is a B8R promoter.
89. The nucleic acid of embodiment 86, wherein the at least one promoter operably linked to the third nucleotide sequence is a B19R promoter.
90. The nucleic acid of embodiment 86, wherein the at least one promoter operably linked to the third nucleotide sequence is a B8R promoter and a B19R promoter.
91. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a second transgene comprising a second nucleotide sequence encoding an IL-12 polypeptide; and
   (d) a third transgene comprising a third nucleotide sequence encoding FLT3L;
   wherein the deletions in the C2L, F3L, B14R, and B29R vaccinia genes are partial deletions; and
   wherein the nucleic acid further comprises:
   (i) a nucleotide sequence comprising at least one promoter operably linked to the second nucleotide sequence, wherein the at least one promoter operably linked to the second nucleotide sequence is a late promoter; and/or
   (ii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter.
92. The nucleic acid of embodiment 91, wherein the late promoter comprises the nucleotide sequence of SEQ ID NO: 561, an F17R promoter, or a D13L promoter.
93. The nucleic acid of embodiment 91, wherein the late promoter comprises the nucleotide sequence of SEQ ID NO: 561.
94. The nucleic acid of any one of embodiments 91-93, wherein the at least one promoter operably linked to the third nucleotide sequence is a B8R promoter.
95. The nucleic acid of any one of embodiments 91-93, wherein the at least one promoter operably linked to the third nucleotide sequence is a B19R promoter.
96. The nucleic acid of any one of embodiments 91-93, wherein the at least one promoter operably linked to the third nucleotide sequence is a B8R promoter and a B19R promoter.
97. The nucleic acid of any one of embodiments 81-96, wherein the second nucleotide sequence is in the same orientation as endogenous vaccinia virus genes that flank the second nucleotide sequence, and the third nucleotide sequence is in the same orientation as endogenous vaccinia genes that flank the third nucleotide sequence.
98. The nucleic acid of any one of embodiments 81-97, wherein the IL-12 polypeptide is membrane-bound.
99. The nucleic acid of any one of embodiments 81-98, wherein the IL-12 polypeptide comprises IL-12 p35 or IL-12 p70.
100. The nucleic acid of any one of embodiments 81-99, wherein the IL-12 polypeptide comprises the amino acid sequence set forth in SEQ ID NO: 212.
101. The nucleic acid of any one of embodiments 81-100, wherein the second nucleotide sequence comprises the sequence set forth in SEQ ID NO: 215.
102. The nucleic acid of any one of embodiments 81-100, wherein the second nucleotide sequence is set forth in SEQ ID NO: 215.
103. The nucleic acid of any one of embodiments 81-102, wherein the FLT3L comprises the amino acid sequence set forth in SEQ ID NO: 213.
104. The nucleic acid of any one of embodiments 81-103, wherein the third nucleotide sequence comprises the sequence set forth in SEQ ID NO: 216.
105. The nucleic acid of any one of embodiments 81-103, wherein the third nucleotide sequence is set forth in SEQ ID NO: 216.
106. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B8R, B14R, B15R, B16R, B17L, B18R, B 19R, and B20R;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4;
   (d) a second transgene comprising a second nucleotide sequence encoding an IL-12 polypeptide; and
   (e) a third transgene comprising a third nucleotide sequence encoding FLT3L;
   wherein the deletions in the C2L, F3L, B14R, and B29R vaccinia genes are partial deletions.
107. The nucleic acid of embodiment 106, further comprising a nucleotide sequence comprising at least one promoter operably linked to the first nucleotide sequence.
108. The nucleic acid of embodiment 107, wherein the at least one promoter operably linked to the first nucleotide sequence is an H5R promoter, a pS promoter, or a LEO promoter.
109. The nucleic acid of embodiment 107, wherein the at least one promoter operably linked to the first nucleotide sequence is an H5R promoter.
110. The nucleic acid of any one of embodiments 106-109, further comprising a nucleotide sequence comprising at least one promoter operably linked to the second nucleotide sequence.
111. The nucleic acid of embodiment 110, wherein the at least one promoter operably linked to the second nucleotide sequence is a late promoter.
112. The nucleic acid of embodiment 111, wherein the late promoter comprises the nucleotide sequence of SEQ ID NO: 561, an F17R promoter, or a D13L promoter.
113. The nucleic acid of embodiment 111, wherein the late promoter comprises the nucleotide sequence of SEQ ID NO: 561.
114. The nucleic acid of any one of embodiments 106-113, further comprising a nucleotide sequence comprising at least one promoter operably linked to the third nucleotide sequence.
115. The nucleic acid of embodiment 114, wherein the at least one promoter operably linked to the third nucleotide sequence is a B8R promoter, a B19R promoter, a E3L promoter, an F 11L promoter, or a B2R promoter.
116. The nucleic acid of embodiment 114, wherein the at least one promoter operably linked to the third nucleotide sequence is a B8R promoter.
117. The nucleic acid of embodiment 114, wherein the at least one promoter operably linked to the third nucleotide sequence is a B19R promoter.
118. The nucleic acid of embodiment 114, wherein the at least one promoter operably linked to the third nucleotide sequence is a B8R promoter and a B19R promoter.
119. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4;
   (d) a second transgene comprising a second nucleotide sequence encoding an IL-12 polypeptide; and
   (e) a third transgene comprising a third nucleotide sequence encoding FLT3L;
   wherein the deletions in the C2L, F3L, B14R, and B29R vaccinia genes are partial deletions; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter, a pS promoter, or a LEO promoter;
   (ii) a nucleotide sequence comprising at least one promoter operably linked to the second nucleotide sequence, wherein the at least one promoter operably linked to the second nucleotide sequence is a late promoter; and/or
   (iii) a nucleotide sequence comprising at least one promoter operably linked to the third nucleotide sequence, wherein the at least one promoter operably linked to the third nucleotide sequence is a B8R promoter, a B19R promoter, a E3L promoter, an F11L promoter, or a B2R promoter.
120. The nucleic acid of embodiment 119, wherein the at least one promoter operably linked to the first nucleotide sequence is an H5R promoter.
121. The nucleic acid of embodiment 119 or 120, wherein the late promoter comprises the nucleotide sequence of SEQ ID NO: 561, an F17R promoter, or a D13L promoter.
122. The nucleic acid of embodiment 119 or 120, wherein the late promoter comprises the nucleotide sequence of SEQ ID NO: 561.
123. The nucleic acid of any one of embodiments 119-122, wherein the at least one promoter operably linked to the third nucleotide sequence is a B8R promoter.
124. The nucleic acid of any one of embodiments 119-122, wherein the at least one promoter operably linked to the third nucleotide sequence is a B19R promoter.
125. The nucleic acid of any one of embodiments 119-122, wherein the at least one promoter operably linked to the third nucleotide sequence is a B8R promoter and a B19R promoter.
126. The nucleic acid of any one of embodiments 106-125, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.
127. The nucleic acid of any one of embodiments 106-126, wherein the IL-12 polypeptide is membrane-bound.
128. The nucleic acid of any one of embodiments 106-127, wherein the IL-12 polypeptide comprises IL-12 p35 or IL-12 p70.
129. The nucleic acid of any one of embodiments 106-128, wherein the first nucleotide sequence encodes an amino acid sequence comprising the amino acid sequence set forth in SEQ ID NO: 211.
130. The nucleic acid of any one of embodiments 106-129, wherein the first nucleotide sequence comprises the sequence set forth in SEQ ID NO: 214.
131. The nucleic acid of any one of embodiments 106-129, wherein the first nucleotide sequence is set forth in SEQ ID NO: 214.
132. The nucleic acid of any one of embodiments 106-131, wherein the IL-12 polypeptide comprises the amino acid sequence set forth in SEQ ID NO: 212.
133. The nucleic acid of any one of embodiments 106-132, wherein the second nucleotide sequence comprises the sequence set forth in SEQ ID NO: 215.
134. The nucleic acid of any one of embodiments 106-132, wherein the second nucleotide sequence is set forth in SEQ ID NO: 215.
135. The nucleic acid of any one of embodiments 106-134, wherein the FLT3L comprises the amino acid sequence set forth in SEQ ID NO: 213.
136. The nucleic acid of any one of embodiments 106-135, wherein the third nucleotide sequence comprises the sequence set forth in SEQ ID NO: 216.
137. The nucleic acid of any one of embodiments 106-135, wherein the third nucleotide sequence is set forth in SEQ ID NO: 216.
138. The nucleic acid of any one of embodiments 106-137, wherein the first transgene is present between the partial C2L and F3L vaccinia genes, and the second transgene and the third transgene are present in the locus of the deletion in the B8R gene.
139. The nucleic acid of embodiment 138, wherein the third transgene is upstream of the second transgene.
140. The nucleic acid of any one of embodiments 1-10, 39-80, and 106-137, wherein the first transgene is present between the partial C2L and F3L vaccinia genes.
141. The nucleic acid of any one of embodiments 11-24, 39-59, and 81-137, wherein the second transgene is present between the partial C2L and F3L vaccinia genes.
142. The nucleic acid of any one of embodiments 25-38 and 60-137, wherein the third transgene is present between the partial C2L and F3L vaccinia genes.
143. The nucleic acid of any one of embodiments 1-10, 39-80, and 106-137, wherein the first transgene is present in the locus of the deletion in the B8R gene.
144. The nucleic acid of any one of embodiments 11-24, 39-59, and 81-137, wherein the second transgene is present in the locus of the deletion in the B8R gene.
145. The nucleic acid of any one of embodiments 25-38 and 60-137, wherein the third transgene is present in the locus of the deletion in the B8R gene.
146. The nucleic acid of any one of embodiments 1-10, 39-80, and 106-137, wherein the first transgene is present between the partial B14R and B29R vaccinia genes.
147. The nucleic acid of any one of embodiments 11-24, 39-59, and 81-137, wherein the second transgene is present between the partial B14R and B29R vaccinia genes.
148. The nucleic acid of any one of embodiments 25-38 and 60-137, wherein the third transgene is present between the partial B14R and B29R vaccinia genes.
149. The nucleic acid of any one of embodiments 39-59 and 106-137, wherein the first transgene and the second transgene are present between the partial C2L and F3L vaccinia genes.
150. The nucleic acid of any one of embodiments 39-59 and 106-137, wherein the first transgene and the second transgene are present in the locus of the deletion in the B8R gene.
151. The nucleic acid of any one of embodiments 39-59 and 106-137, wherein the first transgene and the second transgene are present between the partial B14R and B29R vaccinia genes.
152. The nucleic acid of any one of embodiments 60-80 and 106-137, wherein the first transgene and the third transgene are present between the partial C2L and F3L vaccinia genes.
153. The nucleic acid of any one of embodiments 60-80 and 106-137, wherein the first transgene and the third transgene are present in the locus of the deletion in the B8R gene.
154. The nucleic acid of any one of embodiments 60-80 and 106-137, wherein the first transgene and the third transgene are present between the partial B14R and B29R vaccinia genes.
155. The nucleic acid of any one of embodiments 81-137, wherein the second transgene and the third transgene are present between the partial C2L and F3L vaccinia genes.
156. The nucleic acid of any one of embodiments 81-137, wherein the second transgene and the third transgene are present in the locus of the deletion in the B8R gene.
157. The nucleic acid of any one of embodiments 81-137, wherein the second transgene and the third transgene are present between the partial B14R and B29R vaccinia genes.
158. The nucleic acid of any one of embodiments 39-59 and 106-137, wherein the first transgene is present between the partial C2L and F3L vaccinia genes, and the second transgene is present in the locus of the deletion in the B8R gene.
159. The nucleic acid of any one of embodiments 39-59 and 106-137, wherein the second transgene is present between the partial C2L and F3L vaccinia genes, and the first transgene is present in the locus of the deletion in the B8R gene.
160. The nucleic acid of any one of embodiments 60-80 and 106-137, wherein the first transgene is present between the partial C2L and F3L vaccinia genes, and the third transgene is present in the locus of the deletion in the B8R gene.
161. The nucleic acid of any one of embodiments 60-80 and 106-137, wherein the third transgene is present between the partial C2L and F3L vaccinia genes, and the first transgene is present in the locus of the deletion in the B8R gene.
162. The nucleic acid of any one of embodiments 81-137, wherein the second transgene is present between the partial C2L and F3L vaccinia genes, and the third transgene is present in the locus of the deletion in the B8R gene.
163. The nucleic acid of any one of embodiments 81-137, wherein the third transgene is present between the partial C2L and F3L vaccinia genes, and the second transgene is present in the locus of the deletion in the B8R gene.
164. The nucleic acid of any one of embodiments 39-59 and 106-137, wherein the first transgene is present between the partial C2L and F3L vaccinia genes, and the second transgene is present between the partial B14R and B29R vaccinia genes.
165. The nucleic acid of any one of embodiments 39-59 and 106-137, wherein the second transgene is present between the partial C2L and F3L vaccinia genes, and the first transgene is present between the partial B14R and B29R vaccinia genes.
166. The nucleic acid of any one of embodiments 60-80 and 106-137, wherein the first transgene is present between the partial C2L and F3L vaccinia genes, and the third transgene is present between the partial B14R and B29R vaccinia genes.
167. The nucleic acid of any one of embodiments 60-80 and 106-137, wherein the third transgene is present between the partial C2L and F3L vaccinia genes, and the first transgene is present between the partial B14R and B29R vaccinia genes.
168. The nucleic acid of any one of embodiments 81-137, wherein the second transgene is present between the partial C2L and F3L vaccinia genes, and the third transgene is present between the partial B14R and B29R vaccinia genes.
169. The nucleic acid of any one of embodiments 81-137, wherein the third transgene is present between the partial C2L and F3L vaccinia genes, and the second transgene is present between the partial B14R and B29R vaccinia genes.
170. The nucleic acid of any one of embodiments 39-59 and 106-137, wherein the first transgene is present in the locus of the deletion in the B8R gene, and the second transgene is present between the partial B14R and B29R vaccinia genes.
171. The nucleic acid of any one of embodiments 39-59 and 106-137, wherein the second transgene is present in the locus of the deletion in the B8R gene, and the first transgene is present between the partial B14R and B29R vaccinia genes.
172. The nucleic acid of any one of embodiments 60-80 and 106-137, wherein the first transgene is present in the locus of the deletion in the B8R gene, and the third transgene is present between the partial B14R and B29R vaccinia genes.
173. The nucleic acid of any one of embodiments 60-80 and 106-137, wherein the third transgene is present in the locus of the deletion in the B8R gene, and the first transgene is present between the partial B14R and B29R vaccinia genes.
174. The nucleic acid of any one of embodiments 81-137, wherein the second transgene is present in the locus of the deletion in the B8R gene, and the third transgene is present between the partial B14R and B29R vaccinia genes.
175. The nucleic acid of any one of embodiments 81-137, wherein the third transgene is present in the locus of the deletion in the B8R gene, and the second transgene is present between the partial B14R and B29R vaccinia genes.
176. The nucleic acid of any one of embodiments 106-137, wherein the first transgene, the second transgene, and the third transgene are present between the partial C2L and F3L vaccinia genes.
177. The nucleic acid of any one of embodiments 106-137, wherein the first transgene, the second transgene, and the third transgene are present in the locus of the deletion in the B8R gene.
178. The nucleic acid of any one of embodiments 106-137, wherein the first transgene, the second transgene, and the third transgene are present between the partial B14R and B29R vaccinia genes.
179. The nucleic acid of any one of embodiments 106-137, wherein the first transgene is present between the partial C2L and F3L vaccinia genes, and the second transgene and the third transgene are present in the locus of the deletion in the B8R gene.
180. The nucleic acid of any one of embodiments 106-137, wherein the second transgene is present between the partial C2L and F3L vaccinia genes, and the first transgene and the third transgene are present in the locus of the deletion in the B8R gene.
181. The nucleic acid of any one of embodiments 106-137, wherein the third transgene is present between the partial C2L and F3L vaccinia genes, and the first transgene and the second transgene are present in the locus of the deletion in the B8R gene.
182. The nucleic acid of any one of embodiments 106-137, wherein the first transgene and the second transgene are present between the partial C2L and F3L vaccinia genes, and the third transgene is present in the locus of the deletion in the B8R gene.
183. The nucleic acid of any one of embodiments 106-137, wherein the first transgene and the third transgene are present between the partial C2L and F3L vaccinia genes, and the second transgene is present in the locus of the deletion in the B8R gene.
184. The nucleic acid of any one of embodiments 106-137, wherein the second transgene and the third transgene are present between the partial C2L and F3L vaccinia genes, and the first transgene is present in the locus of the deletion in the B8R gene.
185. The nucleic acid of any one of embodiments 106-137, wherein the first transgene is present within between the partial C2L and F3L vaccinia genes, and the second transgene and the third transgene are present between the partial B14R and B29R vaccinia genes.
186. The nucleic acid of any one of embodiments 106-137, wherein the second transgene is present between the partial C2L and F3L vaccinia genes, and the first transgene and the third transgene are present between the partial B14R and B29R vaccinia genes.
187. The nucleic acid of any one of embodiments 106-137, wherein the third transgene is present between the partial C2L and F3L vaccinia genes, and the first transgene and the second transgene are present between the partial B14R and B29R vaccinia genes.
188. The nucleic acid of any one of embodiments 106-137, wherein the first transgene and the second transgene are present between the partial C2L and F3L vaccinia genes, and the third transgene is present between the partial B14R and B29R vaccinia genes.
189. The nucleic acid of any one of embodiments 106-137, wherein the first transgene and the third transgene are present between the partial C2L and F3L vaccinia genes, and the second transgene is present between the partial B14R and B29R vaccinia genes.
190. The nucleic acid of any one of embodiments 106-137, wherein the second transgene and the third transgene are present between the partial C2L and F3L vaccinia genes, and the first transgene is present between the partial B14R and B29R vaccinia genes.
191. The nucleic acid of any one of embodiments 106-137, wherein the first transgene is present in the locus of the deletion in the B8R gene, and the second transgene and the third transgene are present between the partial B14R and B29R vaccinia genes.
192. The nucleic acid of any one of embodiments 106-137, wherein the second transgene is present in the locus of the deletion in the B8R gene, and the first transgene and the third transgene are present between the partial B14R and B29R vaccinia genes.
193. The nucleic acid of any one of embodiments 106-137, wherein the third transgene is present in the locus of the deletion in the B8R gene, and the first transgene and the second transgene are present between the partial B14R and B29R vaccinia genes.
194. The nucleic acid of any one of embodiments 106-137, wherein the first transgene and the second transgene are present in the locus of the deletion in the B8R gene, and the third transgene is present between the partial B14R and B29R vaccinia genes.
195. The nucleic acid of any one of embodiments 106-137, wherein the first transgene and the third transgene are present in the locus of the deletion in the B8R gene, and the second transgene is present between the partial B14R and B29R vaccinia genes.
196. The nucleic acid of any one of embodiments 106-137, wherein the second transgene and the third transgene are present in the locus of the deletion in the B8R gene, and the first transgene is present between the partial B14R and B29R vaccinia genes.
197. The nucleic acid of any one of embodiments 106-137, wherein the first transgene is present between the partial C2L and F3L vaccinia genes, the second transgene is present in the locus of the deletion in the B8R gene, and the third transgene is present between the partial B14R and B29R vaccinia genes.
198. The nucleic acid of any one of embodiments 106-137, wherein the first transgene is present between the partial C2L and F3L vaccinia genes, the third transgene is present in the locus of the deletion in the B8R gene, and the second transgene is present between the partial B14R and B29R vaccinia genes.
199. The nucleic acid of any one of embodiments 106-137, wherein the second transgene is present between the partial C2L and F3L vaccinia genes, the first transgene is present in the locus of the deletion in the B8R gene, and the third transgene is present between the partial B14R and B29R vaccinia genes.
200. The nucleic acid of any one of embodiments 106-137, wherein the second transgene is present between the partial C2L and F3L vaccinia genes, the third transgene is present in the locus of the deletion in the B8R gene, and the first transgene is present between the partial B14R and B29R vaccinia genes.
201. The nucleic acid of any one of embodiments 106-137, wherein the third transgene is present between the partial C2L and F3L vaccinia genes, the first transgene is present in the locus of the deletion in the B8R gene, and the second transgene is present between the partial B14R and B29R vaccinia genes.
202. The nucleic acid of any one of embodiments 106-137, wherein the third transgene is present between the partial C2L and F3L vaccinia genes, the second transgene is present in the locus of the deletion in the B8R gene, and the first transgene is present between the partial B14R and B29R vaccinia genes.
203. The nucleic acid of any one of embodiments 1-202, wherein the deletion in the B8R gene is a deletion of at least 50% of the B8R gene sequence.
204. The nucleic acid of any one of embodiments 1-202, wherein the deletion in the B8R gene is a deletion of at least 60% of the B8R gene sequence.
205. The nucleic acid of any one of embodiments 1-202, wherein the deletion in the B8R gene is a deletion of at least 70% of the B8R gene sequence.
206. The nucleic acid of any one of embodiments 1-202, wherein the deletion in the B8R gene is a deletion of at least 80% of the B8R gene sequence.
207. The nucleic acid of any one of embodiments 1-202, wherein the deletion in the B8R gene is a deletion of about 75% of the B8R gene sequence.
208. The nucleic acid of any one of embodiments 1-202, wherein the deletion in the B8R gene is a deletion of about 80% of the B8R gene sequence.
209. The nucleic acid of any one of embodiments 1-208, wherein the recombinant vaccinia virus genome is derived from the genome of a Copenhagen strain vaccinia virus.
210. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4;
   (d) a second transgene comprising a second nucleotide sequence encoding an IL-12 polypeptide; and
   (e) a third transgene comprising a third nucleotide sequence encoding FLT3L; wherein the deletions in the C2L, F3L, B14R, and B29R vaccinia genes are partial deletions; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter and a B19R promoter.
211. The nucleic acid of embodiment 210, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.
212. The nucleic acid of embodiment 210 or 211, wherein the first transgene is present between the partial C2L and F3L vaccinia genes, and the second transgene and the third transgene are present in the locus of the deletion in the B8R gene.
213. The nucleic acid of embodiment 210 or 211, wherein the first transgene is present between the partial B14R and B29R vaccinia genes, and the second transgene and the third transgene are present in the locus of the deletion in the B8R gene.
214. The nucleic acid of embodiment 212 or 213, wherein the third transgene is upstream of the second transgene.
215. The nucleic acid of embodiment 212 or 213, wherein the third transgene is downstream of the second transgene.
216. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is present between the partial C2L and F3L vaccinia genes;
   (d) a second transgene comprising a second nucleotide sequence encoding an IL-12 polypeptide, wherein the second nucleotide sequence is in the same orientation as endogenous vaccinia virus genes that flank the second nucleotide sequence, and wherein the second transgene is present in the locus of the deletion in the B8R gene; and
   (e) a third transgene comprising a third nucleotide sequence encoding FLT3L, wherein the third nucleotide sequence is in the same orientation as endogenous vaccinia virus genes that flank the third nucleotide sequence, wherein the third transgene is present in the locus of the deletion in the B8R gene, and wherein the third transgene is upstream of the second transgene;
   wherein the deletions in the C2L, F3L, B14R, and B29R vaccinia genes are partial deletions; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter and a B19R promoter.
217. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is present between the partial C2L and F3L vaccinia genes;
   (d) a second transgene comprising a second nucleotide sequence encoding an IL-12 polypeptide, wherein the second nucleotide sequence is in the same orientation as endogenous vaccinia virus genes that flank the second nucleotide sequence, and
   wherein the second transgene is present in the locus of the deletion in the B8R gene; and
   (e) a third transgene comprising a third nucleotide sequence encoding FLT3L;
   wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is present in the locus of the deletion in the B8R gene, and wherein the third transgene is downstream of the second transgene; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter and a B19R promoter.
218. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is present between the partial B14R and B29R vaccinia genes;
   (d) a second transgene comprising a second nucleotide sequence encoding an IL-12 polypeptide, wherein the second nucleotide sequence is in the same orientation as endogenous vaccinia virus genes that flank the second nucleotide sequence, and 1

wherein the second transgene is present in the locus of the deletion in the B8R gene; and
(e) a third transgene comprising a third nucleotide sequence encoding FLT3L;
wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is present in the locus of the deletion in the B8R gene, and wherein the third transgene is upstream of the second transgene; and
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter and a B19R promoter.
219. A nucleic acid comprising a recombinant vaccinia virus genome, comprising:
   (a) deletions in the following genes: C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R, and optionally a deletion in the B8R gene;
   (b) deletions in the following genes in the 3′ ITR: B21R, B22R, B23R, B24R, B25R, B26R, B27R, B28R, and B29R;
   (c) a first transgene comprising a first nucleotide sequence encoding an antibody or antigen-binding fragment thereof that specifically binds to CTLA-4, wherein the first nucleotide sequence is in the same orientation as endogenous vaccinia virus genes that flank the first nucleotide sequence, and wherein the first transgene is present between the partial B14R and B29R vaccinia genes;
   (d) a second transgene comprising a second nucleotide sequence encoding an IL-12 polypeptide, wherein the second nucleotide sequence is in the same orientation as endogenous vaccinia virus genes that flank the second nucleotide sequence, and
   wherein the second transgene is present in the locus of the deletion in the B8R gene; and
   (e) a third transgene comprising a third nucleotide sequence encoding FLT3L;
   wherein the deletions in the C2L, F3L, B14R, and B29R vaccinia genes are partial deletions, wherein the third nucleotide sequence is in the same orientation as endogenous vaccinia virus genes that flank the third nucleotide sequence, wherein the third transgene is present in the locus of the deletion in the B8R gene, and wherein the third transgene is downstream of the second transgene; and
   wherein the nucleic acid further comprises:
   (i) a nucleotide sequence comprising at least one promoter operably linked to the first nucleotide sequence, wherein the at least one promoter operably linked to the first nucleotide sequence is an H5R promoter;
   (ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
   (iii) a nucleotide sequence comprising at least one promoter operably linked to the third nucleotide sequence, wherein the at least one promoter operably linked to the third nucleotide sequence is a B8R promoter and a B19R promoter.
220. The nucleic acid of any one of embodiments 1-219, wherein the recombinant vaccinia virus genome comprises a vaccinia virus nucleotide sequence of SEQ ID NO: 624.
221. The nucleic acid of any one of embodiments 1-105, wherein the recombinant vaccinia virus genome comprises a vaccinia virus nucleotide sequence of SEQ ID NO: 210.
222. A virus comprising the nucleic acid comprising a recombinant vaccinia virus genome of any one of embodiments 1-221.
223. A packaging cell line comprising the nucleic acid of any one of embodiments 1-221.
224. A packaging cell line comprising the virus of embodiment 222.
225. A pharmaceutical composition comprising the virus of embodiment 222 and a physiologically acceptable carrier.
226. A kit comprising the nucleic acid of any one of embodiments 1-221 and a package insert instructing a user of the kit to express the nucleic acid in a host cell.
227. A kit comprising the virus of embodiment 222 and a package insert instructing a user of the kit to express the virus in a host cell.
228. A kit comprising the virus of embodiment 222 and a package insert instructing a user to administer a therapeutically effective amount of the virus to a mammalian patient having cancer, thereby treating the cancer.
229. The kit of embodiment 228, wherein the mammalian patient is a human patient.
230. A method of treating cancer in a mammalian patient, the method comprising administering to the mammalian patient a therapeutically effective amount of the virus of embodiment 222.
231. A method of treating cancer in a mammalian patient, the method comprising administering to the mammalian patient a therapeutically effective amount of the pharmaceutical composition of embodiment 225.
232. The method of embodiment 230 or 231, wherein the mammalian patient is a human patient.
233. The method of any one of embodiments 230-232, wherein the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.
234. The method of any one of embodiments 230-233, wherein the method further comprises administering to the mammalian patient an immune checkpoint inhibitor.
235. The method of embodiment 234, wherein the immune checkpoint inhibitor is selected from the group consisting of OX40 ligand, ICOS ligand, anti-CD47 antibody or antigen-binding fragment thereof, anti-CD40/CD40L antibody or antigen-binding fragment thereof, anti-Lag3 antibody or antigen-binding fragment thereof, anti-CTLA-4 antibody or antigen-binding fragment thereof, anti-PD-L1 antibody or antigen-binding fragment thereof, anti-PD1 antibody or antigen-binding fragment thereof, and anti-Tim-3 antibody or antigen-binding fragment thereof.
236. The method of embodiment 234, wherein the immune checkpoint inhibitor is an anti-PD1 antibody or antigen-binding fragment thereof or an anti-CTLA-4 antibody or antigen-binding fragment thereof.
237. The method of embodiment 234, wherein the immune checkpoint inhibitor is an anti-PD1 antibody or antigen-binding fragment thereof.
238. The method of embodiment 234, wherein the immune checkpoint inhibitor is an anti-CTLA-4 antibody or antigen-binding fragment thereof.
239. The method of embodiment 234, wherein the immune checkpoint inhibitor is an anti-PD-L1 antibody or antigen-binding fragment thereof.
240. The method of any one of embodiments 219-228, wherein the method further comprises administering to the mammalian patient an interleukin.
241. The method of embodiment 240, wherein the interleukin is selected from the group consisting of IL-1 alpha, IL-1 beta, IL-2, IL-4, IL-7, IL-10, IL-12 p35, IL-12 p40, IL-12 p70, IL-15, IL-18, IL-21, and IL-23.
242. The method of embodiment 240, wherein the interleukin is selected from the group consisting of IL-12 p35, IL-12 p40, and IL-12 p70.
243. The method of any one of embodiments 240-242, wherein the interleukin is membrane-bound.
244. The method of any one of embodiments 230-243, wherein the method further comprises administering to the mammalian patient an interferon.
245. The method of embodiment 244, wherein the interferon is selected from the group consisting of IFN-alpha, IFN-beta, IFN-delta, IFN-epsilon, IFN-tau, IFN-omega, IFN-zeta, and IFN-gamma.
246. The method of any one of embodiments 230-245, wherein the method further comprises administering to the mammalian patient a cytokine.
247. The method of embodiment 246, wherein the cytokine is a TNF superfamily member protein.
248. The method of embodiment 247, wherein the TNF superfamily member protein is selected from the group consisting of TRAIL, Fas ligand, LIGHT (TNFSF-14), TNF-alpha, and 4-1BB ligand.
249. The method of embodiment 246, wherein the cytokine is selected from the group consisting of GM-CSF, Flt3 ligand, CD40 ligand, TGF-beta, VEGF-R2, and cKit.
250. The method of embodiment 246, wherein the cytokine is Flt3 ligand.

### 5.8. Tables 3-45 Referenced in the Application

Lengthy table referenced here
US20220056480A1-20220224-T00001
Please refer to the end of the specification for access instructions.

Lengthy table referenced here
US20220056480A1-20220224-T00002
Please refer to the end of the specification for access instructions.

Lengthy table referenced here
US20220056480A1-20220224-T00003
Please refer to the end of the specification for access instructions.

Lengthy table referenced here
US20220056480A1-20220224-T00004
Please refer to the end of the specification for access instructions.

Lengthy table referenced here
US20220056480A1-20220224-T00005
Please refer to the end of the specification for access instructions.

Lengthy table referenced here
US20220056480A1-20220224-T00006
Please refer to the end of the specification for access instructions.

Lengthy table referenced here
US20220056480A1-20220224-T00007
Please refer to the end of the specification for access instructions.

Lengthy table referenced here
US20220056480A1-20220224-T00008
Please refer to the end of the specification for access instructions.

Lengthy table referenced here
US20220056480A1-20220224-T00009
Please refer to the end of the specification for access instructions.

Lengthy table referenced here
US20220056480A1-20220224-T00010
Please refer to the end of the specification for access instructions.

Lengthy table referenced here
US20220056480A1-20220224-T00011
Please refer to the end of the specification for access instructions.

Lengthy table referenced here
US20220056480A1-20220224-T00012
Please refer to the end of the specification for access instructions.

Lengthy table referenced here
US20220056480A1-20220224-T00013
Please refer to the end of the specification for access instructions.

Lengthy table referenced here
US20220056480A1-20220224-T00014
Please refer to the end of the specification for access instructions.

Lengthy table referenced here
US20220056480A1-20220224-T00015
Please refer to the end of the specification for access instructions.

Lengthy table referenced here
US20220056480A1-20220224-T00016
Please refer to the end of the specification for access instructions.

Lengthy table referenced here
US20220056480A1-20220224-T00017
Please refer to the end of the specification for access instructions.

Lengthy table referenced here
US20220056480A1-20220224-T00018
Please refer to the end of the specification for access instructions.

Lengthy table referenced here
US20220056480A1-20220224-T00019
Please refer to the end of the specification for access instructions.

Lengthy table referenced here
US20220056480A1-20220224-T00020
Please refer to the end of the specification for access instructions.

Lengthy table referenced here
US20220056480A1-20220224-T00021
Please refer to the end of the specification for access instructions.

Lengthy table referenced here
US20220056480A1-20220224-T00022
Please refer to the end of the specification for access instructions.

Lengthy table referenced here
US20220056480A1-20220224-T00023
Please refer to the end of the specification for access instructions.

Lengthy table referenced here
US20220056480A1-20220224-T00024
Please refer to the end of the specification for access instructions.

Lengthy table referenced here
US20220056480A1-20220224-T00025
Please refer to the end of the specification for access instructions.

Lengthy table referenced here
US20220056480A1-20220224-T00026
Please refer to the end of the specification for access instructions.

Lengthy table referenced here
US20220056480A1-20220224-T00027
Please refer to the end of the specification for access instructions.

Lengthy table referenced here
US20220056480A1-20220224-T00028
Please refer to the end of the specification for access instructions.

Lengthy table referenced here
US20220056480A1-20220224-T00029
Please refer to the end of the specification for access instructions.

Lengthy table referenced here
US20220056480A1-20220224-T00030
Please refer to the end of the specification for access instructions.

Lengthy table referenced here
US20220056480A1-20220224-T00031
Please refer to the end of the specification for access instructions.

Lengthy table referenced here
US20220056480A1-20220224-T00032
Please refer to the end of the specification for access instructions.

Lengthy table referenced here
US20220056480A1-20220224-T00033
Please refer to the end of the specification for access instructions.

Lengthy table referenced here
US20220056480A1-20220224-T00034
Please refer to the end of the specification for access instructions.

Lengthy table referenced here
US20220056480A1-20220224-T00035
Please refer to the end of the specification for access instructions.

Lengthy table referenced here
US20220056480A1-20220224-T00036
Please refer to the end of the specification for access instructions.

Lengthy table referenced here
US20220056480A1-20220224-T00037
Please refer to the end of the specification for access instructions.

Lengthy table referenced here
US20220056480A1-20220224-T00038
Please refer to the end of the specification for access instructions.

Lengthy table referenced here
US20220056480A1-20220224-T00039
Please refer to the end of the specification for access instructions.

Lengthy table referenced here
US20220056480A1-20220224-T00040
Please refer to the end of the specification for access instructions.

Lengthy table referenced here
US20220056480A1-20220224-T00041
Please refer to the end of the specification for access instructions.

Lengthy table referenced here
US20220056480A1-20220224-T00042
Please refer to the end of the specification for access instructions.

Lengthy table referenced here
US20220056480A1-20220224-T00043
Please refer to the end of the specification for access instructions.

Lengthy table referenced here
US20220056480A1-20220224-T00044
Please refer to the end of the specification for access instructions.

### 6. EXAMPLES

The following examples are put forth so as to provide those of ordinary skill in the art with a description of how the compositions and methods claimed herein are performed, made, and evaluated, and are intended to be purely exemplary of the invention and are not intended to limit the scope of what the inventor regards as her invention.

### 6.1. Example 1

### Discovery of OncoVac

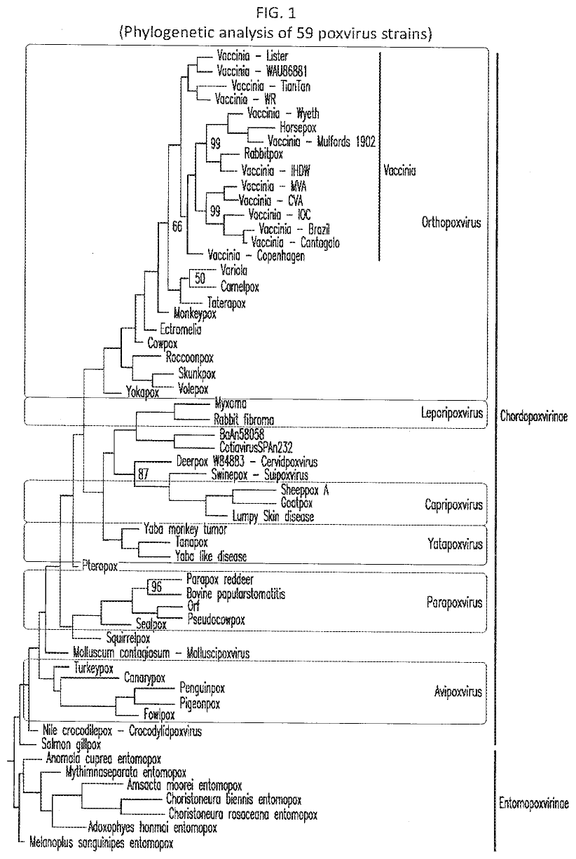

The open reading frames (ORFs) from 59 poxvirus strains were clustered into orthologs and aligned at the amino acid level (see FIG. 1 for phylogenetic analysis). Bayesian analysis was performed to determine relatedness of all strains. Poxviruses are very diverse in gene content and host range. There are several naturally occurring Vaccinia wild-type strains which are different from one another.

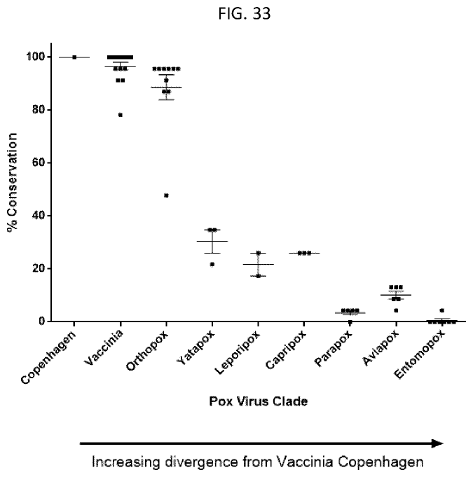

FIG. 33 shows the percentage of genes deleted in CopMD5p3p (Table 2) in various poxvirus genomes. Each dot on the graph represents one poxvirus genome. Homology searches were used to query poxviruses from other clades with amino acid sequences of Table 2 genes from the Copenhagen genome. The amount of deleted genes present in other pox viruses decreases with their increasing divergence.

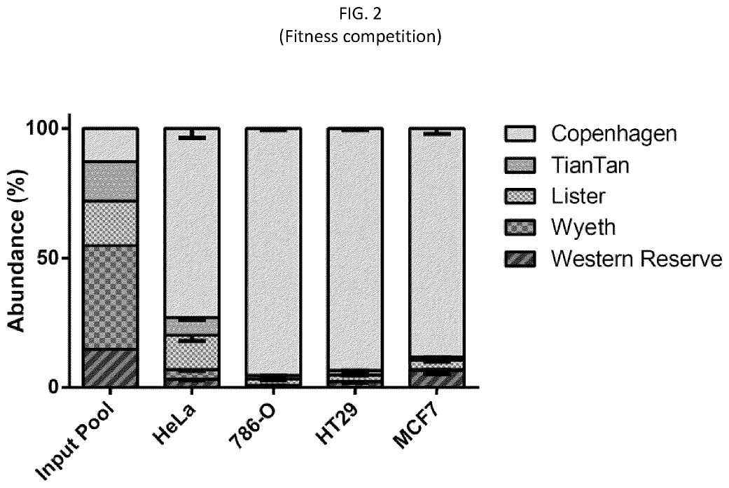

Vaccinia wild type strains were mixed at equal plaque forming unit counts and sequenced with NGS (Input pool). The resulting mixture was passaged three times in different cancer cell lines. The final population was sequenced with Illumina NGS sequencing. Reads (short DNA fragments) were assigned to various strains based on sequence identity and used to calculate the percent of each strain in the final population. The abundance of different viral strains after passaging 5 Vaccinia viruses in different tumor types is shown in FIG. 2. The Copenhagen strain is able to outgrow other strains and therefore replicates faster.

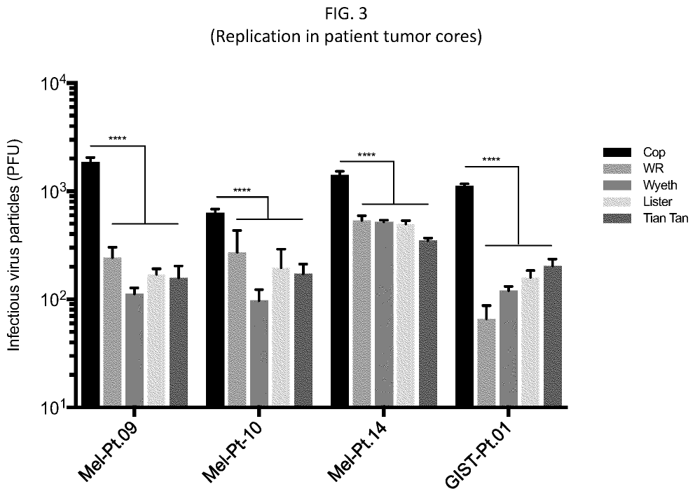

Different Vaccinia wild type strains were also used to infect at low PFU (1×10⁴) various patient tumor cores. Each strain infected on average 4 replicates each containing three 2×2 mm tumor cores. Replication was assessed through virus titering and is expressed as plaque forming units (PFU) as shown in FIG. 3. The Copenhagen strain grows to higher titres than other strains and therefore replicates faster in patient ex-vivo samples. Patient ex-vivo cores are a good mimic of a patient's 3D tumor.

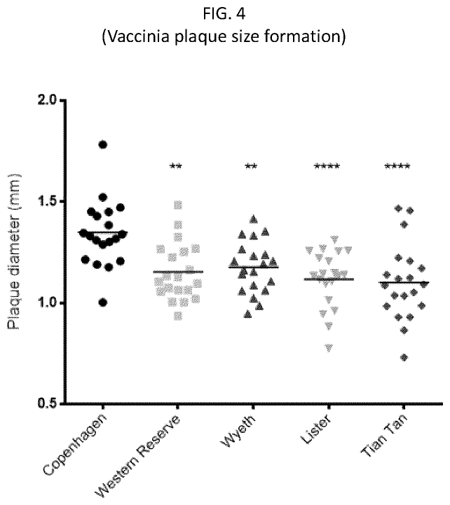

Vaccinia wild-type strains were then subjected to a plaque assay on U2-OS cells with a 3% CMC overlay. Two days past infection, 20-30 plaques for each strain were measured for their size. Plaque size measurements for Copenhagen, Western Reserve, Wyeth, Lister, and Tian Tan are shown in FIG. 4. Plaque formation is affected by the ability of the virus to replicate, spread, and kill. The larger plaque sizes observed for the Copenhagen strain suggest that this strain is superior in these abilities which are important for the development of an oncolytic virus.

Finally, all 59 poxvirus genomes from FIG. 1 were used to find ORFs and clustered into orthologous groups. Groups containing Copenhagen genes were plotted based on location of the gene in the Copenhagen genome (x-axis) and size of the group (y-axis). When all 59 species share the same gene the conservation is considered to be 100%. Genes that are part of the major deletions CopMD5p and CopMD3p were found to be less important for viral replication as their deletion does not impact fitness.

A novel phenotype has been discovered by deleting the previously uncharacterized A47L gene. Deleting A47L induces the Copenhagen virus to create larger plaques

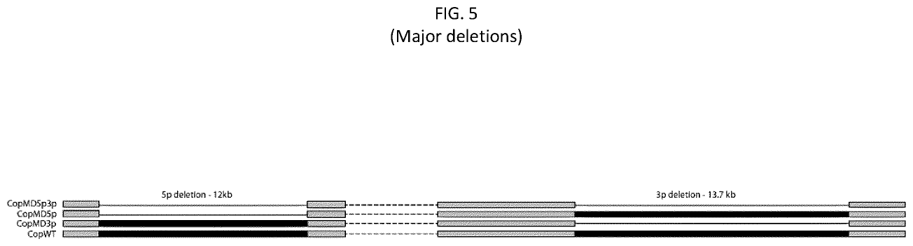

Illumina NGS deep sequencing revealed presence of major deletions during the plaque purification process. CopMD5p and CopMD3p represent clones which were plaque purified and found to harbor major genomic deletions. These 2 clones were used to co-infect a monolayer of HeLa cells at a high MOI (MOI 10) to induce recombination. Random plaque picking and PCR revealed presence of a double deleted CopMD5p3p which contained both genome deletions (see FIG. 5). Thus, two naturally occurring deletions in the wild-type Copenhagen population were discovered. These 2 deletions were combined and purified to give a replicating virus, referred to herein as “CopMD5p3p”, that exhibits deletions in the C2L, C1L, N1L, N2L, M1L, M2L, K1L, K2L, K3L, K4L, K5L, K6L, K7R, F1L, F2L, F3L, B14R, B15R, B16R, B17L, B18R, B19R, and B20R genes, as well as deletions in each of the B21R, B22R, B23R, B24R, B25R, B26R, B27R, B28R, and B29R copies of ITRs. As used herein, “CopWT” refers to wild-type Copenhagen vaccinia virus, “CopMD5p” refers to a Copenhagen vaccinia virus harboring deletions in representative 5′ genes (C2L, C1L, N1L, N2L, M1L, M2L, K1L, K2L, K3L, K4L, K5L, K6L, K7R, F1L, F2L, and F3L), and “CopMD3p” refers to a Copenhagen vaccinia virus harboring deletions in representative 3′ genes (B14R, B15R, B16R, B17L, B18R, B19R, and B20R) as well as deletions in each of the B21R, B22R, B23R, B24R, B25R, B26R, B27R, B28R, and B29R copies of ITRs.

### 6.2. Example 2

### Cancer Cell Death

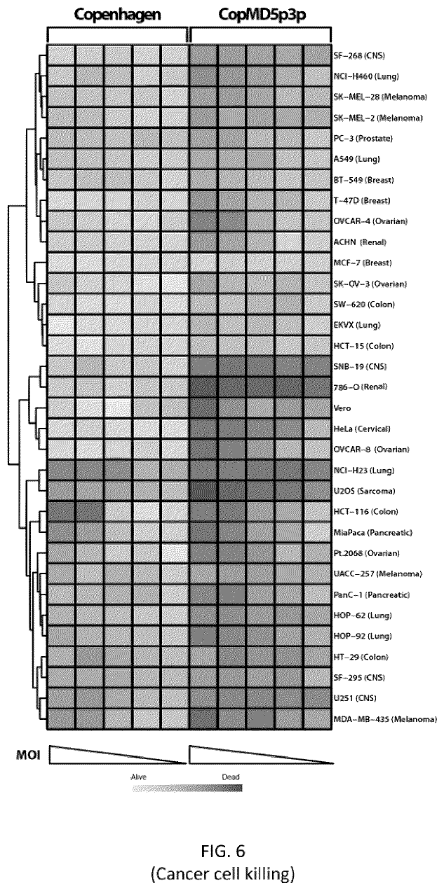

Cancer cells were infected with CopMD5p3p at a range of MOIs (1 to 0.01) in 24-well plates in 4 replicates. Two days post infection with virus, plates were stained with crystal violet. Crystal violet stain was dissolved into SDS and read by spectrophotometry. Data is represented as percent absorbance of non-infected cells (see FIG. 6). This data shows that the majority of cancer cell lines die faster when exposed to the CopMD5p3p virus.

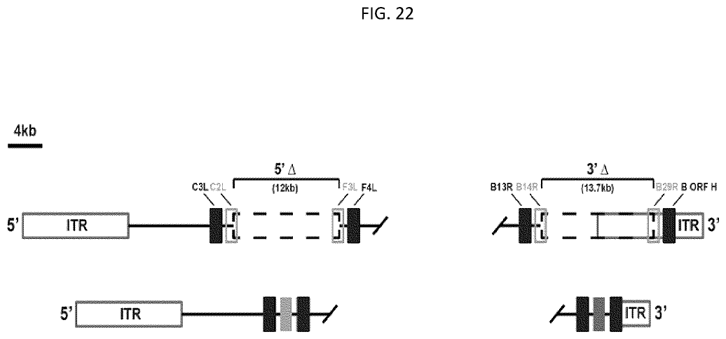

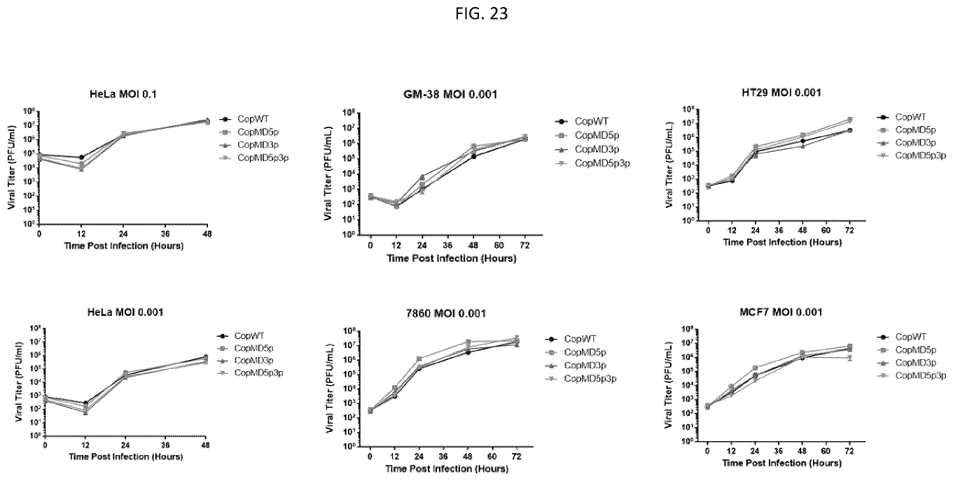

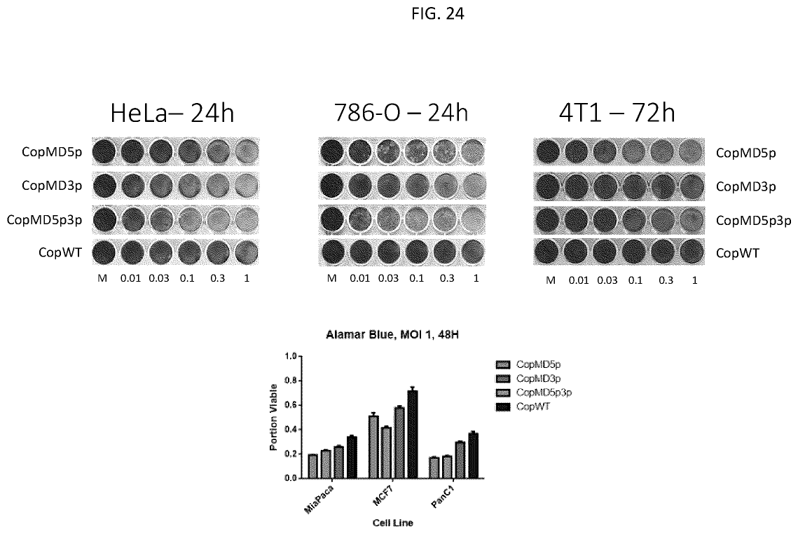

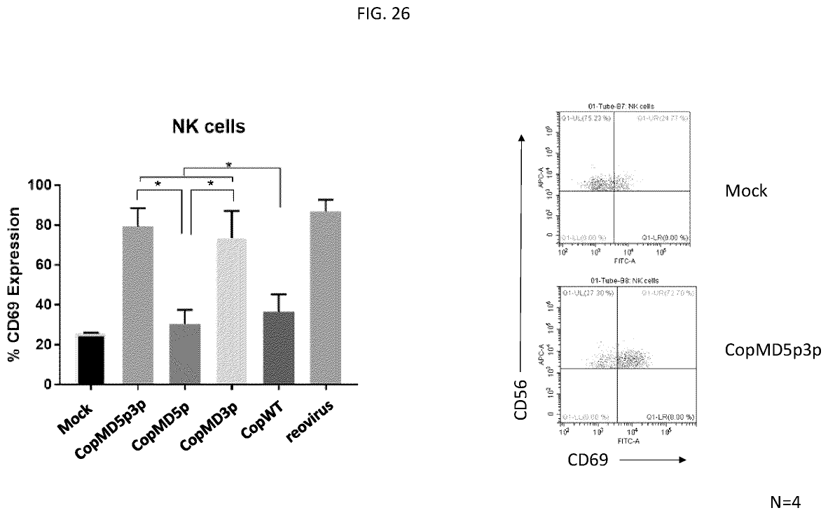

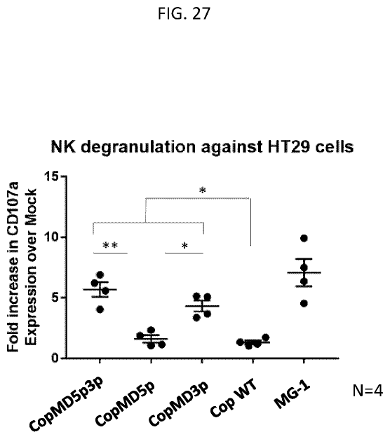

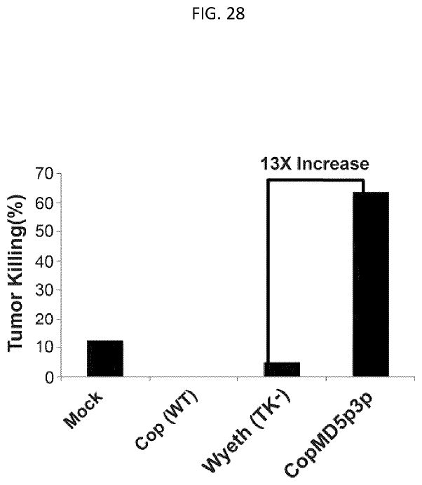

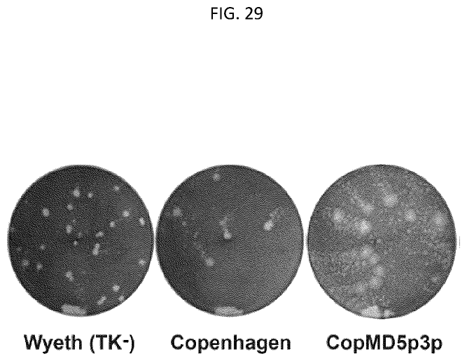

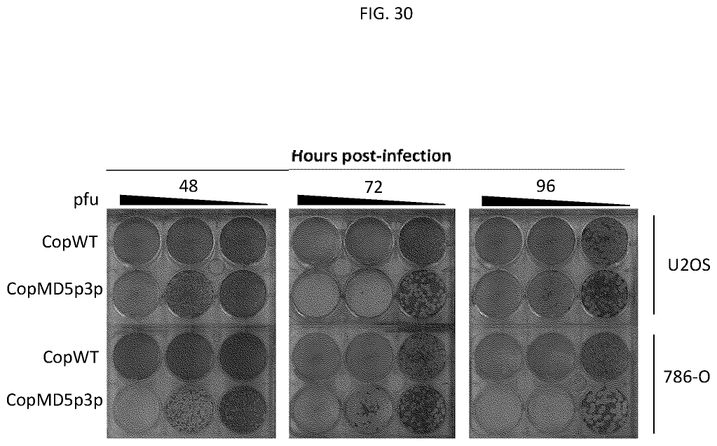

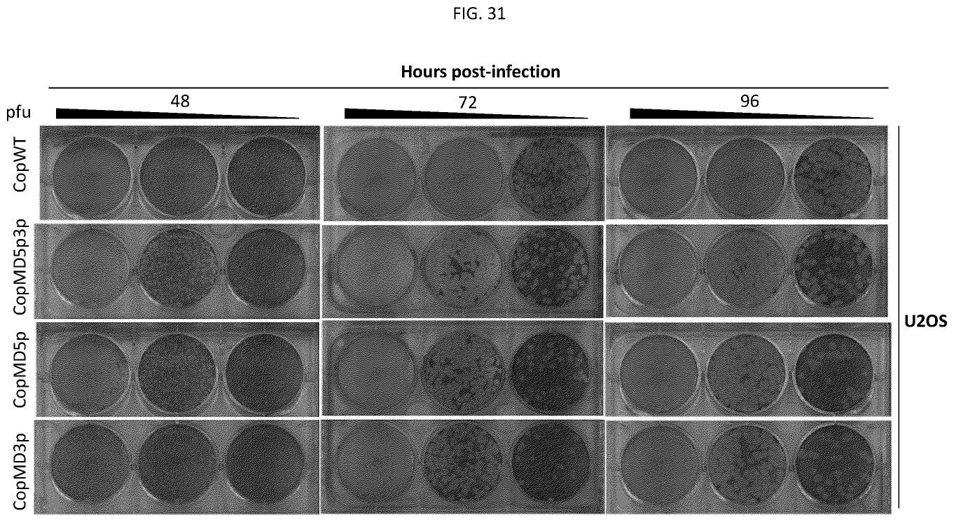

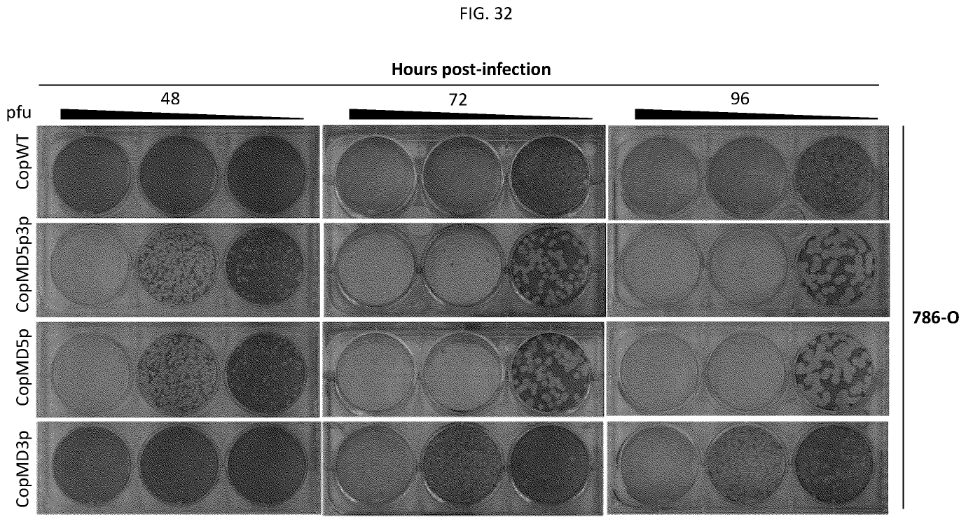

The ability of wild-type Copenhagen vaccinia virus and several modified Copenhagen vaccinia virions to induce an anti-tumor immune response and to propagate in various cancer cell lines is also shown in FIGS. 23, 24 and 26-32.

### 6.3. Example 3

### Growth in Cancer Cells

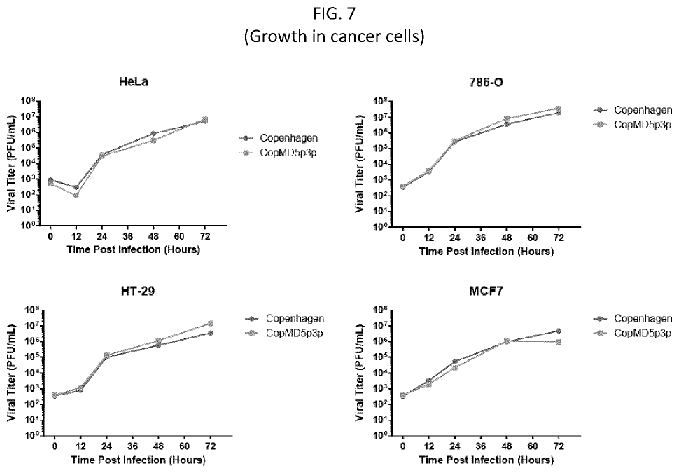

Four cancer cell lines were infected with CopMD5p3p at a low MOI (0.001) in 24-well plates in triplicates, and at different time points, the virus was collected and tittered. Time 0 h represents input. The growth curves of HeLa, 786-O, HT-29, and MCF7 are shown in FIG. 7. This data shows that the modified CopMD5p3p virus is not impaired in its ability to grow in vitro. This means that the virus is replication competent, even in presence of interferon response. The ability to replicate in mammalian cell lines provides another important advantage. As such viruses may be manufactured with enhanced speed and efficiency.

### 6.4. Example 4

### Growth in Patient Tumor Samples

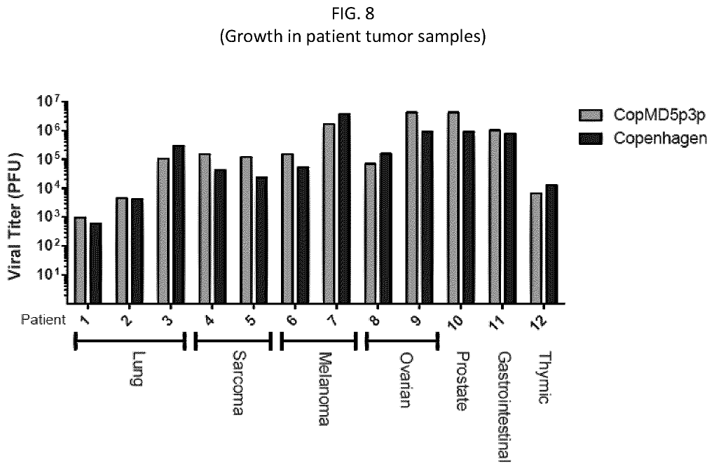

Patient tumor samples were obtained immediately after surgery and cut into 2 mm×2 mm cores. Three cores were infected with a small amount of virus (1×10 ⁴ PFU), either wild-type Copenhagen or CopMD5p3p. After 72 h virus output was assessed by plaque assay and final Viral Titer expressed as PFU (see FIG. 8). This data shows that the modified CopMD5p3p virus can replicate in fresh patient tumor samples. Replication in patient tumor samples is a good model of replication in a patient 3D tumor.

### 6.5. Example 5

### Cyncytia in U2-OS Cells

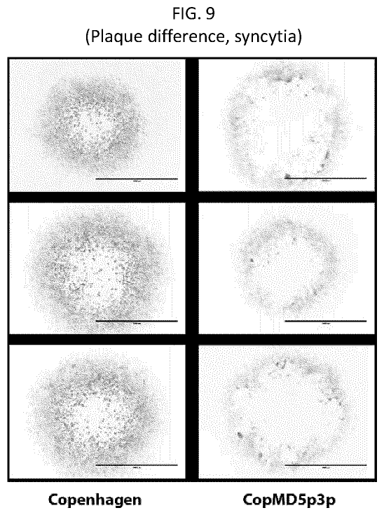

Monolayers of U2-OS cells were infected with either Copenhagen wild-type or CopMD5p3p virus. After 2h, the media was changed for overlay media as done for a plaque assay. At 48 h post infection, pictures were taken with EVOS to assess plaque phenotype (see FIG. 9). Cell fusion, also known as syncytia, is thought to help the virus spread, since uninfected cells merge with infected cells. Additionally, it has been shown that fused cells are immunogenic and in the case of cancer cells can help initiate an anti-tumor immune response.

### 6.6. Example 6

### Syncytia in 786-O Cells

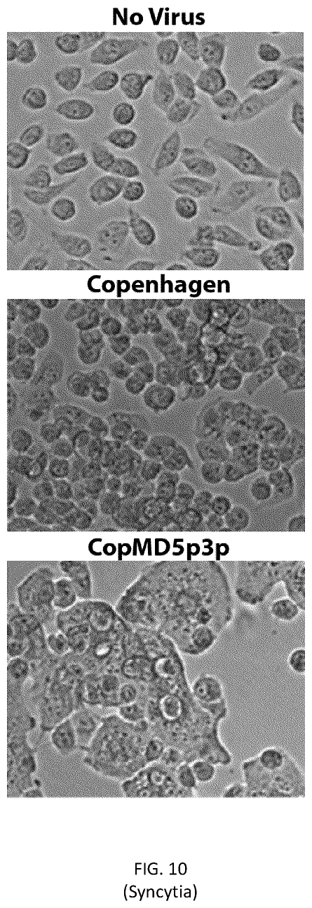

Monolayers of 786-O cells were infected with either Copenhagen wild-type or CopMD5p3p virus. After 24 h pictures were taken with EVOS at 10× magnification (see FIG. 10). This is additional evidence for the occurrence of syncytia. In FIG. 9, the phenotype of a plaque is shown. In the current experiment, monolayers of cells were infected without overlay. Most cells infected by the CopMD5p3p virus have fused.

### 6.7. Example 7

### Tumor Control and Weight Loss in Mouse Model

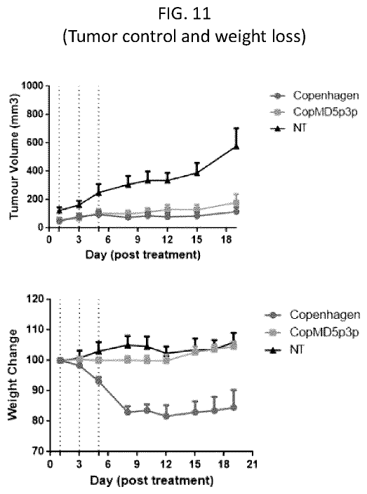

Nude CD-1 mice were seeded with HT-29 human colon cancer xenograft. Once subcutaneous tumors have established an approximate 5 mm×5 mm size, mice were treated three times (dashed lines) 24 h apart with 1×10⁷ PFU of either vaccinia virus intravenously. Mice were measured approximately every other day for tumor size and weight loss (see FIG. 11). This experiment shows that CopMD5p3p is a much safer virus because it does not cause any weight loss or other signs of sickness in immunocompromised nude mice. This experiment also shows CopMD5p3p is able to control tumor growth similarly to the parental Copenhagen wild-type virus.

### 6.8. Example 8

### Pox Lesion Formation

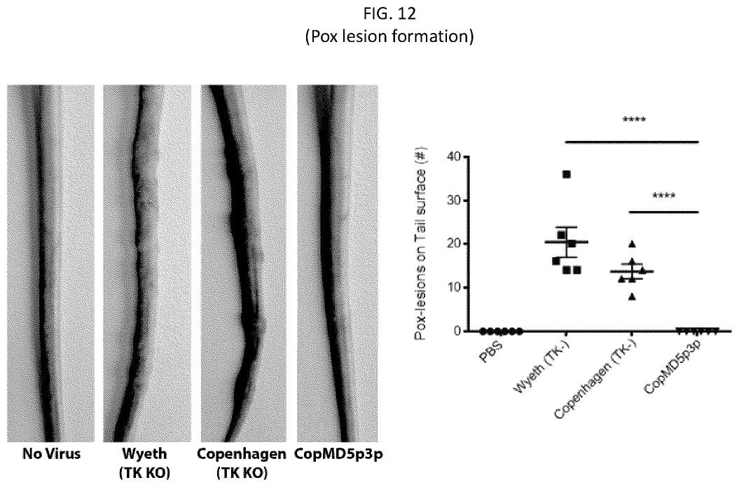

Nude CD-1 mice were treated once with 1×10⁷ PFU of either vaccinia virus intravenously, six mice per group. Two weeks post treatment, mice were sacrificed and pictures of tails were taken. Pox lesions on tails were counted manually on every mouse tail. Representative pictures shown in FIG. 12. This experiment shows that CopMD5p3p is a much safer virus because it does not cause any pox lesions in immunocompromised nude mice. This is important since prior Oncolytic Vaccinia clinical data has shown patients developing pox lesions upon treatment. Knockout of thymidine kinase (TK) is a popular way of increasing the safety of an OV (oncolytic virus), currently present in a Phase III Oncolytic Vaccinia and in FDA approved Oncolytic T-Vec. The data shows that deleting TK does not play a crucial role in this assay, where mice develop pox lesions when challenged with TK deleted viruses, but do not develop pox lesions with CopMD5p3p which has an intact TK.

### 6.9. Example 9

### IVIS Bio-Distribution of Vaccinia after Systemic Administration

Vaccinia viruses wild-type Wyeth, wild-type Copenhagen, and CopMD5p3p were engineered to express Firefly Luciferase (Fluc) through transfection of infected cells with a pSEM1 plasmid replacing TK with Fluc. Viruses were plaque purified and expanded. All viruses are TK knockouts and encode functional Fluc in their TK locus.

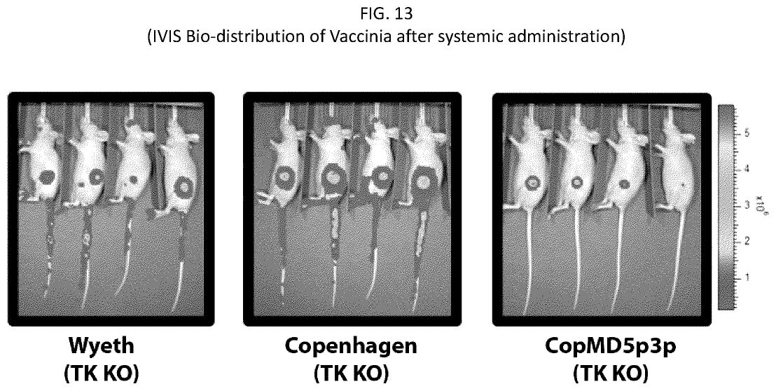

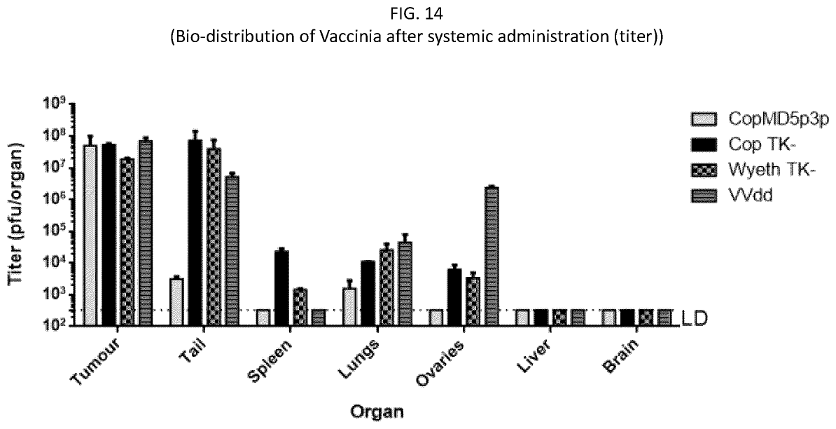

Nude CD-1 mice were then seeded with HT-29 human colon cancer xenograft. Once subcutaneous tumors have established an approximate 5 mm×5 mm size, mice were treated once with 1e7 PFU of either vaccinia Fluc encoding virus intravenously, four mice per group. Four days post treatment, mice were injected i.p. (intraperitoneal) with luciferin and imaged with IVIS for presence of virus (see FIG. 13). This experiment shows that CopMD5p3p is a much safer virus because it is more specific to the tumor. Other viruses show off target replication in the tail, muscle, paws and intra-nasal cavity. CopMD5p3p is only localized in the tumor. As shown in previous FIGS. 12 and 13, there is less detectable CopMD5p3p in the tail compared to the other strains. FIG. 14 shows that CopMD5p3p also has lower titers in other organs when compared to other oncolytic Vaccinia. Since the CopMD5p3p replicates at the same level as the other viruses in the tumor but less in off-target tissues, CopMD5p3p fits the profile of an oncolytic virus better.

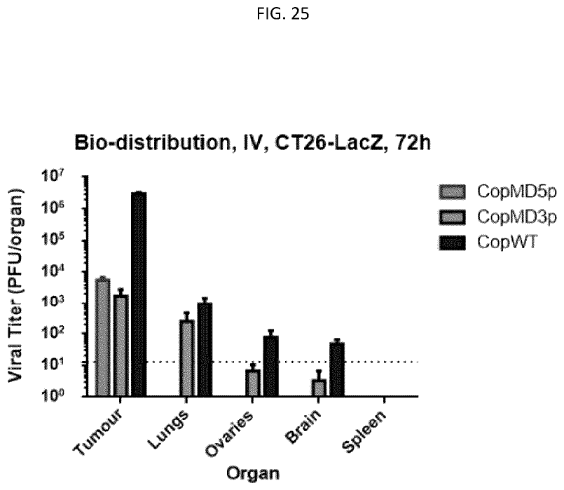

An additional example of the biodistribution of various vaccinia viral vectors, including the wild-type Copenhagen vaccinia virus and several modified Copenhagen vaccinia virions, is shown in FIG. 25.

### 6.10. Example 10

### Immunogenicity of Vaccinia in Human PBMCs

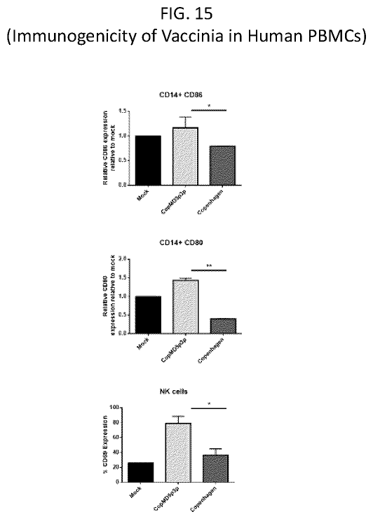

PBMCs were isolated from blood of healthy human donors (n=2). PBMCs were incubated with either Vaccinia for 24 h and checked for early activation markers using Flow Cytometry (see FIG. 15). This experiment shows that CopMD5p3p is more immunogenic and more readily detectable by immune cells. We believe that this is a desirable trait, since OVs replicating in tumor tissue need to activate immune cells for a successful anti-tumor immune response.

### 6.11. Example 11

### Immunogenicity of Vaccinia in Mouse Splenocytes

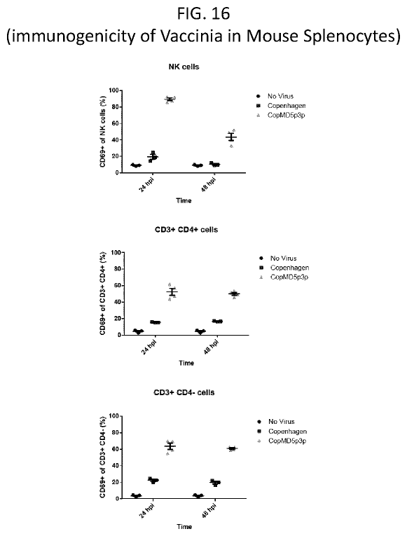

Immune competent Balb/C mice were injected with 1e7 Vaccinia PFU Vaccinia virus intravenously. After one or two days, mice were sacrificed, spleens were harvested and analyzed for immune activation using Flow Cytometry (see FIG. 16). This experiment shows that CopMD5p3p is more immunogenic and more readily detectable by mouse immune cells. This data complements nicely the previous FIG. 15, since most of the in vivo experiments are done in mice.

### 6.12. Example 12

### Immunogenicity of Vaccinia in Human Cells

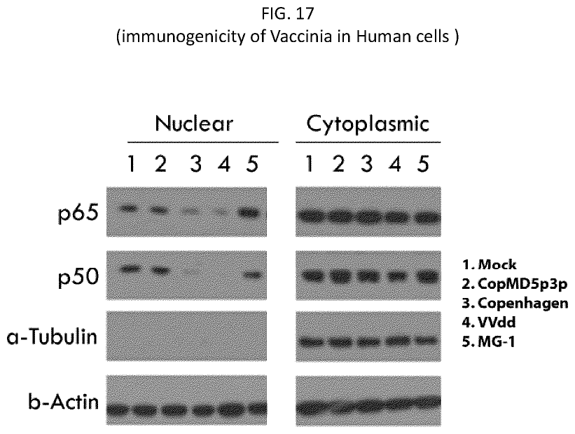

Human cancer cells 786-O were infected at an MOI of 0.01 with either virus. The next day, cells were harvested and nuclei and cytoplasm were separated by cell fractionation. Protein was extracted from each fraction and blotted for NF-kB subunits p65 and p50 (see FIG. 17). NF-kB immune transcription factor initiated an immune response once its subunit p65 and p50 are translocated to the nucleus. Some viruses are immunosuppressive and block this translocation, preventing an immune response. Suppressing NF-kB function is counter-intuitive to the goal of using oncolytic viruses in combination with immunotherapeutic approaches. Thus, CopMD5p3p is a more advantageous virus as it behaves similarly to MG-1.

### 6.13. Example 13

### Synergy with Immune Checkpoint Inhibitor Anti-CTLA-4 Antibody in Aggressive Melanoma Model

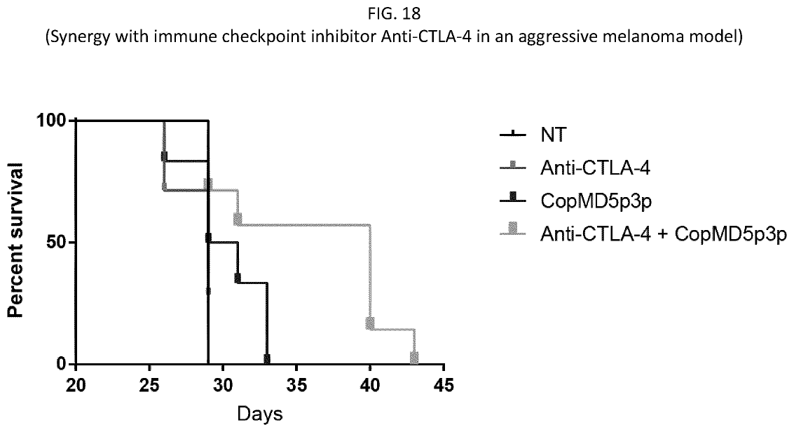

Immune competent C57BL/6 mice were seeded (5×10⁵ cells) subcutaneously with B16-F10 melanoma tumors. Treatment began once subcutaneous tumors have established an approximate 5 mm×5 mm size. Mice treated with CopMD5p3p virus received three 1e7 PFU doses into the tumor (intra-tumor) one day apart. Mice treated with Anti-CTLA-4 received five 100 ug doses of antibody i.p. one day apart. Survival were recorded every other day once treatment started (see FIG. 18). In this experiment we tested if the oncolytic effect of our CopMD5p3p virus can synergize with a well-known checkpoint inhibitor CTLA-4 in a very aggressive melanoma murine model. The median survival of mice treated with virus and checkpoint was higher than any other group. This suggests that CopMD5p3p has some stimulating properties that synergize with checkpoint blockade immunotherapy.

### 6.14. Example 14

### Synergy with Immune Checkpoint Inhibitor Anti-CTLA-4 Antibody

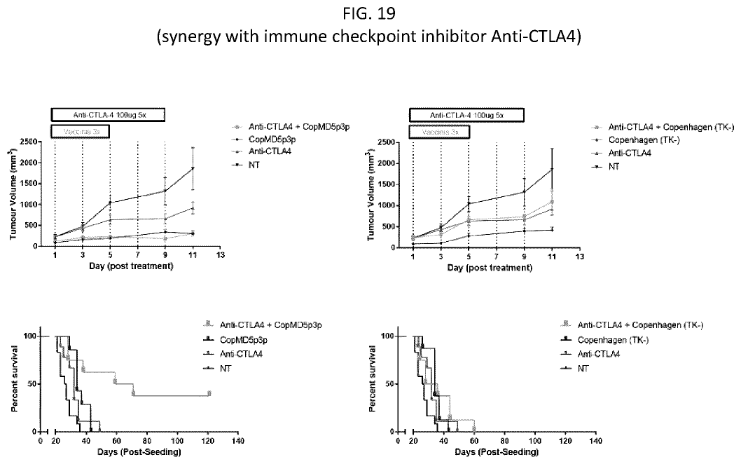

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1e7 PFU doses into the tumour (intra-tumor). Mice treated with Anti-CTLA-4 received five (24 h apart, dashed lines) 100 ug doses of antibody i.p. Tumor size and survival were recorded every other day once treatment started (see FIG. 19). The data shows that a TK knockout Vaccinia virus does not work as well with Anti-CTLA-4 as does CopMD5p3p. This suggests CopMD5p3p is more immunogenic and more capable of generating an anti-tumour immune response.

### 6.15. Example 15

### Synergy with Immune Checkpoint Inhibitor Anti-PD1 Antibody

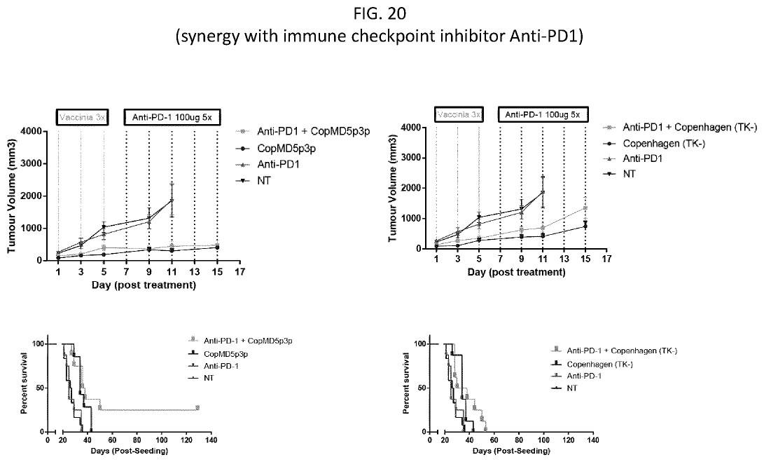

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1e7 PFU doses into the tumor (intra-tumor). Mice treated with Anti-PD1 received five (24 h apart, last five dashed lines) 100 ug doses of antibody i.p. 24 h after the last dose of Vaccinia virus. Tumor size and survival were recorded every other day once treatment started (see FIG. 20). The data shows that a TK knockout Vaccinia virus does not work as well with Anti-PD1 as does CopMD5p3p. This suggests CopMD5p3p is more immunogenic and more capable of generating an anti-tumor immune response.

### 6.16. Example 16

### Synergy with Immune Checkpoint Inhibitor Anti-PD1 Antibody and Anti-CTLA-4 Antibody

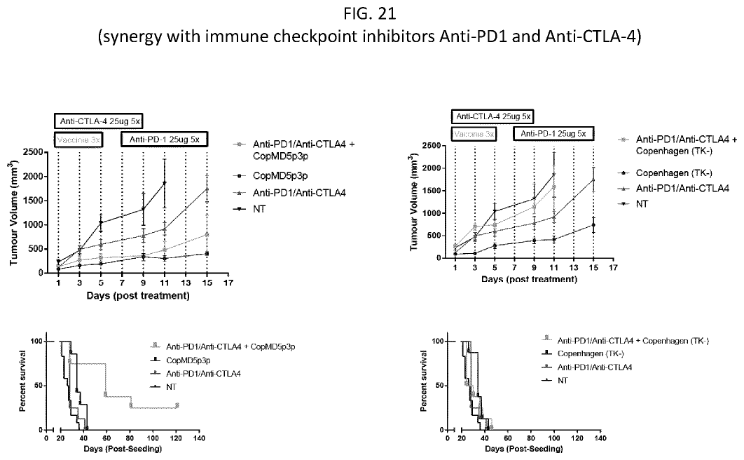

Immune competent Balb/C mice were seeded (5×10⁵ cells) subcutaneously with CT26-LacZ tumors. Treatment began once subcutaneous tumors have established an approximate 5 mm×5 mm size. Mice treated with Vaccinia virus received three (24 h apart, first three dashed lines) 1e7 PFU doses into the tumor (intra-tumor). Mice treated with Anti-CTLA-4 received five (24 h apart, first five dashed lines) 100 ug doses of antibody i.p. Mice treated with Anti-PD1 received five (24 h apart, last five dashed lines) 100 μg doses of antibody i.p. 24 h after the last dose of Vaccinia virus. Tumor size and survival were recorded every other day once treatment started (see FIG. 21). In this experiment we tested whether a lower dose (25 μg instead of 100 μg) of checkpoint inhibitor antibody could work if we blocked both checkpoints simultaneously. The CopMD5p3p still managed to achieve cures in this murine model with a lower dose (50 μg total) of checkpoints. Since checkpoint inhibitors have dose dependent toxicity, it is advantageous that very small doses of checkpoint blockers can still achieve an observable phenotype. As in other experiments, the CopMD5p3p virus manages to cure established tumors, and this effect is not observed with wild-type virus lacking the corresponding deletions of CopMD5p3p.

### 6.17. Example 17

### Administration for the Treatment of a Subject

Using the methods described herein, a clinician of skill in the art can administer to a subject (e.g., a patient) a pharmaceutical composition containing a recombinant orthopoxvirus vector described herein to treat cancer or tumor cells. The cancer may be, for example, leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, or throat cancer, among others.

For instance, a clinician of skill in the art may assess that a patient is suffering from cancer or tumors and may administer to the patient a therapeutically effective amount (e.g., an amount sufficient to decrease the size of the tumor) of a pharmaceutical composition containing the recombinant orthopoxvirus vector disclosed herein. The pharmaceutical composition may be administered to the subject in one or more doses (e.g., 1, 2, 3, 4, 5, 6, 7, 8, 9, 10, 15, 20, 25, or more) per a specified time interval (e.g., weekly, daily, or hourly). The patient may be evaluated between doses to monitor the effectiveness of the therapy and to increase or decrease the dosage based on the patient's response. The pharmaceutical composition may be administered to the patient orally, parenterally (e.g., topically), intravenously, intramuscularly, subcutaneously, or intranasally. The treatment may involve a single dosing of the pharmaceutical composition. The treatment may involve continued dosing of the pharmaceutical composition (e.g., days, weeks, months, or years). The treatment may further involve the use of another therapeutic agent (e.g., an immune checkpoint inhibitor, such as an anti-PD-1 or anti-CTLA-4 antibody or antigen-binding fragment thereof, IL-12, or Flt3 ligand, among other agents).

### 6.18. Example 18

### Targeted Deletions of CopMD5p and CopMD3p in Several Vaccinia Strains

The following protocol for producing modified vaccinia viral vectors utilizes techniques described, e.g., in Rintoul et al. PLoS One. 6(9): e24643 (2011), the disclosure of which is incorporated herein by reference.

Briefly, CopMD5p (Copenhagen vaccinia virus harboring deletions in 5′ genes: (the C2L, C1L, N1L, N2L, M1L, M2L, K1L, K2L, K3L, K4L, K5L, K6L, K7R, F1L, F2L, and F3L genes) and CopMd3p (Copenhagen vaccinia virus harboring deletions in 3′ genes: the B14R, B15R, B16R, B17L, B18R, B19R, and B20R genes) targeting recombinant constructs were synthesized by g-Block technology (IDT, Coralville Iowa). U2OS cells were infected with wildtype vaccinia virus (Wyeth, Western Reserve, Tian Tan, Lister) at an MOI of 0.01 in serum free DMEM for 1.5 hours. Viral supernatant was aspirated and U2OS cells were transfected with PCR amplified CopMD5p or CopMD3p targeting g-Blocks by Lipofectamine 2000 (Invitrogen) in OptiMEM (Gibco). DMEM supplemented with 10% FBS was added to cells 30 minutes after transfection and left overnight. The following day, transfection media was aspirated and fresh DMEM 10% FBS media was added to cells. 48 hours after infection transfection, U2OS cells were harvested and lysed by a single freeze thaw cycle. Serially diluted lysates were plated onto a confluent monolayer of U2OS cells and eGFP positive (CopMD5p targeted) or mCherry positive (CopMD3p targeted) plaques were isolated and purified through 5 rounds of plaque purifications.

Double major deleted vaccinia viruses were generated by co-infection of CopMD5p and CopMD3p deleted vaccinia viruses at an MOI of 5 for each virus in U2OS cells. Cells were harvested the next day and lysed by one round of freeze thaw. Lysates were serially diluted and plated onto a confluent monolayer of U2OS cells and selected for double positive plaques (eGFP+mCherry). Plaques were purified by 5 rounds of plaque purification.

An exemplary scheme for the production of modified orthopoxvirus vectors (e.g., modified vaccinia viral vectors, such as modified Copenhagen vaccinia viral vectors) of the disclosure is shown in FIG. 22.

### 6.19. Example 19

### SKV (CopMD5p3p-B8R-) has Similar Efficacy in Tumour Control Compared to SKV-B8R+

The vaccinia virus (VV) B8R gene encodes a secreted protein with homology to gamma interferon receptor (IFN-γ). In vitro, the B8R protein binds to and neutralizes the antiviral activity of several species of gamma interferon including human and rat gamma interferon; it does not, however, bind significantly to murine IFN-γ. Here we describe the construction and characterization of recombinant VVs lacking the B8R gene. Homologous recombination between the targeting construct and the B8R locus resulted in the replacement of 75% of the B8R gene with the eGFP transgenes flanked by two loxP sites (SKV-GFP).

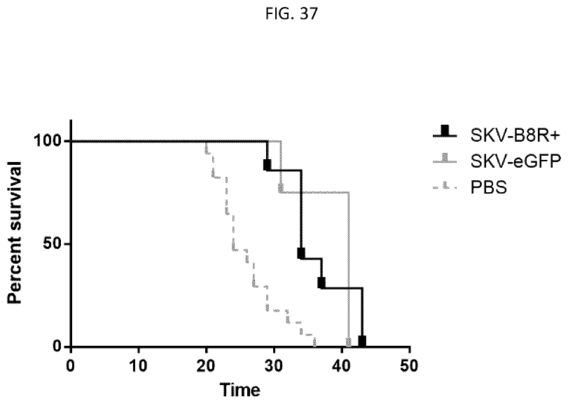

B8R-viruses showed similar efficacy to B8R+viruses. FIG. 37. Survival of mice treated with either SKV or SKV-GFP was assessed. 5×10⁵ CT26-LacZ cells were seeded subcutaneously on day 0. On day 14, 16 and 18 tumors were treated at a dose of 10⁷ pfu with an intratumoural injection of either SKV or SKV-GFP. No significant decrease in efficacy was seen when the viruses injected had a deletion of the B8R locus.

### 6.20. Example 20

### Infection of Normal Versus Cancer Cell Lines of SKV-B8R+Virus

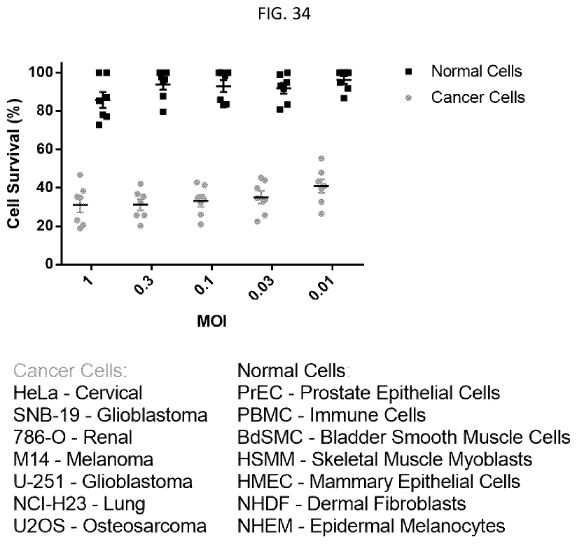

Primary health cell viability was compared to that of cancer cells. Confluent normal or cancer cells were infected at a range of MOI (pfu/cell) for 48 hrs, after which viability was quantified. As indicated in FIG. 34, SKV-B8R+virus preferentially infects cancer cells.

### 6.21. Example 21

### SKV-B8R+does not Impair Interferon Signaling

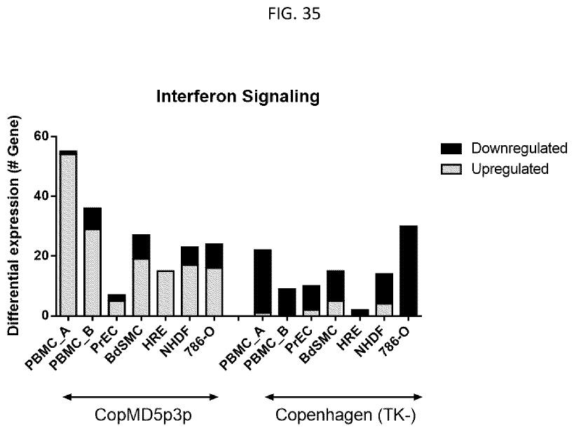

Interferon signaling was assessed by determining the number of genes in the interferon pathway that are unregulated (induced expression) or downregulated (repressed expression) in a variety of normal cell lines and one cancer cell line (786-O). FIG. 35 Confluent monolayers of 1 million cells were infected at an MOI of 3 (3e6 PFU) for 18 h with either SKV-B8R+ (CopMD5p3p) or the parental Copenhagen virus having the TK gene disabled. RNA was sequenced using RNA-seq and gene expression of interferon genes was determined after read mapping and expression normalization. While the SKV-B8R+ (CopMD5p3p) virus mostly induces genes in the interferon pathway the parental Copenhagen represses genes. This suggests SKV-B8R+ (CopMD5p3p) is able to induce Type I Interferon signaling which is critical in viral clearance of normal cells.

### 6.22. Example 22

### B8R Negative Vaccinia Virus Engineered to Express Flt3L, IL-12 TM and anti-hCTLA-4

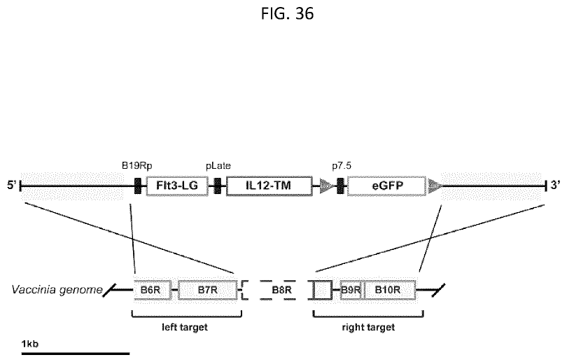

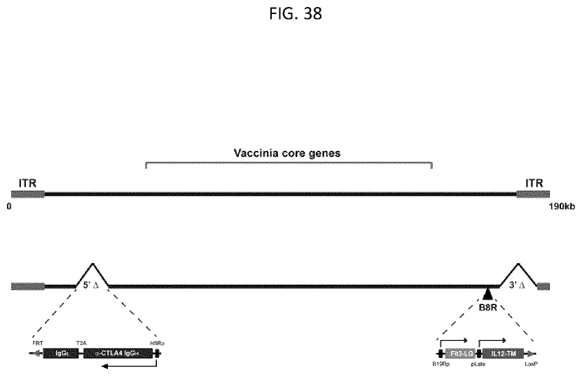

Modified vaccinia viruses containing both the CopMD5p3p and B8R deletions, as described above, were further engineered to express the immunotherapeutic transgenes, Flt3L and IL-12-TM, as well as an antibody to hCTLA-4, as shown in FIG. 38. The Flt3L, IL-12 TM and eGFP transgenes were inserted into the B8R locus on the Copenhagen vaccinia virus genome. See FIG. 36. Homologous recombination between the targeting construct and the B8R locus resulted in the replacement of 75% of the B8R gene with the Flt3-LG, IL-12-TM and eGFP transgenes (SKV-23). This strategy allowed the creation of a B8R knockout virus while inserting transgenes in lieu of B8R. The anti-CTLA-4 IgG was inserted into the SKV-23 backbone by targeting the boundaries of the 5p deletions to insert a heavy and light chain of a human anti-CTLA-4 antibody separated by a T2A proteomic motif (SKV-123v2; see FIG. 38).

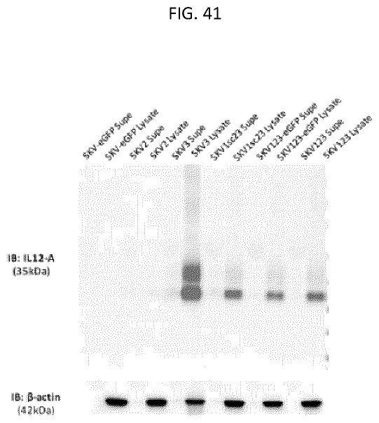

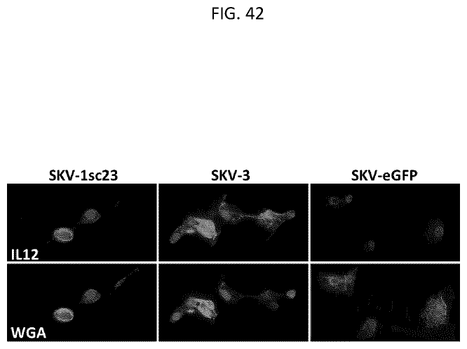

Ability of these engineered viruses to express the membrane bound IL-12 transgene was assessed. Cells were infected with SKV-1sc23, SKV-3 or SKV-GFP engineered vaccinia genes. As depicted in FIG. 42, Vero cells were stained with an IL-12p35 specific antibody. Vero cells were co-labelled with wheat germ agglutinin (WGA) conjugated flouorophore as a counterstain to visualize the cell membrane specific staining patterns. hIL-12 production was further quantified for various SKV viruses expressing transgenes through western blot quantification. See FIG. 41. Monolayers of 1×10⁶ HeLa cells were infected at MOI 0.1 (1×10⁵ pfu) with various Vaccinia SKV expressing different combinations of the three transgenes (anti-hCTLA-4, FTL3L, IL-12). After 48 h post infection, supernatant and lysate were collected and probed for expression of IL-12 p35 subunit. SKV-123 and SKV-3, both viruses engineered to express IL-12 p35 subunit with a transmembrane domain only express the protein on lysate fractions, suggesting the IL-12 does not get secreted into the supernatant.

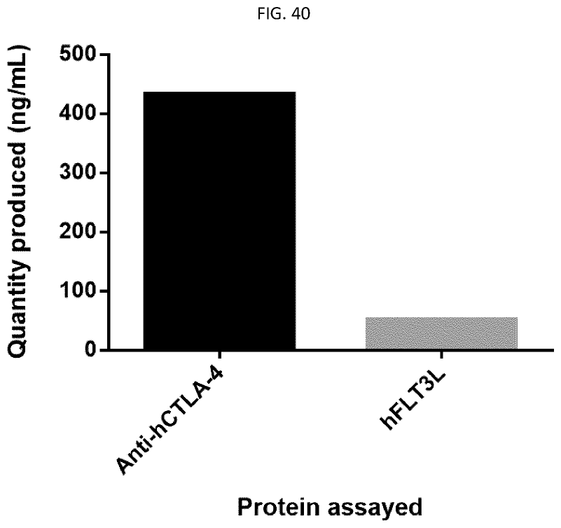

Expression levels of anti-hCTLA-4 and hFLT3L were measured using an ELISA protocol. Monolayers of le6 HeLa cells were infected at MOI 0.1 (1×10⁵ pfu) with SKV-123 expressing all three transgenes. 48 hours post infection, the supernatant was collected and assayed via ELISA for expression of the human anti CTLA-4 antibody and the Flt3 ligand. As depicted in FIG. 40, the SKV-123 virus was capable of producing more antibody against CTLA-4 than Flt3 ligand soluble protein.

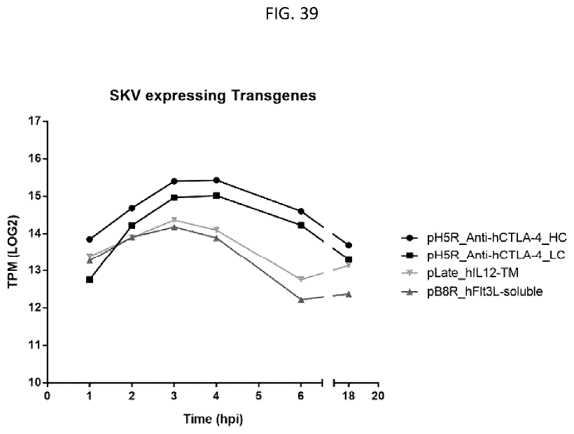

The SKV-123 virus expressing all three transgenes was evaluated in terms of transgene expression kinetics overtime (FIG. 39). Confluent monolayers of 786-O human adenocarcinoma cell lines were infected with SKV-123 virus at an MOI of 3 (3×10⁶ pfu). RNA was sequenced using RNA-seq and gene expression of inserted transgenes were determined after read mapping and expression normalization. The data suggest that transgene expression peaks at 3-4 hr. after cell infection occurs.

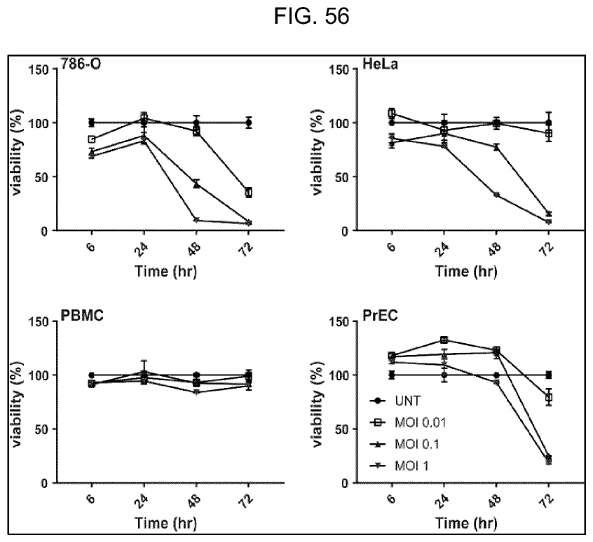

In a separate experiment, expression of the three transgenes from the SKV-123v2 virus was further evaluated in three other cancer cell lines: HeLa human cervical cancer cells (American Type Culture Collection (ATCC) Cat. #: CCL-2), HT-29 human colorectal cancer cells (ATCC Cat. #: HTB-38), and CT26.WT murine colorectal cancer cells (ATCC Cat. #: CRL-2638). SKV-123 virus infection at MOIs of 0.1 or 1.0 resulted in CTLA-4 antibody, FLT3L, and IL-12 transgene product production in each of the HeLa, HT-29, and CT26.WT cancer cells (FIGS. 56-59).

### 6.23. Example 23

### SKV Expressing Murine IL-12 p35 Membrane Bound has Greater Efficacy in Controlling Murine Tumors

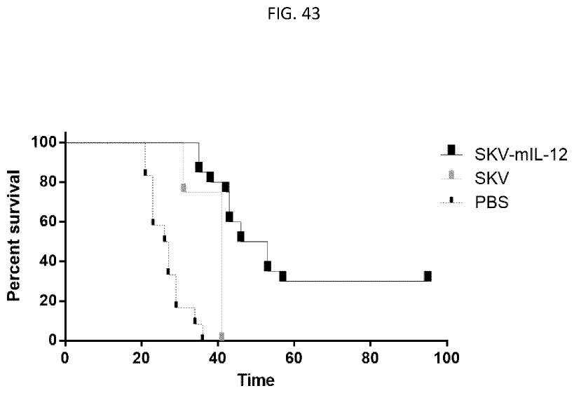

The survival of mice treated with either SKV or SKV-3 virus (expressing murine membrane bound p35 IL-12) was assessed. 5e6 CT26-LacZ cells were seeded subcutaneously on day 0. On days 14, 16 and 18, tumors were treated at a dose of 1e7 pfu with an intratumoural injection of either SKV or SKV-3. Although SKV virus extended survival of mice bearing CT26 colon tumors, SKV-3 expression of IL-12 is able to induce remissions that lead to durable cures. See FIG. 43.

### 6.24. Example 24

### Major Double Deletions in Engineered in Various Vaccinia Strains Enhance Cancer Cell Killing In Vitro

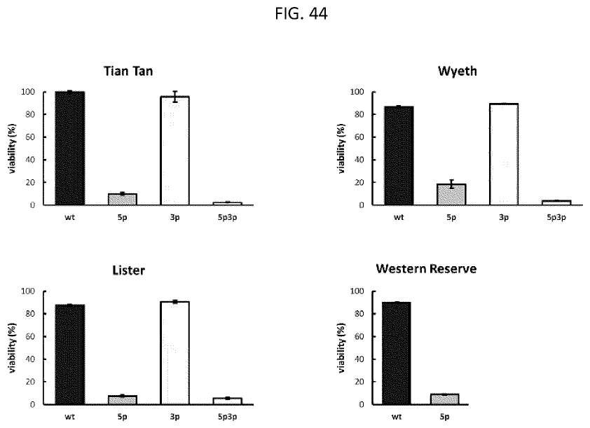

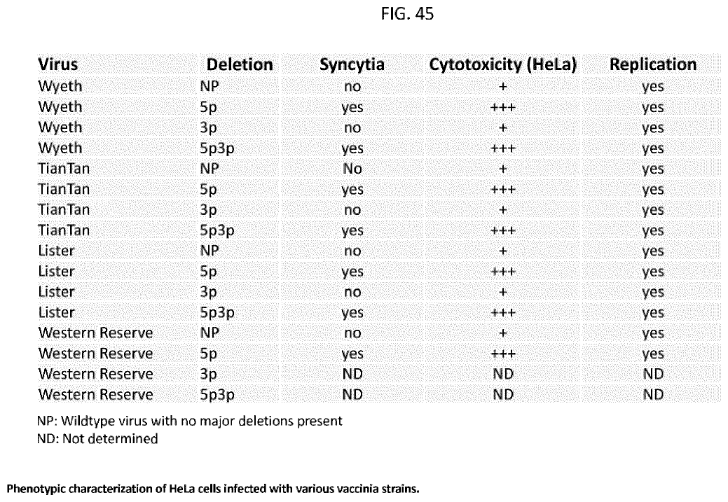

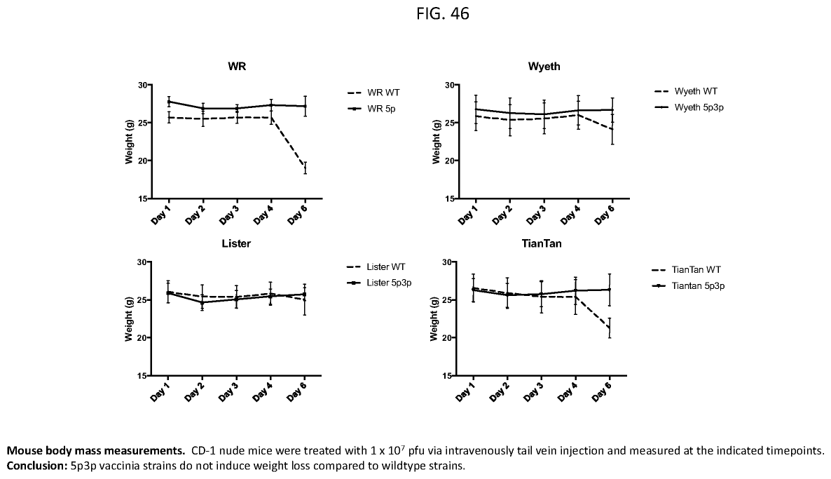

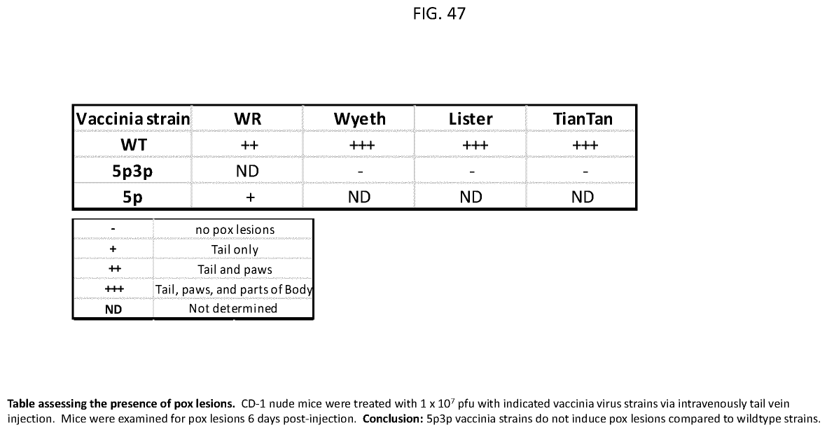

Hela cells were infected at an MOI of 0.1 with the following strains of engineered vaccinia viruses: (1) parental wildtype virus (wt); (2) 5 prime major deleted (5p), (3) 3 prime major deleted (3p), and (4) recombined 5 prime and 3 prime major double deleted (5p3p). Cell viability was quantified by alamar blue assay 72 hours post infection. Both 5p and 5p3p major double deleted vaccinia strains are more cytotoxic in HeLa cells when compared to their parental wildtype and 3p major deleted strains. See FIGS. 44-47.

### 6.25. Example 25

### Efficacy of SKV on Tumor Volume and Survival Benefit in 8 Different Xenograft Mouse Models

The objective of these efficacy studies was to determine the anticancer activity of the modified vaccinia viruses comprising both the CopMD5p3p and B8R deletions (SKV) injected intravenously (IV) and/or intratumorally (IT) in athymic nude mice implanted subcutaneously (SC) with either Mia PaCa-2 human pancreatic tumor cells, PC-3 human prostate tumor cells, U87MG human glioma cells, UACC-62 human melanoma cells, UM-UC-3 human bladder tumor cells, COLO-205 human colon tumor cells, NCI-H460 human lung tumor cells or HT29 human colon adenocarcinoma cells.

Cells were cultured and when the required number of cells were obtained, a total of 45 female, 6-8 week old athymic nude mice were inoculated with tumor cells (Day 0). Each mouse was injected SC in the right flank with ten million cells (1×10⁷ cells) in 0.2 mL of 1:1 Matrigel™. When 25 mice had tumors of approximately 100 to 200 mg (target group mean tumor weight of approximately 200 mg), treatment with Vaccinia virus was started.

The mice were administered 0.05 ml SKV (dose 1e7 pfu) either IT (10 mice) and/or IV (10 mice). Controls (5 mice) were administered 0.05 ml PBS. For IT injections, a 31G ½″ needle attached to a sterile syringe was used to inject each tumor. If the tumor was large or irregularly shaped, the tumor was injected in a different area on each day of injection. Mice were observed twice daily for mortality and moribundity. Tumors were measured twice a week starting on first day of treatment. Tumor volumes (mm³) were calculated using the equation for an ellipsoid sphere (l×w²)/2=mm³, where/and w refer to the larger and smaller dimensions collected at each measurement.

On Day 60 after tumor implantation or earlier, any moribund animal, any animal with excessive body weight loss (>30% of the body weight from first day of treatment) or any animal whose total tumor burden reached 4,000 mg, was ulcerated, or was sloughed off was removed from the study.

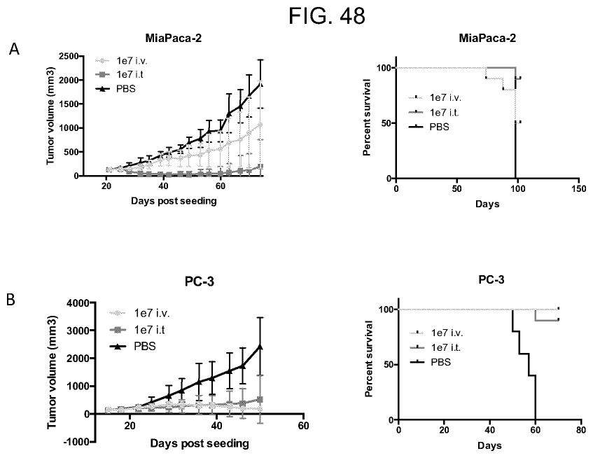

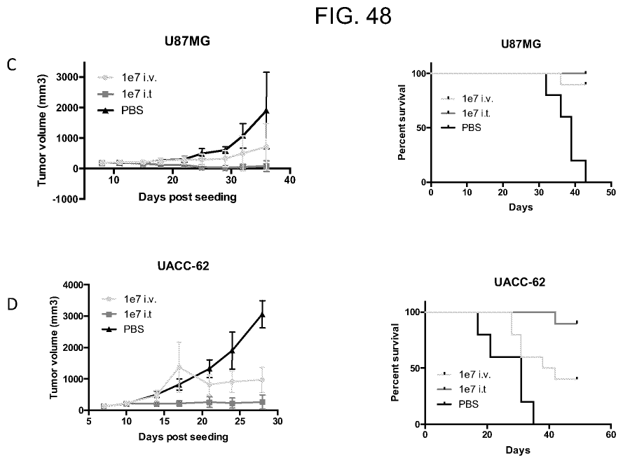

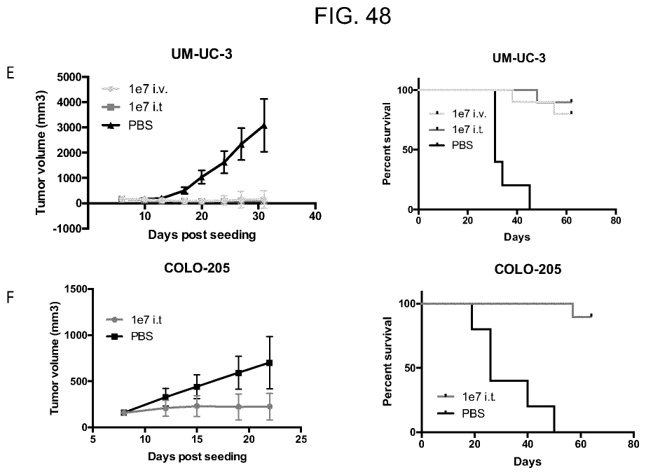

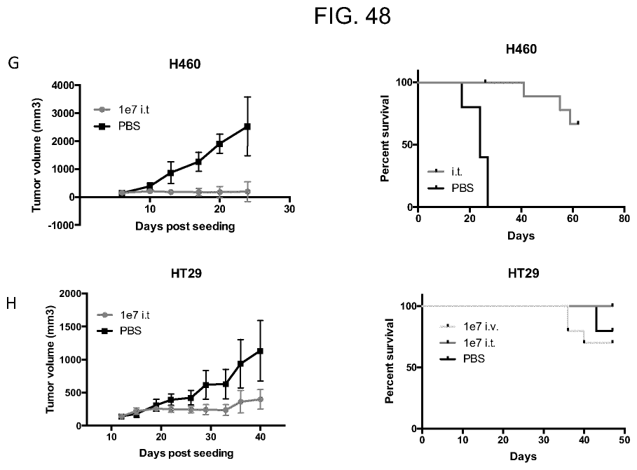

These efficacy studies showed a measurable reduction in tumor volume in the Mia PaCa-2, PC-3, U87MG, UACC-62 and UM-UC-3 xenograft models when a dose of 1e7 pfu SKV was administered IV or IT, as shown in FIGS. 48A-48E as well as in COLO-205, H460 and HT29 xenograft models when a dose of 1e7 pfu SKV was administered IT, as shown in FIGS. 48F-48H. These efficacy studies also showed a significant increase in percent survival (p<0.0001) in the all 8 xenograft models when administered with SKV IV and/or IT compared to control mice treated with PBS alone, as shown in FIGS. 48A-48H.

### 6.26. Example 26

### Efficacy of Various SKV Vectors on Tumor Volume and Survival Benefit in a Syngeneic Mouse Model

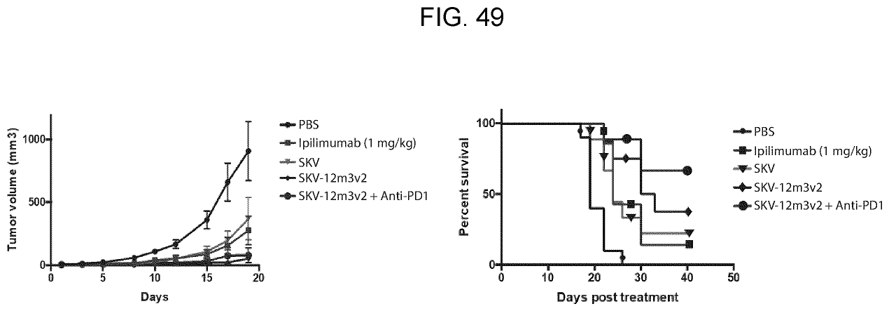

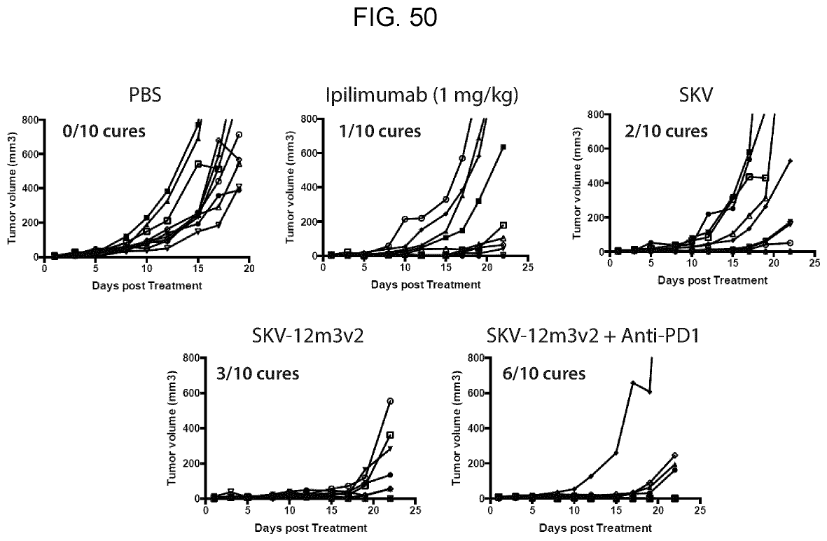

A number of SKV vectors were generated (see Tables 44 and 45 above). The purpose of this study was to compare the efficacy of SKV-12m3v2-eGFP, ipilimumab and SKV in a syngeneic mouse model where all encoded transgenes were active. MC38 colorectal cancer cells (5×10⁵) were re-suspended in 100 μl of 1:1 mixture of Matrigel and serum free DMEM media were injected SC into the right flank of transgenic C57BL/6 mouse expressing human CTLA-4. Animals were then randomized into 5 treatment groups and then treated with PBS, PBS plus ipilimumab, SKV, anti-PD-1 antibody, SKV-12m3v2-eGFP or SKV-12m3v2-eGFP plus anti-PD-1 antibody. SKV-12m3v2-eGFP is SKV expressing the human anti-CTLA-4 antibody, human Flt3 ligand and mouse IL-12 TM p35. Virus was diluted in PBS to deliver 1×10⁸ PFU/mouse in 50 μl and then total volume injected into the center of the tumor creating a single needle track. Antibodies (ipilimumab 20 μg in 100 μl and anti-PD-1 antibody 250 μg in 100 μl) were diluted in sterile PBS to appropriate dose and delivered by ip injection. Mice bearing SC MC38 tumors were treated with 3 doses of either a) ipilimumab at 1 mg/kg, b) SKV at 1×10⁸ PFU or c) SKV-12m3v2-eGFP at 1×10⁸ PFU. Tumors were measured, volumes recorded and basic wellness assessed prior to treatment and then over the course of the study (21 days duration). Results from the study are shown in FIG. 49 (average tumor volume and survival curves) and FIG. 50 (individual tumor volumes). Ipilimumab alone resulted in delayed tumor growth and 1 cure (10%); SKV alone showed efficacy and 2 cures (20%); SKV expressing the three transgenes significantly improved tumor control, increased survival, and resulted in 3 cures (30%); addition of PD-1 antibody doubled the cure rate (30-60%); and PD-1 antibody alone had no effect (data not shown).

Tumor control, as compared to the PBS-treated group at the time of the first humane endpoint on study, was observed in response to treatment with ipilimumab (P<0.05), SKV (P<0.05), SKV-12m3v2-eGFP (P<0.05), and SKV-12m3v2-eGFP +anti-PD-1 antibody (P<0.05). There was no statistically significant difference in tumor control between the different viruses or with the addition of anti-PD-1 antibody. All statistical tests were performed using multiple t-tests with GraphPad Prism 8.2.

Increased survival time compared to the PBS-treated group (median survival 19 days) was observed with treatment with SKV (P<0.005, median survival 20=5 days, Ipilimumab (P<0.001, median survival 30 days), SKV-12m3v2-eGFP (P<0.001, median survival 33 days), and SKV-12m3v2-eGFP +anti-PD-1 antibody (P<0.001). Complete tumor regression was observed in the following treatment groups: Ipilimumab (n=1), SKV (n=2), SKV-12m3v2-eGFP (n=3) and SKV-12m3v2-eGFP (n=6). Treatment with SKV-12m3v2-eGFP +anti-PD-1 antibody showed increased survival benefit compared to treatment with SKV alone (P<0.05); however, there was no statistically significant difference in survival when comparing SKV-12m3v2-eGFP +anti-PD-1 antibody to SKV-12m3v2-eGFP alone. All statistical tests were performed using the log-rank (Mantel-Cox) test with GraphPad Prism 8.2.

When the six mice with complete regression of initial tumors in the SKV-12m3v2-eGFP+anti-PD-1 antibody group were rechallenged with 5×10⁵ MC38 cells on the opposite flank, 2 mice displayed tumor growth and 4 mice remained resistant to tumor growth.

### 6.27. Example 27

### Comparison of Efficacy of SKV Expressing Membrane Bound IL-12p35-TM Subunit Versus IL12p7O-TM Subunit on Tumor Volume MC-38 Mouse Models

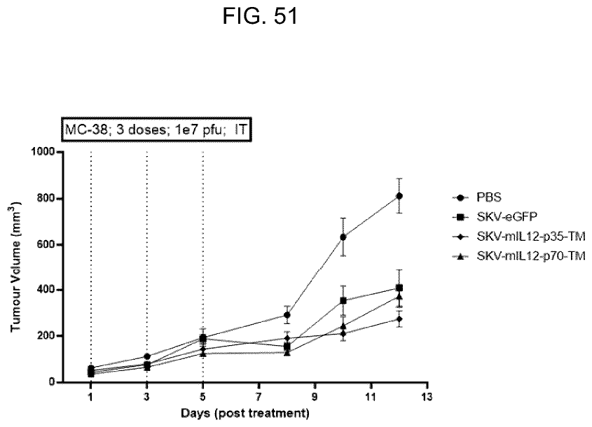

The purpose of this study was to compare the efficacy of SKV-mIL12p35 to SKV-mIL12p70 and SKV in the MC38 tumor model to assess whether there are difference in tumor control and survival related to the immune stimulatory potential of the 2 IL12 subunits. MC38 colorectal cancer cells (5×10⁵) were re-suspended in 100 μl of 1:1 mixture of Matrigel and serum free DMEM media were injected SC into the right flank of C57BL/6 mice. Tumors were allowed to grow for 7 days until they were approximately 3×3 mm. Animals were then randomized into 4 treatment groups and then treated with PBS, SKV, SKV-mIL12p35 or SKV-mIL12p70. Virus was diluted in PBS to deliver 1×10⁷ PFU/mouse in 100 μl and then total volume was injected into the center of the tumor creating a single needle track. Results are shown in FIG. 51. SKV, SKV-mIL12p35 and SKV-mIL12p70 treated mice all showed a reduction in tumor volume in comparison to control PBS treated mice. SKV-mIL12p35 and SKV-mIL12p70 treatment were both more effective than SKV treatment alone. SKV-mIL12p35 and SKV-mIL12p70 treated mice showed comparable reductions in tumor volume.

### 6.28. Example 28

### SKV in a heterologous Prime-Boost Oncolytic Vaccine Regimen

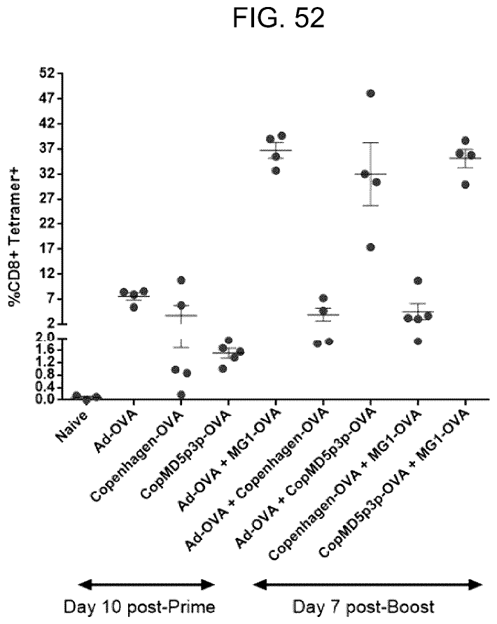

Preliminary results showed that SKV can be used to either prime or boost an immune response in a heterologous vaccine. Ovalbumin (OVA) was used as a foreign antigen in a heterologous prime-boost combination. Animals were primed on day 1, assessed on day 8-10 and then boosted on day 14 and assessed again after the boost at day 21-24. Healthy C57 black mice were treated with PBS (control), Adenovirus expressing OVA, wild-type Copenhagen strain vaccinia virus expressing OVA or CopMD5p3p (SKV backbone) expressing OVA as a prime or a boost. Tetramer analysis was performed evaluating OVA specific responses on Day 21. The mice were assessed at 10 days, showing that CopMD5p3p or SKV backbone but not Copenhagen virus induced immune responses both in prime and boost setting in C57BL/6 mice that were dosed with prime on day 1 and then immune boosted on Day 14 as shown in FIG. 52. CopMD5p3p (SKV backbone) can prime, and the prime response can be boosted by Maraba MG1 oncolytic rhabdovirus. CopMD5p3p (SKV backbone) can boost an Adenovirus prime response. CopMD5p3p (SKV backbone) outperformed parental Copenhagen virus in both priming and boosting an immune response to the foreign antigen.

### 6.29. Example 29

### BioDistribution of SKV-123 Transgenes with IT vs IV Administration

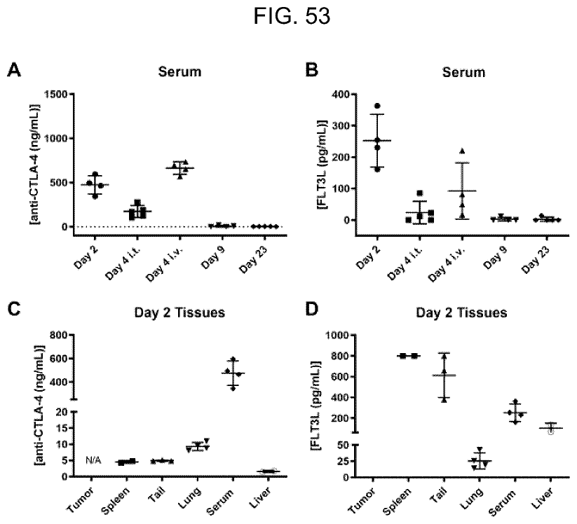

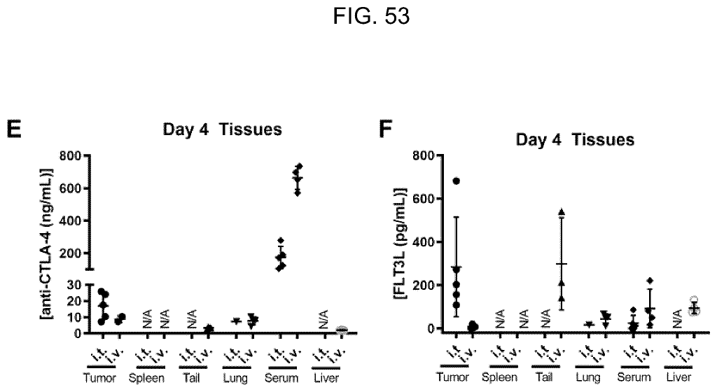

The effect of different routes of administration (IV vs IT) on the biodistribution of the transgenes (Anti-CTLA-4 antibody, FLT3L and IL-12-TM) expressed as a result of treatment with SKV-123 was studied. Female BALB/c mice were engrafted with CT26-LacZ tumor cells (3×10⁵ cells) and subsequently given SKV-123 two weeks later (dose 1×10⁸ PFU) by either IV injection on Study Days 1, 3 and 5, or IT injection on Study Days 1 and 3. Blood and tissues were harvested on Days 2, 4, 6, 8 and 22 for tumor-bearing groups. Spleens were harvested on Days 28 and 43 for non-tumor-bearing groups. Serum was isolated from blood samples (collected from cardiac punctures) and tissues were homogenized for ELISA of FLT3L and anti-CTLA-4 antibody expression. ELISA was performed only on samples that tested positive for viral genomes by PCR. For IL-12-TM biodistribution, tissues were harvested on Days 2, 4, 8, 22, 28, and 43. Tissues were homogenized for Western Blot analysis of IL-12-TM transgene expression. Western Blotting was performed only on samples that tested positive for viral genomes by PCR. Anti-Tubulin was used as a loading control. Tubulin could not be detected in spleen and tail samples, however the presence of protein was confirmed with Ponceau staining of the blot after transfer. SKV-123 given by IV injection on Days 1 and 3 resulted in detectable levels of anti-CTLA-4 antibody and FLT3L in the serum (FIGS. 53A, 53B) and at lower concentrations in the tumors of mice on day 2 (FIGS. 53C, 53D). FLT3L could also be detected on Day 2 in the spleen and tail, and still at Day 4 after IV treatment (FIGS. 53C, 53D). Anti-CTLA-4 antibody remained detectable in the serum on Day 4 regardless of IV or IT treatment (FIG. 53A). IT treatment of SKV-123 resulted in FLT3L concentrations in the tumor on Day 4 (FIG. 53F), while anti-CTLA-4 antibody concentrations were low but detectable (FIG. 53E). Tumor-selective transgene expression has been demonstrated in murine tumor models in which therapeutic payload concentrations were achieved within the tumor (e.g., >7.5 ng/ml FLT3L) without any detectable transgene product detected in the systemic circulation.

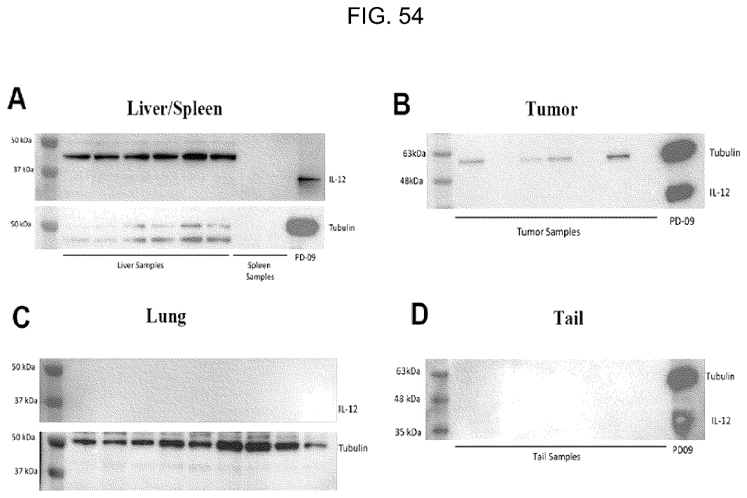

Western blotting for IL-12-TM showed only non-specific bands in liver (FIG. 54A), while no IL-12-TM was detected in the tumor, spleen, lungs or tail of animals (FIGS. 54A-54D). The biodistribution of SKV-123 in immunocompetent tumor-bearing mice was restricted to the tumor in IT treated animals and to the tumor and a limited number of other tissues at very low levels in the first few days following IV treatment.

Viral genomes were not detected in the brain, heart, kidney, ovary, inguinal lymph node, bone marrow, or serum at any of the time points assessed. Viral genomes were only detectable in tumor samples at the Day 4 time point, with the exception of one sample at the Day 9 time point. Genomes were detectable in some tail, lung, spleen, and liver samples, primarily at the Day 2 and 4 time points. No genomes were detected in any samples from the Day 23 harvest group.

Viral titers for SKV-123 virus were highest in the tail at Day 2 post-treatment by IV injection, with detectable low levels in the spleen, lung and liver. At Day 4, titers were only detected in the tail for IV injection groups. Viral titers were detected in the tumor for both administration routes, with higher titers found after IT injection compared to IV injection. Virus was also detected in the lung at comparable levels for both routes of administration.

None of the environmental shedding samples (serum, urine, saliva) examined in this study contained any detectable viral genomes or replicating virus.

### 6.30. Example 30

### Efficacy of SKV-123v2 on Tumor Volume in a Humanized Mouse Model

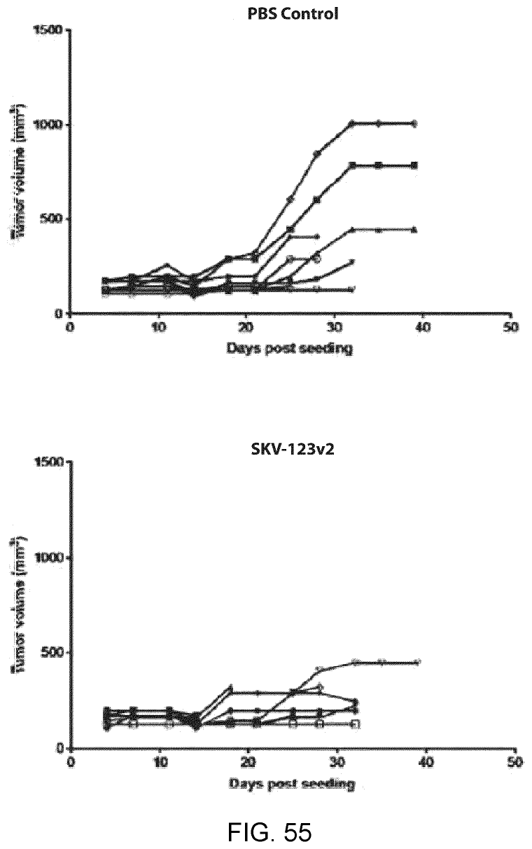

The efficacy of SKV-123v2 on the growth of tumours in a humanized mouse model was studied. NOD scid gamma (NSG) mice are a brand of immunodeficient laboratory mice that can be treated with human PBMCs and as a result develop a human-like immune system. Female NSG mice were implanted with human PBMCs and then 2 weeks later engrafted subcutaneously with 1×10⁷ UM-UC-3 xenograft human bladder tumor cells. Tumor volumes (mm³) were calculated using the equation for an ellipsoid sphere (l×w²)/2=mm³, where l and w refer to the larger and smaller dimensions collected at each measurement. When tumors were a minimum of 100 mm³, the mice were treated with SKV-123v2 at 1×10⁸ PFU or saline control by IV injection every two days for three weeks. Basic mouse wellness was assessed and tumor volume measured over the course of the study. Tumors were measured twice a week starting on Day 4 of implantation and then from the first day of treatment. On Day 21 the SKV-123v2 treated animals had a mean tumor volume of 164 mm³ corresponding to a difference of −8.4% relative to the vehicle-treated control with the maximum effect being observed on Day 32 with a difference of −53.2% relative to the control (FIG. 55).

### 6.31. Example 31

### In Vitro Infectivity Comparison of SKV-123v2 in Tumor and Normal Cells

Viral replication, cytotoxicity, transgene expression and cytokine production following SKV-123v2 virus infection of human normal (PBMC, PrEC) and cancer (786-O, HeLa) cells were characterized in vitro. Human peripheral blood mononuclear cells (PBMCs) were purchased from Lonza (Cat#: CC-2702). Human prostate epithelial cells (PrEC) were purchased from Lonza (Cat. #: CC-2555). 786-O human renal adenocarcinoma cells were purchased from ATCC (Cat. #:CRL-1932). HeLa human cervical cancer cells were purchased from ATCC (Cat. #: CCL-2).

Cells were seeded on 24-well plates. Cell viability and cytokine expression were assessed at four time points: 6 hr, 24 hr, 48 hr and 72 hrs post infection. To determine the number of viable cells/mL and percent viability of each cell suspension, cell suspensions were placed in a ViCell cup at 0.6 mL each and samples were logged into the instrument. Dilutions were prepared. Cells were infected when monolayers were >80% confluent (24 hrs following cell seeding). On the day of infection, virus was diluted to the appropriate doses as outlined in Table 47 below. Infection was conducted in 24-well plates.

TABLE 47
Infection Calculations for 24-Well Plates

| | SKV-123v2 | | | |
|---|---|---|---|---|
| | # | | | |
| | cells/ | # wells | Volume | Volume |
| MOI | well | to make | virus | SFM (μL) |
| MOI 0.01 | 2 × 10⁵ | 55 | 1.64 μL | 2750 |
| | | | of 1:10× | |
| MOI 0.1 | 2 × 10⁵ | 55 | 1.64 μL | 2750 |
| | | | stock | |
| MOI 1 | 2 × 10⁵ | 55 | 16.4 μL | 2733.6 |
| | | | stock | |

Cytokine profiling was conducted at the indicated time points. Infected cell supernatants were aliquoted, stored at −20° C. and sent to Eve Technologies for analysis using the Human Cytokine Array/Chemokine Array 42-Plex with IL-18 (HD42). FLT3L and ipilimumab expression were assessed using ELISA assays. Cell Viability was conducted at each harvest timepoint using Alamar Blue, read out on Fluoroskan to determine % viability of treatment wells relative to untreated control wells. Viral titer was determined to assess replication kinetics between different cell lines.

Normal cells were cultured in the presence of serum growth factors and in a lack of contact inhibition in order to maximize cell viability at the time of infection. Under the tested conditions and at the equivalent cell density used for SKV-123v2 infection experiments, PrECs (prostate epithelial cells) demonstrated a proliferative rate almost as high as HeLa cancer cells. To better define the phenotype of the tested normal cell lines when cultured under these conditions, a proliferation assay measuring BrdU incorporation was conducted on PrEC cells and PBMCs in a companion study. The intermediate replication of SKV-123v2 in PrECs as compared to the cancer cell lines and PBMCs correlated with the relative proliferation rate of this normal cell line observed under in vitro infection conditions.

The results show that 786-O and HeLa cancer cell lines were sensitive to SKV-123v2 virus infection as evidenced by a decrease in cell viability in a dose dependent and time dependent manner (FIG. 56). 786-O cell viability decreased over time at all MOIs tested while HeLa cell viability decreased upon SKV-123v2 virus infection at an MOI of 1 and 0.1. Normal human PBMCs were resistant to infection as evidenced by high cell viability following SKV-123v2 virus infection at MOIs of 1, 0.1, and 0.01 (FIG. 56). Normal human PrECs were less sensitive to SKV-123v2 virus infection when compared to cancer cell lines. No decrease in cell viability was observed at any MOI (1, 0.1 and 0.01) up to 48 hours post-infection. By 72 hr post infection, decreased cell viability was noted upon SKV-123v2 virus infection at an MOI of 1 and 0.1 (FIG. 56).

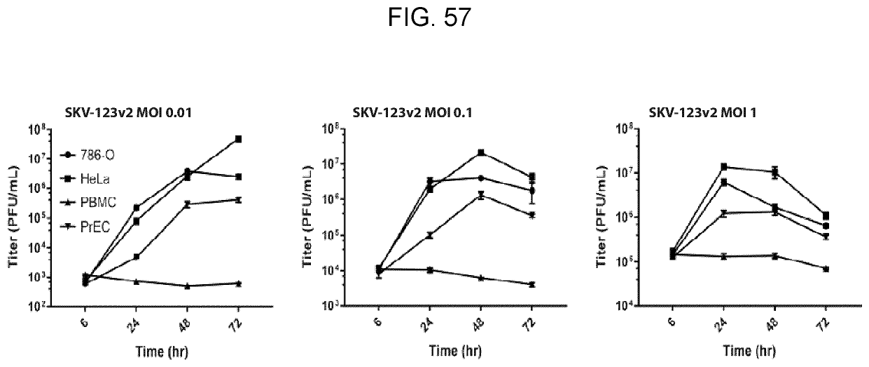

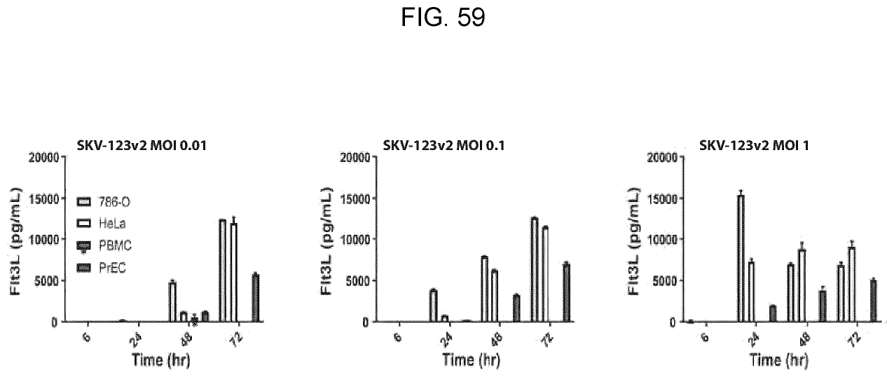

SKV-123v2 viruses infected and replicated in cancer cell lines at higher rates when compared to normal human PrECs (FIG. 57). They infected and replicated in 786-O and HeLa cancer cell lines to levels greater than 10-fold when compared to normal human PrECs. SKV-123v2 viruses did not replicate in normal human PBMCs at MOIs of 1, 0.1, and 0.01 (FIG. 59).

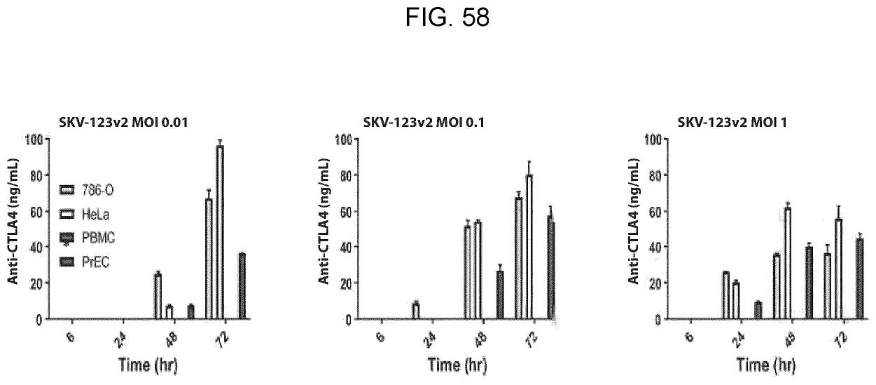

Anti-CTLA-4 antibody and FLT3L (two transgene products produced by SKV-123v2 virus-infected cells) production was monitored in cell supernatant at each MOI and timepoint. SKV-123v2 virus infection resulted in greater production of anti-CTLA-4 antibody and FLT3L in cancer cells when compared to normal cells (PrEC cells or PBMCs) (FIGS. 58 and 59). No anti-CTLA-4 antibody or FLT3L could be detected in PBMC cell supernatant. FLT3L and anti-CTLA-4 antibody transgene product concentrations correlated with cell susceptibility to SKV-123v2 infection.

In the cytokine profiling experiment, higher levels of cytotoxicity were observed in SKV-123v2 virus-infected cancers cells than normal cells.

The cell line PrEC was particularly responsive to viral infection, producing EGF, G-CSF, IL-la, IL-1RA, IL-4, and IL-18 in a context where only low or negligible concentrations of these cytokines were detectable from HeLa, 786-0, and PBMCs. PrEC and 786-0 were the only cell lines that produced IL-8, TGF-α, and TNFα in response to infection; while only PrEC and PBMC produced IP-10. PrEC showed dose-dependent production of EGF, with increased production at higher MOI of virus infection. EGF concentration remained consistent within the same dosing group between 24 to 72 hours with a slight increase at 72 hours for cells infected with MOI 1 of virus. In PrEC cells, IL-la production remained mostly consistent to baseline for 48 hours post-infection, and then increased at 72 hours, with the highest concentrations observed at MOI 1. Likewise, IL-1RA and IL-18 production by PrEC showed dramatic increases in concentration at 72 hours post-infection with low (slight IL-1RA increase at 24 hours in higher MOI 0.1 and 1 infection) or negligible prior to this time point. While production of IL-1RA seemed dose-dependent at 72 hours, MOI 1 of virus resulted in reduced IL-18 production compared to MOI 0.1. PrEC produced reduced IL-4 at 6 hours post-infection in all dosing groups compared to untreated control. However, this IL-4 production increased at 24 hours above baseline and remained high to 72 hours post-infection.

Alternatively, all cell lines, except PrEC, produced MCP-1. In HeLa and 786-0 cells, high concentrations of MCP-1 were produced at baseline and unaffected by viral infection. In PBMCs, MCP-1 was induced above baseline by high doses of virus: MOI 1 of virus at 24 hours and MOI 0.1 and 1 of virus at 72 hours post-infection.

Several cytokines increased at 24 and 72 hours post-infection, but not 48 hours (or only marginally), over baseline. In HeLa cells, these are FGF-2 (at MOI 1, while lower dosing was only able to induce production at 72 hours) and IL-6 (MOI 0.01 and 0.1 only; MOI 1 concentrations were consistent over time). In PrEC cells, these are G-CSF (at MOI 0.01 and 0.1 only; MOI 1 concentrations were consistent over time), TGF-α (at MOI 0.01 and 0.1 only; MOI 1 concentrations were consistent over time), TNFα (at MOI 0.01 and 0.1 only; MOI 1 increased over time), and IP-10 (MOI 0.1 and 0.1 only; MOI 1 produced low concentrations). In 786-0 cells, these are FGF-2 (in MOI 0.1 and 1 only; lowest dose MOI 0.01 only induced production at 72 hours), IL-8 (at MOI 0.01 and 0.1 only; MOI 1 concentrations were consistent over time) and TGF-α (at MOI 0.01 and 0.1 only), TNFα (MOI 0.01 and 0.1 only; MOI concentrations were baseline over time). In PBMCs, the cytokine is IP-10 (at MOI 1 only).

IFNα2 production in HeLa and 786-0 cells peaked at 24 hours post-infection and then dropped over the time-course, while in PrEC cells, IFNα2 production was highest at 72 hours. In all three cell lines, higher dosing at MOI 0.1 or 1 exhibited the lowest cytokine production among the dosing groups.

IFNγ production was low in all cell lines, with 786-0 exhibiting peak production at 24 hours and then decreasing over time.

### 6.32. Example 32

### Generation of Recombinant Vaccinia Viruses

The methods and techniques described in this example were used to generate the vectors described in Table 45. As summarized in Table 45, each of the vectors has been demonstrated to be replication-competent, to express the transgene(s) contained in the vector, and to exhibit cytotoxicity in the cancer cell lines as indicated. Assays as described below were used to generate the data summarized in Table 45.

Recombination

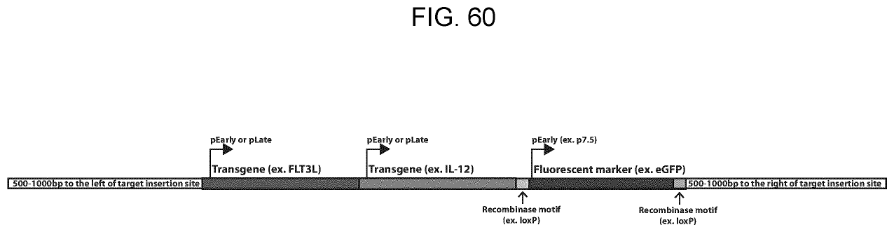

Monolayers of nearly confluent (80-90%) U205 cells are first infected for 2 hrs with vaccinia virus (e.g., SKV virus) to be modified. Following infection, targeting DNA (FIG. 60) is transfected into infected cells. The following day (12-18 hrs post transfection), transfection media is removed and fresh media is added to cells. On the second day (˜48 hrs post transfection) cells are frozen and thawed for plaque purification.

Plaque Purification and Amplification

To identify recombinant vaccinia viruses, plaques are screened using the fluorescent marker. Serial dilutions (1:10) of the infection/transfection mixture from the previous step are added to confluent monolayers of U205 cells for 2 hr after which the media is replaced with overlay media to allow plaque formation. Two days later, fluorescent plaques are picked, serially diluted and added to fresh monolayers of U205 cells followed by overlay media. This process of plaque purification is repeated until all plaques are fluorescent. In order to remove the fluorescent marker, the appropriate recombinase is transfected with the fluorescent virus as outlined in the above step (i.e., Recombination). The process of plaque purification is then continued to select for non-fluorescent plaques.

A plaque of a pure recombinant virus is then used to infect monolayers of another cell line (e.g., HeLa cells) for expansion, at this step no overlay is added. Once visible cytopathic effect is seen on infected cells, cell lysate is collected and the recombinant virus concentration is determined by viral tittering.

Transgene Expression

Recombinant viruses are used to infect a variety of cancer cells (e.g., HeLa, U205, 786-O, etc.) at various concentrations (e.g., MOI 0.01, 0.1 or 1) for various amounts of time (e.g., 6 hrs, 24 hrs, 48 hrs and 72 hrs post infection). Cell lysates (for Western blot) and cell supernatants (for ELISA) are frozen and stored at −80 ° C. for assays relating to transgene production. For transgenes encoding a soluble protein (e.g., FLT3L, anti-CTLA-4 antibody) ELISA kits are used to quantify transgene production. For transgenes encoding cell restricted (e.g., inside the cell or membrane bound) protein products (e.g., membrane bound IL-12), western blots of cell lysates are used to quantify transgene production.

Cancer Cell Line Cytotoxicity

Cancer cell line cytotoxicity is determined by the visible cytopathic effect seen by light microscopy on infected cells. The cell lysate may be collected and the recombinant virus concentration determined by viral titering.

### Some Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Some embodiments are within the claims.

LENGTHY TABLES
The patent application contains a lengthy table section. A copy of the table is available in electronic form from the USPTO web site (https://seqdata.uspto.gov/?pageRequest=docDetail&DocID=US20220056480A1). An electronic copy of the table will also be available from the USPTO upon request and payment of the fee set forth in 37 CFR 1.19(b)(3).

What is claimed is:

1. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to Cytotoxic T-lymphocyte Associated Protein 4 (CTLA-4), wherein the first nucleotide sequence is set forth in SEQ ID NO: 214;
(c) a second transgene comprising a second nucleotide sequence encoding an Interleukin 12 (IL-12) polypeptide, wherein the second nucleotide sequence is set forth in SEQ ID NO: 215; and
(d) a third transgene comprising a third nucleotide sequence encoding FMS-like tyrosine kinase 3 ligand (FLT3L), wherein the third nucleotide sequence is set forth in SEQ ID NO: 216.

2. The nucleic acid of claim 1, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

3. The nucleic acid of claim 1 or 2, further comprising a nucleotide sequence comprising at least one promoter operably linked to the first nucleotide sequence encoding the anti-CTLA-4 antibody.

4. The nucleic acid of claim 3, wherein the at least one promoter operably linked to the first nucleotide sequence encoding the anti-CTLA-4 antibody is an H5R promoter, a pS promoter, or a LEO promoter.

5. The nucleic acid of claim 3, wherein the at least one promoter operably linked to the first nucleotide sequence encoding the anti-CTLA-4 antibody is an H5R promoter.

6. The nucleic acid of any one of claims 1-5, further comprising a nucleotide sequence comprising at least one promoter operably linked to the second nucleotide sequence encoding the IL-12 polypeptide.

7. The nucleic acid of claim 6, wherein the at least one promoter operably linked to the second nucleotide sequence encoding the IL-12 polypeptide is a late promoter.

8. The nucleic acid of claim 7, wherein the late promoter comprises the nucleotide sequence of SEQ ID NO: 561, an F17R promoter, or a D13L promoter.

9. The nucleic acid of claim 7, wherein the late promoter comprises the nucleotide sequence of SEQ ID NO: 561.

10. The nucleic acid of any one of claims 1-9, further comprising a nucleotide sequence comprising at least one promoter operably linked to the third nucleotide sequence encoding FLT3L.

11. The nucleic acid of claim 10, wherein the at least one promoter operably linked to the third nucleotide sequence encoding FLT3L is a B8R promoter, a B19R promoter, a E3L promoter, an F11L promoter, or a B2R promoter.

12. The nucleic acid of claim 10, wherein the at least one promoter operably linked to the third nucleotide sequence encoding FLT3L is a B8R promoter.

13. The nucleic acid of claim 10, wherein the at least one promoter operably linked to the third nucleotide sequence encoding FLT3L is a B19R promoter.

14. The nucleic acid of claim 10, wherein the at least one promoter operably linked to the third nucleotide sequence encoding FLT3L is a B8R promoter and a B19R promoter.

15. The nucleic acid of any one of claims 1-14, wherein the first transgene is present between the partial C2L and F3L vaccinia genes in SEQ ID NO: 210.

16. The nucleic acid of any one of claims 1-14, wherein the second transgene is present between the partial C2L and F3L vaccinia genes in SEQ ID NO: 210.

17. The nucleic acid of any one of claims 1-14, wherein the third transgene is present between the partial C2L and F3L vaccinia genes in SEQ ID NO: 210.

18. The nucleic acid of any one of claims 1-14, wherein the first transgene is present in the locus of the deletion in the B8R gene.

19. The nucleic acid of any one of claims 1-14, wherein the second transgene is present in the locus of the deletion in the B8R gene.

20. The nucleic acid of any one of claims 1-14, wherein the third transgene is present in the locus of the deletion in the B8R gene.

21. The nucleic acid of any one of claims 1-14, wherein the first transgene is present between the partial B14R and B29R vaccinia genes in SEQ ID NO: 210.

22. The nucleic acid of any one of claims 1-14, wherein the second transgene is present between the partial B14R and B29R vaccinia genes in SEQ ID NO: 210.

23. The nucleic acid of any one of claims 1-14, wherein the third transgene is present between the partial B14R and B29R vaccinia genes in SEQ ID NO: 210.

24. The nucleic acid of any one of claims 1-14, wherein the first transgene and the second transgene are present between the partial C2L and F3L vaccinia genes in SEQ ID NO: 210.

25. The nucleic acid of any one of claims 1-14, wherein the first transgene and the second transgene are present in the locus of the deletion in the B8R gene.

26. The nucleic acid of any one of claims 1-14, wherein the first transgene and the second transgene are present between the partial B14R and B29R vaccinia genes in SEQ ID NO: 210.

27. The nucleic acid of any one of claims 1-14, wherein the first transgene and the third transgene are present between the partial C2L and F3L vaccinia genes in SEQ ID NO: 210.

28. The nucleic acid of any one of claims 1-14, wherein the first transgene and the third transgene are present in the locus of the deletion in the B8R gene.

29. The nucleic acid of any one of claims 1-14, wherein the first transgene and the third transgene are present between the partial B14R and B29R vaccinia genes in SEQ ID NO: 210.

30. The nucleic acid of any one of claims 1-14, wherein the second transgene and the third transgene are present between the partial C2L and F3L vaccinia genes in SEQ ID NO: 210.

31. The nucleic acid of any one of claims 1-14, wherein the second transgene and the third transgene are present in the locus of the deletion in the B8R gene.

32. The nucleic acid of any one of claims 1-14, wherein the second transgene and the third transgene are present between the partial B14R and B29R vaccinia genes in SEQ ID NO: 210.

33. The nucleic acid of any one of claims 1-14, wherein the first transgene is present between the partial C2L and F3L vaccinia genes in SEQ ID NO: 210, and the second transgene is present in the locus of the deletion in the B8R gene.

34. The nucleic acid of any one of claims 1-14, wherein the second transgene is present between the partial C2L and F3L vaccinia genes in SEQ ID NO: 210, and the first transgene is present in the locus of the deletion in the B8R gene.

35. The nucleic acid of any one of claims 1-14, wherein the first transgene is present between the partial C2L and F3L vaccinia genes in SEQ ID NO: 210, and the third transgene is present in the locus of the deletion in the B8R gene.

36. The nucleic acid of any one of claims 1-14, wherein the third transgene is present between the partial C2L and F3L vaccinia genes in SEQ ID NO: 210, and the first transgene is present in the locus of the deletion in the B8R gene.

37. The nucleic acid of any one of claims 1-14, wherein the second transgene is present between the partial C2L and F3L vaccinia genes in SEQ ID NO: 210, and the third transgene is present in the locus of the deletion in the B8R gene.

38. The nucleic acid of any one of claims 1-14, wherein the third transgene is present between the partial C2L and F3L vaccinia genes in SEQ ID NO: 210, and the second transgene is present in the locus of the deletion in the B8R gene.

39. The nucleic acid of any one of claims 1-14, wherein the first transgene is present between the partial C2L and F3L vaccinia genes in SEQ ID NO: 210, and the second transgene is present between the partial B14R and B29R vaccinia genes in SEQ ID NO: 210.

40. The nucleic acid of any one of claims 1-14, wherein the second transgene is present between the partial C2L and F3L vaccinia genes in SEQ ID NO: 210, and the first transgene is present between the partial B14R and B29R vaccinia genes in SEQ ID NO: 210.

41. The nucleic acid of any one of claims 1-14, wherein the first transgene is present between the partial C2L and F3L vaccinia genes in SEQ ID NO: 210, and the third transgene is present between the partial B14R and B29R vaccinia genes in SEQ ID NO: 210.

42. The nucleic acid of any one of claims 1-14, wherein the third transgene is present between the partial C2L and F3L vaccinia genes in SEQ ID NO: 210, and the first transgene is present between the partial B14R and B29R vaccinia genes in SEQ ID NO: 210.

43. The nucleic acid of any one of claims 1-14, wherein the second transgene is present between the partial C2L and F3L vaccinia genes in SEQ ID NO: 210, and the third transgene is present between the partial B14R and B29R vaccinia genes in SEQ ID NO: 210.

44. The nucleic acid of any one of claims 1-14, wherein the third transgene is present between the partial C2L and F3L vaccinia genes in SEQ ID NO: 210, and the second transgene is present between the partial B14R and B29R vaccinia genes in SEQ ID NO: 210.

45. The nucleic acid of any one of claims 1-14, wherein the first transgene is present in the locus of the deletion in the B8R gene, and the second transgene is present between the partial B14R and B29R vaccinia genes in SEQ ID NO: 210.

46. The nucleic acid of any one of claims 1-14, wherein the second transgene is present in the locus of the deletion in the B8R gene, and the first transgene is present between the partial B14R and B29R vaccinia genes in SEQ ID NO: 210.

47. The nucleic acid of any one of claims 1-14, wherein the first transgene is present in the locus of the deletion in the B8R gene, and the third transgene is present between the partial B14R and B29R vaccinia genes in SEQ ID NO: 210.

48. The nucleic acid of any one of claims 1-14, wherein the third transgene is present in the locus of the deletion in the B8R gene, and the first transgene is present between the partial B14R and B29R vaccinia genes in SEQ ID NO: 210.

49. The nucleic acid of any one of claims 1-14, wherein the second transgene is present in the locus of the deletion in the B8R gene, and the third transgene is present between the partial B14R and B29R vaccinia genes in SEQ ID NO: 210.

50. The nucleic acid of any one of claims 1-14, wherein the third transgene is present in the locus of the deletion in the B8R gene, and the second transgene is present between the partial B14R and B29R vaccinia genes in SEQ ID NO: 210.

51. The nucleic acid of any one of claims 1-14, wherein the first transgene, the second transgene, and the third transgene are present between the partial C2L and F3L vaccinia genes in SEQ ID NO: 210.

52. The nucleic acid of any one of claims 1-14, wherein the first transgene, the second transgene, and the third transgene are present in the locus of the deletion in the B8R gene.

53. The nucleic acid of any one of claims 1-14, wherein the first transgene, the second transgene, and the third transgene are present between the partial B14R and B29R vaccinia genes in SEQ ID NO: 210.

54. The nucleic acid of any one of claims 1-14, wherein the first transgene is present between the partial C2L and F3L vaccinia genes in SEQ ID NO: 210, and the second transgene and the third transgene are present in the locus of the deletion in the B8R gene.

55. The nucleic acid of any one of claims 1-14, wherein the second transgene is present between the partial C2L and F3L vaccinia genes in SEQ ID NO: 210, and the first transgene and the third transgene are present in the locus of the deletion in the B8R gene.

56. The nucleic acid of any one of claims 1-14, wherein the third transgene is present between the partial C2L and F3L vaccinia genes in SEQ ID NO: 210, and the first transgene and the second transgene are present in the locus of the deletion in the B8R gene.

57. The nucleic acid of any one of claims 1-14, wherein the first transgene and the second transgene are present between the partial C2L and F3L vaccinia genes in SEQ ID NO: 210, and the third transgene is present in the locus of the deletion in the B8R gene.

58. The nucleic acid of any one of claims 1-14, wherein the first transgene and the third transgene are present between the partial C2L and F3L vaccinia genes in SEQ ID NO: 210, and the second transgene is present in the locus of the deletion in the B8R gene.

59. The nucleic acid of any one of claims 1-14, wherein the second transgene and the third transgene are present between the partial C2L and F3L vaccinia genes in SEQ ID NO: 210, and the first transgene is present in the locus of the deletion in the B8R gene.

60. The nucleic acid of any one of claims 1-14, wherein the first transgene is present between the partial C2L and F3L vaccinia genes in SEQ ID NO: 210, and the second transgene and the third transgene are present between the partial B14R and B29R vaccinia genes in SEQ ID NO: 210.

61. The nucleic acid of any one of claims 1-14, wherein the second transgene is present between the partial C2L and F3L vaccinia genes in SEQ ID NO: 210, and the first transgene and the third transgene are present between the partial B14R and B29R vaccinia genes in SEQ ID NO: 210.

62. The nucleic acid of any one of claims 1-14, wherein the third transgene is present between the partial C2L and F3L vaccinia genes in SEQ ID NO: 210, and the first transgene and the second transgene are present between the partial B14R and B29R vaccinia genes in SEQ ID NO: 210.

63. The nucleic acid of any one of claims 1-14, wherein the first transgene and the second transgene are present between the partial C2L and F3L vaccinia genes in SEQ ID NO: 210, and the third transgene is present between the partial B14R and B29R vaccinia genes in SEQ ID NO: 210.

64. The nucleic acid of any one of claims 1-14, wherein the first transgene and the third transgene are present between the partial C2L and F3L vaccinia genes in SEQ ID NO: 210, and the second transgene is present between the partial B14R and B29R vaccinia genes in SEQ ID NO: 210.

65. The nucleic acid of any one of claims 1-14, wherein the second transgene and the third transgene are present between the partial C2L and F3L vaccinia genes in SEQ ID NO: 210, and the first transgene is present between the partial B14R and B29R vaccinia genes in SEQ ID NO: 210.

66. The nucleic acid of any one of claims 1-14, wherein the first transgene is present in the locus of the deletion in the B8R gene, and the second transgene and the third transgene are present between the partial B14R and B29R vaccinia genes in SEQ ID NO: 210.

67. The nucleic acid of any one of claims 1-14, wherein the second transgene is present in the locus of the deletion in the B8R gene, and the first transgene and the third transgene are present between the partial B14R and B29R vaccinia genes in SEQ ID NO: 210.

68. The nucleic acid of any one of claims 1-14, wherein the third transgene is present in the locus of the deletion in the B8R gene, and the first transgene and the second transgene are present between the partial B14R and B29R vaccinia genes in SEQ ID NO: 210.

69. The nucleic acid of any one of claims 1-14, wherein the first transgene and the second transgene are present in the locus of the deletion in the B8R gene, and the third transgene is present between the partial B14R and B29R vaccinia genes in SEQ ID NO: 210.

70. The nucleic acid of any one of claims 1-14, wherein the first transgene and the third transgene are present in the locus of the deletion in the B8R gene, and the second transgene is present between the partial B14R and B29R vaccinia genes in SEQ ID NO: 210.

71. The nucleic acid of any one of claims 1-14, wherein the second transgene and the third transgene are present in the locus of the deletion in the B8R gene, and the first transgene is present between the partial B14R and B29R vaccinia genes in SEQ ID NO: 210.

72. The nucleic acid of any one of claims 1-14, wherein the first transgene is present between the partial C2L and F3L vaccinia genes in SEQ ID NO: 210, the second transgene is present in the locus of the deletion in the B8R gene, and the third transgene is present between the partial B14R and B29R vaccinia genes in SEQ ID NO: 210.

73. The nucleic acid of any one of claims 1-14, wherein the first transgene is present between the partial C2L and F3L vaccinia genes in SEQ ID NO: 210, the third transgene is present in the locus of the deletion in the B8R gene, and the second transgene is present between the partial B14R and B29R vaccinia genes in SEQ ID NO: 210.

74. The nucleic acid of any one of claims 1-14, wherein the second transgene is present between the partial C2L and F3L vaccinia genes in SEQ ID NO: 210, the first transgene is present in the locus of the deletion in the B8R gene, and the third transgene is present between the partial B14R and B29R vaccinia genes in SEQ ID NO: 210.

75. The nucleic acid of any one of claims 1-14, wherein the second transgene is present between the partial C2L and F3L vaccinia genes in SEQ ID NO: 210, the third transgene is present in the locus of the deletion in the B8R gene, and the first transgene is present between the partial B14R and B29R vaccinia genes in SEQ ID NO: 210.

76. The nucleic acid of any one of claims 1-14, wherein the third transgene is present between the partial C2L and F3L vaccinia genes in SEQ ID NO: 210, the first transgene is present in the locus of the deletion in the B8R gene, and the second transgene is present between the partial B14R and B29R vaccinia genes in SEQ ID NO: 210.

77. The nucleic acid of any one of claims 1-14, wherein the third transgene is present between the partial C2L and F3L vaccinia genes in SEQ ID NO: 210, the second transgene is present in the locus of the deletion in the B8R gene, and the first transgene is present between the partial B14R and B29R vaccinia genes in SEQ ID NO: 210.

78. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214;
(c) a second transgene comprising a second nucleotide sequence encoding an IL-12 polypeptide, wherein the second nucleotide sequence is set forth in SEQ ID NO: 215; and
(d) a third transgene comprising a third nucleotide sequence encoding FLT3L, wherein the third nucleotide sequence is set forth in SEQ ID NO: 216;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter and a B19R promoter.

79. The nucleic acid of claim 78, wherein the first nucleotide sequence is in the same orientation as endogenous vaccinia virus genes that flank the first nucleotide sequence, the second nucleotide sequence is in the same orientation as endogenous vaccinia virus genes that flank the second nucleotide sequence, and the third nucleotide sequence is in the same orientation as endogenous vaccinia virus genes that flank the third nucleotide sequence.

80. The nucleic acid of claim 78 or 79, wherein the first transgene is present between the partial C2L and F3L vaccinia genes in SEQ ID NO: 210, and the second transgene and the third transgene are present in the locus of the deletion in the B8R gene.

81. The nucleic acid of claim 78 or 79, wherein the first transgene is present between the partial B14R and B29R vaccinia genes in SEQ ID NO: 210, and the second transgene and the third transgene are present in the locus of the deletion in the B8R gene.

82. The nucleic acid of claim 80 or 81, wherein the third transgene is upstream of the second transgene.

83. The nucleic acid of claim 80 or 81, wherein the third transgene is downstream of the second transgene.

84. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

85. The nucleic acid of claim 84, wherein the at least one promoter operably linked to the third nucleotide sequence is a B8R promoter and a B19R promoter.

86. The nucleic acid of claim 85, wherein the B8R promoter comprises the nucleotide sequence of SEQ ID NO: 564 and the B19R promoter comprises the nucleotide sequence of SEQ ID NO: 565.

87. The nucleic acid of any one of claims 84-86, wherein the at least one promoter operatively linked to the first nucleotide sequence is an H5R early promoter or an H5R late promoter.

88. The nucleic acid of claim 87, wherein the at least one promoter operatively linked to the first nucleotide sequence is an H5R early promoter and an H5R late promoter.

89. The nucleic acid of claim 88, wherein the H5R early promoter comprises the nucleotide sequence of SEQ ID NO: 553 and the H5R late promoter comprises the nucleotide sequence of SEQ ID NO: 554.

90. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

91. The nucleic acid of claim 90, wherein the at least one promoter operably linked to the third nucleotide sequence is a B8R promoter and a B19R promoter.

92. The nucleic acid of claim 91, wherein the B8R promoter comprises the nucleotide sequence of SEQ ID NO: 564 and the B19R promoter comprises the nucleotide sequence of SEQ ID NO: 565.

93. The nucleic acid of any one of claims 90-92, wherein the at least one promoter operatively linked to the first nucleotide sequence is an H5R early promoter or an H5R late promoter.

94. The nucleic acid of claim 93, wherein the at least one promoter operatively linked to the first nucleotide sequence is an H5R early promoter and an H5R late promoter.

95. The nucleic acid of claim 94, wherein the H5R early promoter comprises the nucleotide sequence of SEQ ID NO: 553 and the H5R late promoter comprises the nucleotide sequence of SEQ ID NO: 554.

96. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes in SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

97. The nucleic acid of claim 96, wherein the at least one promoter operably linked to the third nucleotide sequence is a B8R promoter and a B19R promoter.

98. The nucleic acid of claim 97, wherein the B8R promoter comprises the nucleotide sequence of SEQ ID NO: 564 and the B19R promoter comprises the nucleotide sequence of SEQ ID NO: 565.

99. The nucleic acid of any one of claims 96-98, wherein the at least one promoter operatively linked to the first nucleotide sequence is an H5R early promoter or an H5R late promoter.

100. The nucleic acid of claim 99, wherein the at least one promoter operatively linked to the first nucleotide sequence is an H5R early promoter and an H5R late promoter.

101. The nucleic acid of claim 100, wherein the H5R early promoter comprises the nucleotide sequence of SEQ ID NO: 553 and the H5R late promoter comprises the nucleotide sequence of SEQ ID NO: 554.

102. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

103. The nucleic acid of claim 102, wherein the at least one promoter operably linked to the third nucleotide sequence is a B8R promoter and a B19R promoter.

104. The nucleic acid of claim 103, wherein the B8R promoter comprises the nucleotide sequence of SEQ ID NO: 564 and the B19R promoter comprises the nucleotide sequence of SEQ ID NO: 565.

105. The nucleic acid of any one of claims 102-104, wherein the at least one promoter operatively linked to the first nucleotide sequence is an H5R early promoter or an H5R late promoter.

106. The nucleic acid of claim 105, wherein the at least one promoter operatively linked to the first nucleotide sequence is an H5R early promoter and an H5R late promoter.

107. The nucleic acid of claim 106, wherein the H5R early promoter comprises the nucleotide sequence of SEQ ID NO: 553 and the H5R late promoter comprises the nucleotide sequence of SEQ ID NO: 554.

108. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

109. The nucleic acid of claim 108, wherein the at least one promoter operably linked to the third nucleotide sequence is a B8R promoter and a B19R promoter.

110. The nucleic acid of claim 109, wherein the B8R promoter comprises the nucleotide sequence of SEQ ID NO: 564 and the B19R promoter comprises the nucleotide sequence of SEQ ID NO: 565.

111. The nucleic acid of any one of claims 108-110, wherein the nucleotide sequence of the pS comprises the nucleotide sequence of SEQ ID NO: 555, SEQ ID NO: 556, or SEQ ID NO: 557.

112. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

113. The nucleic acid of claim 112, wherein the at least one promoter operably linked to the third nucleotide sequence is a B8R promoter and a B19R promoter.

114. The nucleic acid of claim 113, wherein the B8R promoter comprises the nucleotide sequence of SEQ ID NO: 564 and the B19R promoter comprises the nucleotide sequence of SEQ ID NO: 565.

115. The nucleic acid of any one of claims 112-114, wherein the nucleotide sequence of the pS comprises the nucleotide sequence of SEQ ID NO: 555, SEQ ID NO: 556, or SEQ ID NO: 557.

116. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

117. The nucleic acid of claim 116, wherein the at least one promoter operably linked to the third nucleotide sequence is a B8R promoter and a B19R promoter.

118. The nucleic acid of claim 117, wherein the B8R promoter comprises the nucleotide sequence of SEQ ID NO: 564 and the B19R promoter comprises the nucleotide sequence of SEQ ID NO: 565.

119. The nucleic acid of any one of claims 116-118, wherein the nucleotide sequence of the pS comprises the nucleotide sequence of SEQ ID NO: 555, SEQ ID NO: 556, or SEQ ID NO: 557.

120. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a B8R promoter or a B19R promoter.

121. The nucleic acid of claim 120, wherein the at least one promoter operably linked to the third nucleotide sequence is a B8R promoter and a B19R promoter.

122. The nucleic acid of claim 121, wherein the B8R promoter comprises the nucleotide sequence of SEQ ID NO: 564 and the B19R promoter comprises the nucleotide sequence of SEQ ID NO: 565.

123. The nucleic acid of any one of claims 120-122, wherein the nucleotide sequence of the pS comprises the nucleotide sequence of SEQ ID NO: 555, SEQ ID NO: 556, or SEQ ID NO: 557.

124. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a B8R promoter or a B19R promoter.

125. The nucleic acid of claim 124, wherein the at least one promoter operably linked to the third nucleotide sequence is a B8R promoter and a B19R promoter.

126. The nucleic acid of claim 125, wherein the B8R promoter comprises the nucleotide sequence of SEQ ID NO: 564 and the B19R promoter comprises the nucleotide sequence of SEQ ID NO: 565.

127. The nucleic acid of any one of claims 124-126, wherein the at least one promoter operatively linked to the first nucleotide sequence is an H5R early promoter or an H5R late promoter.

128. The nucleic acid of claim 127, wherein the at least one promoter operatively linked to the first nucleotide sequence is an H5R early promoter and an H5R late promoter.

129. The nucleic acid of claim 128, wherein the H5R early promoter comprises the nucleotide sequence of SEQ ID NO: 553 and the H5R late promoter comprises the nucleotide sequence of SEQ ID NO: 554.

130. The nucleic acid of any one of claims 124-129, nucleotide sequence of the F17R promoter comprises the nucleotide sequence of SEQ ID NO: 563.

131. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene, of the vaccinia virus nucleotide sequence of SEQ ID NO: 210 and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a B8R promoter or a B19R promoter.

132. The nucleic acid of claim 131, wherein the at least one promoter operably linked to the third nucleotide sequence is a B8R promoter and a B19R promoter.

133. The nucleic acid of claim 132, wherein the B8R promoter comprises the nucleotide sequence of SEQ ID NO: 564 and the B19R promoter comprises the nucleotide sequence of SEQ ID NO: 565.

134. The nucleic acid of any one of claims 131-133, wherein the at least one promoter operatively linked to the first nucleotide sequence is an H5R early promoter or an H5R late promoter.

135. The nucleic acid of claim 134, wherein the at least one promoter operatively linked to the first nucleotide sequence is an H5R early promoter and an H5R late promoter.

136. The nucleic acid of claim 135, wherein the H5R early promoter comprises the nucleotide sequence of SEQ ID NO: 553 and the H5R late promoter comprises the nucleotide sequence of SEQ ID NO: 554.

137. The nucleic acid of any one of claims 131-136, nucleotide sequence of the F17R promoter comprises the nucleotide sequence of SEQ ID NO: 563.

138. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a B8R promoter or a B19R promoter.

139. The nucleic acid of claim 138, wherein the at least one promoter operably linked to the third nucleotide sequence is a B8R promoter and a B19R promoter.

140. The nucleic acid of claim 139, wherein the B8R promoter comprises the nucleotide sequence of SEQ ID NO: 564 and the B19R promoter comprises the nucleotide sequence of SEQ ID NO: 565.

141. The nucleic acid of any one of claims 138-140, wherein the at least one promoter operatively linked to the first nucleotide sequence is an H5R early promoter or an H5R late promoter.

142. The nucleic acid of claim 141, wherein the at least one promoter operatively linked to the first nucleotide sequence is an H5R early promoter and an H5R late promoter.

143. The nucleic acid of claim 142, wherein the H5R early promoter comprises the nucleotide sequence of SEQ ID NO: 553 and the H5R late promoter comprises the nucleotide sequence of SEQ ID NO: 554.

144. The nucleic acid of any one of claims 138-143, nucleotide sequence of the F17R promoter comprises the nucleotide sequence of SEQ ID NO: 563.

145. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a B8R promoter and a B19R promoter.

146. The nucleic acid of claim 145, wherein the at least one promoter operably linked to the third nucleotide sequence is a B8R promoter and a B19R promoter.

147. The nucleic acid of claim 146, wherein the B8R promoter comprises the nucleotide sequence of SEQ ID NO: 564 and the B19R promoter comprises the nucleotide sequence of SEQ ID NO: 565.

148. The nucleic acid of any one of claims 145-147, wherein the at least one promoter operatively linked to the first nucleotide sequence is an H5R early promoter or an H5R late promoter.

149. The nucleic acid of claim 148, wherein the at least one promoter operatively linked to the first nucleotide sequence is an H5R early promoter and an H5R late promoter.

150. The nucleic acid of claim 149, wherein the H5R early promoter comprises the nucleotide sequence of SEQ ID NO: 553 and the H5R late promoter comprises the nucleotide sequence of SEQ ID NO: 554.

151. The nucleic acid of any one of claims 145-150, nucleotide sequence of the F17R promoter comprises the nucleotide sequence of SEQ ID NO: 563.

152. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a E3L promoter.

153. The nucleic acid of claim 152, wherein the at least one promoter operatively linked to the first nucleotide sequence is an H5R early promoter or an H5R late promoter.

154. The nucleic acid of claim 153, wherein the at least one promoter operatively linked to the first nucleotide sequence is an H5R early promoter and an H5R late promoter.

155. The nucleic acid of claim 154, wherein the H5R early promoter comprises the nucleotide sequence of SEQ ID NO: 553 and the H5R late promoter comprises the nucleotide sequence of SEQ ID NO: 554.

156. The nucleic acid of any one of claims 152-155, nucleotide sequence of the E3L promoter comprises the nucleotide sequence of SEQ ID NO: 567.

157. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a E3L promoter.

158. The nucleic acid of claim 157, wherein the at least one promoter operatively linked to the first nucleotide sequence is an H5R early promoter or an H5R late promoter.

159. The nucleic acid of claim 158, wherein the at least one promoter operatively linked to the first nucleotide sequence is an H5R early promoter and an H5R late promoter.

160. The nucleic acid of claim 159, wherein the H5R early promoter comprises the nucleotide sequence of SEQ ID NO: 553 and the H5R late promoter comprises the nucleotide sequence of SEQ ID NO: 554.

161. The nucleic acid of any one of claims 157-160, nucleotide sequence of the E3L promoter comprises the nucleotide sequence of SEQ ID NO: 567.

162. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a E3L promoter.

163. The nucleic acid of claim 162, wherein the at least one promoter operatively linked to the first nucleotide sequence is an H5R early promoter or an H5R late promoter.

164. The nucleic acid of claim 163, wherein the at least one promoter operatively linked to the first nucleotide sequence is an H5R early promoter and an H5R late promoter.

165. The nucleic acid of claim 164, wherein the H5R early promoter comprises the nucleotide sequence of SEQ ID NO: 553 and the H5R late promoter comprises the nucleotide sequence of SEQ ID NO: 554.

166. The nucleic acid of any one of claims 162-165, nucleotide sequence of the E3L promoter comprises the nucleotide sequence of SEQ ID NO: 567.

167. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is an H5R promoter;
(ii) a nucleotide sequence comprising at least one promoter operably linked to the second nucleotide sequence, wherein the at least one promoter operably linked to the second nucleotide sequence is a late promoter that comprises the nucleotide sequence of SEQ ID NO: 561; and
(iii) a nucleotide sequence comprising at least one promoter operably linked to the third nucleotide sequence, wherein the at least one promoter operably linked to the third nucleotide sequence is a E3L promoter.

168. The nucleic acid of claim 167, wherein the at least one promoter operatively linked to the first nucleotide sequence is an H5R early promoter or an H5R late promoter.

169. The nucleic acid of claim 168, wherein the at least one promoter operatively linked to the first nucleotide sequence is an H5R early promoter and an H5R late promoter.

170. The nucleic acid of claim 169, wherein the H5R early promoter comprises the nucleotide sequence of SEQ ID NO: 553 and the H5R late promoter comprises the nucleotide sequence of SEQ ID NO: 554.

171. The nucleic acid of any one of claims 167-170, nucleotide sequence of the E3L promoter comprises the nucleotide sequence of SEQ ID NO: 567.

172. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence an F17R promoter; and
(iii) a nucleotide sequence comprising at least one promoter operably linked to the third nucleotide sequence, wherein the at least one promoter operably linked to the third nucleotide sequence is a E3L promoter.

173. The nucleic acid of claim 172, wherein the nucleotide sequence of the pS promoter comprises the nucleotide sequence of SEQ ID NO: 555, SEQ ID NO: 556, or SEQ ID NO: 557.

174. The nucleic acid of claim 172 or 173, wherein the nucleotide sequence of the F17R promoter comprises the nucleotide sequence of SEQ ID NO: 563.

175. The nucleic acid of any one of claims 172-174, wherein the nucleotide sequence of the E3L promoter comprises the nucleotide sequence of SEQ ID NO:

567.

176. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial C2L and F3L vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a E3L promoter.

177. The nucleic acid of claim 176, wherein the nucleotide sequence of the pS promoter comprises the nucleotide sequence of SEQ ID NO: 555, SEQ ID NO: 556, or SEQ ID NO: 557.

178. The nucleic acid of claim 176 or 177, wherein the nucleotide sequence of the F17R promoter comprises the nucleotide sequence of SEQ ID NO: 563.

179. The nucleic acid of any one of claims 176-178, wherein the nucleotide sequence of the E3L promoter comprises the nucleotide sequence of SEQ ID NO:

567.

180. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is upstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a E3L promoter.

181. The nucleic acid of claim 180, wherein the nucleotide sequence of the pS promoter comprises the nucleotide sequence of SEQ ID NO: 555, SEQ ID NO: 556, or SEQ ID NO: 557.

182. The nucleic acid of claim 170 or 181, wherein the nucleotide sequence of the F17R promoter comprises the nucleotide sequence of SEQ ID NO: 563.

183. The nucleic acid of any one of claims 180-182, wherein the nucleotide sequence of the E3L promoter comprises the nucleotide sequence of SEQ ID NO:

567.

184. A nucleic acid comprising a recombinant vaccinia virus genome, comprising:
(a) a vaccinia virus nucleotide sequence of SEQ ID NO: 210, which comprises partial C2L, F3L, B14R, and B29R vaccinia genes and which comprises a deletion in the B8R gene;
(b) a first transgene comprising a first nucleotide sequence encoding an antibody that specifically binds to CTLA-4, wherein the first nucleotide sequence is set forth in SEQ ID NO: 214, and wherein the first nucleotide sequence is in the same orientation as endogenous vaccinia virus genes that flank the first nucleotide sequence and the first transgene is present between the partial B14R and B29R vaccinia genes of the vaccinia virus nucleotide sequence of SEQ ID NO: 210;
(c) a second transgene comprising a second nucleotide sequence encoding an IL-12 polypeptide, wherein the second nucleotide sequence is set forth in SEQ ID NO: 215, and wherein the second nucleotide sequence is in the same orientation as endogenous vaccinia virus genes that flank the second nucleotide sequence and the second transgene is present in the locus of the deletion in the B8R gene of the vaccinia virus nucleotide sequence of SEQ ID NO: 210; and
(d) a third transgene comprising a third nucleotide sequence encoding FLT3L, wherein the third nucleotide sequence is set forth in SEQ ID NO: 216, wherein the third nucleotide sequence is in the same orientation as endogenous vaccinia virus genes that flank the third nucleotide sequence and the third transgene is present in the locus of the deletion in the B8R gene of the vaccinia virus nucleotide sequence of SEQ ID NO: 210, and wherein the third transgene is downstream of the second transgene;
wherein the nucleic acid further comprises:
(i) a nucleotide sequence comprising at least one promoter operably linked to the first nucleotide sequence, wherein the at least one promoter operably linked to the first nucleotide sequence is a pS promoter;
(ii) a nucleotide sequence comprising at least one promoter operably linked to the second nucleotide sequence, wherein the at least one promoter operably linked to the second nucleotide sequence is an F17R promoter; and
(iii) a nucleotide sequence comprising at least one promoter operably linked to the third nucleotide sequence, wherein the at least one promoter operably linked to the third nucleotide sequence is a E3L promoter.

185. The nucleic acid of claim 184, wherein the nucleotide sequence of the pS promoter comprises the nucleotide sequence of SEQ ID NO: 555, SEQ ID NO: 556, or SEQ ID NO: 557.

186. The nucleic acid of claim 174 or 185, wherein the nucleotide sequence of the F17R promoter comprises the nucleotide sequence of SEQ ID NO: 563.

187. The nucleic acid of any one of claims 184-186, wherein the nucleotide sequence of the E3L promoter comprises the nucleotide sequence of SEQ ID NO:

567.

188. A virus comprising the nucleic acid comprising a recombinant vaccinia virus genome of any one of claims 1-187.

189. A packaging cell line comprising the nucleic acid of any one of claims 1-187.

190. A packaging cell line comprising the virus of claim 188.

191. A pharmaceutical composition comprising the virus of claim 188 and a physiologically acceptable carrier.

192. A kit comprising the nucleic acid of any one of claims 1-187 and a package insert instructing a user of the kit to express the nucleic acid in a host cell.

193. A kit comprising the virus of claim 188 and a package insert instructing a user of the kit to express the virus in a host cell.

194. A kit comprising the virus of claim 188 and a package insert instructing a user to administer a therapeutically effective amount of the virus to a mammalian patient having cancer, thereby treating the cancer.

195. The kit of claim 194, wherein the mammalian patient is a human patient.

196. A method of treating cancer in a mammalian patient, the method comprising administering to the mammalian patient a therapeutically effective amount of the virus of claim 188.

197. A method of treating cancer in a mammalian patient, the method comprising administering to the mammalian patient a therapeutically effective amount of the pharmaceutical composition of claim 191.

198. The method of claim 196 or 197, wherein the mammalian patient is a human patient.

199. The method of any one of claims 196-198, wherein the cancer is selected from the group consisting of leukemia, lymphoma, liver cancer, bone cancer, lung cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, and throat cancer.

200. The method of claim 199, wherein the method further comprises administering to the mammalian patient an anti-PD1 antibody or an anti-PD-L1 antibody.
